(19) United States
(12) Patent Application Publication
(10) Pub. No.: US 2025/0287945 A1
(43) Pub. Date: Sep. 18, 2025

(54) COMPOSITIONS FOR FUNGAL CONTROL AND RELATED METHODS

(71) Applicant: Flagship Pioneering Innovations VII, LLC, Cambridge, MA (US)

(72) Inventors: Hope Hsiao-Wang DANISON, Somerville, MA (US); David Barry KOLESKY, Arlington, MA (US); Sindhu Manubhai KRISHNANKUTTY, Stow, MA (US); Antonio Diego MARTINEZ, Beverly, MA (US); Yajie NIU, Lexington, MA (US); Michka Gabrielle SHARPE, Cambridge, MA (US)

(73) Assignee: Flagship Pioneering Innovations VII, LLC, Cambridge, MA (US)

(21) Appl. No.: 18/291,201

(22) PCT Filed: Jul. 22, 2022
(86) PCT No.: PCT/US2022/074082

§ 371 Date: Jan. 22, 2024

Related U.S. Application Data
(60) Provisional application No. 63/225,356, filed on Jul. 23, 2021.

(51) Int. Cl. A01N 37/46; A01P 3/00; A61K 36/062; A61K 36/07; A61K 38/16; A61P 31/10

(52) U.S. Cl. CPC A01N 37/46; A01P 3/00; A61K 36/062; A61K 36/07; A61K 38/16; A61P 31/10

(57) ABSTRACT

The disclosure provides conidial germination-inhibiting (CGI) factors, CGI factor precursors, CGI factor fragments, and CGI factor motifs and compositions including the CGI factors, CGI factor precursors, CGI factor fragments, and CGI factor motifs. The disclosure also provides recombinant DNA constructs and vectors encoding the CGI factors, CGI factor precursors, CGI factor fragments, and CGI factor motifs, and transgenic organisms including recombinant DNA constructs or vectors encoding the CGI factors, CGI factor precursors, CGI factor fragments, and CGI factor motifs. In addition, the disclosure provides methods of controlling or inhibiting fungal growth and infection, as well as methods of treating a fungal disease using the CGI factors, CGI factor precursors, CGI factor fragments, and CGI factor motifs.

### CROSS-REFERENCE TO RELATED APPLICATION

This application is a national stage application under 35 U.S.C. § 371 of International Application No. PCT/US2022/074082, filed internationally on Jul. 22, 2022, which claims the benefit of U.S. Provisional Application No. 63/225,356, filed Jul. 23, 2021, the disclosures of which are hereby incorporated by reference in their entirety.

### REFERENCE TO AN ELECTRONIC SEQUENCE LISTING

The content of the electronic sequence listing (237212000100SUBSEQLIST.xml; Size: 5,861,171 bytes; and Date of Creation: May 7, 2024) is herein incorporated by reference in its entirety.

### FIELD OF THE DISCLOSURE

The present disclosure relates to antifungal or fungicidal agents, compositions and organisms comprising the antifungal or fungicidal agents, and methods of inhibiting or controlling fungi, such as fungal pathogens, using antifungal or fungicidal agents.

### BACKGROUND OF THE DISCLOSURE

The fungal kingdom encompasses a diverse group of organisms, some of which can act as pathogens for a variety of hosts. Pathogenic fungi can have detrimental effects on both human and animal health, either by direct infection, or by indirect effects from a secreted toxin. Food rot and crop loss due to uncontrolled fungal pathogens of plants or plant products can also lead to significant agricultural and economic losses. Thus, a need exists for agents capable of controlling or inhibiting growth of fungal pathogens, as well as for compositions comprising the agents that can be used to control or inhibit fungal growth and treat fungal infections of biological systems.

### BRIEF SUMMARY OF THE DISCLOSURE

In one aspect of the disclosure, provided herein are methods of decreasing growth or reproduction of a fungus, comprising providing a fungus with an antifungal composition that comprises an effective amount of at least one conidial germination-inhibiting (CGI) factor, CGI factor precursor, CGI factor fragment, or CGI factor motif, wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, and SEQ ID NO: 5707-5731, or wherein the CGI factor motif comprises at least one of SEQ ID NO: 1921-1956, and wherein the amino acid sequence of the CGI factor is not that of an alpha pheromone natively expressed by the fungus, or wherein the nucleotide sequence encoding the at least one CGI factor, CGI factor precursor, CGI factor fragment, or CGI factor motif does not occur in the genome of the fungus; and optionally, an agriculturally or pharmaceutically acceptable carrier; whereby the growth or reproduction of the fungus is decreased, relative to a control fungus not provided with the antifungal composition.

In another aspect of the disclosure, provided herein are recombinant DNA constructs comprising a heterologous promoter operably linked to a nucleic acid molecule comprising a nucleotide sequence that encodes a conidial germination-inhibiting (CGI) factor, a CGI factor precursor, or a CGI factor fragment, wherein the nucleotide sequence (a) encodes at least one CGI factor comprising an amino acid sequence that has at least 80% sequence identity with at least one of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, at least one CGI factor precursor, or at least one CGI factor fragment, or (b) encodes at least one CGI factor motif that comprises at least one of SEQ ID NO: 1921-1956; and wherein the nucleotide sequence optionally has codons optimized for heterologous expression. Related embodiments include cells or organisms, for example, a transgenic plant or plant part (e.g., rootstock or scion), in which such a recombinant DNA construct is heterologously expressed.

In another aspect of the disclosure, provided herein are methods of preventing or reducing disease caused by a fungal pathogen of a plant, comprising providing to a plant an antifungal composition that comprises an effective amount of at least one conidial germination-inhibiting (CGI) factor, CGI factor precursor, CGI factor fragment, or CGI factor motif, wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, and SEQ ID NO: 5707-5731, or wherein the CGI factor motif comprises at least one of SEQ ID NO: 1921-1956, and wherein the amino acid sequence of the CGI factor is not that of an alpha pheromone natively expressed by the fungal pathogen, or wherein the nucleotide sequence encoding the at least one CGI factor, CGI factor precursor, CGI factor fragment, or CGI factor motif does not occur in the genome of the fungal pathogen; and optionally, an agriculturally acceptable carrier; whereby disease caused by the fungal pathogen is prevented or decreased in the plant, relative to a control plant not provided with the antifungal composition.

Other aspects of the disclosure are related to methods of preventing or treating fungal diseases in organisms such as plants and animals, such as non-human animals; antifungal compositions formulated for use in agriculture or as therapeutics; methods of preventing or treating fungal infection or growth on a surface, including non-living surfaces; and compositions, such as a substrate or matrix, having antifungal properties, e.g., resistance to fungal contamination or growth.

### BRIEF DESCRIPTION OF THE FIGURES

FIGS. 1A-1B depict the experimental setup for a conidial germination inhibition assay. FIG. 1A depicts bright-field images showing the effect of control treatments on conidia germination at 40× (top row) and 400× (bottom row) magnification. Untreated conidia (left panels) and conidia treated with 50% (w/v) ethanol (right panels) were used as negative control conditions, and 100 μM fenpiclonil (middle panels) was used as the positive control condition. Asterisks in the 400× images highlight germinating conidia. FIG. 1B shows an exemplary 96-well plate layout for a conidial germination-inhibiting assay. As shown (from left to right): Un, untreated; eth, 50% ethanol; fen, fenpiclocil; 6, peptide106; 7, peptide107; 12, peptide112; 13, peptide113; 14, peptide114; and 15, peptide115.

FIGS. 2A-2E show results of experiments testing Fusarium and Botrytis conidial germination inhibition by candidate CGI factors. FIG. 2A depicts the resazurin fluorescence for Fusarium conidia (top panel) or Botrytis conidia (bottom panel) incubated with a concentration gradient of candidate CGI factors or controls (labeled on left). FIG. 2B shows the resazurin fluorescence quantification for Fusarium conidia incubated with 10 μM, 100 μM, 375 μM and 1 mM candidate CGI factors or controls (labeled on x-axis). FIG. 2C shows the resazurin fluorescence for Botrytis conidia incubated with 10 μM, 100 μM, 375 μM and 1 mM candidate CGI factors or controls (labeled on x-axis). FIG. 2D shows the resazurin fluorescence for Fusarium conidia incubated with 100 μM or 375 μM candidate CGI factors or controls (labeled on x-axis). FIG. 2E shows resazurin fluorescence for Botrytis conidia incubated with 100 μM or 375 μM candidate CGI factors or controls (labeled on x-axis). A pipetting error occurred during processing of the 375 μM untreated and 50% ethanol conditions shown in FIGS. 2C and 2E. In FIGS. 2A-2E, Pep106, peptide106; Pep107, peptide107; Pep112, peptide112; Pep113, peptide113; Pep114, peptide114; Untreated and 50% EtOH treated were used as negative control conditions; and Fenpiclonil was used as the positive control condition.

FIG. 3 shows the resazurin fluorescence quantification for Fusarium and Botrytis conidia incubated with 100 μM of selected candidate CGI factors and controls. Three replicates of each fungal species were evaluated for each condition. As shown: Fus, Fusarium; Bo, Botrytis; D and E, empty wells; and Fenp, fenpiclonil. In FIG. 3, Pep106, peptide106; Pep107, peptide107; Pep112, peptide112; Pep113, peptide113; Pep114, peptide114; and Pep115, peptide115; Untreated and 50% EtOH treated were used as negative control conditions; and 100 μM Fenpiclonil was used as the positive control condition.

FIG. 4 shows the resazurin fluorescence quantification for Fusarium and Botrytis conidia incubated with 100 μM of selected candidate CGI factors. Four replicates of each fungal species were evaluated for each condition. As shown: Fus, Fusarium; Bo, Botrytis; D and E, empty wells; and Fenp, fenpiclonil. In FIG. 4, Pep106, peptide106; Pep107, peptide107; Pep112, peptide112; Pep113, peptide113; Pep114, peptide114; and Pep115, peptide115; Untreated and 50% EtOH treated were used as negative control conditions; and 100 μM Fenpiclonil was used as the positive control condition.

FIG. 5 shows the CGI factor amino acid motif (SEQ ID NO: 1921). The height of letters in the motif indicates the degree of conservation, with taller letters being more conserved. The Y axis is the stack height, which is an expression of the relative entropy of the position; the height of a letter indicates the estimated probability or degree of conservation, with taller letters being more conserved. For this motif, the predicted probability of a given amino acid occurring at the specific position listed is as follows. Position 1, W: 1.000. Position 2, T: 0.011, E: 0.014, K: 0.025, Q: 0.027, S: 0.040, R: 0.043, G: 0.098, and H: 0.742. Position 3, W: 1.000. Position 4, G: 0.047, I: 0.087, V: 0.103, and L: 0.763. Position 5, A: 0.022, T: 0.040, K: 0.066, E: 0.067, N: 0.071, S: 0.128, R: 0.283, and Q: 0.323. Position 6, I: 0.051, F: 0.171, and L: 0.778. Position 7, E: 0.013, A: 0.015, Y: 0.016, Q: 0.020, M: 0.036, F: 0.041, S: 0.043, G: 0.068, D: 0.107, R: 0.301, and K: 0.328. Position 8, N: 0.010, T: 0.015, L: 0.020, A: 0.021, Y: 0.025, I: 0.025, W: 0.031, V: 0.039, M: 0.045, K: 0.089, R: 0.110, and P: 0.556. Position 9, G: 1.000. Position 10, A: 0.034, E: 0.182, and Q: 0.784. Position 11, P: 1.000. Position 12, F: 0.028, L: 0.093, I: 0.182, and M: 0.697. Position 13, Y: 1.000. The top X axis indicates the position k of the amino acid in the sequence. The first row, lower X axis, is the insert probability, i.e., the probability of observing one or more letters inserted between the letter corresponding to position k and the letter corresponding to position (k+1). The second row, lower X axis, is the insert length, i.e., the expected length of an insertion (if present) following position k. The third row, lower X axis, is the occupancy, occ(k), i.e., the probability of observing a letter at position k; the probability of observing a gap character or deletion relative to the model is [1−occ(k)]. This motif is characterized by having instability only at the last 3 amino acid residues (positions 11, 12, and/or 13), for example, where the CGI factor polypeptide lacks the position 13 Y residue.

FIGS. 6A-6C show Fusarium viability colorimetric assay (using resazurin) results from three experiments. FIG. 6A shows the results of the first experiment, in which the native alpha pheromones from Fusarium spp. (Fusarium ph., WCTWKGQPCW (SEQ ID NO: 2182)), Botrytis spp. (Botrytis Ph., WCGRPGQPC (SEQ ID NO: 2183)), and Saccharomyces cerevisiae (Yeast Ph., WHWLQLKPGQPMY (SEQ ID NO: 2184)), as well as a synthetic peptide (Scr. Yeast 1, WKMGQYHQLPPLW (SEQ ID NO: 2185)), and a modified Saccharomyces cerevisiae alpha pheromone with a C-terminus glycine cap (Yeast Pep-Gly, WHWLQLKPGQPMYG (SEQ ID NO: 2186)) were tested at a concentration of 375 μM. FIG. 6B shows the results of the second experiment, in which the native alpha pheromones from Fusarium spp. ((Fusarium ph., WCTWKGQPCW (SEQ ID NO: 2182)), Botrytis spp. (Botrytis Ph., WCGRPGQPC (SEQ ID NO: 2183)), and Saccharomyces cerevisiae (Yeast Ph., WHWLQLKPGQPMY (SEQ ID NO: 2184)), as well as a synthetic peptide (Scr. Yeast 1, WKMGQYHQLPPLW (SEQ ID NO: 2185)), and a modified Saccharomyces cerevisiae alpha pheromone with a C-terminus glycine cap (Yeast Pep-Gly, WHWLQLKPGQPMYG (SEQ ID NO: 2186)) were tested at a concentration of 375 μM and viability was measured at 51 hours after addition of resazurin. FIG. 6C shows the results of the third experiment, in which the native alpha pheromones from Fusarium spp. (HH1, WCTWKGQPCW (SEQ ID NO: 2182)), Botrytis spp. (HH2, WCGRPGQPC (SEQ ID NO: 2183)), and Saccharomyces cerevisiae (HH3, WHWLQLKPGQPMY (SEQ ID NO: 2184)), as well as a synthetic peptide (HH31, WKMGQYHQLPPLW (SEQ ID NO: 2185)), a modified Saccharomyces cerevisiae alpha pheromone with a C-terminus glycine cap (HH35, WHWLQLKPGQPMYG (SEQ ID NO: 2186)), a modified Saccharomyces cerevisiae alpha pheromone with a N-terminus glycine cap (HH36, GWHWLQLKPGQPMY (SEQ ID NO: 2187)), a peptide having the sequence of contiguous tandem copies of the Saccharomyces cerevisiae alpha pheromone (HH37, WHWLQLKPGQPMYWHWLQLKPGQPMY (SEQ ID NO: 2188)), and a peptide having the sequence of tandem copies of the Saccharomyces cerevisiae alpha pheromone separated by a four-glycine linking segment (HH38, WHWLQLKPGQPMYGGGGSWHWLQLKPGQPMY (SEQ ID NO: 2189)) were tested at concentrations of 175 μM and 375 μM. In FIGS. 6A-6C, the controls used were: untreated, 50% ethanol (negative control), and the fungicide fenpiclonil (positive control).

FIG. 7 shows the results of experiments to test fungal lesion size on Nicotiana benthamiana leaves after treatment with Botrytis cinerea conidial suspension followed by 5 mM MES buffer (“MES”) or 275 micromolar CGI factor “HH38” 9SEQ ID NO: 2189) in 5 mM MES buffer (or no addition as “Untreated” control).

### DETAILED DESCRIPTION

Unless defined otherwise, all technical and scientific terms used have the same meaning as commonly understood by one of ordinary skill in the art to which this invention belongs. Conventional methods are used for the procedures described herein, such as those provided in the art, and demonstrated in the Examples and various general references. Unless otherwise stated, nucleic acid sequences described herein are given, when read from left to right, in the 5′ to 3′ direction. Nucleic acid sequences may be provided as DNA or as RNA, as specified; disclosure of one necessarily defines the other, as is known to one of ordinary skill in the art. Furthermore, because of known codon degeneracy, different nucleic acid sequences can encode the same polypeptide sequence, and such modified nucleic acid sequences (e.g., for the purposes of codon optimization for a given species) are within the scope of the present disclosure.

The term “comprise” is intended to mean “include”. Where a term is provided in the singular, it also contemplates aspects of the invention described by the plural of that term. The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The following description sets forth exemplary methods, parameters, and the like. It should be recognized, however, that such description is not intended as a limitation on the scope of the present disclosure but is instead provided as a description of exemplary embodiments.

### Antifungal or Fungicidal Peptides

An aspect of the disclosure provides a conidial germination-inhibiting (CGI) factor, a CGI factor precursor, a CGI factor fragment, or a CGI factor motif for fungal control, wherein fungal control includes inhibition or reduction of conidial germination, fungal growth, or fungal reproduction, or is fungicidal (able to kill the fungus). A “CGI factor fragment” refers to an amino acid sequence that is reduced by one amino acid or two amino acids relative to a CGI factor, while retaining the function of the CGI factor. A “CGI factor precursor” is a polypeptide that includes at least one copy of a CGI factor (in embodiments, two or more copies of a CGI factor or multiple different CGI factors) and that is processed to the mature CGI factor(s), e.g., through proteolytic cleavage. CGI factor precursors include precursors with sequences encoded natively in one or more fungal genomes, as well as precursors having synthetic sequences. An “active” CGI factor, CGI factor precursor, or CGI factor fragment refers to a CGI factor, CGI factor precursor, or CGI factor fragment that is able to inhibit fungal conidial germination activity (conidial germination inhibitory), fungal growth, or fungal reproduction. A “toxic” CGI factor, CGI factor precursor, or CGI factor fragment refers to a CGI factor, CGI factor precursor, or CGI factor fragment that is able to kill a fungus or that decreases the number of viable cells in a population of fungal cells (i.e., is fungicidal). A CGI factor, CGI factor precursor, or CGI factor fragment may be active without being toxic, or may be toxic without being active, or may be both active and toxic. Both active and/or toxic CGI factors, CGI factor precursors, or CGI factor fragments are CGI factors, CGI factor precursors, or CGI factor fragments of the present disclosure.

The CGI factor motif includes the sequence WX₁WX₂X₃X₄X₅X₆GX-PX₈Y (SEQ ID NO: 1921). In this sequence, X₁ is selected from the group of T, E, K, Q, S, R, G and H; X₂ is selected from the group of G, I, V and L; X₃ is selected from the group of A, T, K, E, N, S, R and Q; X₄ is selected from the group of I, F and L; X₅ is selected from the group of E, A, Y, Q, M, F, S, G, D, R and K; X₆ is selected from the group of N, T, L, A, Y, I, W, V, M, K, R and P; X₇ is selected from the group of A, E, and Q; and X₈ is selected from the group of F, L, I, and M. The CGI factor motif may be SEQ ID NO: 1922, SEQ ID NO: 1923, SEQ ID NO: 1924, SEQ ID NO: 1925, SEQ ID NO: 1926, SEQ ID NO: 1927, SEQ ID NO: 1928, SEQ ID NO: 1929, SEQ ID NO: 1930, SEQ ID NO: 1931, SEQ ID NO: 1932, SEQ ID NO: 1933, SEQ ID NO: 1934, SEQ ID NO: 1935, SEQ ID NO: 1936, SEQ ID NO: 1937, SEQ ID NO: 1938, SEQ ID NO: 1939, SEQ ID NO: 1940, SEQ ID NO: 1941, SEQ ID NO: 1942, SEQ ID NO: 1943, SEQ ID NO: 1944, SEQ ID NO: 1945, SEQ ID NO: 1946, SEQ ID NO: 1947, SEQ ID NO: 1948, SEQ ID NO: 1949, SEQ ID NO: 1950, SEQ ID NO: 1951, SEQ ID NO: 1952, SEQ ID NO: 1953, SEQ ID NO: 1954, SEQ ID NO: 1955, or SEQ ID NO: 1956, each of which represents a motif subset of the sequence WX₁WX₂X₃X₄X₅X₆GX₇PX₈Y (SEQ ID NO: 1921).

In some embodiments, the CGI factor, CGI factor precursor, CGI factor fragment, or CGI factor motif is applied to a plant, a plant part, a harvested part of a plant, a seed, or an area to be planted to control any variety of fungal plant pathogens. Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses. Examples of commercially important cultivated crops, trees, and plants include: alfalfa (Medicago sativa), almonds (Prunus dulcis), apples (Malus x domestica), apricots (Prunus armeniaca, P. brigantine, P. mandshurica, P. mume, P. sibirica), artichoke (Cynara cardunculus var. scolymus), asparagus (Asparagus officinalis), avocado (Persea americana), bananas (Musa spp.), barley (Hordeum vulgare), beans (Phaseolus spp.), blueberries and cranberries (Vaccinium spp.), Brazil nut (Bertholletia excelsa), cacao (Theobroma cacao), calamansi (Citrus x microcarpa), canola and rapeseed or oilseed rape, (Brassica napus), Polish canola (Brassica rapa), and related cruciferous vegetables including broccoli, kale, cabbage, and turnips (Brassica carinata, B. juncea, B. oleracea, B. napus, B. nigra, and B. rapa, and hybrids of these), carnation (Dianthus caryophyllus), carrots (Daucus carota sativus), cashew (Anacardium occidentale), cassava (Manihot esculentum), celery (Apium graveolens), cherry (Prunus avium), chestnut (Castanea spp.), chickpea or garbanzo (Cicer arietinum), chicory (Cichorium intybus), chili peppers and other capsicum peppers (Capsicum annuum, C. frutescens, C. chinense, C. pubescens, C. baccatum), chrysanthemums (Chrysanthemum spp.), citron (Citrus medica), coconut (Cocos nucifera), coffee (wild and domesticated Coffea spp. including Coffea arabica, Coffea canephora, and Coffea liberica), cotton (Gossypium hirsutum L.), cowpea (Vigna unguiculata and other Vigna spp.), fava beans (Vicia faba), cucumber (Cucumis sativus), currants and gooseberries (Ribes spp.), date (Phoenix dactylifera), duckweeds (family Lemnoideae), eggplant or aubergine (Solanum melongena), elderberries (Sambucus spp.), eucalyptus (Eucalyptus spp.), flax (Linum usitatissumum L.), geraniums (Pelargonium spp.), ginger (Zingiber officinale), ginseng (Panax spp.), grapefruit (Citrus x paradisi), grapes (Vitis spp.) including wine grapes (Vitis vinifera and hybrids thereof), guava (Psidium guajava), hazelnut (Corylus avellana, Corylus spp.), hemp and cannabis (Cannabis sativa and Cannabis spp.), hops (Humulus lupulus), horseradish (Armoracia rusticana), irises (Iris spp.), jackfruit (Artocarpus heterophyllus), kiwifruits (Actinidia spp.), kumquat (Citrus japonica), lemon (Citrus limon), lentil (Lens culinaris), lettuce (Lactuca sativa), limes (Citrus spp.), lychee (Litchi chinensis), macadamias (Macadamia spp.), maize or corn (Zea mays L.), mandarin (Citrus reticulata), mango (Mangifera indica), mangosteen (Garcinia mangostana), melon (Cucumis melo), millets (Setaria spp., Echinochloa spp., Eleusine spp., Panicum spp., Pennisetum spp.), oats (Avena sativa), oil palm (Ellis quineensis), okra (Abelmoschus esculentus), olive (Olea europaea), onion (Allium cepa) and other alliums (Allium spp.), orange (Citrus sinensis), papaya (Carica papaya), parsnip (Pastinaca sativa), passionfruit (Passiflora edulis), pecan (Carya illinoinensis), peaches and nectarines (Prunus persica), pear (Pyrus spp.), pea (Pisum sativum), peanut (Arachis hypogaea), peonies (Paeonia spp.), persimmons (Diospyros kaki, Diospyros spp.), petunias (Petunia spp.), pineapple (Ananas comosus), pistachio (Pistacia vera), plantains (Musa spp.), plum (Prunus domestica), poinsettia (Euphorbia pulcherrima), pomelo (Citrus maxima), poplar (Populus spp.), potato (Solanum tuberosum), pumpkins and squashes (Cucurbita pepo, C. maxima, C. moschata), quince (Cydonia oblonga), raspberries (Rubus idaeus, Rubus occidentalis, Rubus spp.), rhubarbs (Rheum spp.), rice (Oryza sativa L.), roses (Rosa spp.), rubber (Hevea brasiliensis), rye (Secale cereale), safflower (Carthamus tinctorius L), satsuma (Citrus unshiu), sesame seed (Sesame indium), sorghum (Sorghum bicolor), sour orange (Citrus x aurantium), soursop (Annona muricata), soybean (Glycine max L.), strawberries (Fragaria spp., Fragaria x ananassa), sugar beet (Beta vulgaris), sugarcanes (Saccharum spp.), sunflower (Helianthus annuus), sweet potato (Ipomoea batatas), tamarind (Tamarindus indica), tangerine (Citrus tangerina), tea (Camellia sinensis), tobacco (Nicotiana tabacum L.), tomatillo (Physalis philadelphica), tomato (Solanum lycopersicum or Lycopersicon esculentum), tulips (Tulipa spp.), walnuts (Juglans spp. L.), watermelon (Citrulus lanatus), wheat (Triticum aestivum), and yams (Discorea spp.). Wild relatives of domesticated plants are also of interest.

Exemplary diseases which may be treated, with causative pathogen shown in parenthesis, include Alternaria Leaf and Fruit Spot (Alternaria alternata), Anthracnose (Colletotrichum acutatum), Leaf Blight (Seimatosporium lichenicola), Leaf Rust (Tranzschelia discolor), Scab (Cladosporium carpophilum), Shot Hole (Wilsonomyces carpophilus), Brown Rot Blossom Blight (Monilinia laxa, M. fructicola), Black Sigatoka (Mycosphaerella fijiensis), Yellow Sigatoka (Mycosphaerella musicola), Alternaria Fruit Rot (Alternaria spp.), Anthracnose Fruit Rot (Colletotrichum gloeosporoides), Botryosphaeria Canker (Botryosphaeria spp.), Leaf Spot and Blotch (Mycosphaerella spp., Septoria spp.), Mummyberry (Monilinia vaccinii-corymbosi), Phomopsis Leaf Spot, Twig Blight and Stem Canker (Phomopsis vaccinii), Powdery Mildew (Sphaerotheca spp.), Septoria Blight (Septoria spp.), Spur Blight (Didymella spp., Phoma spp.), Anthracnose (Spaceloma necator, Elsinoë veneta), Botryosphaeria Canker (Botryosphaeria dothidea), Colletotrichum Rot (Colletotrichum gloeosporioides), Leaf Spot and Blotch (Mycosphaerella spp., Septoria rubi, Sphaerulina rubi), Powdery Mildew (Sphaerotheca macularis, Microphaera spp., Oidium spp.), Rosette or Double Blossom of Blackberries (Cercosporella rubi), Spur Blight (Didymella applanata), Blackberry Rust (Phragmidium spp.), Anthracnose (Colletotrichum fragariae), Leather Rot (Phytophthora cactorum), Powdery Mildew (Sphaerotheca macularis), Botrytis grey mould on Foliage (Botrytis cinerea), Seedling Root Rot, Basal Stem Rot (Rhizoctonia solani), Cottonball (Monilinia oxycocci), Fruit Rots (Physalospora vaccinia, Glomerella cingulata, Coleophoma empetri), Lophodermium Twig Blight (Lophodermium spp.), Fairy Ring Suppression (Psilocybe spp.), Albinism (Alternaria alternata pv citri), Alternaria Leaf and Fruit Spot (Alternaria citri), Anthracnose (Colletotrichum acutatum, C. gloeosporioides), Cercospora Leaf Spot (Cercospora spp.), Diplodia Stem-End Rot (Diplodia natalensis), Greasy Spot (Mycosphaerella citri), Melanose (Diaporthe citri), Penicillium Decays, Green Mold, Whisker Mold, Blue Mold (Penicillium spp.), Phomopsis Stem-End Rot (Phomopsis citrii), Post Bloom Fruit Drop (PFD) (Colletotrichum acutatum), Powdery Mildew (Erysiphe spp.), Scab (Elsinoe fawcettii), Sweet Orange Scab (Elsinoe australis), Black Spot (Guignardia citricarpa), Black Rot (Guignardia bidwellii), Downy Mildew (Plasmopara viticola), Phomopsis Cane and Leaf Spot (Phomopsis viticola), Powdery Mildew (Uncinula necator), Botrytis Bunch Rot (Botrytis cinerea), Aspergillus Crown Rot (Aspergillus niger), Pythium Damping Off (Pythium spp.), Stem Rot/White Mold (Sclerotium rolfsii), Rhizoctonia Peg and Pod Rot (Rhizoctonia solani), Stem Rot/White Mold (Sclerotium rolfsii), Cylindrocladium Black Rot (Cylindocladium crotalariae), Pythium Pod Rot (Pythium myriotylum), Alternaria Late Blight (Alternaria alternata), Botryosphaeria Panicle and Shoot Blight (Botryosphaeria dothidea), Septoria Leaf Spot (Septoria pistaciarum), Scab (Cladosporium carpophilum), Alternaria Spot and Fruit Rot (Alternaria alternata), Anthracnose (Colletotrichum prunicola, C. gloeosporioides), Leaf Rust (Tranzschelia discolor), Powdery Mildew (Sphaerotheca pannosa, Podosphaera clandestina), Shot Hole (Wilsonomyces carpophilus), Alternaria Leaf Spot (Alternaria spp., A. alternata), Ascochyta Leaf Spot (Ascochyta cynarae), Phyllostica Leaf Spot (Phyllostica spp.), Rust (Uromyces betae, Puccinia helianthi), White Rust (Albugo tragopogonis), Anthracnose (Colletotrichum acutatum, Glomerella cingulata), Eastern Filbert Blight (Anisogramma anomale), Late Blight (Alternaria alternata), Scab (Cladosporium carpophilum), Septoria Leaf Spot (Septoria pistaciarum), Shot Hole (Wilsonomyces carpophilus), Blossom Blight (Monilinia laxa, M. fructicola), Powdery Mildew (Erysiphe spp.), Rust (Puccinia spp.), Alternaria black spot (Alternaria brassicae), Black leg/Phoma (Leptosphaeria maculans), Cercospora leaf spot (C. brassicicola), Head rot (Rhizoctonia solani), Leaf spot and pod rot (Alternaria alternata), Powdery mildew (Erysiphe polygoni), Southern blight (Sclerotium rolfsii), Anthracnose leaf blight (Colletotrichum graminicola), Gray leaf spot (Cercospora sorghi), Northern corn leaf blight (Setosphaeria turcica), Northern corn leaf spot (Cochliobolus carbonum), Common Rust (Puccinia sorghi), Southern Rust (P. polysora), Southern corn leaf blight (Cochliobolus heterostrophus), Eye spot (Aureobasidium zeae), Physoderma brown spot (P. maydis), Yellow Leaf Blight (Phyllosticta maydis), Ascochyta blight (A. gossypii), Rust (Puccinia schedonnardi, P. cacabata), Rhizoctonia leaf and stem diseases (R. solani), Target spot (Corynespora cassiicola), Southern blight (Sclerotium rolfsii), Rhizoctonia limb rot (R. solani), Cylindrocladium black rot (C. crotalaria), White mold (Sclerotinia minor), Early leaf spot (Cercospora arachidicola), Late leaf spot (Cercosporidium personatum), Web blotch (Phoma arachidicola), Rust (Puccinia arachidis), Pepper Spot (Leptospherulina crassiasca), Southern stem rot (Sclerotium rolfsii), Rhizoctonia limb rot (R. solani), Cylindrocladium black rot (C. crotalaria), White mold (Sclerotinia minor), Anthracnose (Colletotrichum lindemuthianum), Ascochyta blight (A. phaseolorum), Cercospora leaf blotch (C. cruenta), Downy mildew (Phytophthora nicotianae), Rust (Uromyces appendiculatus), Anthracnose (ripe rot) (C. gloeosporoides), Mummy berry (M. vacciniicorymbosi), Rust (Pucciniastrum vaccinii), Septoria leaf spot (Septoria albopunctata), Downy mildew (Peronospora parasitica), Alternaria leaf blight (A. dauci), Cercospora leaf spot (C. carotae), Basal stalk rot (Rhizoctonia solani), Early blight (Cercospora apii), Late blight (Septoria apicola), Verticillium brown spot and dry bubble, Pink rot (Sclerotinia sclerotiorum), Lophodermium leaf/twig blight (L. hypophyllum), Upright dieback (Phomopsis vaccinii), Anthracnose (Colletotrichum spp.), Downy mildew (Pseudoperonospora cubensis), Target spot (Corynespora cassiicola), Alternaria leaf blight (A. cucumerina), Alternaria leaf spot (A. alternata), Cercospora leaf spot (C. citrullina), Gummy stem blight/vine decline (Didymella bryoniae), Powdery mildew (Sphaerotheca only), Scab (Cladosporium cucumerinum), Anthracnose (Colletotrichum spp.), Botrytis leaf mold (Botrytis cinerea), Cercospora leaf spot (Cercospora spp.), Powdery mildew (Leveillula taurica), Purple blotch (Alternaria porri), Botrytis neck rot, Downy mildew (Peronospora destructor), Early leaf spot (Cercospora arachidicola), Late leaf spot (Cercosporidium personatum), Pepper spot (Leptosphaerulina crassiasca), Black dot (Colletotrichum coccodes), Botrytis vine rot (B. cinerea), Early blight (Alternaria solani), Late blight (Phytophthora infestans), Anthracnose (Colletotrichum truncatum), Cercospora leaf blight (C. kikuchii), Diaporthe pod and stem rot (D. phaseolorum), Frogeye leaf spot (Cercospora sojina), Purple seed stain (C. kikuchii), Septoria brown spot (S. glycines), Rust (Phakopsora pachyrhizi), Stem canker (Diaporthe phaseolorum), Early blight (Alternaria solani), Gray leaf mold (Fluvia fluva; Cladosporium), Gray leaf spot (Stemphyllium botryosum), Late blight (Phytophthora infestans), Septoria leaf spot (S. lycopersici), Target spot (Corynespora cassiicola), Alternaria fruit rot (black mold) (A. alternata), Anthracnose (Colletotrichum spp.), Botrytis gray mold (B. cinerea), Late blight fruit rot (P. infestans), Rhizoctonia fruit rot (R. solani), Anthracnose (Colletotrichum gloeosporioides), Anthracnose (Colletotrichum acutatum), Blossom blight/brown rot (Monilinia spp.), Scab (Venturia carpophila), Shot hole (Wilsonomyces carpophilus), Leaf curl (Taphrina deformans), Black knot (cherry, plum) (Apiosporina morbosa), Cherry leaf spot (Blumeriella jaapii), Scab (Cladosporium carpophilum), Interior needle blight (Mycosphaerella spp. and Phaeocryptopus nudus), Swiss needlecast (Phaeocryptopus gaeumannii), Interior needle blight (Mycosphaerella spp. and Phaeocryptopus nudus), Scleroderris canker (Gremmeniella abietina), Leaf rust (Thekopsora minima), Powdery mildew (Erysiphe necator), Alternaria rot (A. alternata), Angular leaf spot (Mycosphearella angulata), Anthracnose (Elsinoe ampelina), Black Rot (Guignardia bidwellii), Leaf Blight (Pseudocercospora vitis), Phomopsis cane and leaf spot (P. viticola), Rotbrenner (Pseudopezicula tracheiphila), Septoria leaf spot (S. ampelina), Apple Scab (Venturia inaequalis), Pear Scab (V. piris), Alternaria blotch, Alternaria rot (Alternaria spp.), Cedar apple rust (Gymnosporangium juniper-virginianae), Powdery mildew (Podosphaera leucotricha), Quince rust (Gymnosporangium spp.), Flyspeck and Sooty blotch, Bitter rot (Glomerella cingulata), Black rot (Botryosphaeria obtusa), Brooks fruit spot (Mycosphaerella pomi), White rot (Botryosphaeria dothidea), Alternaria rot and surface mold, Bitter rot Blue mold, Bull's-eye rot, Gray mold, Phacidiopycnis rot, Rhizopus rot, Speck rot, Sphaeropsis rot, White rot, Damping off (Pythium spp.), Root Rot (Phytophthora spp.), Leather rot (P. cactorum), Red stele (P. fragariae), Vascular collapse (P. cactorum), Basal stem rot (Phytophthora spp.), Crown rot (Phytophthora capsici), Downy Mildew (Peronospora effusa; P. farinosa), White rust (Albugo occidentalis), Pink rot (Phytophthora erythroseptica), Pythium leak, Pythium seedling disease (Pythium spp.), Phytophthora root and stem rot (Phytophthora megasperma), Pythium damping off (Pythium spp.), Collar rot, Crown rot, Root rot (Phytophthora spp.), Crown rot, Spear rot (Phytophthora spp.), Root Rot (Phytophthora cinnamomi), Downy mildew (Peronospora parasitica), Brown rot, Citrus foot rot, Gummosis, Root rot, Trunk canker (Phytophthora spp.), or Downy mildew (Bremia lactucae). From an agricultural or horticultural perspective, and for the purposes of this application, some of the pathogens and diseases listed above are considered “fungal” although the causative pathogen is technically an oomycete (phylum Oomycota), including, but not limited to Pythion spp., Phytophthora spp., Peronospora spp., Plasmopara spp., Albugo spp., and Bremia spp.

When applying to a harvested part of a plant (also referred to herein as post-harvest), application may be by a variety of treatment methods, e.g. dip, drip, drench, spray, or fog. In alternative embodiments, the harvested plant part has applied to it a composition, such as a film or membrane, containing the CGI factor, CGI factor precursor, or CGI factor fragment, or is packaged in a container that includes the CGI factor, CGI factor precursor, or CGI factor fragment. Such treatments, compositions, and containers are further useful for protecting foodstuffs (e.g., processed food products such as bakery goods or processed fruit or vegetables) from fungal growth and spoilage. Any of the plants or plant parts described above may be treated post-harvest. In some embodiments, plants treated post-harvest will include alfalfa, almonds, apples, apricots, artichoke, asparagus, avocado, bananas, barley, beans, blueberries and cranberries, Brazil nut, cacao, calamansi, canola and rapeseed or oilseed rape, Polish canola, and related cruciferous vegetables including broccoli, kale, cabbage, and turnips, carnation, carrots, cashew, cassava, celery, cherry, chestnut, chickpea or garbanzo, chicory, chili peppers and other capsicum peppers, chrysanthemums, citron, coconut, coffee, cotton, cowpea, fava beans, cucumber, currants and gooseberries, date, duckweeds, eggplant or aubergine, elderberries, eucalyptus, flax, geraniums, ginger, ginseng, grapefruit, grapes including wine grapes, guava, hazelnut, hemp and cannabis, hops, horseradish, irises, jackfruit, kiwifruits, kumquat, lemon, lentil, lettuce, limes, lychee, macadamias, maize or corn, mandarin, mango, mangosteen, melon, millets, oats, oil palm, okra, olive, onion and other alliums, orange, papaya, parsnip, passionfruit, pecan, peaches and nectarines, pear, pea, peanut, peonies, persimmons, petunias, pineapple, pistachio, plantains, plum, poinsettia, pomelo, poplar, potato, pumpkins and squashes, quince, raspberries, rhubarbs, rice, roses, rubber, rye, safflower, satsuma, sesame seed, sorghum, sour orange, soursop, soybean, strawberries, sugar beet, sugarcanes, sunflower, sweet potato, tamarind, tangerine, tea, tobacco, tomatillo, tomato, tulips, walnuts, watermelon, wheat, and yams.

In some embodiments, the CGI factor, CGI factor precursor, CGI factor fragment, or CGI factor motif is active and/or toxic to a structural element fungal pathogen (e.g., a fungal pathogen that infests or damages human-built structures such as buildings or other human-created artifacts, or components thereof). In some embodiments, the CGI factor, CGI factor precursor, CGI factor fragment, or CGI factor motif inhibits growth or reproduction of or is toxic to a fungus that damages wood or other materials useful in human-built structures or artifacts; examples include wood-decaying fungi that cause brown rot, white rot, or soft rot, or fungi that cause dry rot in human-built structures or buildings. In some embodiments, the structural element fungal pathogen is dry rot fungus (Serpula lacrymans), cellar rot fungus (Coniphora puteana), a wet rot fungus (Antrodia vaillantii, A. xantha, Asterostroma spp., Donkioporia expansa, Paxillus panuoides, Phellinus contignuus, Tyromyces placentus), or a fungus that colonizes water-damaged structural materials, e.g., Penicillium chrysogenum, Aspergillus versicolor, Chaetomium spp., Acremonium spp., Ulocladium spp., Stachybotrys spp., Arthrinium phaeospermum, Aureobasidium pullulans, Cladosporium herbarum, Trichoderma spp., Aspergillus fumigatus, Aspergillus melleus, Aspergillus niger, Aspergillus ochraceus, Mucor racemosus, or Mucor spinosus.

### Recombinant DNA Constructs and Vectors

An aspect of the disclosure includes a recombinant DNA construct including: a heterologous promoter operably linked to a nucleic acid molecule including a nucleotide sequence that encodes a conidial germination-inhibiting (CGI) factor, a CGI factor precursor, or a CGI factor fragment, wherein the nucleotide sequence (a) encodes at least one CGI factor including an amino acid sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with at least one of SEQ ID NO: 961, SEQ ID NO: 962, SEQ ID NO: 963, SEQ ID NO: 964, SEQ ID NO: 965, SEQ ID NO: 966, SEQ ID NO: 967, SEQ ID NO: 968, SEQ ID NO: 969, SEQ ID NO: 970, SEQ ID NO: 971, SEQ ID NO: 972, SEQ ID NO: 973, SEQ ID NO: 974, SEQ ID NO: 975, SEQ ID NO: 976, SEQ ID NO: 977, SEQ ID NO: 978, SEQ ID NO: 979, SEQ ID NO: 980, SEQ ID NO: 981, SEQ ID NO: 982, SEQ ID NO: 983, SEQ ID NO: 984, SEQ ID NO: 985, SEQ ID NO: 986, SEQ ID NO: 987, SEQ ID NO: 988, SEQ ID NO: 989, SEQ ID NO: 990, SEQ ID NO: 991, SEQ ID NO: 992, SEQ ID NO: 993, SEQ ID NO: 994, SEQ ID NO: 995, SEQ ID NO: 996, SEQ ID NO: 997, SEQ ID NO: 998, SEQ ID NO: 999, SEQ ID NO: 1000, SEQ ID NO: 1001, SEQ ID NO: 1002, SEQ ID NO: 1003, SEQ ID NO: 1004, SEQ ID NO: 1005, SEQ ID NO: 1006, SEQ ID NO: 1007, SEQ ID NO: 1008, SEQ ID NO: 1009, SEQ ID NO: 1010, SEQ ID NO: 1011, SEQ ID NO: 1012, SEQ ID NO: 1013, SEQ ID NO: 1014, SEQ ID NO: 1015, SEQ ID NO: 1016, SEQ ID NO: 1017, SEQ ID NO: 1018, SEQ ID NO: 1019, SEQ ID NO: 1020, SEQ ID NO: 1021, SEQ ID NO: 1022, SEQ ID NO: 1023, SEQ ID NO: 1024, SEQ ID NO: 1025, SEQ ID NO: 1026, SEQ ID NO: 1027, SEQ ID NO: 1028, SEQ ID NO: 1029, SEQ ID NO: 1030, SEQ ID NO: 1031, SEQ ID NO: 1032, SEQ ID NO: 1033, SEQ ID NO: 1034, SEQ ID NO: 1035, SEQ ID NO: 1036, SEQ ID NO: 1037, SEQ ID NO: 1038, SEQ ID NO: 1039, SEQ ID NO: 1040, SEQ ID NO: 1041, SEQ ID NO: 1042, SEQ ID NO: 1043, SEQ ID NO: 1044, SEQ ID NO: 1045, SEQ ID NO: 1046, SEQ ID NO: 1047, SEQ ID NO: 1048, SEQ ID NO: 1049, SEQ ID NO: 1050, SEQ ID NO: 1051, SEQ ID NO: 1052, SEQ ID NO: 1053, SEQ ID NO: 1054, SEQ ID NO: 1055, SEQ ID NO: 1056, SEQ ID NO: 1057, SEQ ID NO: 1058, SEQ ID NO: 1059, SEQ ID NO: 1060, SEQ ID NO: 1061, SEQ ID NO: 1062, SEQ ID NO: 1063, SEQ ID NO: 1064, SEQ ID NO: 1065, SEQ ID NO: 1066, SEQ ID NO: 1067, SEQ ID NO: 1068, SEQ ID NO: 1069, SEQ ID NO: 1070, SEQ ID NO: 1071, SEQ ID NO: 1072, SEQ ID NO: 1073, SEQ ID NO: 1074, SEQ ID NO: 1075, SEQ ID NO: 1076, SEQ ID NO: 1077, SEQ ID NO: 1078, SEQ ID NO: 1079, SEQ ID NO: 1080, SEQ ID NO: 1081, SEQ ID NO: 1082, SEQ ID NO: 1083, SEQ ID NO: 1084, SEQ ID NO: 1085, SEQ ID NO: 1086, SEQ ID NO: 1087, SEQ ID NO: 1088, SEQ ID NO: 1089, SEQ ID NO: 1090, SEQ ID NO: 1091, SEQ ID NO: 1092, SEQ ID NO: 1093, SEQ ID NO: 1094, SEQ ID NO: 1095, SEQ ID NO: 1096, SEQ ID NO: 1097, SEQ ID NO: 1098, SEQ ID NO: 1099, SEQ ID NO: 1100, SEQ ID NO: 1101, SEQ ID NO: 1102, SEQ ID NO: 1103, SEQ ID NO: 1104, SEQ ID NO: 1105, SEQ ID NO: 1106, SEQ ID NO: 1107, SEQ ID NO: 1108, SEQ ID NO: 1109, SEQ ID NO: 1110, SEQ ID NO: 1111, SEQ ID NO: 1112, SEQ ID NO: 1113, SEQ ID NO: 1114, SEQ ID NO: 1115, SEQ ID NO: 1116, SEQ ID NO: 1117, SEQ ID NO: 1118, SEQ ID NO: 1119, SEQ ID NO: 1120, SEQ ID NO: 1121, SEQ ID NO: 1122, SEQ ID NO: 1123, SEQ ID NO: 1124, SEQ ID NO: 1125, SEQ ID NO: 1126, SEQ ID NO: 1127, SEQ ID NO: 1128, SEQ ID NO: 1129, SEQ ID NO: 1130, SEQ ID NO: 1131, SEQ ID NO: 1132, SEQ ID NO: 1133, SEQ ID NO: 1134, SEQ ID NO: 1135, SEQ ID NO: 1136, SEQ ID NO: 1137, SEQ ID NO: 1138, SEQ ID NO: 1139, SEQ ID NO: 1140, SEQ ID NO: 1141, SEQ ID NO: 1142, SEQ ID NO: 1143, SEQ ID NO: 1144, SEQ ID NO: 1145, SEQ ID NO: 1146, SEQ ID NO: 1147, SEQ ID NO: 1148, SEQ ID NO: 1149, SEQ ID NO: 1150, SEQ ID NO: 1151, SEQ ID NO: 1152, SEQ ID NO: 1153, SEQ ID NO: 1154, SEQ ID NO: 1155, SEQ ID NO: 1156, SEQ ID NO: 1157, SEQ ID NO: 1158, SEQ ID NO: 1159, SEQ ID NO: 1160, SEQ ID NO: 1161, SEQ ID NO: 1162, SEQ ID NO: 1163, SEQ ID NO: 1164, SEQ ID NO: 1165, SEQ ID NO: 1166, SEQ ID NO: 1167, SEQ ID NO: 1168, SEQ ID NO: 1169, SEQ ID NO: 1170, SEQ ID NO: 1171, SEQ ID NO: 1172, SEQ ID NO: 1173, SEQ ID NO: 1174, SEQ ID NO: 1175, SEQ ID NO: 1176, SEQ ID NO: 1177, SEQ ID NO: 1178, SEQ ID NO: 1179, SEQ ID NO: 1180, SEQ ID NO: 1181, SEQ ID NO: 1182, SEQ ID NO: 1183, SEQ ID NO: 1184, SEQ ID NO: 1185, SEQ ID NO: 1186, SEQ ID NO: 1187, SEQ ID NO: 1188, SEQ ID NO: 1189, SEQ ID NO: 1190, SEQ ID NO: 1191, SEQ ID NO: 1192, SEQ ID NO: 1193, SEQ ID NO: 1194, SEQ ID NO: 1195, SEQ ID NO: 1196, SEQ ID NO: 1197, SEQ ID NO: 1198, SEQ ID NO: 1199, SEQ ID NO: 1200, SEQ ID NO: 1201, SEQ ID NO: 1202, SEQ ID NO: 1203, SEQ ID NO: 1204, SEQ ID NO: 1205, SEQ ID NO: 1206, SEQ ID NO: 1207, SEQ ID NO: 1208, SEQ ID NO: 1209, SEQ ID NO: 1210, SEQ ID NO: 1211, SEQ ID NO: 1212, SEQ ID NO: 1213, SEQ ID NO: 1214, SEQ ID NO: 1215, SEQ ID NO: 1216, SEQ ID NO: 1217, SEQ ID NO: 1218, SEQ ID NO: 1219, SEQ ID NO: 1220, SEQ ID NO: 1221, SEQ ID NO: 1222, SEQ ID NO: 1223, SEQ ID NO: 1224, SEQ ID NO: 1225, SEQ ID NO: 1226, SEQ ID NO: 1227, SEQ ID NO: 1228, SEQ ID NO: 1229, SEQ ID NO: 1230, SEQ ID NO: 1231, SEQ ID NO: 1232, SEQ ID NO: 1233, SEQ ID NO: 1234, SEQ ID NO: 1235, SEQ ID NO: 1236, SEQ ID NO: 1237, SEQ ID NO: 1238, SEQ ID NO: 1239, SEQ ID NO: 1240, SEQ ID NO: 1241, SEQ ID NO: 1242, SEQ ID NO: 1243, SEQ ID NO: 1244, SEQ ID NO: 1245, SEQ ID NO: 1246, SEQ ID NO: 1247, SEQ ID NO: 1248, SEQ ID NO: 1249, SEQ ID NO: 1250, SEQ ID NO: 1251, SEQ ID NO: 1252, SEQ ID NO: 1253, SEQ ID NO: 1254, SEQ ID NO: 1255, SEQ ID NO: 1256, SEQ ID NO: 1257, SEQ ID NO: 1258, SEQ ID NO: 1259, SEQ ID NO: 1260, SEQ ID NO: 1261, SEQ ID NO: 1262, SEQ ID NO: 1263, SEQ ID NO: 1264, SEQ ID NO: 1265, SEQ ID NO: 1266, SEQ ID NO: 1267, SEQ ID NO: 1268, SEQ ID NO: 1269, SEQ ID NO: 1270, SEQ ID NO: 1271, SEQ ID NO: 1272, SEQ ID NO: 1273, SEQ ID NO: 1274, SEQ ID NO: 1275, SEQ ID NO: 1276, SEQ ID NO: 1277, SEQ ID NO: 1278, SEQ ID NO: 1279, SEQ ID NO: 1280, SEQ ID NO: 1281, SEQ ID NO: 1282, SEQ ID NO: 1283, SEQ ID NO: 1284, SEQ ID NO: 1285, SEQ ID NO: 1286, SEQ ID NO: 1287, SEQ ID NO: 1288, SEQ ID NO: 1289, SEQ ID NO: 1290, SEQ ID NO: 1291, SEQ ID NO: 1292, SEQ ID NO: 1293, SEQ ID NO: 1294, SEQ ID NO: 1295, SEQ ID NO: 1296, SEQ ID NO: 1297, SEQ ID NO: 1298, SEQ ID NO: 1299, SEQ ID NO: 1300, SEQ ID NO: 1301, SEQ ID NO: 1302, SEQ ID NO: 1303, SEQ ID NO: 1304, SEQ ID NO: 1305, SEQ ID NO: 1306, SEQ ID NO: 1307, SEQ ID NO: 1308, SEQ ID NO: 1309, SEQ ID NO: 1310, SEQ ID NO: 1311, SEQ ID NO: 1312, SEQ ID NO: 1313, SEQ ID NO: 1314, SEQ ID NO: 1315, SEQ ID NO: 1316, SEQ ID NO: 1317, SEQ ID NO: 1318, SEQ ID NO: 1319, SEQ ID NO: 1320, SEQ ID NO: 1321, SEQ ID NO: 1322, SEQ ID NO: 1323, SEQ ID NO: 1324, SEQ ID NO: 1325, SEQ ID NO: 1326, SEQ ID NO: 1327, SEQ ID NO: 1328, SEQ ID NO: 1329, SEQ ID NO: 1330, SEQ ID NO: 1331, SEQ ID NO: 1332, SEQ ID NO: 1333, SEQ ID NO: 1334, SEQ ID NO: 1335, SEQ ID NO: 1336, SEQ ID NO: 1337, SEQ ID NO: 1338, SEQ ID NO: 1339, SEQ ID NO: 1340, SEQ ID NO: 1341, SEQ ID NO: 1342, SEQ ID NO: 1343, SEQ ID NO: 1344, SEQ ID NO: 1345, SEQ ID NO: 1346, SEQ ID NO: 1347, SEQ ID NO: 1348, SEQ ID NO: 1349, SEQ ID NO: 1350, SEQ ID NO: 1351, SEQ ID NO: 1352, SEQ ID NO: 1353, SEQ ID NO: 1354, SEQ ID NO: 1355, SEQ ID NO: 1356, SEQ ID NO: 1357, SEQ ID NO: 1358, SEQ ID NO: 1359, SEQ ID NO: 1360, SEQ ID NO: 1361, SEQ ID NO: 1362, SEQ ID NO: 1363, SEQ ID NO: 1364, SEQ ID NO: 1365, SEQ ID NO: 1366, SEQ ID NO: 1367, SEQ ID NO: 1368, SEQ ID NO: 1369, SEQ ID NO: 1370, SEQ ID NO: 1371, SEQ ID NO: 1372, SEQ ID NO: 1373, SEQ ID NO: 1374, SEQ ID NO: 1375, SEQ ID NO: 1376, SEQ ID NO: 1377, SEQ ID NO: 1378, SEQ ID NO: 1379, SEQ ID NO: 1380, SEQ ID NO: 1381, SEQ ID NO: 1382, SEQ ID NO: 1383, SEQ ID NO: 1384, SEQ ID NO: 1385, SEQ ID NO: 1386, SEQ ID NO: 1387, SEQ ID NO: 1388, SEQ ID NO: 1389, SEQ ID NO: 1390, SEQ ID NO: 1391, SEQ ID NO: 1392, SEQ ID NO: 1393, SEQ ID NO: 1394, SEQ ID NO: 1395, SEQ ID NO: 1396, SEQ ID NO: 1397, SEQ ID NO: 1398, SEQ ID NO: 1399, SEQ ID NO: 1400, SEQ ID NO: 1401, SEQ ID NO: 1402, SEQ ID NO: 1403, SEQ ID NO: 1404, SEQ ID NO: 1405, SEQ ID NO: 1406, SEQ ID NO: 1407, SEQ ID NO: 1408, SEQ ID NO: 1409, SEQ ID NO: 1410, SEQ ID NO: 1411, SEQ ID NO: 1412, SEQ ID NO: 1413, SEQ ID NO: 1414, SEQ ID NO: 1415, SEQ ID NO: 1416, SEQ ID NO: 1417, SEQ ID NO: 1418, SEQ ID NO: 1419, SEQ ID NO: 1420, SEQ ID NO: 1421, SEQ ID NO: 1422, SEQ ID NO: 1423, SEQ ID NO: 1424, SEQ ID NO: 1425, SEQ ID NO: 1426, SEQ ID NO: 1427, SEQ ID NO: 1428, SEQ ID NO: 1429, SEQ ID NO: 1430, SEQ ID NO: 1431, SEQ ID NO: 1432, SEQ ID NO: 1433, SEQ ID NO: 1434, SEQ ID NO: 1435, SEQ ID NO: 1436, SEQ ID NO: 1437, SEQ ID NO: 1438, SEQ ID NO: 1439, SEQ ID NO: 1440, SEQ ID NO: 1441, SEQ ID NO: 1442, SEQ ID NO: 1443, SEQ ID NO: 1444, SEQ ID NO: 1445, SEQ ID NO: 1446, SEQ ID NO: 1447, SEQ ID NO: 1448, SEQ ID NO: 1449, SEQ ID NO: 1450, SEQ ID NO: 1451, SEQ ID NO: 1452, SEQ ID NO: 1453, SEQ ID NO: 1454, SEQ ID NO: 1455, SEQ ID NO: 1456, SEQ ID NO: 1457, SEQ ID NO: 1458, SEQ ID NO: 1459, SEQ ID NO: 1460, SEQ ID NO: 1461, SEQ ID NO: 1462, SEQ ID NO: 1463, SEQ ID NO: 1464, SEQ ID NO: 1465, SEQ ID NO: 1466, SEQ ID NO: 1467, SEQ ID NO: 1468, SEQ ID NO: 1469, SEQ ID NO: 1470, SEQ ID NO: 1471, SEQ ID NO: 1472, SEQ ID NO: 1473, SEQ ID NO: 1474, SEQ ID NO: 1475, SEQ ID NO: 1476, SEQ ID NO: 1477, SEQ ID NO: 1478, SEQ ID NO: 1479, SEQ ID NO: 1480, SEQ ID NO: 1481, SEQ ID NO: 1482, SEQ ID NO: 1483, SEQ ID NO: 1484, SEQ ID NO: 1485, SEQ ID NO: 1486, SEQ ID NO: 1487, SEQ ID NO: 1488, SEQ ID NO: 1489, SEQ ID NO: 1490, SEQ ID NO: 1491, SEQ ID NO: 1492, SEQ ID NO: 1493, SEQ ID NO: 1494, SEQ ID NO: 1495, SEQ ID NO: 1496, SEQ ID NO: 1497, SEQ ID NO: 1498, SEQ ID NO: 1499, SEQ ID NO: 1500, SEQ ID NO: 1501, SEQ ID NO: 1502, SEQ ID NO: 1503, SEQ ID NO: 1504, SEQ ID NO: 1505, SEQ ID NO: 1506, SEQ ID NO: 1507, SEQ ID NO: 1508, SEQ ID NO: 1509, SEQ ID NO: 1510, SEQ ID NO: 1511, SEQ ID NO: 1512, SEQ ID NO: 1513, SEQ ID NO: 1514, SEQ ID NO: 1515, SEQ ID NO: 1516, SEQ ID NO: 1517, SEQ ID NO: 1518, SEQ ID NO: 1519, SEQ ID NO: 1520, SEQ ID NO: 1521, SEQ ID NO: 1522, SEQ ID NO: 1523, SEQ ID NO: 1524, SEQ ID NO: 1525, SEQ ID NO: 1526, SEQ ID NO: 1527, SEQ ID NO: 1528, SEQ ID NO: 1529, SEQ ID NO: 1530, SEQ ID NO: 1531, SEQ ID NO: 1532, SEQ ID NO: 1533, SEQ ID NO: 1534, SEQ ID NO: 1535, SEQ ID NO: 1536, SEQ ID NO: 1537, SEQ ID NO: 1538, SEQ ID NO: 1539, SEQ ID NO: 1540, SEQ ID NO: 1541, SEQ ID NO: 1542, SEQ ID NO: 1543, SEQ ID NO: 1544, SEQ ID NO: 1545, SEQ ID NO: 1546, SEQ ID NO: 1547, SEQ ID NO: 1548, SEQ ID NO: 1549, SEQ ID NO: 1550, SEQ ID NO: 1551, SEQ ID NO: 1552, SEQ ID NO: 1553, SEQ ID NO: 1554, SEQ ID NO: 1555, SEQ ID NO: 1556, SEQ ID NO: 1557, SEQ ID NO: 1558, SEQ ID NO: 1559, SEQ ID NO: 1560, SEQ ID NO: 1561, SEQ ID NO: 1562, SEQ ID NO: 1563, SEQ ID NO: 1564, SEQ ID NO: 1565, SEQ ID NO: 1566, SEQ ID NO: 1567, SEQ ID NO: 1568, SEQ ID NO: 1569, SEQ ID NO: 1570, SEQ ID NO: 1571, SEQ ID NO: 1572, SEQ ID NO: 1573, SEQ ID NO: 1574, SEQ ID NO: 1575, SEQ ID NO: 1576, SEQ ID NO: 1577, SEQ ID NO: 1578, SEQ ID NO: 1579, SEQ ID NO: 1580, SEQ ID NO: 1581, SEQ ID NO: 1582, SEQ ID NO: 1583, SEQ ID NO: 1584, SEQ ID NO: 1585, SEQ ID NO: 1586, SEQ ID NO: 1587, SEQ ID NO: 1588, SEQ ID NO: 1589, SEQ ID NO: 1590, SEQ ID NO: 1591, SEQ ID NO: 1592, SEQ ID NO: 1593, SEQ ID NO: 1594, SEQ ID NO: 1595, SEQ ID NO: 1596, SEQ ID NO: 1597, SEQ ID NO: 1598, SEQ ID NO: 1599, SEQ ID NO: 1600, SEQ ID NO: 1601, SEQ ID NO: 1602, SEQ ID NO: 1603, SEQ ID NO: 1604, SEQ ID NO: 1605, SEQ ID NO: 1606, SEQ ID NO: 1607, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1616, SEQ ID NO: 1617, SEQ ID NO: 1618, SEQ ID NO: 1619, SEQ ID NO: 1620, SEQ ID NO: 1621, SEQ ID NO: 1622, SEQ ID NO: 1623, SEQ ID NO: 1624, SEQ ID NO: 1625, SEQ ID NO: 1626, SEQ ID NO: 1627, SEQ ID NO: 1628, SEQ ID NO: 1629, SEQ ID NO: 1630, SEQ ID NO: 1631, SEQ ID NO: 1632, SEQ ID NO: 1633, SEQ ID NO: 1634, SEQ ID NO: 1635, SEQ ID NO: 1636, SEQ ID NO: 1637, SEQ ID NO: 1638, SEQ ID NO: 1639, SEQ ID NO: 1640, SEQ ID NO: 1641, SEQ ID NO: 1642, SEQ ID NO: 1643, SEQ ID NO: 1644, SEQ ID NO: 1645, SEQ ID NO: 1646, SEQ ID NO: 1647, SEQ ID NO: 1648, SEQ ID NO: 1649, SEQ ID NO: 1650, SEQ ID NO: 1651, SEQ ID NO: 1652, SEQ ID NO: 1653, SEQ ID NO: 1654, SEQ ID NO: 1655, SEQ ID NO: 1656, SEQ ID NO: 1657, SEQ ID NO: 1658, SEQ ID NO: 1659, SEQ ID NO: 1660, SEQ ID NO: 1661, SEQ ID NO: 1662, SEQ ID NO: 1663, SEQ ID NO: 1664, SEQ ID NO: 1665, SEQ ID NO: 1666, SEQ ID NO: 1667, SEQ ID NO: 1668, SEQ ID NO: 1669, SEQ ID NO: 1670, SEQ ID NO: 1671, SEQ ID NO: 1672, SEQ ID NO: 1673, SEQ ID NO: 1674, SEQ ID NO: 1675, SEQ ID NO: 1676, SEQ ID NO: 1677, SEQ ID NO: 1678, SEQ ID NO: 1679, SEQ ID NO: 1680, SEQ ID NO: 1681, SEQ ID NO: 1682, SEQ ID NO: 1683, SEQ ID NO: 1684, SEQ ID NO: 1685, SEQ ID NO: 1686, SEQ ID NO: 1687, SEQ ID NO: 1688, SEQ ID NO: 1689, SEQ ID NO: 1690, SEQ ID NO: 1691, SEQ ID NO: 1692, SEQ ID NO: 1693, SEQ ID NO: 1694, SEQ ID NO: 1695, SEQ ID NO: 1696, SEQ ID NO: 1697, SEQ ID NO: 1698, SEQ ID NO: 1699, SEQ ID NO: 1700, SEQ ID NO: 1701, SEQ ID NO: 1702, SEQ ID NO: 1703, SEQ ID NO: 1704, SEQ ID NO: 1705, SEQ ID NO: 1706, SEQ ID NO: 1707, SEQ ID NO: 1708, SEQ ID NO: 1709, SEQ ID NO: 1710, SEQ ID NO: 1711, SEQ ID NO: 1712, SEQ ID NO: 1713, SEQ ID NO: 1714, SEQ ID NO: 1715, SEQ ID NO: 1716, SEQ ID NO: 1717, SEQ ID NO: 1718, SEQ ID NO: 1719, SEQ ID NO: 1720, SEQ ID NO: 1721, SEQ ID NO: 1722, SEQ ID NO: 1723, SEQ ID NO: 1724, SEQ ID NO: 1725, SEQ ID NO: 1726, SEQ ID NO: 1727, SEQ ID NO: 1728, SEQ ID NO: 1729, SEQ ID NO: 1730, SEQ ID NO: 1731, SEQ ID NO: 1732, SEQ ID NO: 1733, SEQ ID NO: 1734, SEQ ID NO: 1735, SEQ ID NO: 1736, SEQ ID NO: 1737, SEQ ID NO: 1738, SEQ ID NO: 1739, SEQ ID NO: 1740, SEQ ID NO: 1741, SEQ ID NO: 1742, SEQ ID NO: 1743, SEQ ID NO: 1744, SEQ ID NO: 1745, SEQ ID NO: 1746, SEQ ID NO: 1747, SEQ ID NO: 1748, SEQ ID NO: 1749, SEQ ID NO: 1750, SEQ ID NO: 1751, SEQ ID NO: 1752, SEQ ID NO: 1753, SEQ ID NO: 1754, SEQ ID NO: 1755, SEQ ID NO: 1756, SEQ ID NO: 1757, SEQ ID NO: 1758, SEQ ID NO: 1759, SEQ ID NO: 1760, SEQ ID NO: 1761, SEQ ID NO: 1762, SEQ ID NO: 1763, SEQ ID NO: 1764, SEQ ID NO: 1765, SEQ ID NO: 1766, SEQ ID NO: 1767, SEQ ID NO: 1768, SEQ ID NO: 1769, SEQ ID NO: 1770, SEQ ID NO: 1771, SEQ ID NO: 1772, SEQ ID NO: 1773, SEQ ID NO: 1774, SEQ ID NO: 1775, SEQ ID NO: 1776, SEQ ID NO: 1777, SEQ ID NO: 1778, SEQ ID NO: 1779, SEQ ID NO: 1780, SEQ ID NO: 1781, SEQ ID NO: 1782, SEQ ID NO: 1783, SEQ ID NO: 1784, SEQ ID NO: 1785, SEQ ID NO: 1786, SEQ ID NO: 1787, SEQ ID NO: 1788, SEQ ID NO: 1789, SEQ ID NO: 1790, SEQ ID NO: 1791, SEQ ID NO: 1792, SEQ ID NO: 1793, SEQ ID NO: 1794, SEQ ID NO: 1795, SEQ ID NO: 1796, SEQ ID NO: 1797, SEQ ID NO: 1798, SEQ ID NO: 1799, SEQ ID NO: 1800, SEQ ID NO: 1801, SEQ ID NO: 1802, SEQ ID NO: 1803, SEQ ID NO: 1804, SEQ ID NO: 1805, SEQ ID NO: 1806, SEQ ID NO: 1807, SEQ ID NO: 1808, SEQ ID NO: 1809, SEQ ID NO: 1810, SEQ ID NO: 1811, SEQ ID NO: 1812, SEQ ID NO: 1813, SEQ ID NO: 1814, SEQ ID NO: 1815, SEQ ID NO: 1816, SEQ ID NO: 1817, SEQ ID NO: 1818, SEQ ID NO: 1819, SEQ ID NO: 1820, SEQ ID NO: 1821, SEQ ID NO: 1822, SEQ ID NO: 1823, SEQ ID NO: 1824, SEQ ID NO: 1825, SEQ ID NO: 1826, SEQ ID NO: 1827, SEQ ID NO: 1828, SEQ ID NO: 1829, SEQ ID NO: 1830, SEQ ID NO: 1831, SEQ ID NO: 1832, SEQ ID NO: 1833, SEQ ID NO: 1834, SEQ ID NO: 1835, SEQ ID NO: 1836, SEQ ID NO: 1837, SEQ ID NO: 1838, SEQ ID NO: 1839, SEQ ID NO: 1840, SEQ ID NO: 1841, SEQ ID NO: 1842, SEQ ID NO: 1843, SEQ ID NO: 1844, SEQ ID NO: 1845, SEQ ID NO: 1846, SEQ ID NO: 1847, SEQ ID NO: 1848, SEQ ID NO: 1849, SEQ ID NO: 1850, SEQ ID NO: 1851, SEQ ID NO: 1852, SEQ ID NO: 1853, SEQ ID NO: 1854, SEQ ID NO: 1855, SEQ ID NO: 1856, SEQ ID NO: 1857, SEQ ID NO: 1858, SEQ ID NO: 1859, SEQ ID NO: 1860, SEQ ID NO: 1861, SEQ ID NO: 1862, SEQ ID NO: 1863, SEQ ID NO: 1864, SEQ ID NO: 1865, SEQ ID NO: 1866, SEQ ID NO: 1867, SEQ ID NO: 1868, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, SEQ ID NO: 1876, SEQ ID NO: 1877, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, SEQ ID NO: 1894, SEQ ID NO: 1895, SEQ ID NO: 1896, SEQ ID NO: 1897, SEQ ID NO: 1898, SEQ ID NO: 1899, SEQ ID NO: 1900, SEQ ID NO: 1901, SEQ ID NO: 1902, SEQ ID NO: 1903, SEQ ID NO: 1904, SEQ ID NO: 1905, SEQ ID NO: 1906, SEQ ID NO: 1907, SEQ ID NO: 1908, SEQ ID NO: 1909, SEQ ID NO: 1910, SEQ ID NO: 1911, SEQ ID NO: 1912, SEQ ID NO: 1913, SEQ ID NO: 1914, SEQ ID NO: 1915, SEQ ID NO: 1916, SEQ ID NO: 1917, SEQ ID NO: 1918, SEQ ID NO: 1919, SEQ ID NO: 1920, SEQ ID NO: 1957, SEQ ID NO: 1958, SEQ ID NO: 1959, SEQ ID NO: 1960, SEQ ID NO: 1961, SEQ ID NO: 1962, SEQ ID NO: 1963, SEQ ID NO: 1964, SEQ ID NO: 1965, SEQ ID NO: 1966, SEQ ID NO: 1967, SEQ ID NO: 1968, SEQ ID NO: 1969, SEQ ID NO: 1970, SEQ ID NO: 1971, SEQ ID NO: 1972, SEQ ID NO: 1973, SEQ ID NO: 1974, SEQ ID NO: 1975, SEQ ID NO: 1976, SEQ ID NO: 1977, SEQ ID NO: 1978, SEQ ID NO: 1979, SEQ ID NO: 1980, SEQ ID NO: 1981, SEQ ID NO: 1982, SEQ ID NO: 1983, SEQ ID NO: 1984, SEQ ID NO: 1985, SEQ ID NO: 1986, SEQ ID NO: 1987, SEQ ID NO: 1988, SEQ ID NO: 1989, SEQ ID NO: 1990, SEQ ID NO: 1991, SEQ ID NO: 1992, SEQ ID NO: 1993, SEQ ID NO: 1994, SEQ ID NO: 1995, SEQ ID NO: 1996, SEQ ID NO: 1997, SEQ ID NO: 1998, SEQ ID NO: 1999, SEQ ID NO: 2000, SEQ ID NO: 2001, SEQ ID NO: 2002, SEQ ID NO: 2003, SEQ ID NO: 2004, SEQ ID NO: 2005, SEQ ID NO: 2006, SEQ ID NO: 2007, SEQ ID NO: 2008, SEQ ID NO: 2009, SEQ ID NO: 2010, SEQ ID NO: 2011, SEQ ID NO: 2012, SEQ ID NO: 2013, SEQ ID NO: 2014, SEQ ID NO: 2015, SEQ ID NO: 2016, SEQ ID NO: 2017, SEQ ID NO: 2018, SEQ ID NO: 2019, SEQ ID NO: 2020, SEQ ID NO: 2021, SEQ ID NO: 2022, SEQ ID NO: 2023, SEQ ID NO: 2024, SEQ ID NO: 2025, SEQ ID NO: 2026, SEQ ID NO: 2027, SEQ ID NO: 2028, SEQ ID NO: 2029, SEQ ID NO: 2030, SEQ ID NO: 2031, SEQ ID NO: 2032, SEQ ID NO: 2033, SEQ ID NO: 2034, SEQ ID NO: 2035, SEQ ID NO: 2036, SEQ ID NO: 2037, SEQ ID NO: 2038, SEQ ID NO: 2039, SEQ ID NO: 2040, SEQ ID NO: 2041, SEQ ID NO: 2042, SEQ ID NO: 2043, SEQ ID NO: 2044, SEQ ID NO: 2045, SEQ ID NO: 2046, SEQ ID NO: 2047, SEQ ID NO: 2048, SEQ ID NO: 2049, SEQ ID NO: 2050, SEQ ID NO: 2051, SEQ ID NO: 2052, SEQ ID NO: 2053, SEQ ID NO: 2054, SEQ ID NO: 2055, SEQ ID NO: 2056, SEQ ID NO: 2057, SEQ ID NO: 2058, SEQ ID NO: 2059, SEQ ID NO: 2060, SEQ ID NO: 2061, SEQ ID NO: 2062, SEQ ID NO: 2063, SEQ ID NO: 2064, SEQ ID NO: 2065, SEQ ID NO: 2066, SEQ ID NO: 2067, SEQ ID NO: 2068, SEQ ID NO: 2069, SEQ ID NO: 2070, SEQ ID NO: 2071, SEQ ID NO: 2072, SEQ ID NO: 2073, SEQ ID NO: 2074, SEQ ID NO: 2075, SEQ ID NO: 2076, SEQ ID NO: 2077, SEQ ID NO: 2078, SEQ ID NO: 2079, SEQ ID NO: 2080, SEQ ID NO: 2081, SEQ ID NO: 2082, SEQ ID NO: 2083, SEQ ID NO: 2084, SEQ ID NO: 2085, SEQ ID NO: 2086, SEQ ID NO: 2087, SEQ ID NO: 2088, SEQ ID NO: 2089, SEQ ID NO: 2090, SEQ ID NO: 2091, SEQ ID NO: 2092, SEQ ID NO: 2093, SEQ ID NO: 2094, SEQ ID NO: 2095, SEQ ID NO: 2096, SEQ ID NO: 2097, SEQ ID NO: 2098, SEQ ID NO: 2099, SEQ ID NO: 2100, SEQ ID NO: 2101, SEQ ID NO: 2102, SEQ ID NO: 2103, SEQ ID NO: 2104, SEQ ID NO: 2105, SEQ ID NO: 2106, SEQ ID NO: 2107, SEQ ID NO: 2108, SEQ ID NO: 2109, SEQ ID NO: 2110, SEQ ID NO: 2111, SEQ ID NO: 2112, SEQ ID NO: 2113, SEQ ID NO: 2114, SEQ ID NO: 2115, SEQ ID NO: 2116, SEQ ID NO: 2117, SEQ ID NO: 2118, SEQ ID NO: 2119, SEQ ID NO: 2120, SEQ ID NO: 2121, SEQ ID NO: 2122, SEQ ID NO: 2123, SEQ ID NO: 2124, SEQ ID NO: 2125, SEQ ID NO: 2126, SEQ ID NO: 2127, SEQ ID NO: 2128, SEQ ID NO: 2129, SEQ ID NO: 2130, SEQ ID NO: 2131, SEQ ID NO: 2132, SEQ ID NO: 2133, SEQ ID NO: 2134, SEQ ID NO: 2135, SEQ ID NO: 2136, SEQ ID NO: 2137, SEQ ID NO: 2138, SEQ ID NO: 2139, SEQ ID NO: 2140, SEQ ID NO: 2141, SEQ ID NO: 2142, SEQ ID NO: 2143, SEQ ID NO: 2144, SEQ ID NO: 2145, SEQ ID NO: 2146, SEQ ID NO: 2147, SEQ ID NO: 2148, SEQ ID NO: 2149, SEQ ID NO: 2150, SEQ ID NO: 2151, SEQ ID NO: 2152, SEQ ID NO: 2153, SEQ ID NO: 2154, SEQ ID NO: 2155, SEQ ID NO: 2156, SEQ ID NO: 2157, SEQ ID NO: 2158, SEQ ID NO: 2159, SEQ ID NO: 2160, SEQ ID NO: 2161, SEQ ID NO: 2162, SEQ ID NO: 2163, SEQ ID NO: 2164, SEQ ID NO: 2165, SEQ ID NO: 2166, SEQ ID NO: 2167, SEQ ID NO: 2168, SEQ ID NO: 2169, SEQ ID NO: 2170, SEQ ID NO: 2171, SEQ ID NO: 2172, SEQ ID NO: 2173, SEQ ID NO: 2174, SEQ ID NO: 2175, SEQ ID NO: 2176, SEQ ID NO: 2177, SEQ ID NO: 2178, SEQ ID NO: 2179, SEQ ID NO: 2180, or SEQ ID NO: 2181, at least one CGI factor precursor, or at least one CGI factor fragment, (b) is a synthetic sequence of (a) that has codons optimized for heterologous expression; or (c) encodes at least one CGI factor motif. The nucleotide sequence encoding the at least one CGI factor of the preceding embodiment may include SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, SEQ ID NO: 588, SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, SEQ ID NO: 615, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, SEQ ID NO: 624, SEQ ID NO: 625, SEQ ID NO: 626, SEQ ID NO: 627, SEQ ID NO: 628, SEQ ID NO: 629, SEQ ID NO: 630, SEQ ID NO: 631, SEQ ID NO: 632, SEQ ID NO: 633, SEQ ID NO: 634, SEQ ID NO: 635, SEQ ID NO: 636, SEQ ID NO: 637, SEQ ID NO: 638, SEQ ID NO: 639, SEQ ID NO: 640, SEQ ID NO: 641, SEQ ID NO: 642, SEQ ID NO: 643, SEQ ID NO: 644, SEQ ID NO: 645, SEQ ID NO: 646, SEQ ID NO: 647, SEQ ID NO: 648, SEQ ID NO: 649, SEQ ID NO: 650, SEQ ID NO: 651, SEQ ID NO: 652, SEQ ID NO: 653, SEQ ID NO: 654, SEQ ID NO: 655, SEQ ID NO: 656, SEQ ID NO: 657, SEQ ID NO: 658, SEQ ID NO: 659, SEQ ID NO: 660, SEQ ID NO: 661, SEQ ID NO: 662, SEQ ID NO: 663, SEQ ID NO: 664, SEQ ID NO: 665, SEQ ID NO: 666, SEQ ID NO: 667, SEQ ID NO: 668, SEQ ID NO: 669, SEQ ID NO: 670, SEQ ID NO: 671, SEQ ID NO: 672, SEQ ID NO: 673, SEQ ID NO: 674, SEQ ID NO: 675, SEQ ID NO: 676, SEQ ID NO: 677, SEQ ID NO: 678, SEQ ID NO: 679, SEQ ID NO: 680, SEQ ID NO: 681, SEQ ID NO: 682, SEQ ID NO: 683, SEQ ID NO: 684, SEQ ID NO: 685, SEQ ID NO: 686, SEQ ID NO: 687, SEQ ID NO: 688, SEQ ID NO: 689, SEQ ID NO: 690, SEQ ID NO: 691, SEQ ID NO: 692, SEQ ID NO: 693, SEQ ID NO: 694, SEQ ID NO: 695, SEQ ID NO: 696, SEQ ID NO: 697, SEQ ID NO: 698, SEQ ID NO: 699, SEQ ID NO: 700, SEQ ID NO: 701, SEQ ID NO: 702, SEQ ID NO: 703, SEQ ID NO: 704, SEQ ID NO: 705, SEQ ID NO: 706, SEQ ID NO: 707, SEQ ID NO: 708, SEQ ID NO: 709, SEQ ID NO: 710, SEQ ID NO: 711, SEQ ID NO: 712, SEQ ID NO: 713, SEQ ID NO: 714, SEQ ID NO: 715, SEQ ID NO: 716, SEQ ID NO: 717, SEQ ID NO: 718, SEQ ID NO: 719, SEQ ID NO: 720, SEQ ID NO: 721, SEQ ID NO: 722, SEQ ID NO: 723, SEQ ID NO: 724, SEQ ID NO: 725, SEQ ID NO: 726, SEQ ID NO: 727, SEQ ID NO: 728, SEQ ID NO: 729, SEQ ID NO: 730, SEQ ID NO: 731, SEQ ID NO: 732, SEQ ID NO: 733, SEQ ID NO: 734, SEQ ID NO: 735, SEQ ID NO: 736, SEQ ID NO: 737, SEQ ID NO: 738, SEQ ID NO: 739, SEQ ID NO: 740, SEQ ID NO: 741, SEQ ID NO: 742, SEQ ID NO: 743, SEQ ID NO: 744, SEQ ID NO: 745, SEQ ID NO: 746, SEQ ID NO: 747, SEQ ID NO: 748, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, SEQ ID NO: 776, SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, SEQ ID NO: 788, SEQ ID NO: 789, SEQ ID NO: 790, SEQ ID NO: 791, SEQ ID NO: 792, SEQ ID NO: 793, SEQ ID NO: 794, SEQ ID NO: 795, SEQ ID NO: 796, SEQ ID NO: 797, SEQ ID NO: 798, SEQ ID NO: 799, SEQ ID NO: 800, SEQ ID NO: 801, SEQ ID NO: 802, SEQ ID NO: 803, SEQ ID NO: 804, SEQ ID NO: 805, SEQ ID NO: 806, SEQ ID NO: 807, SEQ ID NO: 808, SEQ ID NO: 809, SEQ ID NO: 810, SEQ ID NO: 811, SEQ ID NO: 812, SEQ ID NO: 813, SEQ ID NO: 814, SEQ ID NO: 815, SEQ ID NO: 816, SEQ ID NO: 817, SEQ ID NO: 818, SEQ ID NO: 819, SEQ ID NO: 820, SEQ ID NO: 821, SEQ ID NO: 822, SEQ ID NO: 823, SEQ ID NO: 824, SEQ ID NO: 825, SEQ ID NO: 826, SEQ ID NO: 827, SEQ ID NO: 828, SEQ ID NO: 829, SEQ ID NO: 830, SEQ ID NO: 831, SEQ ID NO: 832, SEQ ID NO: 833, SEQ ID NO: 834, SEQ ID NO: 835, SEQ ID NO: 836, SEQ ID NO: 837, SEQ ID NO: 838, SEQ ID NO: 839, SEQ ID NO: 840, SEQ ID NO: 841, SEQ ID NO: 842, SEQ ID NO: 843, SEQ ID NO: 844, SEQ ID NO: 845, SEQ ID NO: 846, SEQ ID NO: 847, SEQ ID NO: 848, SEQ ID NO: 849, SEQ ID NO: 850, SEQ ID NO: 851, SEQ ID NO: 852, SEQ ID NO: 853, SEQ ID NO: 854, SEQ ID NO: 855, SEQ ID NO: 856, SEQ ID NO: 857, SEQ ID NO: 858, SEQ ID NO: 859, SEQ ID NO: 860, SEQ ID NO: 861, SEQ ID NO: 862, SEQ ID NO: 863, SEQ ID NO: 864, SEQ ID NO: 865, SEQ ID NO: 866, SEQ ID NO: 867, SEQ ID NO: 868, SEQ ID NO: 869, SEQ ID NO: 870, SEQ ID NO: 871, SEQ ID NO: 872, SEQ ID NO: 873, SEQ ID NO: 874, SEQ ID NO: 875, SEQ ID NO: 876, SEQ ID NO: 877, SEQ ID NO: 878, SEQ ID NO: 879, SEQ ID NO: 880, SEQ ID NO: 881, SEQ ID NO: 882, SEQ ID NO: 883, SEQ ID NO: 884, SEQ ID NO: 885, SEQ ID NO: 886, SEQ ID NO: 887, SEQ ID NO: 888, SEQ ID NO: 889, SEQ ID NO: 890, SEQ ID NO: 891, SEQ ID NO: 892, SEQ ID NO: 893, SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, SEQ ID NO: 913, SEQ ID NO: 914, SEQ ID NO: 915, SEQ ID NO: 916, SEQ ID NO: 917, SEQ ID NO: 918, SEQ ID NO: 919, SEQ ID NO: 920, SEQ ID NO: 921, SEQ ID NO: 922, SEQ ID NO: 923, SEQ ID NO: 924, SEQ ID NO: 925, SEQ ID NO: 926, SEQ ID NO: 927, SEQ ID NO: 928, SEQ ID NO: 929, SEQ ID NO: 930, SEQ ID NO: 931, SEQ ID NO: 932, SEQ ID NO: 933, SEQ ID NO: 934, SEQ ID NO: 935, SEQ ID NO: 936, SEQ ID NO: 937, SEQ ID NO: 938, SEQ ID NO: 939, SEQ ID NO: 940, SEQ ID NO: 941, SEQ ID NO: 942, SEQ ID NO: 943, SEQ ID NO: 944, SEQ ID NO: 945, SEQ ID NO: 946, SEQ ID NO: 947, SEQ ID NO: 948, SEQ ID NO: 949, SEQ ID NO: 950, SEQ ID NO: 951, SEQ ID NO: 952, SEQ ID NO: 953, SEQ ID NO: 954, SEQ ID NO: 955, SEQ ID NO: 956, SEQ ID NO: 957, SEQ ID NO: 958, SEQ ID NO: 959, SEQ ID NO: 960, or SEQ ID NOs: 3362-5698. In additional embodiments of this aspect, the CGI factor may include SEQ ID NO: 2182, SEQ ID NO: 2183, SEQ ID NO: 2184, SEQ ID NO: 2185, SEQ ID NO: 2186, SEQ ID NO: 2187, SEQ ID NO: 2188, SEQ ID NO: 2189, SEQ ID NO: 2194, SEQ ID NO: 2195, SEQ ID NO: 2196, SEQ ID NO: 2197, SEQ ID NO: 2198, SEQ ID NO: 2199, SEQ ID NO: 2200, SEQ ID NO: 2201, SEQ ID NO: 2202, SEQ ID NO: 2203, SEQ ID NO: 2204, SEQ ID NO: 2205, SEQ ID NO: 2206, SEQ ID NO: 2207, SEQ ID NO: 2208, SEQ ID NO: 2209, SEQ ID NO: 2210, SEQ ID NO: 2215, SEQ ID NO: 2216, SEQ ID NO: 2217, SEQ ID NO: 2218, SEQ ID NO: 2219, SEQ ID NO: 2220, SEQ ID NO: 2221, SEQ ID NO: 2222, SEQ ID NO: 2223, SEQ ID NO: 2224, SEQ ID NO: 2225, SEQ ID NO: 2226, SEQ ID NO: 2227, SEQ ID NO: 2228, SEQ ID NO: 2229, SEQ ID NO: 2230, SEQ ID NO: 2231, SEQ ID NO: 2232, SEQ ID NO: 2233, SEQ ID NO: 2234, SEQ ID NO: 2235, SEQ ID NO: 2236, SEQ ID NO: 2237, SEQ ID NO: 2238, SEQ ID NO: 2239, SEQ ID NO: 2240, SEQ ID NO: 2241, SEQ ID NO: 2242, or SEQ ID NO: 2243. In some embodiments of this aspect, the CGI factor motif includes at least one of SEQ ID NO: 1921, SEQ ID NO: 1922, SEQ ID NO: 1923, SEQ ID NO: 1924, SEQ ID NO: 1925, SEQ ID NO: 1926, SEQ ID NO: 1927, SEQ ID NO: 1928, SEQ ID NO: 1929, SEQ ID NO: 1930, SEQ ID NO: 1931, SEQ ID NO: 1932, SEQ ID NO: 1933, SEQ ID NO: 1934, SEQ ID NO: 1935, SEQ ID NO: 1936, SEQ ID NO: 1937, SEQ ID NO: 1938, SEQ ID NO: 1939, SEQ ID NO: 1940, SEQ ID NO: 1941, SEQ ID NO: 1942, SEQ ID NO: 1943, SEQ ID NO: 1944, SEQ ID NO: 1945, SEQ ID NO: 1946, SEQ ID NO: 1947, SEQ ID NO: 1948, SEQ ID NO: 1949, SEQ ID NO: 1950, SEQ ID NO: 1951, SEQ ID NO: 1952, SEQ ID NO: 1953, SEQ ID NO: 1954, SEQ ID NO: 1955, or SEQ ID NO: 1956. In still further embodiments of this aspect, the CGI factor may include SEQ ID NO: 2457, SEQ ID NO: 2458, SEQ ID NO: 2459, SEQ ID NO: 2460, SEQ ID NO: 2461, SEQ ID NO: 2462, SEQ ID NO: 2463, SEQ ID NO: 2464, SEQ ID NO: 2465, SEQ ID NO: 2466, SEQ ID NO: 2467, SEQ ID NO: 2468, SEQ ID NO: 2469, SEQ ID NO: 2470, SEQ ID NO: 2471, SEQ ID NO: 2472, SEQ ID NO: 2473, SEQ ID NO: 2474, SEQ ID NO: 2475, SEQ ID NO: 2476, SEQ ID NO: 2477, SEQ ID NO: 2478, SEQ ID NO: 2479, SEQ ID NO: 2480, SEQ ID NO: 2481, SEQ ID NO: 2482, SEQ ID NO: 2483, SEQ ID NO: 2484, SEQ ID NO: 2485, SEQ ID NO: 2486, SEQ ID NO: 2487, SEQ ID NO: 2488, SEQ ID NO: 2489, SEQ ID NO: 2490, SEQ ID NO: 2491, SEQ ID NO: 2492, SEQ ID NO: 2493, SEQ ID NO: 2494, SEQ ID NO: 2495, SEQ ID NO: 2496, SEQ ID NO: 2497, SEQ ID NO: 2498, SEQ ID NO: 2499, SEQ ID NO: 2500, SEQ ID NO: 2501, SEQ ID NO: 2502, SEQ ID NO: 2503, SEQ ID NO: 2504, SEQ ID NO: 2505, SEQ ID NO: 2506, SEQ ID NO: 2507, SEQ ID NO: 2508, SEQ ID NO: 2509, SEQ ID NO: 2510, SEQ ID NO: 2511, SEQ ID NO: 2512, SEQ ID NO: 2513, SEQ ID NO: 2514, SEQ ID NO: 2515, SEQ ID NO: 2516, SEQ ID NO: 2517, SEQ ID NO: 2518, SEQ ID NO: 2519, SEQ ID NO: 2520, SEQ ID NO: 2521, SEQ ID NO: 2522, SEQ ID NO: 2523, SEQ ID NO: 2524, SEQ ID NO: 2525, SEQ ID NO: 2526, SEQ ID NO: 2527, SEQ ID NO: 2528, SEQ ID NO: 2529, SEQ ID NO: 2530, SEQ ID NO: 2531, SEQ ID NO: 2532, SEQ ID NO: 2533, SEQ ID NO: 2534, SEQ ID NO: 2535, SEQ ID NO: 2536, SEQ ID NO: 2537, SEQ ID NO: 2538, SEQ ID NO: 2539, SEQ ID NO: 2540, SEQ ID NO: 2541, SEQ ID NO: 2542, SEQ ID NO: 2543, SEQ ID NO: 2544, SEQ ID NO: 2545, SEQ ID NO: 2546, SEQ ID NO: 2547, SEQ ID NO: 2548, SEQ ID NO: 2549, SEQ ID NO: 2550, SEQ ID NO: 2551, SEQ ID NO: 2552, SEQ ID NO: 2553, SEQ ID NO: 2554, SEQ ID NO: 2555, SEQ ID NO: 2556, SEQ ID NO: 2557, SEQ ID NO: 2558, SEQ ID NO: 2559, SEQ ID NO: 2560, SEQ ID NO: 2561, SEQ ID NO: 2562, SEQ ID NO: 2563, SEQ ID NO: 2564, SEQ ID NO: 2565, SEQ ID NO: 2566, SEQ ID NO: 2567, SEQ ID NO: 2568, SEQ ID NO: 2569, SEQ ID NO: 2570, SEQ ID NO: 2571, SEQ ID NO: 2572, SEQ ID NO: 2573, SEQ ID NO: 2574, SEQ ID NO: 2575, SEQ ID NO: 2576, SEQ ID NO: 2577, SEQ ID NO: 2578, SEQ ID NO: 2579, SEQ ID NO: 2580, SEQ ID NO: 2581, SEQ ID NO: 2582, SEQ ID NO: 2583, SEQ ID NO: 2584, SEQ ID NO: 2585, SEQ ID NO: 2586, SEQ ID NO: 2587, SEQ ID NO: 2588, SEQ ID NO: 2589, SEQ ID NO: 2590, SEQ ID NO: 2591, SEQ ID NO: 2592, SEQ ID NO: 2593, SEQ ID NO: 2594, SEQ ID NO: 2595, SEQ ID NO: 2596, SEQ ID NO: 2597, SEQ ID NO: 2598, SEQ ID NO: 2599, SEQ ID NO: 2600, SEQ ID NO: 2601, SEQ ID NO: 2602, SEQ ID NO: 2603, SEQ ID NO: 2604, SEQ ID NO: 2605, SEQ ID NO: 2606, SEQ ID NO: 2607, SEQ ID NO: 2608, SEQ ID NO: 2609, SEQ ID NO: 2610, SEQ ID NO: 2611, SEQ ID NO: 2612, SEQ ID NO: 2613, SEQ ID NO: 2614, SEQ ID NO: 2615, SEQ ID NO: 2616, SEQ ID NO: 2617, SEQ ID NO: 2618, SEQ ID NO: 2619, SEQ ID NO: 2620, SEQ ID NO: 2621, SEQ ID NO: 2622, SEQ ID NO: 2623, SEQ ID NO: 2624, SEQ ID NO: 2625, SEQ ID NO: 2626, SEQ ID NO: 2627, SEQ ID NO: 2628, SEQ ID NO: 2629, SEQ ID NO: 2630, SEQ ID NO: 2631, SEQ ID NO: 2632, SEQ ID NO: 2633, SEQ ID NO: 2634, SEQ ID NO: 2635, SEQ ID NO: 2636, SEQ ID NO: 2637, SEQ ID NO: 2638, SEQ ID NO: 2639, SEQ ID NO: 2640, SEQ ID NO: 2641, SEQ ID NO: 2642, SEQ ID NO: 2643, SEQ ID NO: 2644, SEQ ID NO: 2645, SEQ ID NO: 2646, SEQ ID NO: 2647, SEQ ID NO: 2648, SEQ ID NO: 2649, SEQ ID NO: 2650, SEQ ID NO: 2651, SEQ ID NO: 2652, SEQ ID NO: 2653, SEQ ID NO: 2654, SEQ ID NO: 2655, SEQ ID NO: 2656, SEQ ID NO: 2657, SEQ ID NO: 2658, SEQ ID NO: 2659, SEQ ID NO: 2660, SEQ ID NO: 2661, SEQ ID NO: 2662, SEQ ID NO: 2663, SEQ ID NO: 2664, SEQ ID NO: 2665, SEQ ID NO: 2666, SEQ ID NO: 2667, SEQ ID NO: 2668, SEQ ID NO: 2669, SEQ ID NO: 2670, SEQ ID NO: 2671, SEQ ID NO: 2672, SEQ ID NO: 2673, SEQ ID NO: 2674, SEQ ID NO: 2675, SEQ ID NO: 2676, SEQ ID NO: 2677, SEQ ID NO: 2678, SEQ ID NO: 2679, SEQ ID NO: 2680, SEQ ID NO: 2681, SEQ ID NO: 2682, SEQ ID NO: 2683, SEQ ID NO: 2684, SEQ ID NO: 2685, SEQ ID NO: 2686, SEQ ID NO: 2687, SEQ ID NO: 2688, SEQ ID NO: 2689, SEQ ID NO: 2690, SEQ ID NO: 2691, SEQ ID NO: 2692, SEQ ID NO: 2693, SEQ ID NO: 2694, SEQ ID NO: 2695, SEQ ID NO: 2696, SEQ ID NO: 2697, SEQ ID NO: 2698, SEQ ID NO: 2699, SEQ ID NO: 2700, SEQ ID NO: 2701, SEQ ID NO: 2702, SEQ ID NO: 2703, SEQ ID NO: 2704, SEQ ID NO: 2705, SEQ ID NO: 2706, SEQ ID NO: 2707, SEQ ID NO: 2708, SEQ ID NO: 2709, SEQ ID NO: 2710, SEQ ID NO: 2711, SEQ ID NO: 2712, SEQ ID NO: 2713, SEQ ID NO: 2714, SEQ ID NO: 2715, SEQ ID NO: 2716, SEQ ID NO: 2717, SEQ ID NO: 2718, SEQ ID NO: 2719, SEQ ID NO: 2720, SEQ ID NO: 2721, SEQ ID NO: 2722, SEQ ID NO: 2723, SEQ ID NO: 2724, SEQ ID NO: 2725, SEQ ID NO: 2726, SEQ ID NO: 2727, SEQ ID NO: 2728, SEQ ID NO: 2729, SEQ ID NO: 2730, SEQ ID NO: 2731, SEQ ID NO: 2732, SEQ ID NO: 2733, SEQ ID NO: 2734, SEQ ID NO: 2735, SEQ ID NO: 2736, SEQ ID NO: 2737, SEQ ID NO: 2738, SEQ ID NO: 2739, SEQ ID NO: 2740, SEQ ID NO: 2741, SEQ ID NO: 2742, SEQ ID NO: 2743, SEQ ID NO: 2744, SEQ ID NO: 2745, SEQ ID NO: 2746, SEQ ID NO: 2747, SEQ ID NO: 2748, SEQ ID NO: 2749, SEQ ID NO: 2750, SEQ ID NO: 2751, SEQ ID NO: 2752, SEQ ID NO: 2753, SEQ ID NO: 2754, SEQ ID NO: 2755, SEQ ID NO: 2756, SEQ ID NO: 2757, SEQ ID NO: 2758, SEQ ID NO: 2759, SEQ ID NO: 2760, SEQ ID NO: 2761, SEQ ID NO: 2762, SEQ ID NO: 2763, SEQ ID NO: 2764, SEQ ID NO: 2765, SEQ ID NO: 2766, SEQ ID NO: 2767, SEQ ID NO: 2768, SEQ ID NO: 2769, SEQ ID NO: 2770, SEQ ID NO: 2771, SEQ ID NO: 2772, SEQ ID NO: 2773, SEQ ID NO: 2774, SEQ ID NO: 2775, SEQ ID NO: 2776, SEQ ID NO: 2777, SEQ ID NO: 2778, SEQ ID NO: 2779, SEQ ID NO: 2780, SEQ ID NO: 2781, SEQ ID NO: 2782, SEQ ID NO: 2783, SEQ ID NO: 2784, SEQ ID NO: 2785, SEQ ID NO: 2786, SEQ ID NO: 2787, SEQ ID NO: 2788, SEQ ID NO: 2789, SEQ ID NO: 2790, SEQ ID NO: 2791, SEQ ID NO: 2792, SEQ ID NO: 2793, SEQ ID NO: 2794, SEQ ID NO: 2795, SEQ ID NO: 2796, SEQ ID NO: 2797, SEQ ID NO: 2798, SEQ ID NO: 2799, SEQ ID NO: 2800, SEQ ID NO: 2801, SEQ ID NO: 2802, SEQ ID NO: 2803, SEQ ID NO: 2804, SEQ ID NO: 2805, SEQ ID NO: 2806, SEQ ID NO: 2807, SEQ ID NO: 2808, SEQ ID NO: 2809, SEQ ID NO: 2810, SEQ ID NO: 2811, SEQ ID NO: 2812, SEQ ID NO: 2813, SEQ ID NO: 2814, SEQ ID NO: 2815, SEQ ID NO: 2816, SEQ ID NO: 2817, SEQ ID NO: 2818, SEQ ID NO: 2819, SEQ ID NO: 2820, SEQ ID NO: 2821, SEQ ID NO: 2822, SEQ ID NO: 2823, SEQ ID NO: 2824, SEQ ID NO: 2825, SEQ ID NO: 2826, SEQ ID NO: 2827, SEQ ID NO: 2828, SEQ ID NO: 2829, SEQ ID NO: 2830, SEQ ID NO: 2831, SEQ ID NO: 2832, SEQ ID NO: 2833, SEQ ID NO: 2834, SEQ ID NO: 2835, SEQ ID NO: 2836, SEQ ID NO: 2837, SEQ ID NO: 2838, SEQ ID NO: 2839, SEQ ID NO: 2840, SEQ ID NO: 2841, SEQ ID NO: 2842, SEQ ID NO: 2843, SEQ ID NO: 2844, SEQ ID NO: 2845, SEQ ID NO: 2846, SEQ ID NO: 2847, SEQ ID NO: 2848, SEQ ID NO: 2849, SEQ ID NO: 2850, SEQ ID NO: 2851, SEQ ID NO: 2852, SEQ ID NO: 2853, SEQ ID NO: 2854, SEQ ID NO: 2855, SEQ ID NO: 2856, SEQ ID NO: 2857, SEQ ID NO: 2858, SEQ ID NO: 2859, SEQ ID NO: 2860, SEQ ID NO: 2861, SEQ ID NO: 2862, SEQ ID NO: 2863, SEQ ID NO: 2864, SEQ ID NO: 2865, SEQ ID NO: 2866, SEQ ID NO: 2867, SEQ ID NO: 2868, SEQ ID NO: 2869, SEQ ID NO: 2870, SEQ ID NO: 2871, SEQ ID NO: 2872, SEQ ID NO: 2873, SEQ ID NO: 2874, SEQ ID NO: 2875, SEQ ID NO: 2876, SEQ ID NO: 2877, SEQ ID NO: 2878, SEQ ID NO: 2879, SEQ ID NO: 2880, SEQ ID NO: 2881, SEQ ID NO: 2882, SEQ ID NO: 2883, SEQ ID NO: 2884, SEQ ID NO: 2885, SEQ ID NO: 2886, SEQ ID NO: 2887, SEQ ID NO: 2888, SEQ ID NO: 2889, SEQ ID NO: 2890, SEQ ID NO: 2891, SEQ ID NO: 2892, SEQ ID NO: 2893, SEQ ID NO: 2894, SEQ ID NO: 2895, SEQ ID NO: 2896, SEQ ID NO: 2897, SEQ ID NO: 2898, SEQ ID NO: 2899, SEQ ID NO: 2900, SEQ ID NO: 2901, SEQ ID NO: 2902, SEQ ID NO: 2903, SEQ ID NO: 2904, SEQ ID NO: 2905, SEQ ID NO: 2906, SEQ ID NO: 2907, SEQ ID NO: 2908, SEQ ID NO: 2909, SEQ ID NO: 2910, SEQ ID NO: 2911, SEQ ID NO: 2912, SEQ ID NO: 2913, SEQ ID NO: 2914, SEQ ID NO: 2915, SEQ ID NO: 2916, SEQ ID NO: 2917, SEQ ID NO: 2918, SEQ ID NO: 2919, SEQ ID NO: 2920, SEQ ID NO: 2921, SEQ ID NO: 2922, SEQ ID NO: 2923, SEQ ID NO: 2924, SEQ ID NO: 2925, SEQ ID NO: 2926, SEQ ID NO: 2927, SEQ ID NO: 2928, SEQ ID NO: 2929, SEQ ID NO: 2930, SEQ ID NO: 2931, SEQ ID NO: 2932, SEQ ID NO: 2933, SEQ ID NO: 2934, SEQ ID NO: 2935, SEQ ID NO: 2936, SEQ ID NO: 2937, SEQ ID NO: 2938, SEQ ID NO: 2939, SEQ ID NO: 2940, SEQ ID NO: 2941, SEQ ID NO: 2942, SEQ ID NO: 2943, SEQ ID NO: 2944, SEQ ID NO: 2945, SEQ ID NO: 2946, SEQ ID NO: 2947, SEQ ID NO: 2948, SEQ ID NO: 2949, SEQ ID NO: 2950, SEQ ID NO: 2951, SEQ ID NO: 2952, SEQ ID NO: 2953, SEQ ID NO: 2954, SEQ ID NO: 2955, SEQ ID NO: 2956, SEQ ID NO: 2957, SEQ ID NO: 2958, SEQ ID NO: 2959, SEQ ID NO: 2960, SEQ ID NO: 2961, SEQ ID NO: 2962, SEQ ID NO: 2963, SEQ ID NO: 2964, SEQ ID NO: 2965, SEQ ID NO: 2966, SEQ ID NO: 2967, SEQ ID NO: 2968, SEQ ID NO: 2969, SEQ ID NO: 2970, SEQ ID NO: 2971, SEQ ID NO: 2972, SEQ ID NO: 2973, SEQ ID NO: 2974, SEQ ID NO: 2975, SEQ ID NO: 2976, SEQ ID NO: 2977, SEQ ID NO: 2978, SEQ ID NO: 2979, SEQ ID NO: 2980, SEQ ID NO: 2981, SEQ ID NO: 2982, SEQ ID NO: 2983, SEQ ID NO: 2984, SEQ ID NO: 2985, SEQ ID NO: 2986, SEQ ID NO: 2987, SEQ ID NO: 2988, SEQ ID NO: 2989, SEQ ID NO: 2990, SEQ ID NO: 2991, SEQ ID NO: 2992, SEQ ID NO: 2993, SEQ ID NO: 2994, SEQ ID NO: 2995, SEQ ID NO: 2996, SEQ ID NO: 2997, SEQ ID NO: 2998, SEQ ID NO: 2999, SEQ ID NO: 3000, SEQ ID NO: 3001, SEQ ID NO: 3002, SEQ ID NO: 3003, SEQ ID NO: 3004, SEQ ID NO: 3005, SEQ ID NO: 3006, SEQ ID NO: 3007, SEQ ID NO: 3008, SEQ ID NO: 3009, SEQ ID NO: 3010, SEQ ID NO: 3011, SEQ ID NO: 3012, SEQ ID NO: 3013, SEQ ID NO: 3014, SEQ ID NO: 3015, SEQ ID NO: 3016, SEQ ID NO: 3017, SEQ ID NO: 3018, SEQ ID NO: 3019, SEQ ID NO: 3020, SEQ ID NO: 3021, SEQ ID NO: 3022, SEQ ID NO: 3023, SEQ ID NO: 3024, SEQ ID NO: 3025, SEQ ID NO: 3026, SEQ ID NO: 3027, SEQ ID NO: 3028, SEQ ID NO: 3029, SEQ ID NO: 3030, SEQ ID NO: 3031, SEQ ID NO: 3032, SEQ ID NO: 3033, SEQ ID NO: 3034, SEQ ID NO: 3035, SEQ ID NO: 3036, SEQ ID NO: 3037, SEQ ID NO: 3038, SEQ ID NO: 3039, SEQ ID NO: 3040, SEQ ID NO: 3041, SEQ ID NO: 3042, SEQ ID NO: 3043, SEQ ID NO: 3044, SEQ ID NO: 3045, SEQ ID NO: 3046, SEQ ID NO: 3047, SEQ ID NO: 3048, SEQ ID NO: 3049, SEQ ID NO: 3050, SEQ ID NO: 3051, SEQ ID NO: 3052, SEQ ID NO: 3053, SEQ ID NO: 3054, SEQ ID NO: 3055, SEQ ID NO: 3056, SEQ ID NO: 3057, SEQ ID NO: 3058, SEQ ID NO: 3059, SEQ ID NO: 3060, SEQ ID NO: 3061, SEQ ID NO: 3062, SEQ ID NO: 3063, SEQ ID NO: 3064, SEQ ID NO: 3065, SEQ ID NO: 3066, SEQ ID NO: 3067, SEQ ID NO: 3068, SEQ ID NO: 3069, SEQ ID NO: 3070, SEQ ID NO: 3071, SEQ ID NO: 3072, SEQ ID NO: 3073, SEQ ID NO: 3074, SEQ ID NO: 3075, SEQ ID NO: 3076, SEQ ID NO: 3077, SEQ ID NO: 3078, SEQ ID NO: 3079, SEQ ID NO: 3080, SEQ ID NO: 3081, SEQ ID NO: 3082, SEQ ID NO: 3083, SEQ ID NO: 3084, SEQ ID NO: 3085, SEQ ID NO: 3086, SEQ ID NO: 3087, SEQ ID NO: 3088, SEQ ID NO: 3089, SEQ ID NO: 3090, SEQ ID NO: 3091, SEQ ID NO: 3092, SEQ ID NO: 3093, SEQ ID NO: 3094, SEQ ID NO: 3095, SEQ ID NO: 3096, SEQ ID NO: 3097, SEQ ID NO: 3098, SEQ ID NO: 3099, SEQ ID NO: 3100, SEQ ID NO: 3101, SEQ ID NO: 3102, SEQ ID NO: 3103, SEQ ID NO: 3104, SEQ ID NO: 3105, SEQ ID NO: 3106, SEQ ID NO: 3107, SEQ ID NO: 3108, SEQ ID NO: 3109, SEQ ID NO: 3110, SEQ ID NO: 3111, SEQ ID NO: 3112, SEQ ID NO: 3113, SEQ ID NO: 3114, SEQ ID NO: 3115, SEQ ID NO: 3116, SEQ ID NO: 3117, SEQ ID NO: 3118, SEQ ID NO: 3119, SEQ ID NO: 3120, SEQ ID NO: 3121, SEQ ID NO: 3122, SEQ ID NO: 3123, SEQ ID NO: 3124, SEQ ID NO: 3125, SEQ ID NO: 3126, SEQ ID NO: 3127, SEQ ID NO: 3128, SEQ ID NO: 3129, SEQ ID NO: 3130, SEQ ID NO: 3131, SEQ ID NO: 3132, SEQ ID NO: 3133, SEQ ID NO: 3134, SEQ ID NO: 3135, SEQ ID NO: 3136, SEQ ID NO: 3137, SEQ ID NO: 3138, SEQ ID NO: 3139, SEQ ID NO: 3140, SEQ ID NO: 3141, SEQ ID NO: 3142, SEQ ID NO: 3143, SEQ ID NO: 3144, SEQ ID NO: 3145, SEQ ID NO: 3146, SEQ ID NO: 3147, SEQ ID NO: 3148, SEQ ID NO: 3149, SEQ ID NO: 3150, SEQ ID NO: 3151, SEQ ID NO: 3152, SEQ ID NO: 3153, SEQ ID NO: 3154, SEQ ID NO: 3155, SEQ ID NO: 3156, SEQ ID NO: 3157, SEQ ID NO: 3158, SEQ ID NO: 3159, SEQ ID NO: 3160, SEQ ID NO: 3161, SEQ ID NO: 3162, SEQ ID NO: 3163, SEQ ID NO: 3164, SEQ ID NO: 3165, SEQ ID NO: 3166, SEQ ID NO: 3167, SEQ ID NO: 3168, SEQ ID NO: 3169, SEQ ID NO: 3170, SEQ ID NO: 3171, SEQ ID NO: 3172, SEQ ID NO: 3173, SEQ ID NO: 3174, SEQ ID NO: 3175, SEQ ID NO: 3176, SEQ ID NO: 3177, SEQ ID NO: 3178, SEQ ID NO: 3179, SEQ ID NO: 3180, SEQ ID NO: 3181, SEQ ID NO: 3182, SEQ ID NO: 3183, SEQ ID NO: 3184, SEQ ID NO: 3185, SEQ ID NO: 3186, SEQ ID NO: 3187, SEQ ID NO: 3188, SEQ ID NO: 3189, SEQ ID NO: 3190, SEQ ID NO: 3191, SEQ ID NO: 3192, SEQ ID NO: 3193, SEQ ID NO: 3194, SEQ ID NO: 3195, SEQ ID NO: 3196, SEQ ID NO: 3197, SEQ ID NO: 3198, SEQ ID NO: 3199, SEQ ID NO: 3200, SEQ ID NO: 3201, SEQ ID NO: 3202, SEQ ID NO: 3203, SEQ ID NO: 3204, SEQ ID NO: 3205, SEQ ID NO: 3206, SEQ ID NO: 3207, SEQ ID NO: 3208, SEQ ID NO: 3209, SEQ ID NO: 3210, SEQ ID NO: 3211, SEQ ID NO: 3212, SEQ ID NO: 3213, SEQ ID NO: 3214, SEQ ID NO: 3215, SEQ ID NO: 3216, SEQ ID NO: 3217, SEQ ID NO: 3218, SEQ ID NO: 3219, SEQ ID NO: 3220, SEQ ID NO: 3221, SEQ ID NO: 3222, SEQ ID NO: 3223, SEQ ID NO: 3224, SEQ ID NO: 3225, SEQ ID NO: 3226, SEQ ID NO: 3227, SEQ ID NO: 3228, SEQ ID NO: 3229, SEQ ID NO: 3230, SEQ ID NO: 3231, SEQ ID NO: 3232, SEQ ID NO: 3233, SEQ ID NO: 3234, SEQ ID NO: 3235, SEQ ID NO: 3236, SEQ ID NO: 3237, SEQ ID NO: 3238, SEQ ID NO: 3239, SEQ ID NO: 3240, SEQ ID NO: 3241, SEQ ID NO: 3242, SEQ ID NO: 3243, SEQ ID NO: 3244, SEQ ID NO: 3245, SEQ ID NO: 3246, SEQ ID NO: 3247, SEQ ID NO: 3248, SEQ ID NO: 3249, SEQ ID NO: 3250, SEQ ID NO: 3251, SEQ ID NO: 3252, SEQ ID NO: 3253, SEQ ID NO: 3254, SEQ ID NO: 3255, SEQ ID NO: 3256, SEQ ID NO: 3257, SEQ ID NO: 3258, SEQ ID NO: 3259, SEQ ID NO: 3260, SEQ ID NO: 3261, SEQ ID NO: 3262, SEQ ID NO: 3263, SEQ ID NO: 3264, SEQ ID NO: 3265, SEQ ID NO: 3266, SEQ ID NO: 3267, SEQ ID NO: 3268, SEQ ID NO: 3269, SEQ ID NO: 3270, SEQ ID NO: 3271, SEQ ID NO: 3272, SEQ ID NO: 3273, SEQ ID NO: 3274, SEQ ID NO: 3275, SEQ ID NO: 3276, SEQ ID NO: 3277, SEQ ID NO: 3278, SEQ ID NO: 3279, SEQ ID NO: 3280, SEQ ID NO: 3281, SEQ ID NO: 3282, SEQ ID NO: 3283, SEQ ID NO: 3284, SEQ ID NO: 3285, SEQ ID NO: 3286, SEQ ID NO: 3287, SEQ ID NO: 3288, SEQ ID NO: 3289, SEQ ID NO: 3290, SEQ ID NO: 3291, SEQ ID NO: 3292, SEQ ID NO: 3293, SEQ ID NO: 3294, SEQ ID NO: 3295, SEQ ID NO: 3296, SEQ ID NO: 3297, SEQ ID NO: 3298, SEQ ID NO: 3299, SEQ ID NO: 3300, SEQ ID NO: 3301, SEQ ID NO: 3302, SEQ ID NO: 3303, SEQ ID NO: 3304, SEQ ID NO: 3305, SEQ ID NO: 3306, SEQ ID NO: 3307, SEQ ID NO: 3308, SEQ ID NO: 3309, SEQ ID NO: 3310, SEQ ID NO: 3311, SEQ ID NO: 3312, SEQ ID NO: 3313, SEQ ID NO: 3314, SEQ ID NO: 3315, SEQ ID NO: 3316, SEQ ID NO: 3317, SEQ ID NO: 3318, SEQ ID NO: 3319, SEQ ID NO: 3320, SEQ ID NO: 3321, SEQ ID NO: 3322, SEQ ID NO: 3323, SEQ ID NO: 3324, SEQ ID NO: 3325, SEQ ID NO: 3326, SEQ ID NO: 3327, SEQ ID NO: 3328, SEQ ID NO: 3329, SEQ ID NO: 3330, SEQ ID NO: 3331, SEQ ID NO: 3332, SEQ ID NO: 3333, SEQ ID NO: 3334, SEQ ID NO: 3335, SEQ ID NO: 3336, SEQ ID NO: 3337, SEQ ID NO: 3338, SEQ ID NO: 3339, SEQ ID NO: 3340, SEQ ID NO: 3341, SEQ ID NO: 3342, SEQ ID NO: 3343, SEQ ID NO: 3344, SEQ ID NO: 3345, SEQ ID NO: 3346, SEQ ID NO: 3347, SEQ ID NO: 3348, SEQ ID NO: 3349, SEQ ID NO: 3350, SEQ ID NO: 3351, SEQ ID NO: 3352, SEQ ID NO: 3353, SEQ ID NO: 3354, SEQ ID NO: 3355, SEQ ID NO: 3356, SEQ ID NO: 3357, SEQ ID NO: 3358, SEQ ID NO: 3359, SEQ ID NO: 3360, or SEQ ID NO: 3361. In yet additional embodiments of this aspect, the CGI factor may include SEQ ID NO: 5707, SEQ ID NO: 5708, SEQ ID NO: 5709, SEQ ID NO: 5710, SEQ ID NO: 5711, SEQ ID NO: 5712, SEQ ID NO: 5713, SEQ ID NO: 5714, SEQ ID NO: 5715, SEQ ID NO: 5716, SEQ ID NO: 5717, SEQ ID NO: 5718, SEQ ID NO: 5719, SEQ ID NO: 5720, SEQ ID NO: 5721, SEQ ID NO: 5722, SEQ ID NO: 5723, SEQ ID NO: 5724, SEQ ID NO: 5725, SEQ ID NO: 5726, SEQ ID NO: 5727, SEQ ID NO: 5728, SEQ ID NO: 5729, SEQ ID NO: 5730, or SEQ ID NO: 5731. In further embodiments of this aspect, which may be combined with any preceding embodiment, the CGI factor, CGI factor precursor, or CGI factor fragment is active and/or toxic. In additional embodiments of this aspect, which may be combined with any preceding embodiment, the recombinant DNA construct includes (a) at least one copy of a CGI factor, (b) at least one copy each of two or more CGI factors, (c) at least one CGI factor precursor, (d) at least one CGI factor fragment, (e) at least one CGI factor motif, or (f) any combination of (a) to (e). In still further embodiments of this aspect, which may be combined with any preceding embodiment, the heterologous promoter is a bacterial promoter, a fungal promoter, an algal promoter, an animal promoter, or a plant promoter. In yet additional embodiments of this aspect, which may be combined with any preceding embodiment, the heterologous promoter is a plant expressible promoter, i.e., a promoter that is functional for driving expression in a plant cell. In some embodiments of this aspect, the plant expressible promoter is selected from the group of promoters of a ubiquitin promoter, a cestrum yellow virus promoter, a corn TrpA promoter, a OsMADS 6 promoter, a maize H3 histone promoter, a corn sucrose synthetase 1 promoter, a corn alcohol dehydrogenase 1 promoter, a corn heat shock protein promoter, a maize mtl promoter, a pea small subunit RuBP carboxylase promoter, a rice actin promoter, a rice cyclophilin promoter, a Ti plasmid mannopine synthase promoter, a Ti plasmid nopaline synthase promoter, a petunia chalcone isomerase promoter, a bean glycine rich protein 1 promoter, a potato patatin promoter, a lectin promoter, a CaMV 35S promoter, or a S-E9 small subunit RuBP carboxylase promoter. In some embodiments, the heterologous promoter is an inducible promoter, a tissue-specific promoter, a temporally specific promoter, or a developmentally specific promoter. Tissue-specific promoters are useful for limiting expression of the recombinant DNA construct and encoded CGI factor, CGI factor precursor, CGI factor fragment, or CGI factor motif to specific tissues (e.g., root, leaf, tuber, fruit, or seed) of a plant. In some embodiments, the heterologous promoter is a plant miRNA promoter, which can be inducible, tissue-specific, temporally specific, or developmentally specific; see, e.g., the tissue-specific promoters disclosed in U.S. Pat. No. 8,334,430 and the temporally specific promoters disclosed in U.S. Pat. No. 8,314,290. In some embodiments, the recombinant DNA construct includes further elements that are useful for expression control, such as expression-enhancing elements, transcript-stabilizing sequences, riboswitches, or recognition sites for miRNAs or siRNAs. For example, including a recognition site for a miRNA that is natively expressed in a specific tissue of a plant is expected to reduce or eliminate expression of the CGI factor, CGI factor precursor, CGI factor fragment, or CGI factor motif in that specific tissue. In additional embodiments of this aspect, the recombinant DNA construct further includes a nucleotide sequence encoding at least one secretion signal peptide functional in a cell. Still another aspect of the disclosure relates to a recombinant vector including the recombinant DNA construct of any one of the preceding embodiments. In some embodiments of this aspect, the vector includes a left T-DNA border and a right T-DNA border flanking the recombinant DNA construct. In some embodiments, the vector further comprises additional sequences flanking the recombinant DNA construct. The additional sequences may correspond to selectable markers, transposon ends, homologous arms, restriction sites, or other sequences suitable for downstream uses of the vector. In additional embodiments of this aspect, the vector is a bacterial, viral, or viroid vector.

A further aspect of the disclosure relates to an RNA transcript (e.g., a messenger RNA) resulting from the transcription of the recombinant DNA construct of any one of the preceding embodiments.

An additional aspect of the disclosure relates to a CGI factor, a CGI factor precursor, a CGI factor fragment, or a CGI factor motif encoded by the recombinant DNA construct of any one of the preceding embodiments.

### Transgenic Cells and Organisms

Yet another aspect of the disclosure relates to a transgenic cell including the recombinant vector of any one of the preceding claims. In further embodiments of this aspect, the cell is selected from a bacterial cell, a fungal cell, an algal cell, an animal cell, or a plant cell. In additional embodiments of this aspect, the transgenic cell is a plant cell. In some embodiments of this aspect, the plant cell is a dicot plant cell. In further embodiments of this aspect, the dicot plant cell is selected from the group of a soybean cell, a sunflower cell, a tomato cell, a potato cell, a Brassica spp. cell, a cotton cell, a sugar beet cell, or a tobacco cell. In additional embodiments of this aspect, the plant cell is a monocot plant cell. In further embodiments of this aspect, the monocot plant cell is selected from the group of a barley cell, a maize cell, an oat cell, a rice cell, a sorghum cell, a sugar cane cell, or a wheat cell. In yet another embodiment of this aspect, which may be combined with any of the preceding embodiments that has a transgenic cell, the CGI factor, the CGI factor precursor, the CGI factor fragment, or the CGI factor motif is (a) transiently expressed, or (b) stably expressed.

An additional aspect of the disclosure relates to a transgenic plant including the transgenic plant cell of any of the preceding embodiments. In some embodiments, the transgenic plant is chimeric, having some cells that are transgenic (e.g., expressing a recombinant DNA construct as disclosed herein) and some cells that are not transgenic. Related embodiments include a grafted plant, wherein the rootstock is transgenic (e.g., expressing a recombinant DNA construct as disclosed herein) and the grafted scion is not transgenic; or wherein the rootstock is not transgenic and the scion is transgenic. In embodiments, the modified genome is the nuclear genome of the plant; in other embodiments, the modified genome is the genome of the plant's chloroplasts or mitochondria. In some embodiments of this aspect, the transgenic plant is a dicot plant. In further embodiments of this aspect, the dicot plant is selected from the group of a soybean plant, a sunflower plant, a tomato plant, a Brassica spp. plant, a cotton plant, a sugar beet plant, or a tobacco plant. In additional embodiments of this aspect, the transgenic plant is a monocot plant. In further embodiments of this aspect, the monocot plant is selected from the group of a barley plant, a maize plant, an oat plant, a rice plant, a sorghum plant, a sugar cane plant, or a wheat plant. In yet another embodiment of this aspect, the plant has improved resistance to the fungal pathogen, in comparison to a control plant that does not include the transgenic plant cell. In still another embodiment of this aspect, the fungal pathogen is one of an Aspergillus species; Magnaporthe oryzae; Botrytis cinerea; a Puccinia species; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani. In another embodiment of this aspect, which may be combined with any of the preceding embodiments, the nucleotide sequence encoding the CGI factor, the CGI factor precursor, the CGI factor fragment, or the CGI factor motif does not occur in the genome of the fungal pathogen.

Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses. Examples of commercially important cultivated crops, trees, and plants include: alfalfa (Medicago sativa), almonds (Prunus dulcis), apples (Malus x domestica), apricots (Prunus armeniaca, P. brigantine, P. mandshurica, P. mume, P. sibirica), artichoke (Cynara cardunculus var. scolymus), asparagus (Asparagus officinalis), avocado (Persea americana), bananas (Musa spp.), barley (Hordeum vulgare), beans (Phaseolus spp.), blueberries and cranberries (Vaccinium spp.), Brazil nut (Bertholletia excelsa), cacao (Theobroma cacao), calamansi (Citrus x microcarpa), canola and rapeseed or oilseed rape, (Brassica napus), Polish canola (Brassica rapa), and related cruciferous vegetables including broccoli, kale, cabbage, and turnips (Brassica carinata, B. juncea, B. oleracea, B. napus, B. nigra, and B. rapa, and hybrids of these), carnation (Dianthus caryophyllus), carrots (Daucus carota sativus), cashew (Anacardium occidentale), cassava (Manihot esculentum), celery (Apium graveolens), cherry (Prunus avium), chestnut (Castanea spp.), chickpea or garbanzo (Cicer arietinum), chicory (Cichorium intybus), chili peppers and other capsicum peppers (Capsicum annuum, C. frutescens, C. chinense, C. pubescens, C. baccatum), chrysanthemums (Chrysanthemum spp.), citron (Citrus medica), coconut (Cocos nucifera), coffee (wild and domesticated Coffea spp. including Coffea arabica, Coffea canephora, and Coffea liberica), cotton (Gossypium hirsutum L.), cowpea (Vigna unguiculata and other Vigna spp.), fava beans (Vicia faba), cucumber (Cucumis sativus), currants and gooseberries (Ribes spp.), date (Phoenix dactylifera), duckweeds (family Lemnoideae), eggplant or aubergine (Solanum melongena), elderberries (Sambucus spp.), eucalyptus (Eucalyptus spp.), flax (Linum usitatissumum L.), geraniums (Pelargonium spp.), ginger (Zingiber officinale), ginseng (Panax spp.), grapefruit (Citrus x paradisi), grapes (Vitis spp.) including wine grapes (Vitis vinifera and hybrids thereof), guava (Psidium guajava), hazelnut (Corylus avellana, Corylus spp.), hemp and cannabis (Cannabis sativa and Cannabis spp.), hops (Humulus lupulus), horseradish (Armoracia rusticana), irises (Iris spp.), jackfruit (Artocarpus heterophyllus), kiwifruits (Actinidia spp.), kumquat (Citrus japonica), lemon (Citrus limon), lentil (Lens culinaris), lettuce (Lactuca sativa), limes (Citrus spp.), lychee (Litchi chinensis), macadamias (Macadamia spp.), maize or corn (Zea mays L.), mandarin (Citrus reticulata), mango (Mangifera indica), mangosteen (Garcinia mangostana), melon (Cucumis melo), millets (Setaria spp., Echinochloa spp., Eleusine spp., Panicum spp., Pennisetum spp.), oats (Avena sativa), oil palm (Ellis quineensis), okra (Abelmoschus esculentus), olive (Olea europaea), onion (Allium cepa) and other alliums (Allium spp.), orange (Citrus sinensis), papaya (Carica papaya), parsnip (Pastinaca sativa), passionfruit (Passiflora edulis), pecan (Carya illinoinensis), peaches and nectarines (Prunus persica), pear (Pyrus spp.), pea (Pisum sativum), peanut (Arachis hypogaea), peonies (Paeonia spp.), persimmons (Diospyros kaki, Diospyros spp.), petunias (Petunia spp.), pineapple (Ananas comosus), pistachio (Pistacia vera), plantains (Musa spp.), plum (Prunus domestica), poinsettia (Euphorbia pulcherrima), pomelo (Citrus maxima), poplar (Populus spp.), potato (Solanum tuberosum), pumpkins and squashes (Cucurbita pepo, C. maxima, C. moschata), quince (Cydonia oblonga), raspberries (Rubus idaeus, Rubus occidentalis, Rubus spp.), rhubarbs (Rheum spp.), rice (Oryza sativa L.), roses (Rosa spp.), rubber (Hevea brasiliensis), rye (Secale cereale), safflower (Carthamus tinctorius L), satsuma (Citrus unshiu), sesame seed (Sesame indium), sorghum (Sorghum bicolor), sour orange (Citrus x aurantium), soursop (Annona muricata), soybean (Glycine max L.), strawberries (Fragaria spp., Fragaria x ananassa), sugar beet (Beta vulgaris), sugarcanes (Saccharum spp.), sunflower (Helianthus annuus), sweet potato (Ipomoea batatas), tamarind (Tamarindus indica), tangerine (Citrus tangerina), tea (Camellia sinensis), tobacco (Nicotiana tabacum L.), tomatillo (Physalis philadelphica), tomato (Solanum lycopersicum or Lycopersicon esculentum), tulips (Tulipa spp.), walnuts (Juglans spp. L.), watermelon (Citrulus lanatus), wheat (Triticum aestivum), and yams (Discorea spp.). Wild relatives of domesticated plants are also of interest.

A further aspect of the disclosure relates to a transgenic seed of the transgenic plant of any of the preceding embodiments, wherein said seed includes the recombinant DNA construct.

An additional aspect of the disclosure relates to an F1 progeny plant having as at least one parent the transgenic plant of any of the preceding embodiments, wherein the F1 progeny plant includes any of the recombinant DNA constructs of the preceding embodiments.

Yet another aspect of the disclosure relates to a harvested product produced from the transgenic plant of any of the preceding embodiments, wherein the harvested product includes the recombinant DNA construct. In some embodiments of this aspect, the harvested product is a fruit, a leaf, a stem, a flower, a root, a tuber, or a seed.

An additional aspect of the disclosure relates to a plant having a genome that is modified to express a heterologous DNA sequence that encodes a polypeptide including at least one conidial germination-inhibiting (CGI) factor, CGI factor precursor, CGI factor fragment, or CGI factor motif optionally fused to at least one plant secretion signal peptide, wherein the CGI factor, the CGI factor precursor, the CGI factor fragment, or the CGI factor motif inhibits conidial germination, growth, or reproduction of a fungal pathogen of the plant and wherein the CGI factor includes an amino acid sequence that has at least 80% sequence identity with a sequence selected from the group of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif includes at least one of SEQ ID NO: 1921-1956, and wherein the plant has improved resistance to the fungal pathogen, in comparison to a control plant that does not express the heterologous DNA sequence. The nucleotide sequence encoding the at least one CGI factor may include SEQ ID NO: 1-960 or 3362-5698. In additional embodiments of this aspect, the nucleotide sequence of the at least one CGI factor, CGI factor precursor, CGI factor fragment, or CGI factor motif does not occur in the genome of the fungal pathogen. In further embodiments of this aspect, which may be combined with any preceding embodiment, the fungal pathogen is one of an Aspergillus species; Magnaporthe oryzae; Botrytis cinerea; a Puccinia species; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani.

Yet another aspect of the disclosure relates to a plant including a cell containing a recombinant DNA construct for expressing a CGI factor, a CGI factor precursor, a CGI factor fragment, or a CGI factor motif and including: a heterologous promoter that is functional in the cell and is operably linked to a nucleic acid molecule including (a) a nucleotide sequence that encodes at least one conidial germination-inhibiting (CGI) factor, CGI factor precursor, CGI factor fragment, or CGI factor motif, and (b) a nucleotide sequence encoding at least one secretion signal peptide functional in the cells; wherein the CGI factor includes an amino acid sequence that has at least 80% sequence identity with a sequence selected from the group of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif includes at least one of SEQ ID NO: 1921-1956; and wherein the nucleotide sequence encoding the at least one CGI factor, CGI factor precursor, CGI factor fragment, or CGI factor motif optionally includes at least one codon optimized for expression in the cell. The nucleotide sequence encoding the at least one CGI factor may include SEQ ID NO: 1-960 or 3362-5698. In some embodiments of this aspect, the CGI factor, the CGI factor precursor, the CGI factor fragment, or the CGI factor motif is active and/or toxic. In further embodiments of this aspect, which may be combined with any preceding embodiment, the cell is a cell of the plant. In additional embodiments of this aspect, which may be combined with any preceding embodiment that has a cell that is not a cell of the plant, the cell is a bacterial or a fungal cell in or on the plant. In some embodiments of this aspect, which may be combined with any preceding embodiment, the plant has improved resistance to the fungal pathogen, in comparison to a control plant that does not include the cell. In yet another embodiment of this aspect, the fungal pathogen is one of an Aspergillus species; Magnaporthe oryzae; Botrytis cinerea; a Puccinia species; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani. In still another embodiment of this aspect, which may be combined with any preceding embodiment, the nucleotide sequence encoding the at least one CGI factor, CGI factor precursor, CGI factor fragment, or CGI factor motif does not occur in the genome of the fungal pathogen.

### Compositions

Yet another aspect of the disclosure relates to an antifungal or fungicidal composition including the CGI factor, the CGI factor precursor, the CGI factor fragment, or the CGI factor motif of any one of the preceding embodiments and an agriculturally acceptable carrier. In some embodiments of this aspect, the composition is formulated as one of a seed treatment, a foliar spray treatment, a foliar drench treatment, a Ready-To-Use (RTU) formulation, a produce coating, a suspension concentrate, a tank-mix, an aerosol, a root dip, a drench, a fog, a soil treatment, an irrigation formulation, or a sprinkler formulation. In further embodiments of this aspect, the agriculturally acceptable carrier includes a solid carrier, a liquid carrier, a gel carrier, a suspension, or an emulsion. In additional embodiments of this aspect, the agriculturally acceptable carrier includes one or more of an adjuvant, an inert component, a dispersant, a surfactant, a tackifier, a binder, or a stabilizer. Adjuvants and other components useful in agricultural formulations are described, e.g., in the Compendium of Herbicidal Adjuvants, 13^{th} edition, 2016; available at siu-weeds[dot]com/adjuvants/index-adj[dot]html. In some embodiments of this aspect, such agricultural formulations further include one or more additional components, such as an herbicide, insecticide, nematicide, fungicide (other than the CGI factor, CGI factor precursor, CGI factor fragment, or CGI factor motifs herein disclosed), attractant, or bait. In some embodiments of this aspect, the composition is formulated for application to human-built structures (e.g., buildings, fencing, walls) or artifacts (e.g., furniture, clothing, fabrics) or for incorporation in materials useful for making human-built structures or artifacts. In some embodiments of this aspect, the composition is incorporated as an addition to food or feed, e.g., products processed from plants. In additional embodiments of this aspect, the compositions are formulated as slow-release or controlled-release formulations.

An additional aspect of the disclosure relates to an antifungal composition including an effective amount of at least one conidial germination-inhibiting (CGI) factor, CGI factor precursor, CGI factor fragment, or CGI factor motif, and that includes (a) an amino acid sequence of a CGI factor that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731; or (b) an amino acid sequence of a CGI factor motif; and a carrier. In some embodiments of this aspect, the CGI factor motif includes at least one of SEQ ID NO: 1921-1956. In additional embodiments of this aspect, which may be combined with any preceding embodiment, the CGI factor, CGI factor precursor, or CGI factor fragment is active and/or toxic. In further embodiments of this aspect, which may be combined with any preceding embodiment, the carrier is selected from an agriculturally acceptable carrier, or a pharmaceutically acceptable carrier. In additional embodiments of this aspect, which may be combined with any preceding embodiment, the composition is formulated as a liquid, a gel, an emulsion, a suspension, an encapsulation, a solid, a powder, a coating, a spray, a soil drench, granules, a seed coat, or a bait. In some embodiments of this aspect, such agricultural formulations further include one or more additional components, such as an herbicide, insecticide, nematicide, fungicide (other than the CGI factor, CGI factor precursor, CGI factor fragment, or CGI factor motifs herein disclosed), attractant, or bait. In some embodiments of this aspect, the composition is formulated for application to human-built structures (e.g., buildings, fencing, walls) or artifacts (e.g., furniture, clothing, fabrics) or for incorporation in materials useful for making human-built structures or artifacts. In some embodiments of this aspect, the composition is incorporated as an addition to food or feed, e.g., products processed from plants. In additional embodiments of this aspect, the compositions are formulated as slow-release or controlled-release formulations.

Still another aspect of the disclosure relates to a composition having antifungal properties, including a substrate or matrix that is complexed with at least one conidial germination-inhibiting (CGI) factor, CGI factor precursor, CGI factor fragment, or CGI factor motif that is active and/or toxic, wherein the CGI factor includes an amino acid sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif includes at least one of SEQ ID NO: 1921-1956. In some embodiments of this aspect, the CGI factor, the CGI factor precursor, the CGI factor fragment, or the CGI factor motif is active and/or toxic. In further embodiments of this aspect, which may be combined with any preceding embodiment, the complexation between the substrate or matrix with the at least one CGI factor, CGI factor precursor, CGI factor fragment, or CGI factor motif is through: (a) covalent bonding, (b) non-covalent bonding, or (c) a combination of (a) and (b). In some embodiments of this aspect, the substrate or matrix includes polypeptides. In additional embodiments of this aspect, which may be combined with any preceding embodiment that has a substrate or matrix, the substrate or matrix includes self-assembling peptides.

Any suitable substrate or matrix known to those in the art may be applied to the present disclosure. In some embodiments, the substrate or matrix comprises polypeptides. In some embodiments, the polypeptides are self-assembling peptides. In some embodiments, the self-assembling peptide is (M)(YEYK)ₙYEY (SEQ ID NO: 2194), where n=3 or n is between 3-10, and where methionine is the terminal and optional amino acid, is covalently or non-covalently linked to one or more CGI peptides. Self-assembling peptide have been known to those of ordinary skill in the art, as demonstrated by Miki et al. (2021) Nature Communications, 21:3412, DOI: 10.1038/s41467-021-23794-6, which is specifically and entirely incorporated by reference herein for everything it teaches. In some embodiments, the complexation between the substrate or matrix and at least one CGI peptide is through covalent bonding. In some embodiments, the complexation between the substrate or matrix and at least one CGI peptide is through non-covalent bonding. In some embodiments, the complexation between the substrate or matrix and at least one CGI peptide is through a combination of covalent bonding and non-covalent bonding.

### Methods of Providing Fungal Resistance

A further aspect of the disclosure related to methods of providing an organism with resistance to a fungal pathogen of the organism, including expressing in a cell of the organism the recombinant DNA construct of any one of the preceding embodiments, wherein the heterologous promoter is functional in the organism. In further embodiments of this aspect, the nucleotide sequence encoding the CGI factor, the CGI factor precursor, the CGI factor fragment, or the CGI factor motif does not occur in the genome of the fungal pathogen. In additional embodiments of this aspect, the recombinant DNA construct is introduced into the cell of the organism by (a) transfection; (b) by inheritance from a parent cell; or (c) by fusion with a donor cell including the recombinant DNA construct. In yet further embodiments of this aspect, the organism is a plant, and wherein the recombinant DNA construct is provided to the plant by (a) transformation, or (b) inheritance from at least one parent plant that contained the recombinant DNA construct. Transformation may be stable or transient. In still another embodiment of this aspect, the plant is selected from the group of a maize plant, a soybean plant, a wheat plant, a rice plant, a cotton plant, a potato plant, a tomato plant, a Brassica spp. plant, or a sugar beet plant.

An additional aspect of the disclosure relates to methods of providing an organism with resistance to a fungal pathogen of the organism, including contacting the organism with the vector of any of the preceding embodiments. In further embodiments of this aspect, the nucleotide sequence encoding the CGI factor, the CGI factor precursor, the CGI factor fragment, or the CGI factor motif does not occur in the genome of the fungal pathogen.

Still another aspect of the disclosure relates to methods of providing an organism with resistance to a fungal pathogen of the organism, including contacting the organism with the cell of any of the preceding embodiments. In some embodiments of this aspect, the nucleotide sequence encoding the CGI factor, the CGI factor precursor, the CGI factor fragment, or the CGI factor motif does not occur in the genome of the fungal pathogen.

Still another aspect of the disclosure related to methods of producing a disease-resistant plant, including: introducing into a plant the recombinant DNA construct of any one of the preceding embodiments, wherein the CGI factor, CGI factor precursor, the CGI factor fragment, or the CGI factor motif is expressed in the plant; editing the plant to express a CGI factor including an amino acid sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with at least one of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, a CGI factor precursor, or a CGI factor fragment; or editing the plant to express a CGI factor motif, thereby producing a disease-resistant transgenic plant. In some embodiments of this aspect, the CGI factor motif includes at least one of SEQ ID NO: 1921-1956. The nucleotide sequence encoding the at least one CGI factor may include SEQ ID NO: 1-960 or 3362-5698. In additional embodiments of this aspect, which may be combined with any preceding embodiment, the CGI factor, CGI factor precursor, or CGI factor fragment is active and/or toxic. In further embodiments of this aspect, which may be combined with any preceding embodiment, editing the plant is performed using zinc finger-nucleases (ZFNs), transcription activator-like effector nucleases (TALENs), oligonucleotide-directed mutagenesis (ODM), or a clustered regularly interspaced short palindromic repeats (CRISPR) nuclease (e.g., Cas9, Cas12), or by gene writing (see, e.g., PCT Patent Application Publication WO2020/047124). In still another embodiment of this aspect, which may be combined with any preceding embodiment, the introducing step is achieved by (a) transforming the plant; or (b) crossing a first plant including the recombinant DNA construct with a second plant. In yet another embodiment of this aspect, which may be combined with any preceding embodiment, the introducing step includes transforming the plant, and wherein transforming the plant includes bacterially mediated transformation, micro-projectile-mediated transformation, sonication, electroporation, nanoparticle-mediated transformation, or liposome- or spheroplast-mediated vector delivery. An additional embodiment of this aspect, which may be combined with any preceding embodiment, includes the plant being a maize plant, a soybean plant, a wheat plant, a rice plant, a cotton plant, a potato plant, a tomato plant, a Brassica spp. plant, or a sugar beet plant.

Some aspects of the disclosure relate to methods of providing an organism with resistance to a fungal pathogen of the organism, including contacting the organism with the antifungal composition of any one of the preceding embodiments. In further embodiments of this aspect, the amino acid sequence of the CGI factor is not that of an alpha pheromone natively expressed by the fungal pathogen, or the nucleotide sequence encoding the CGI factor, the CGI factor precursor, the CGI factor fragment, or the CGI factor motif does not occur in the genome of the fungal pathogen.

In some embodiments, the methods of the present disclosure provide resistance to a fungal pathogen selected from the group consisting of Aspergillus, Candida, Coccidioides, Histoplasma, or Blastomyces fungus. In some embodiments, the fungal pathogen is a Mucoromycotina fungus, a Candida species (e.g., C. albicans, C. tropicalis, C. krusei, C. glabrata and C. pseudotropicalis), an Aspergillus species (e.g., A. fumigatus, A. flavus and A. niger.), Magnaporthe oryzae, Botrytis cinerea, Puccinia spp., Fusarium graminearum, Fusarium oxysporum, Blumeria graminis, Mycosphaerella graminicola, Colletotrichum spp., Ustilago maydis, Melampsora lini, Phakopsora pachyrhizi, or Rhizoctonia solani. In some embodiments, the methods of the present disclosure provide resistance to a fungal pathogen of plants. In some embodiments, the fungal pathogen of plants is Magnaporthe oryzae; Botrytis cinerea; Puccinia spp.; Fusarium graminearum; Fusarium oxysporum; Blumeria graminis; Mycosphaerella graminicola; Colletotrichum spp.; Ustilago maydis; Melampsora lini; Phakopsora pachyrhizi; or Rhizoctonia solani.

### Methods of Controlling or Preventing Fungal Growth

A further aspect of the disclosure relates to methods of controlling a fungal pathogen, including delivering to the fungal pathogen or an environment thereof a composition including an effective amount of the CGI factor, the CGI factor precursor, the CGI factor fragment, or the CGI factor motif of any one of the preceding embodiments.

A further aspect of the disclosure relates to methods for controlling a fungal pathogen, the method including: applying, to the fungal pathogen or a locus containing the fungal pathogen, a composition including a conidial germination-inhibiting (CGI) factor, a CGI factor precursor, a CGI factor fragment, or a CGI factor motif derived from a least one of the fungal pathogen, a fungus in the same genus as the fungal pathogen, a fungus in a different genus than the fungal pathogen, or a mixture thereof. In some embodiments of this aspect, the CGI factor has an amino acid sequence selected from the group of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731 or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity thereto; or wherein the CGI factor motif includes at least one of SEQ ID NO: 1921-1956. The nucleotide sequence encoding the at least one CGI factor may include SEQ ID NO: 1-960 or 3362-5698. In additional embodiments of this aspect, which may be combined with any preceding embodiment, the CGI factor, the CGI factor precursor, the CGI factor fragment, or the CGI factor motif is active and/or toxic. As used herein, a CGI factor, CGI factor precursor, CGI factor fragment, or CGI factor motif “derived from” a fungal pathogen refers to a CGI factor, CGI factor precursor, CGI factor fragment, or CGI factor motif directly obtained (e.g., extracted) or indirectly obtained (e.g., a synthetic CGI factor or precursor thereof having a sequence that is based on one or more fungal pathogen CGI factor sequences) from a fungal pathogen. In some embodiments, the amino acid sequence of a CGI factor, CGI factor precursor, CGI factor fragment, or CGI factor motif “derived from” a fungal pathogen corresponds to the amino acid sequence of a naturally occurring fungal alpha pheromone peptide, e.g., has an amino acid sequence that is identical or near identical (e.g., >90% sequence identity) to a genomically encoded expressed or putative alpha pheromone peptide sequence of the fungal pathogen. In embodiments, the amino acid sequence of a CGI factor, CGI factor precursor, CGI factor fragment, or CGI factor motif “derived from” a fungal pathogen includes combinations of more than one naturally occurring fungal alpha pheromone peptide, e.g., homodimers of a single fungal alpha pheromone peptide or heterodimers of two different alpha pheromone peptides, or multimers of one or more alpha pheromone peptide, with or without additional amino acids (e.g., flanking or linking amino acids adjacent to or in between the alpha pheromone monomers). In other embodiments, the sequence of a CGI factor, CGI factor precursor, CGI factor fragment, or CGI factor motif “derived from” a fungal pathogen is shorter or longer relative to that sequence in the fungal pathogen. For example, a synthetic CGI factor or CGI factor precursor can include additional amino acids inserted into the sequence of a naturally occurring fungal alpha pheromone (or alpha pheromone precursor) sequence; in embodiments, the additional amino acids provide a desired characteristic or function, relative to a polypeptide lacking the additional amino acids, such as, but not limited to: increased solubility, modified charge, improved detectability or selectability (e.g., using a detectable or selectable sequence such as a reporter or epitope), increased stability, increased cell penetration, or modified cellular or tissue location (e.g., localization to a cellular organelle or to a particular tissue). In some embodiments, a synthetic CGI factor or CGI factor precursor includes sequence derived from multiple naturally occurring CGI factors identified from one or more fungal pathogens (e.g., a synthetic CGI factor that is a heterodimer or other multimer wherein the unit sequences are CGI factor sequences identified from different fungal species, optionally with linker amino acids joining the unit sequences). Additionally, the CGI factor, CGI factor precursor, CGI factor fragment, or CGI factor motif “derived from” a fungal pathogen may have additional amino acid residues added to it, for example, a linker sequence, a signal peptide sequence, or similar.

An additional aspect of the disclosure relates to methods of controlling growth or reproduction of a fungus, including providing the fungus with a composition including an effective amount of at least one conidial germination-inhibiting (CGI) factor, CGI factor precursor, CGI factor fragment, or CGI factor motif, wherein the CGI factor includes an amino acid sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif includes at least one of SEQ ID NO: 1921-1956, and wherein the amino acid sequence of the CGI factor is not that of an alpha pheromone natively expressed by the fungus, or wherein the nucleotide sequence of the at least one CGI factor, CGI factor precursor, CGI factor fragment, or CGI factor motif does not occur in the genome of the fungus. The nucleotide sequence encoding the at least one CGI factor may include SEQ ID NO: 1-960 or 3362-5698. In some embodiments of this aspect, the CGI factor, the CGI factor precursor, the CGI factor fragment, or the CGI factor motif is active and/or toxic. In further embodiments of this aspect, which may be combined with any preceding embodiment, the composition is provided to the fungus by directly contacting the fungus with the composition, or by delivering the composition to the environment of the fungus.

Yet another aspect of the disclosure relates to methods of preventing growth of a fungus on a surface, including treating the surface with a composition including an effective amount of at least one conidial germination-inhibiting (CGI) factor, CGI factor precursor, CGI factor fragment, or CGI factor motif, wherein the CGI factor includes an amino acid sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif includes at least one of SEQ ID NO: 1921-1956. The nucleotide sequence encoding the at least one CGI factor may include SEQ ID NO: 1-960 or 3362-5698. In some embodiments of this aspect, the CGI factor, the CGI factor precursor, the CGI factor fragment, or the CGI factor motif is active and/or toxic. In further embodiments of this aspect, which may be combined with any preceding embodiment, the surface is a non-living surface or is the surface of a living organism. In additional embodiments of this aspect, which may be combined with any preceding embodiment, the nucleotide sequence of the at least one CGI factor, CGI factor precursor, CGI factor fragment, or CGI factor motif does not occur in the genome of the fungus.

Also provided herein is a method of preventing growth of a fungus on a surface or within a structure (e.g., a human-built structure or artifact). In some embodiments, the method comprises treating the surface or structure with a composition (e.g., a paint, coating, spray, or dip) comprising an effective amount of at least one CGI factor, CGI factor precursor, CGI factor fragment, or CGI factor motif that inhibits growth or reproduction of the fungus. In some embodiments, the CGI factor comprises an amino acid sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 961-1920, or from the group consisting of SEQ ID NO: 1957-2189, or from the group consisting of SEQ ID NO: 2194-2210, or from the group consisting of SEQ ID NO: 2215-2243, or from the group consisting of SEQ ID NO: 2457-3361, or from the group consisting of SEQ ID NO: 5707-5731. In embodiments, the nucleotide sequence encoding the at least one CGI factor includes at least one sequence selected from the group consisting of SEQ ID NO: 1-960 or 3362-5698. In some embodiments, the DNA sequence of the at least one CGI factor, CGI factor precursor, CGI factor fragment, or CGI factor motif does not occur in the genome of the fungus.

In some embodiments, the surface is a non-living surface. In some embodiments, the surface is a surface of a living organism. In embodiments, the structure is a human-built structure or artifact, such as a building, fence, wall, furniture, fabric, or components thereof.

### Methods of Treating a Fungal Disease

A further aspect of the disclosure relates to methods of treating a subject with a fungal disease including administering to a subject an antifungal or fungicidal composition including the CGI factor, the CGI factor precursor, the CGI factor fragment, or the CGI factor motif of any of the preceding embodiments and a pharmaceutically acceptable carrier. In some embodiments of this aspect, the subject is a mammal; in other embodiments the subject is a vertebrate such as a bird, reptile, fish, or amphibian, or is an invertebrate such as an insect. In additional embodiments of this aspect, the mammal is a human. In further embodiments of this aspect, the mammal is a domestic animal or livestock. In still further embodiments of this aspect, which may be combined with any preceding embodiment, the fungal disease is caused by a fungal pathogen selected from the group of Aspergillus, Candida, Coccidioides, Histoplasma, Cryptococcus, Pneumocystis, or Blastomyces fungus. In some embodiments, the fungal disease is an infection of a Mucoromycotina fungus, a Candida species (e.g., C. albicans, C. auris, C. tropicalis, C. krusei, C. glabrata, C. parapsilosis, and C. pseudotropicalis), a Coccidioides species (e.g., C. immitis or C. posadasii), an Aspergillus species (e.g., A. fumigatus, A. flavus, and A. niger), a Mucor species, a Rhizomucor species, a Malassezia species (e.g., M. furfur, M. globose, and M. restricta), Magnaporthe oryzae, Botrytis cinerea, Puccinia spp., Fusarium graminearum; Fusarium oxysporum, Blumeria graminis, Mycosphaerella graminicola, Colletotrichum spp., Ustilago maydis; Melampsora lini, Phakopsora pachyrhizi, or Rhizoctonia solani. In yet another embodiment of this aspect, which may be combined with any preceding embodiment, the fungal disease is aspergillosis, blastomycosis, candidiasis, coccidioidomycosis, histoplasmosis, mucormycosis, mycetoma, ringworm, sporotrichosis, paracoccidioidomycosis, talaromycosis, chromoblastomycosis fusariosis, emergomycosis, scedosporiosis, or fungal meningitis. In additional embodiments of this aspect, which may be combined with any preceding embodiments, the antifungal or fungicidal composition is administered intravenously, intramuscularly, subcutaneously, topically, orally, transdermally, intraperitoneally, intraorbitally, by implantation, by inhalation, intrathecally, intraventricularly, or intranasally.

As used herein, the term “subject” refers to an organism, such as an animal, plant, or microbe. In some embodiments, the subject is a mammal. In some embodiments, the mammal is a human. In other embodiments, the subject is a domestic animal or livestock. In some embodiments, the subject is a non-mammal. In some embodiments, the non-mammal is a reptile, an insect, an amphibian, a bird, or a fish. In embodiments, the subject is a vertebrate animal (e.g., mammal, bird, cartilaginous or bony fish, reptile, or amphibian). In embodiments, the subject is a human; including adults and non-adults (infants and children). In embodiments, the subject is a non-human mammal, such as a non-human primate (e.g., monkeys, apes), ungulate (e.g., cattle, buffalo, bison, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g, insects, arachnids, crustaceans), a nematode, an annelid, a helminth, or a mollusc. In embodiments, the subject is an organism that is part of a symbiosis, such as part of the microbiome of an animal or a plant. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses. Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses.

### Enumerated Embodiments

1. A recombinant DNA construct comprising:
a heterologous promoter operably linked to a nucleic acid molecule comprising a nucleotide sequence that encodes a conidial germination-inhibiting (CGI) factor, a CGI factor precursor, or a CGI factor fragment,
wherein the nucleotide sequence
   (a) encodes at least one CGI factor comprising an amino acid sequence that has at least 80% sequence identity with at least one of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731 at least one CGI factor precursor, or at least one CGI factor fragment,
   (b) is a synthetic sequence of (a) that has codons optimized for heterologous expression; or
   (c) encodes at least one CGI factor motif.
      2. The recombinant DNA construct of embodiment 2, wherein the CGI factor motif comprises at least one of SEQ ID NO: 1921-1956.
      3. The recombinant DNA construct of embodiment 1 or embodiment 2, wherein the CGI factor, CGI factor precursor, or CGI factor fragment is active and/or toxic.
      4. The recombinant DNA construct of any one of embodiments 1-3, wherein the recombinant DNA construct comprises (a) at least one copy of a CGI factor, (b) at least one copy each of two or more CGI factors, (c) at least one CGI factor precursor, (d) at least one CGI factor fragment, (e) at least one CGI factor motif, or (f) any combination of (a) to (e).
      5. The recombinant DNA construct of any one of embodiments 1-4, wherein the heterologous promoter is a bacterial promoter, a fungal promoter, an algal promoter, an animal promoter, or a plant promoter.
      6. The recombinant DNA construct of any one of embodiments 1-5, wherein the heterologous promoter is a plant expressible promoter.
      7. The recombinant DNA construct of embodiment 6, wherein the plant expressible promoter is selected from the group of promoters consisting of a ubiquitin promoter, a cestrum yellow virus promoter, a corn TrpA promoter, a OsMADS 6 promoter, a maize H3 histone promoter, a corn sucrose synthetase 1 promoter, a corn alcohol dehydrogenase 1 promoter, a corn heat shock protein promoter, a maize mtl promoter, a pea small subunit RuBP carboxylase promoter, a rice actin promoter, a rice cyclophilin promoter, a Ti plasmid mannopine synthase promoter, a Ti plasmid nopaline synthase promoter, a petunia chalcone isomerase promoter, a bean glycine rich protein 1 promoter, a potato patatin promoter, a lectin promoter, a CaMV 35S promoter, and a S-E9 small subunit RuBP carboxylase promoter.
      8. The recombinant DNA construct of any one of embodiments 1-7, wherein the recombinant DNA construct further comprises a nucleotide sequence encoding at least one secretion signal peptide functional in a cell.
      9. A method of providing an organism with resistance to a fungal pathogen of the organism, comprising expressing in a cell of the organism the recombinant DNA construct of any one of embodiments 1-8, wherein the heterologous promoter is functional in the organism.
      10. The method of embodiment 9, wherein the nucleotide sequence encoding the CGI factor, the CGI factor precursor, the CGI factor fragment, or the CGI factor motif does not occur in the genome of the fungal pathogen.
      11. The method of embodiment 9, wherein the recombinant DNA construct is introduced into the cell of the organism by (a) transfection; (b) by inheritance from a parent cell; or (c) by fusion with a donor cell comprising the recombinant DNA construct.
      12. The method of embodiment 9, wherein the organism is a plant, and wherein the recombinant DNA construct is provided to the plant by (a) transformation, or (b) inheritance from at least one parent plant that contained the recombinant DNA construct.
      13. The method of embodiment 12, wherein the plant is selected from the group consisting of a maize plant, a soybean plant, a wheat plant, a rice plant, a cotton plant, a potato plant, a tomato plant, a Brassica spp. plant, and a sugar beet plant.
      14. A CGI factor, a CGI factor precursor, a CGI factor fragment, or a CGI factor motif encoded by the recombinant DNA construct of any one of embodiments 1-8.
      15. An antifungal or fungicidal composition comprising the CGI factor, the CGI factor precursor, the CGI factor fragment, or the CGI factor motif of embodiment 14 and an agriculturally acceptable carrier.
      16. The antifungal or fungicidal composition of embodiment 15, wherein the composition is formulated as one of a seed treatment, a foliar spray treatment, a foliar drench treatment, a Ready-To-Use (RTU) formulation, a produce coating, a suspension concentrate, a tank-mix, an aerosol, a root dip, a drench, a fog, a soil treatment, an irrigation formulation, or a sprinkler formulation. 17. The antifungal or fungicidal composition of embodiment 15, wherein the agriculturally acceptable carrier comprises a solid carrier, a liquid carrier, a gel carrier, a suspension, or an emulsion.
      18. The antifungal or fungicidal composition of embodiment 15, wherein the agriculturally acceptable carrier comprises one or more of an adjuvant, an inert component, a dispersant, a surfactant, a tackifier, a binder, or a stabilizer.
      19. A recombinant vector comprising the recombinant DNA construct of any one of embodiments 1-8.
      20. The vector of embodiment 19, wherein the vector comprises a left T-DNA border and a right T-DNA border flanking the recombinant DNA construct.
      21. The vector of embodiment 19, wherein the vector is a viral vector.
      22. A method of providing an organism with resistance to a fungal pathogen of the organism, comprising contacting the organism with the vector of embodiment 19.
      23. The method of embodiment 22, wherein the nucleotide sequence encoding the CGI factor, the CGI factor precursor, the CGI factor fragment, or the CGI factor motif does not occur in the genome of the fungal pathogen.
      24. An RNA transcript resulting from the transcription of the recombinant DNA construct of any one of embodiments 1-8.
      25. A transgenic cell comprising the recombinant vector of embodiment 19.
      26. The transgenic cell of embodiment 25, wherein the cell is selected from a bacterial cell, a fungal cell, an algal cell, an animal cell, or a plant cell.
      27. The transgenic cell of embodiment 26, wherein the transgenic cell is a plant cell.
      28. The transgenic cell of embodiment 27, wherein the plant cell is a dicot plant cell.
      29. The transgenic cell of embodiment 28, wherein the dicot plant cell is selected from the group consisting of a soybean cell, a sunflower cell, a tomato cell, a Brassica spp. cell, a cotton cell, a sugar beet cell, and a tobacco cell.
      30. The transgenic cell of embodiment 27, wherein the plant cell is a monocot plant cell.
      31. The transgenic cell of embodiment 30, wherein the monocot plant cell is selected from the group consisting of a barley cell, a maize cell, an oat cell, a rice cell, a sorghum cell, a sugar cane cell, and a wheat cell.
      32. The transgenic cell of any one of embodiments 25-31, wherein the CGI factor, the CGI factor precursor, the CGI factor fragment, or the CGI factor motif is (a) transiently expressed, or (b) stably expressed.
      33. A method of providing an organism with resistance to a fungal pathogen of the organism, comprising contacting the organism with the cell of embodiment 25.
      34. The method of embodiment 33, wherein the nucleotide sequence encoding the CGI factor, the CGI factor precursor, the CGI factor fragment, or the CGI factor motif does not occur in the genome of the fungal pathogen.
      35. A transgenic plant comprising the transgenic plant cell of embodiment 27.
      36. The transgenic plant of embodiment 35, wherein the transgenic plant is a dicot plant.
      37. The transgenic plant of embodiment 36, wherein the dicot plant is selected from the group consisting of a soybean plant, a sunflower plant, a tomato plant, a Brassica spp. plant, a cotton plant, a sugar beet plant, and a tobacco plant.
      38. The transgenic plant of embodiment 35, wherein the transgenic plant is a monocot plant.
      39. The transgenic plant of embodiment 38, wherein the monocot plant is selected from the group consisting of a barley plant, a maize plant, an oat plant, a rice plant, a sorghum plant, a sugar cane plant, and a wheat plant.
      40. The transgenic plant of embodiment 35, wherein the plant has improved resistance to the fungal pathogen, in comparison to a control plant that does not comprise the transgenic plant cell.
      41. The transgenic plant of embodiment 39, wherein the fungal pathogen is one of an Aspergillus species; Magnaporthe oryzae; Botrytis cinerea; a Puccinia species; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani.
      42. The transgenic plant of any one of embodiments 35-41, wherein the nucleotide sequence encoding the CGI factor, the CGI factor precursor, the CGI factor fragment, or the CGI factor motif does not occur in the genome of the fungal pathogen.
      43. A transgenic seed of the transgenic plant of embodiment 35, wherein said seed comprises the recombinant DNA construct.
      44. An F1 progeny plant having as at least one parent the transgenic plant of embodiment 35, wherein the F1 progeny plant comprises the recombinant DNA construct.
      45. A harvested product produced from the transgenic plant of embodiment 35, wherein the harvested product comprises the recombinant DNA construct. 46. The harvested product of embodiment 45, wherein the harvested product is a fruit, a leaf, a stem, a flower, a root, a tuber, or a seed.
      47. A method of producing a disease-resistant plant, comprising:
introducing into a plant the recombinant DNA construct of any one of embodiments 1-8, wherein the CGI factor, CGI factor precursor, the CGI factor fragment, or the CGI factor motif is expressed in the plant;
editing the plant to express a CGI factor comprising an amino acid sequence that has at least 80% sequence identity with at least one of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, a CGI factor precursor, or a CGI factor fragment; or
editing the plant to express a CGI factor motif,
thereby producing a disease-resistant transgenic plant that is resistant to diseases caused by a fungus or oomycete.
   48. The method of embodiment 47, wherein the CGI factor motif comprises at least one of SEQ ID NO: 1921-1956.
   49. The method of embodiment 47 or embodiment 48, wherein the CGI factor, CGI factor precursor, or CGI factor fragment inhibits growth of and/or is toxic to the fungus or oomycete.
   50. The method of any one of embodiments 47-49, wherein editing the plant is performed using zinc finger-nucleases (ZFNs), transcription activator-like effector nucleases (TALENs), oligonucleotide-directed mutagenesis (ODM), or a clustered regularly interspaced short palindromic repeats (CRISPR)/Cas nuclease.
   51. The method of any one of embodiments 47-49, wherein the introducing step is achieved by (a) transforming the plant; or (b) crossing a first plant comprising the recombinant DNA construct with a second plant.
   52. The method of any one of embodiments 47-49, wherein the introducing step comprises transforming the plant, and wherein transforming the plant comprises bacterially mediated transformation, micro-projectile-mediated transformation, sonication, electroporation, nanoparticle-mediated transformation, or liposome- or spheroplast-mediated vector delivery.
   53. The method of any one of embodiments 47-52, wherein the plant is a maize plant, a soybean plant, a wheat plant, a rice plant, a cotton plant, a potato plant, a tomato plant, a Brassica spp. plant, or a sugar beet plant.
   54. A method of controlling a fungal pathogen, comprising delivering to the fungal pathogen or an environment thereof a composition comprising an effective amount of the CGI factor, the CGI factor precursor, the CGI factor fragment, or the CGI factor motif of embodiment 14.
   55. An antifungal composition comprising
(a) an effective amount of at least one conidial germination-inhibiting (CGI) factor, CGI factor precursor, CGI factor fragment, or CGI factor motif, and that comprises
   (i) a polypeptide comprising an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731; or
   (ii) a polypeptide comprising the amino acid sequence of a CGI factor motif; and
(b) a carrier.
   56. The antifungal composition of embodiment 53, wherein the CGI factor motif comprises at least one of SEQ ID NO: 1921-1956.
   57. The antifungal composition of embodiment 55 or embodiment 56, wherein the CGI factor, CGI factor precursor, or CGI factor fragment is inhibits growth of and/or is toxic to a fungus or oomycete.
   58. The antifungal composition of any one of embodiments 55-57, wherein the carrier is selected from an agriculturally acceptable carrier, or a pharmaceutically acceptable carrier.
   59. The antifungal composition of any one of embodiments 55-58, wherein the composition is formulated as a liquid, a gel, an emulsion, a suspension, an encapsulation, a solid, a powder, a coating, a spray, a soil drench, granules, a seed coat, or a bait.
   60. A method of providing an organism with resistance to a fungal pathogen of the organism, comprising contacting the organism with the antifungal composition of any one of embodiments 55-59.
   61. The method of embodiment 60, wherein the nucleotide sequence encoding the CGI factor, the CGI factor precursor, the CGI factor fragment, or the CGI factor motif does not occur in the genome of the fungal pathogen.
   62. A method for controlling a fungal pathogen, the method comprising:
applying, to the fungal pathogen or a locus containing the fungal pathogen, a composition comprising a conidial germination-inhibiting (CGI) factor, a CGI factor precursor, a CGI factor fragment, or a CGI factor motif derived from a least one of the fungal pathogen, a fungus in the same genus as the fungal pathogen, a fungus in a different genus than the fungal pathogen, or a mixture thereof.
   63. The method of embodiment 62, wherein the CGI factor has an amino acid sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731 or an amino acid sequence having at least 80% sequence identity thereto; or wherein the CGI factor motif comprises at least one of SEQ ID NO: 1921-1956.
   64. The method of embodiment 62 or embodiment 63, wherein the CGI factor, the CGI factor precursor, the CGI factor fragment, or the CGI factor motif is active and/or toxic.
   65. A plant having a genome that is modified to express a heterologous DNA sequence that encodes a polypeptide comprising at least one conidial germination-inhibiting (CGI) factor, CGI factor precursor, CGI factor fragment, or CGI factor motif optionally fused to at least one plant secretion signal peptide, wherein the CGI factor, the CGI factor precursor, the CGI factor fragment, or the CGI factor motif inhibits conidial germination, growth, or reproduction of a fungal pathogen of the plant and wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif comprises at least one of SEQ ID NO: 1921-1956, and wherein the plant has improved resistance to the fungal pathogen, in comparison to a control plant that does not express the heterologous DNA sequence.
   66. The plant of embodiment 65, wherein the nucleotide sequence of the at least one CGI factor, CGI factor precursor, CGI factor fragment, or CGI factor motif does not occur in the genome of the fungal pathogen.
   67. The plant of embodiment 65 or embodiment 66, wherein the fungal pathogen is one of an Aspergillus species; Magnaporthe oryzae; Botrytis cinerea; a Puccinia species; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani.
   68. A plant comprising a cell containing a recombinant DNA construct for expressing a CGI factor, a CGI factor precursor, a CGI factor fragment, or a CGI factor motif and comprising:
a heterologous promoter that is functional in the cell and is operably linked to a nucleic acid molecule comprising (a) a nucleotide sequence that encodes at least one conidial germination-inhibiting (CGI) factor, CGI factor precursor, CGI factor fragment, or CGI factor motif, and (b) a nucleotide sequence encoding at least one secretion signal peptide functional in the cells;
wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif comprises at least one of SEQ ID NO: 1921-1956; and
wherein the nucleotide sequence encoding the at least one CGI factor, CGI factor precursor, CGI factor fragment, or CGI factor motif optionally includes at least one codon optimized for expression in the cell.
   69. The plant of embodiment 68, wherein the CGI factor, the CGI factor precursor, the CGI factor fragment, or the CGI factor motif is active and/or toxic.
   70. The plant of embodiment 68 or embodiment 69, wherein the cell is a cell of the plant.
   71. The plant of embodiment 68 or embodiment 69, wherein the cell is a bacterial or a fungal cell in or on the plant.
   72. The plant of any one of embodiments 68-71, wherein the plant has improved resistance to the fungal pathogen, in comparison to a control plant that does not comprise the cell.
   73. The plant of embodiment 72, wherein the fungal pathogen is one of an Aspergillus species; Magnaporthe oryzae; Botrytis cinerea; a Puccinia species; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani.
   74. The plant of any one of embodiments 68-73, wherein the nucleotide sequence encoding the at least one CGI factor, CGI factor precursor, CGI factor fragment, or CGI factor motif does not occur in the genome of the fungal pathogen.
   75. A method of controlling growth or reproduction of a fungus, comprising providing the fungus with a composition comprising an effective amount of at least one conidial germination-inhibiting (CGI) factor, CGI factor precursor, CGI factor fragment, or CGI factor motif, wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif comprises at least one of SEQ ID NO: 1921-1956, and wherein the nucleotide sequence of the at least one CGI factor, CGI factor precursor, CGI factor fragment, or CGI factor motif does not occur in the genome of the fungus.
   76. The method of embodiment 75, wherein the CGI factor, the CGI factor precursor, the CGI factor fragment, or the CGI factor motif is active and/or toxic.
   77. The method of embodiment 75 or embodiment 76, wherein the composition is provided to the fungus by directly contacting the fungus with the composition, or by delivering the composition to the environment of the fungus.
   78. A method of preventing growth of a fungus on a surface, comprising treating the surface with a composition comprising an effective amount of at least one conidial germination-inhibiting (CGI) factor, CGI factor precursor, CGI factor fragment, or CGI factor motif, wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif comprises at least one of SEQ ID NO: 1921-1956.
   79. The method of embodiment 78, wherein the CGI factor, the CGI factor precursor, the CGI factor fragment, or the CGI factor motif is active and/or toxic.
   80. The method of 78 or embodiment 79, wherein the surface is a non-living surface or is the surface of a living organism.
   81. The method of any one of embodiments 78-80, wherein the nucleotide sequence of the at least one CGI factor, CGI factor precursor, CGI factor fragment, or CGI factor motif does not occur in the genome of the fungus.
   82. A composition having antifungal properties, comprising a substrate or matrix that is complexed with at least one conidial germination-inhibiting (CGI) factor, CGI factor precursor, CGI factor fragment, or CGI factor motif that is active and/or toxic, wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731, or wherein the CGI factor motif comprises at least one of SEQ ID NO: 1921-1956.
   83. The composition of embodiment 82, wherein the CGI factor, the CGI factor precursor, the CGI factor fragment, or the CGI factor motif is active and/or toxic.
   84. The composition of embodiment 82 or embodiment 83, wherein the complexation between the substrate or matrix with the at least one CGI factor, CGI factor precursor, CGI factor fragment, or CGI factor motif is through: (a) covalent bonding, (b) non-covalent bonding, or (c) a combination of (a) and (b).
   85. The composition of embodiment 84, wherein the substrate or matrix comprises polypeptides. 86. The composition of embodiment 84 or embodiment 85, wherein the substrate or matrix comprises self-assembling peptides.
   87. A method of treating a subject with a fungal disease comprising administering to a subject an antifungal or fungicidal composition comprising the CGI factor, the CGI factor precursor, the CGI factor fragment, or the CGI factor motif of embodiment 13 and a pharmaceutically acceptable carrier.
   88. The method of embodiment 87, wherein the subject is a mammal.
   89. The method of embodiment 88, wherein the mammal is a human.
   90. The method of embodiment 88, wherein the mammal is a domestic animal or livestock.
   91. The method of any one of embodiments 87-90, wherein the fungal disease is caused by a fungal pathogen selected from the group consisting of Aspergillus, Candida, Coccidioides, Histoplasma, Cryptococcus, Pneumocystis, and Blastomyces fungus.
   92. The method of any one of embodiments 87-91, wherein the fungal disease is aspergillosis, blastomycosis, candidiasis, coccidioidomycosis, histoplasmosis, mucormycosis, mycetoma, ringworm, sporotrichosis, paracoccidioidomycosis, talaromycosis, chromoblastomycosis fusariosis, emergomycosis, scedosporiosis, or fungal meningitis.
   93. The method of any one of embodiments 87-92, wherein the antifungal or fungicidal composition is administered intravenously, intramuscularly, subcutaneously, topically, orally, transdermally, intraperitoneally, intraorbitally, by implantation, by inhalation, intrathecally, intraventricularly, or intranasally.

### EXAMPLES

The presently disclosed subject matter will be better understood by reference to the following Examples, which are provided as exemplary of the invention, and not by way of limitation.

### Example 1

This example describes the design of a conidial germination-inhibiting (CGI) factor.

### Materials and Methods

A pUC plasmid was prepared for expression of CGI peptides. The plasmid contained a T7 LacOperator promoter (SEQ ID NO: 2245) for driving the transcription and translation of one or more CGI peptides. The T7 promoter was codon optimized for driving transcription and translation of the CGI peptides. The plasmid includes a linear polynucleotide sequence containing, from 5′ to 3′, a 5′ peptide secretion sequence, a N-terminus His-tag sequence SEQ ID NO: 2246), a protease cleavage site SEQ ID NO: 2247), and a CGI peptide sequence. The plasmid also contained a transcription termination sequence (SEQ ID NO: 2244).

### Example 2

This example describes the expression of a CGI factor in bacteria.

### Materials and Methods

E. coli (BL21 (DE3)) harboring a pUC plasmid containing the a CGI factor construct were cultured as previously described in Example 1. A single bacterial colony was inoculated in lysogeny broth (LB) medium to produce a starter culture. The starter culture was then placed in a shaker at 37° C. until the optical density at 600 nm (OD600) reached between 0.4-0.8. Afterwards, 40-400 μM isopropyl β-d-1-thiogalactopyranoside (IPTG) was used to induce the expression of the plasmid promoter overnight at 37° C.

### Example 3

This example describes the purification of a CGI factor.

### Materials and Methods

Purification of recombinant peptides was performed as previously described (Nallamsetty, S., and Waugh, D. S. (2007) Biochem Biophys Res Commun. 364 (3): 639-44)). The bacterial cell suspension was lysed using a homogenizer at 10,000-11,000 psi. Lysed cell suspensions were then centrifuged for 30 min at 15,000×g. Cell debris was filtered using a 0.45 mm polyethersulfone membrane before chromatography. The supernatant was then applied to nickel-nitrilotriacetic acid (Ni-NTA) resin column equilibrated in 50 mM sodium phosphate buffer (pH 7.7), 150 mM sodium chloride and 25 mM imidazole. The column was washed with equilibration buffer until a stable baseline was reached. The bound fusion protein was then eluted with a linear gradient over 10 column volumes into 50 mM sodium phosphate buffer (pH 7.7), 150 mM sodium chloride and 250 mM imidazole. Protein fractions were pooled, and the resulting sample was concentrated approximately tenfold. Any additional cell debris was precipitated by centrifuging at 5,000×g for 10 min. His6-TEV protease was then added to remove the protein tag from the fusion protein, and digested overnight at 41° C. Efficient cleavage was confirmed by SDS-PAGE gel analysis.

### Example 4

This example describes the design and validation of peptide inhibitors of fungal pathogens.

### Materials and Methods

### Conidial Germination-Inhibiting (CGI) Factors

To identify new compounds and targets to kill pathogenic fungi, a series of short peptides were designed based on fungal mating pheromones. The sequences for peptides 105-119 used in this study are SEQ ID NOs: 2196-2210, respectively. The peptides were expressed and purified as described in Examples 1-3.

### Conidial Germination Inhibition Colorimetric Assay

Biomass was generated by inoculating Fusarium or Botrytis spores into potato dextrose agar plates followed by incubation of the inoculated plates in sealed plastic boxes for seven days. After the incubation, the plates were flooded with 15 mL of sterile water, using a pipette to aspirate water over the mycelia and to cause the conidia to go into suspension. Then, 15 mL of the conidial suspension were transferred to a falcon tube and amended with 0.5% (w/v) D-glucose. Microconidia were quantified using a glass hemacytometer (Weber Scientific, Cat no. 3048-11). 10 μL of the microconidia suspension were transferred into the glass hemocytometer chamber underneath the coverslip. The number of conidia in each set of 16-corner squares of the hemocytometer were counted, and the average number of counts was multiplied by 10\hat{ }4 to obtain the number of conidia per mL in the original suspension.

For microconidia treatment, a 2 mM fenpiclonil (Millipore Sigma, Cat. no. 36532) solution stock solution was prepared using 50% (w/v) ethanol in water. The peptide solutions were prepared using 50% (w/v) ethanol in water. 96-well plates (VWR, Cat. no. 734-2781) were prepared by adding the treatment solution and microconidia to each well to obtain desired treatment concentration and 1×10\hat{ }6 conidia/mL in a final volume of 150 μL per well. Potato dextrose broth (PDB; Alpha Biosciences, Cat. no. P16-126) was used to adjust the volume. The plates were then covered and incubated at 30° C. for 18 hours and 200 rpm. Afterwards, 15 μL of Prestoblue cell viability reagent (Thermofisher Scientific, Cat no. A13262) was added to each well, and the plate was further incubated for 7 hours. Then, the fluorescence at 590 nm emission after excitation at 560 nm was recorded using a spectrophotometer (BioTek Synergy H1 Microplate Reader, Fischer Scientific, Cat. no. 11-120-533).

### Results

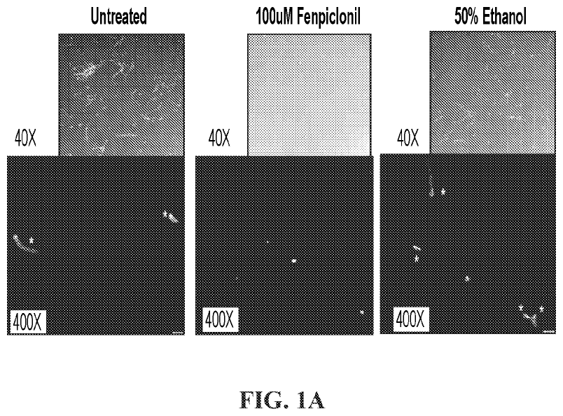

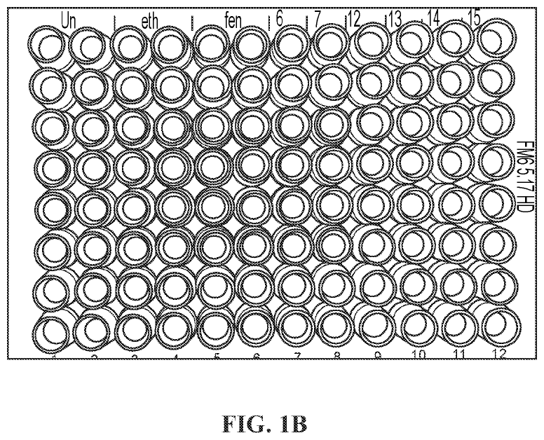

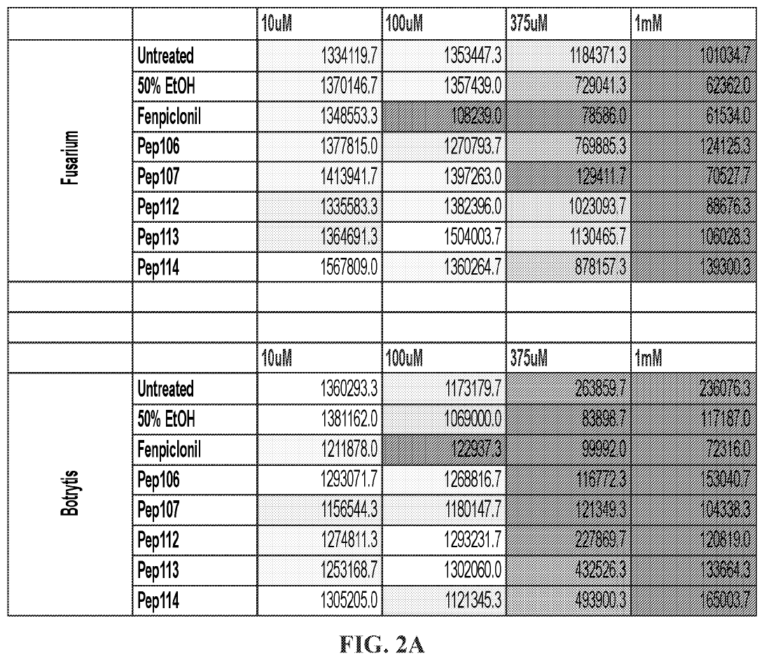

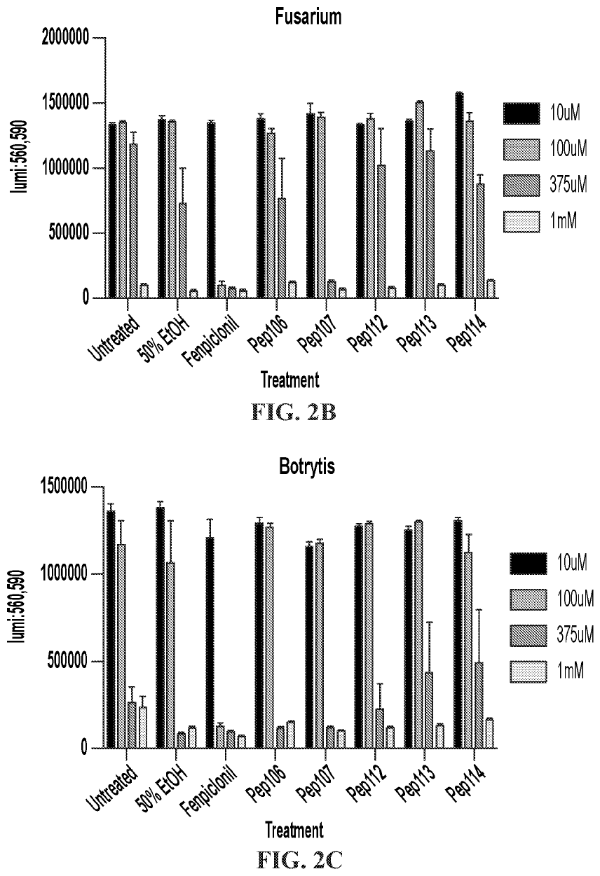

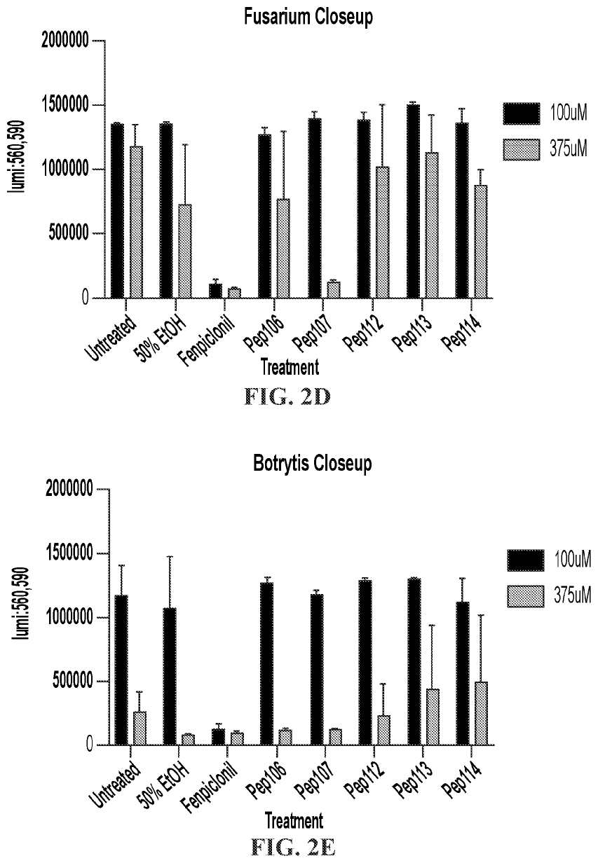

The peptides 105-119 (SEQ ID NOs: 2196-2210, respectively) were prepared and evaluated for their effect on fungal asexual reproduction. Untreated microconidia, and microconidia treated with 50% (w/v) ethanol in water were used as negative controls for the conidial germination inhibition assay, while the fungicide compound fenpiclonil was used as a positive control (FIG. 1A). FIG. 2A shows an exemplary layout of a 96-well plate as used in the conidial germination inhibition assay, while FIG. 2B shows the validation of the resazurin-based cell viability reagent used in this study.

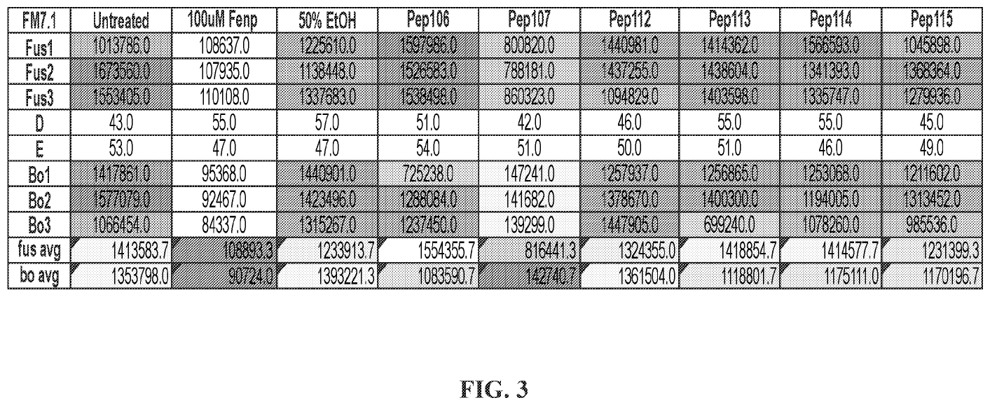

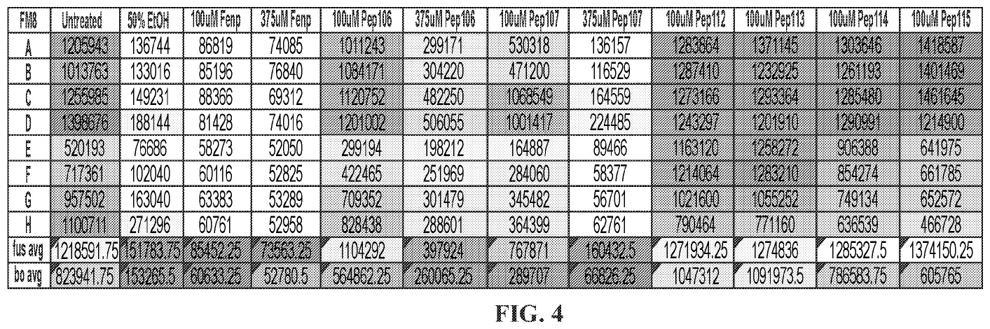

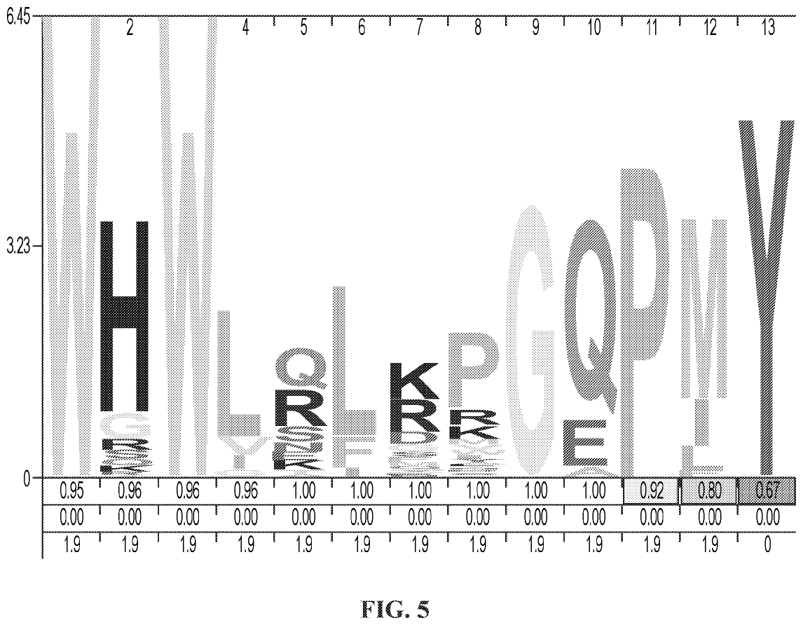

Treatment of Fusarium or Botrytis conidia with the candidate CGI factors resulted in inhibition of conidial germination (FIG. 2A). This inhibition was concentration dependent (FIGS. 2B-2C). Peptide106, which is derived from a Botrytis pheromone, resulted in a decrease of Botrytis conidial germination (FIGS. 2D-2E). Peptide107, which is derived from a Saccharomyces cerevisiae pheromone, inhibited germination of both Botrytis and Fusarium conidia (FIGS. 2D-2E), with the strongest inhibition observed at 375 μM concentration (FIGS. 3 and 4).

### CONCLUSIONS

Fungal pheromones typically promote fungi growth. Here, it was demonstrated that peptides derived from fungal pheromones unexpectedly inhibited growth of Botrytis and Fusarium. Novel CGI factors based on fungal pheromones could be applied to controlling fungal infection of plants.

### Example 5

This example describes the antifungal activity of tandem CGI factors when applied to seeds.

### Materials and Methods

### Expression and Purification of Tandem CGI Factors

A plasmid vector is prepared for tandem CGI expression. The plasmid vector contains (from 5′ to 3′): a T7 LacOperator promoter (SEQ ID NO: 2245); a Shine-Delgarno sequence (SEQ ID NO: 2251); a linear polynucleotide sequence encoding a N-terminus His-tag sequence (SEQ ID NO: 2246); a TEV protease cleavage tag (SEQ ID NO: 2247); a first CGI peptide sequence; a P2A cleavage peptide (if not used in bacteria) or a Translation Initiation Region (TIR; if used in bacteria) (SEQ ID NO: 2252); a second CGI peptide sequence; and a T7 transcription termination sequence (SEQ ID NO: 2244).

E. coli (BL21 (DE3)) harboring the tandem CGI plasmid are prepared as described in Example 1, and the tandem GCI factor is expressed and purified as described in Examples 2-3.

### Seed Treatment Application

The first and second peptides are produced in stoichiometric and equimolar ratios before and upon purification. Corn or Tomato seeds are soaked in 10 mL of CGI peptide solution in a 50 mL conical tube and incubated on a shaker for 30 minutes. Coated seeds are left to dry overnight in a fume hood

### Results

Tandem CGI are prepared from the GCI factors corresponding to peptides 105-119 as described in Example 4. The tandem CGIs prepared in this study are SEQ ID NO: 1957, SEQ ID NO: 1958, SEQ ID NO: 1959, SEQ ID NO: 1960, SEQ ID NO: 1961, SEQ ID NO: 1962, SEQ ID NO: 1963, SEQ ID NO: 1964, SEQ ID NO: 1965, SEQ ID NO: 1966, SEQ ID NO: 1967, SEQ ID NO: 1968, SEQ ID NO: 1969, SEQ ID NO: 1970, SEQ ID NO: 1971, SEQ ID NO: 1972, SEQ ID NO: 1973, SEQ ID NO: 1974, SEQ ID NO: 1975, SEQ ID NO: 1976, SEQ ID NO: 1977, SEQ ID NO: 1978, SEQ ID NO: 1979, SEQ ID NO: 1980, SEQ ID NO: 1981, SEQ ID NO: 1982, SEQ ID NO: 1983, SEQ ID NO: 1984, SEQ ID NO: 1985, SEQ ID NO: 1986, SEQ ID NO: 1987, SEQ ID NO: 1988, SEQ ID NO: 1989, SEQ ID NO: 1990, SEQ ID NO: 1991, SEQ ID NO: 1992, SEQ ID NO: 1993, SEQ ID NO: 1994, SEQ ID NO: 1995, SEQ ID NO: 1996, SEQ ID NO: 1997, SEQ ID NO: 1998, SEQ ID NO: 1999, SEQ ID NO: 2000, SEQ ID NO: 2001, SEQ ID NO: 2002, SEQ ID NO: 2003, SEQ ID NO: 2004, SEQ ID NO: 2005, SEQ ID NO: 2006, SEQ ID NO: 2007, SEQ ID NO: 2008, SEQ ID NO: 2009, SEQ ID NO: 2010, SEQ ID NO: 2011, SEQ ID NO: 2012, SEQ ID NO: 2013, SEQ ID NO: 2014, SEQ ID NO: 2015, SEQ ID NO: 2016, SEQ ID NO: 2017, SEQ ID NO: 2018, SEQ ID NO: 2019, SEQ ID NO: 2020, SEQ ID NO: 2021, SEQ ID NO: 2022, SEQ ID NO: 2023, SEQ ID NO: 2024, SEQ ID NO: 2025, SEQ ID NO: 2026, SEQ ID NO: 2027, SEQ ID NO: 2028, SEQ ID NO: 2029, SEQ ID NO: 2030, SEQ ID NO: 2031, SEQ ID NO: 2032, SEQ ID NO: 2033, SEQ ID NO: 2034, SEQ ID NO: 2035, SEQ ID NO: 2036, SEQ ID NO: 2037, SEQ ID NO: 2038, SEQ ID NO: 2039, SEQ ID NO: 2040, SEQ ID NO: 2041, SEQ ID NO: 2042, SEQ ID NO: 2043, SEQ ID NO: 2044, SEQ ID NO: 2045, SEQ ID NO: 2046, SEQ ID NO: 2047, SEQ ID NO: 2048, SEQ ID NO: 2049, SEQ ID NO: 2050, SEQ ID NO: 2051, SEQ ID NO: 2052, SEQ ID NO: 2053, SEQ ID NO: 2054, SEQ ID NO: 2055, SEQ ID NO: 2056, SEQ ID NO: 2057, SEQ ID NO: 2058, SEQ ID NO: 2059, SEQ ID NO: 2060, SEQ ID NO: 2061, SEQ ID NO: 2062, SEQ ID NO: 2063, SEQ ID NO: 2064, SEQ ID NO: 2065, SEQ ID NO: 2066, SEQ ID NO: 2067, SEQ ID NO: 2068, SEQ ID NO: 2069, SEQ ID NO: 2070, SEQ ID NO: 2071, SEQ ID NO: 2072, SEQ ID NO: 2073, SEQ ID NO: 2074, SEQ ID NO: 2075, SEQ ID NO: 2076, SEQ ID NO: 2077, SEQ ID NO: 2078, SEQ ID NO: 2079, SEQ ID NO: 2080, SEQ ID NO: 2081, SEQ ID NO: 2082, SEQ ID NO: 2083, SEQ ID NO: 2084, SEQ ID NO: 2085, SEQ ID NO: 2086, SEQ ID NO: 2087, SEQ ID NO: 2088, SEQ ID NO: 2089, SEQ ID NO: 2090, SEQ ID NO: 2091, SEQ ID NO: 2092, SEQ ID NO: 2093, SEQ ID NO: 2094, SEQ ID NO: 2095, SEQ ID NO: 2096, SEQ ID NO: 2097, SEQ ID NO: 2098, SEQ ID NO: 2099, SEQ ID NO: 2100, SEQ ID NO: 2101, SEQ ID NO: 2102, SEQ ID NO: 2103, SEQ ID NO: 2104, SEQ ID NO: 2105, SEQ ID NO: 2106, SEQ ID NO: 2107, SEQ ID NO: 2108, SEQ ID NO: 2109, SEQ ID NO: 2110, SEQ ID NO: 2111, SEQ ID NO: 2112, SEQ ID NO: 2113, SEQ ID NO: 2114, SEQ ID NO: 2115, SEQ ID NO: 2116, SEQ ID NO: 2117, SEQ ID NO: 2118, SEQ ID NO: 2119, SEQ ID NO: 2120, SEQ ID NO: 2121, SEQ ID NO: 2122, SEQ ID NO: 2123, SEQ ID NO: 2124, SEQ ID NO: 2125, SEQ ID NO: 2126, SEQ ID NO: 2127, SEQ ID NO: 2128, SEQ ID NO: 2129, SEQ ID NO: 2130, SEQ ID NO: 2131, SEQ ID NO: 2132, SEQ ID NO: 2133, SEQ ID NO: 2134, SEQ ID NO: 2135, SEQ ID NO: 2136, SEQ ID NO: 2137, SEQ ID NO: 2138, SEQ ID NO: 2139, SEQ ID NO: 2140, SEQ ID NO: 2141, SEQ ID NO: 2142, SEQ ID NO: 2143, SEQ ID NO: 2144, SEQ ID NO: 2145, SEQ ID NO: 2146, SEQ ID NO: 2147, SEQ ID NO: 2148, SEQ ID NO: 2149, SEQ ID NO: 2150, SEQ ID NO: 2151, SEQ ID NO: 2152, SEQ ID NO: 2153, SEQ ID NO: 2154, SEQ ID NO: 2155, SEQ ID NO: 2156, SEQ ID NO: 2157, SEQ ID NO: 2158, SEQ ID NO: 2159, SEQ ID NO: 2160, SEQ ID NO: 2161, SEQ ID NO: 2162, SEQ ID NO: 2163, SEQ ID NO: 2164, SEQ ID NO: 2165, SEQ ID NO: 2166, SEQ ID NO: 2167, SEQ ID NO: 2168, SEQ ID NO: 2169, SEQ ID NO: 2170, SEQ ID NO: 2171, SEQ ID NO: 2172, SEQ ID NO: 2173, SEQ ID NO: 2174, SEQ ID NO: 2175, SEQ ID NO: 2176, SEQ ID NO: 2177, SEQ ID NO: 2178, SEQ ID NO: 2179, SEQ ID NO: 2180, and/or SEQ ID NO: 2181.

### Example 6

This example describes the antifungal activity of a single CGI peptide when applied to seeds.

### Materials and Methods

### Purification and Expression of a CGI Peptide

A CGI peptide plasmid is prepared. The plasmid contains (from 5′ to 3′): a T7 LacOperator promoter (SEQ ID NO: 2245); a Shine-Delgarno sequence (SEQ ID NO: 2251); a linear polynucleotide sequence encoding a N-terminus His-tag sequence SEQ ID NO: 2246), a TEV protease cleavage tag SEQ ID NO: 2247), and a peptide sequence; and a T7 transcription termination sequence SEQ ID NO: 2244).

E. coli (BL21 (DE3)) harboring the CGI peptide plasmid are prepared as described in Example 1, and the tandem CGI factor as expressed and purified as described in Examples 2-3.

### Seed Treatment Application

Corn or Tomato seeds are soaked in 10 mL of CGI peptide solution in a 50 mL conical tube and incubated on a shaker for 30 minutes. Coated seeds are left to dry overnight in a fume hood.

### Example 7

This example describes the delivery of a CGI factor to a seed.

### Materials and Methods

### Purification and Expression of CGI Factors

E. coli (BL21 (DE3)) harboring a pUC plasmid containing a CGI factor is produced and purified as described in Examples 1-3. The CGI factor is then formulated for use as a seed treatment or plantable composition. In addition to pesticidal minicells, the formulation can include other pesticides, surfactants, film-forming polymers, carriers, antifreeze agents, and/or formulary additives. When used together, these components provide a composition that is storage-stable and suitable for use in normal seed treatment equipment, such as slurry seed treaters, direct treaters, on-farm hopper-boxes, or planter-boxes.

For the seed treatment formulation, 40% of a CGI peptide is prepared in 3% EP/PO block co-polymer, tristyrylphenol, 0.5% ethoxylate, 5% calcium salt with pigment red, 0.2% silicone oil and water, and incubated with rice seeds overnight under a negative flow laboratory hood.

Formulated versus unformulated rice seeds are then analyzed to determine the percent coverage for fungal growth.

### Example 8

This example describes the application of CGI factors to a post-harvest crop.

### Materials and Methods

Strawberries are obtained and sorted based on size. Fruits of the smallest size are surface sterilized by submersion in 70% ethanol for 1 minute. The fruits are then triple-rinsed with water, and the excess moisture is removed prior to application of the CGI factors.

For CGI factor application, the treatment solutions (CGI and controls) are prepared. For each treatment condition, six strawberries are arranged on a petri dish and placed in a fume hood. Each strawberry is sprayed with 2 mL of the treatment solution, covering the entire fruit, including the hull. Alternatively, the fruits are submerged one-by-one in the treatment solution. Treated fruit are then left to dry on a fume hood for 15 minutes.

Following treatment of the strawberries, a conidial suspension is prepared by flooding a 7-day-old Botrytis plate with 10 mL water. The solution is then pipetted into a tube, and 50 mg D-glucose is added. The treated strawberries are transferred to a new petri dish in a plastic container, and 3 mL of the conidial suspension is added to each petri dish, gently rolling the strawberries to spread the suspension on the surface of the fruit. The plastic containers are then covered and incubated at room temperature for 7 days. At the end of the incubation period, the strawberries are evaluated to determine efficacy of the treatment solutions as compared to an untreated control sample.

### Results

Organic strawberries are at risk of fungal infection at any point after harvest. Wounding can occur during delivery or packaging, which increases the fruit's susceptibility to fungal pathogens. Additionally, tightly packaged strawberries are under a constant load, and experience varying temperatures when transported between field, grocery stores, and a consumer's home. Due to the highly perishable nature of this fruit, there is significant loss in quantity and quality of organic strawberries post-harvest.

To address this issue, CGI factors are applied, by either spray or immersion treatment, to healthy strawberries before packaging to prevent fungal infection. The CGI factors will remain present on the fruit to inhibit spore germination until optionally washed off by a consumer.

### Example 9

This example describes the design and validation of peptide inhibitors of fungal pathogens.

### Materials and Methods

### Conidial Germination-Inhibiting (CGI) Factors

To identify new compounds and targets to kill pathogenic fungi, a series of short peptides including SEQ ID NO: 2196, SEQ ID NO: 2197, SEQ ID NO: 2198, SEQ ID NO: 2199, SEQ ID NO: 2200, SEQ ID NO: 2201, SEQ ID NO: 2202, SEQ ID NO: 2203, SEQ ID NO: 2204, SEQ ID NO: 2205, SEQ ID NO: 2206, SEQ ID NO: 2207, SEQ ID NO: 2208, SEQ ID NO: 2209, SEQ ID NO: 2210, SEQ ID NO: 2211, SEQ ID NO: 2212, SEQ ID NO: 2213, and SEQ ID NO: 2214 were designed based on fungal mating pheromones (Table 1). The peptides included peptides modified by addition of glycine residues at either the N terminus or the C terminus, or that were tandem copies, optionally separated by one or more amino acids (e.g., a linker segment, such as multiple glycine residues). The peptides were expressed and purified as described in Examples 1-3.

TABLE 1
Short peptides designed based on fungal mating pheromones.

| | | SEQ ID |
|---|---|---|
| Description | Sequence | NO: |
| F. graminearum Alpha-pheromone | WCTWKGQPCW | 2182 |
| B. cinerea Alpha-pheromone | WCGRPGQPC | 2183 |
| S. cerevisiae Alpha-pheromone | WHWLQLKPGQPMY | 2184 |
| F. graminearum Alpha-pheromone 2 | TWQKCWWPGC | 2199 |
| F. graminearum Alpha-pheromone 3 | PKWTWQGCCW | 2200 |
| F. graminearum Alpha-pheromone 4 | QTWWPGCKWC | 2201 |
| F. graminearum Alpha-pheromone 5 | TQWCWWGKCP | 2202 |
| B. cinerea Alpha-pheromone 2 | CPWCQGGRP | 2203 |
| B. cinerea Alpha-pheromone 3 | PGPCWRGCQ | 2204 |
| B. cinerea Alpha-pheromone 4 | GWQCRPPCG | 2205 |
| B. cinerea Alpha-pheromone 5 | CGQRGPPCW | 2206 |
| S. cerevisiae Alpha-pheromone 2 | WKMGQYHQLPPLW | 2207 |
| S. cerevisiae Alpha-pheromone 3 | MYGKPHLWQLQWP | 2208 |
| S. cerevisiae Alpha-pheromone 4 | QMWPGPWHKQYLL | 2209 |
| S. cerevisiae Alpha-pheromone 5 | PGQMKWPHWLLYQ | 2210 |
| S. cerevisiae Alpha-pheromone 6 (pep-gly) | WHWLQLKPGQPMYG | 2186 |
| S. cerevisiae Alpha-pheromone 7 (gly-pep) | GWHWLQLKPGQPMY | 2187 |
| S. cerevisiae Alpha-pheromone 8 (pep-pep) | WHWLQLKPGQPMYWHWLQ | 2188 |
| | LKPGQPMY | |
| S. cerevisiae Alpha-Pheromone 9 | WHWLQLKPGQPMYGGGGS | 2189 |
| (pep-GGGG-pep) | WHWLQLKPGQPMY | |

### Fusarium Viability Colorimetric Assay

A Fusarium conidial suspension was prepared from a 7-day culture of Fusarium biomass grown on potato dextrose agar for 7 days, and quantified by hemocytometer, as described in Example 4. Solutions of fenpiclonil (Millipore Sigma, Cat. no. 36532) and of the individual peptides were prepared in 50% (w/v) ethanol in water, and tested using a PrestoBlue™ (resazurin) viability colorimetric assay as described in Example 4.

The Fusarium conidial suspension, peptide or control treatment solution, and potato dextrose broth (Alpha Biosciences, catalogue no. P16-126) were added to each well of a 96-well plate to obtain the desired treatment concentration and 1×10\hat{ }6 conidia/mL in a final volume of 150 microliters per well. The plates were covered and incubated at 30° C. for 18 hours and 200 rpm. Fifteen microliters of PrestoBlue™ Cell Viability Reagent (Thermofisher Scientific, Cat no. A13262) were added to each well, and the plate was further incubated before measuring fluorescence at 590 nm emission with excitation at 560 nm using a spectrophotometer (BioTek Synergy H1 Microplate Reader, Fischer Scientific, Cat. no. 11-120-533).

### Botrytis Viability Colorimetric Assay

A Botrytis conidial suspension was prepared from a 7-day culture of Botrytis cinarea (strain CBS 261.71) biomass grown on potato dextrose agar for 7 days, and quantified by hemocytometer, as described in Example 4. Solutions of fenpiclonil (Millipore Sigma, Cat. no. 36532) and of the individual peptides were prepared in 50% (w/v) ethanol in water, and tested using a PrestoBlue™ (resazurin) viability colorimetric assay as described in Example 4.

The Botrytis conidial suspension, peptide or control treatment solution, and potato dextrose broth (Alpha Biosciences, catalogue no. P16-126) were added to each well of a 96-well plate to obtain the desired treatment concentration and 1×10\hat{ }6 conidia/mL in a final volume of 150 microliters per well. The plates were covered and incubated at 30° C. for 24 hours and 200 rpm. Fifteen microliters of PrestoBlue™ Cell Viability Reagent (Thermofisher Scientific, Cat no. A13262) were added to each well, and the plate was further incubated for another hour before measuring fluorescence at 590 nm emission with excitation at 560 nm using a spectrophotometer (BioTek Synergy H1 Microplate Reader, Fischer Scientific, Cat. no. 11-120-533).

### Results

### Fusarium Viability Colorimetric Assay

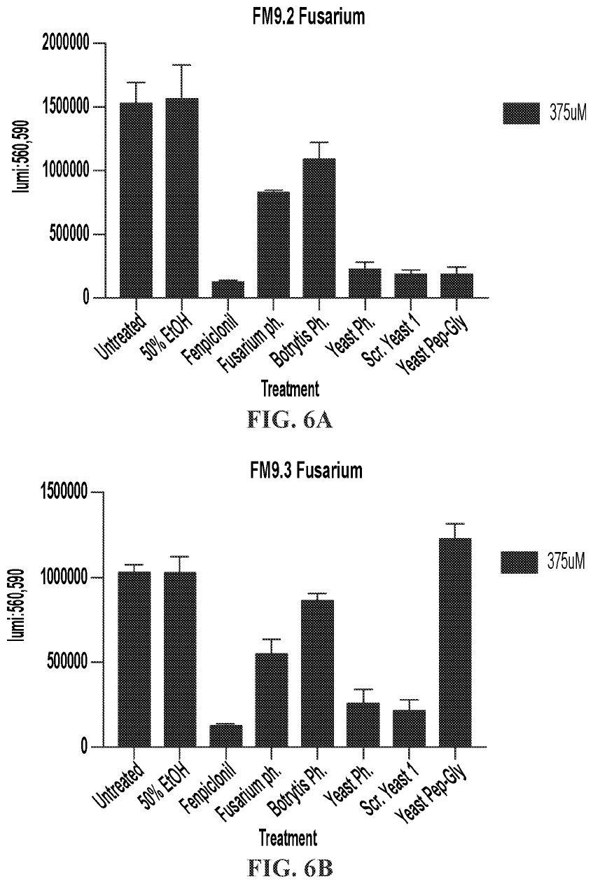

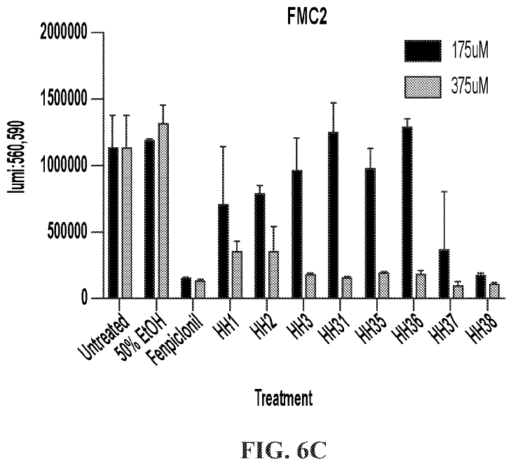

Multiple separate Fusarium viability experiments were run. In the first experiment, the peptides tested for the ability to inhibit conidial germination and/or decrease fungal cell viability included the native alpha pheromones from Fusarium sp. (WCTWKGQPCW, SEQ ID NO: 2182), Botrytis sp. (WCGRPGQPC, SEQ ID NO: 2183), and Saccharomyces cerevisiae (WHWLQLKPGQPMY, SEQ ID NO: 2184), as well as a synthetic peptide having the amino acid sequence WKMGQYHQLPPLW (SEQ ID NO: 2185), which is based on a rearrangement of the amino acid sequence of the Saccharomyces cerevisiae alpha pheromone) and a modified Saccharomyces cerevisiae alpha pheromone with a C-terminus glycine cap that has the amino acid sequence WHWLQLKPGQPMYG (SEQ ID NO: 2186). Results are shown in FIG. 6A. All of the CGI peptides tested demonstrated an ability at a concentration of 375 micromolar to reduce Fusarium cell viability in the treated samples, in comparison to the samples that were untreated or treated only with the 50% (w/v) ethanol in water carrier solution. Notably, in this experiment, the Saccharomyces cerevisiae alpha pheromone (SEQ ID NO: 2184) as well as the synthetic peptide (SEQ ID NO: 2185) and the C-terminus glycine-capped Saccharomyces cerevisiae alpha pheromone (SEQ ID NO: 2186) greatly decreased Fusarium cell viability to about the same level as did the positive control (fenpiclonil) fungicidal treatment. These results demonstrated that an alpha pheromone encoded in the genome of one fungal genus (Saccharomyces) and its derivatives were unexpectedly effective in reducing viability of cells of a different fungal genus (Fusarium).

In a second experiment carried out similarly to the first, the same peptides were again tested at a concentration of 375 micromolar; viability was measured at a later timepoint (51 hours after addition of the resazurin). Results are shown in FIG. 6B. Similar results were observed, with the Fusarium alpha pheromone (SEQ ID NO: 2182) and Botrytis alpha pheromone (SEQ ID NO: 2183) again decreasing Fusarium cell viability, in comparison to the samples that were untreated or treated only with the 50% (w/v) ethanol in water carrier solution. Notably, the Saccharomyces cerevisiae alpha pheromone (SEQ ID NO: 2184) and synthetic peptide (SEQ ID NO: 2185) again greatly decreased Fusarium cell viability to about the same level as did the positive control (fenpiclonil) fungicidal treatment. These results confirmed that an alpha pheromone encoded in the genome of one fungal genus (Saccharomyces) and its derivatives were unexpectedly effective in reducing viability of cells of a different fungal genus (Fusarium).

In a third experiment run similarly to the first and second, eight putative conidial germination-inhibiting peptides were tested at 175 micromolar and at 375 micromolar. The tested peptides again included the native alpha pheromones from Fusarium sp. (WCTWKGQPCW, SEQ ID NO: 2182), Botrytis sp. (WCGRPGQPC, SEQ ID NO: 2183), and Saccharomyces cerevisiae (WHWLQLKPGQPMY, SEQ ID NO: 2184), the synthetic peptide (SEQ ID NO: 2185) and the C-terminus glycine-capped Saccharomyces cerevisiae alpha pheromone (SEQ ID NO: 2186), as well as three peptides not previously tested: a modified Saccharomyces cerevisiae alpha pheromone with a N-terminus glycine cap (GWHWLQLKPGQPMY, SEQ ID NO: 2187), a peptide having the sequence of contiguous tandem copies of the Saccharomyces cerevisiae alpha pheromone (WHWLQLKPGQPMYWHWLQLKPGQPMY, SEQ ID NO: 2188), and a peptide having the sequence of tandem copies of the Saccharomyces cerevisiae alpha pheromone separated by a four-glycine linking segment (WHWLQLKPGQPMYGGGGSWHWLQLKPGQPMY, SEQ ID NO: 2189). Results are shown in FIG. 6C. The observed results were similar to those obtained in the first and second experiments. At a concentration of 375 micromolar, the Fusarium alpha pheromone (SEQ ID NO: 2182) and Botrytis alpha pheromone (SEQ ID NO: 2183) again decreased Fusarium cell viability, in comparison to the samples that were untreated or treated only with the 50% (w/v) ethanol in water carrier solution; at the lower concentration of 175 micromolar, this effect was less pronounced. At 375 micromolar, the Saccharomyces cerevisiae alpha pheromone (SEQ ID NO: 2184) and its derivatives (SEQ ID NO: 2188 and 2189) again greatly decreased Fusarium cell viability to about the same level as did the positive control (fenpiclonil) fungicidal treatment, but at 175 micromolar showed no significant effects on Fusarium cell viability, Notably, the longer Saccharomyces cerevisiae alpha pheromone-derived tandem peptides (tandem copies without a spacer, SEQ ID NO: 2188) and (tandem copies with a spacer, SEQ ID NO: 2189) were effective at both 175 micromolar and 375 micromolar in reducing Fusarium cell viability to about the same level as did the positive control (fenpiclonil) fungicidal treatment.

Experiments 4 and 5 were performed following similar protocols and with the same CGI peptides as in the third experiment. Results from these experiments are provided as mean percent inhibition and deviation from the mean (four replicates) in Table 2.

TABLE 2
Results of Fusarium viability colorimetric
assay experiments four and five.

| | Percent Inhibition (Fusarium) | | | |
|---|---|---|---|---|
| | Experiment 4 | | Experiment 5 | |
| Treatment | Mean | σM | Mean | σM |
| Ethanol | −0.42 | 0.94 | −36.51 | 6.53 |
| Fenpiclonil | 82.25 | 0.66 | 79.19 | 0.21 |
| WCTWKGQPCW | 47.78 | 0.62 | 28.39 | 2.43 |
| (SEQ ID NO: 2182) | | | | |
| WCGRPGQPC | 46.67 | 0.62 | 42.81 | 1.88 |
| (SEQ ID NO: 2183) | | | | |
| WHWLQLKPGQPMY | 66.33 | 1.73 | 62.01 | 3.41 |
| (SEQ ID NO: 2184) | | | | |
| WKMGQYHQLPPLW | 70.84 | 1.70 | 69.60 | 0.42 |
| (SEQ ID NO: 2185) | | | | |
| WHWLQLKPGQPMYG | 64.21 | 1.87 | 66.45 | 0.24 |
| (SEQ ID NO: 2186) | | | | |
| GWHWLQLKPGQPMY | (not | | 66.96 | 0.46 |
| (SEQ ID NO: 2187) | tested) | | | |
| WHWLQLKPGQPMYW | | | 71.82 | 1.05 |
| HWLQLKPGQPMY | | | | |
| (SEQ ID NO: 2188) | | | | |
| WHWLQLKPGQPMYGGG | | | 79.77 | 0.38 |
| GSWHWLQLKPGQPMY | | | | |
| (SEQ ID NO: 2189) | | | | |

### Botrytis Viability Colorimetric Assay

Results are provided as mean percent inhibition and deviation from the mean (four replicates) in Table 3. The results were generally similar to those observed in the Fusarium viability assay. At a concentration of 375 micromolar, the Fusarium alpha pheromone (SEQ ID NO: 2182) and Saccharomyces cerevisiae alpha pheromone (SEQ ID NO: 2184) decreased Botrytis cell viability, in comparison to the samples that were untreated or treated only with the 50% (w/v) ethanol in water carrier solution. In this experiment, treatment with the Saccharomyces cerevisiae alpha pheromone (SEQ ID NO: 2184) and its derivatives (SEQ ID NO: 2188 and 2189) decreased Fusarium cell viability by roughly about two-thirds of the viability decrease obtained using the positive control (fenpiclonil) fungicidal treatment. As had been observed in the Fusarium viability experiments, at 375 micromolar, the longer Saccharomyces cerevisiae alpha pheromone-derived tandem peptides (tandem copies without a spacer, SEQ ID NO: 2188) and (tandem copies with a spacer, SEQ ID NO: 2189) were the most effective peptides tested, reducing Botrytis cell viability as well as did the fenpiclonil treatment. These results confirmed that an alpha pheromone encoded in the genome of one fungal genus (Saccharomyces) and its derivatives were unexpectedly effective in reducing viability of cells of a different fungal genus (Botrytis).

TABLE 3
Results of Botrytis viability
colorimetric assay.

| | Percent Inhibition | |
|---|---|---|
| | (Botrytis) | |
| Treatment | Mean | σM |
| Ethanol | −83.17 | 6.92 |
| Fenpiclonil | 75.80 | 0.51 |
| WCTWKGQPCW | 8.56 | 5.25 |
| (SEQ ID NO: 2182) | | |
| WCGRPGQPC | −22.63 | 3.85 |
| (SEQ ID NO: 2183) | | |
| WHWLQLKPGQPMY | 45.14 | 0.84 |
| (SEQ ID NO: 2184) | | |
| WKMGQYHQLPPLW | 52.30 | 0.59 |
| (SEQ ID NO: 2185) | | |
| WHWLQLKPGQPMYG | 47.30 | 0.46 |
| (SEQ ID NO: 2186) | | |
| GWHWLQLKPGQPMY | 66.96 | 53.93 |
| (SEQ ID NO: 2187) | | |
| WHWLQLKPGQPMYWHWLQLKPG | 71.82 | 74.73 |
| QPMY (SEQ ID NO: 2188) | | |
| WHWLQLKPGQPMYGGGGSWHWL | 79.77 | 78.04 |
| QLKPGQPMY | | |
| (SEQ ID NO: 2189) | | |

### Example 10

This example describes novel synthetic peptide inhibitors of fungal growth or viability, or of conidial germination. More specifically, this example describes examples of synthetic peptides that are designed to inhibit conidial germination, and/or to decrease cell viability, of fungi.

An embodiment of these synthetic peptides is a synthetic peptide that includes an amino acid sequence including the sequences at least two different CGI peptides (e.g., alpha pheromone peptides from different source organisms or encoded in different source genomes, or derivatives of these alpha pheromone peptides), optionally further including additional amino acids, such as amino acids forming a spacer or linker sequence.

Another embodiment of these synthetic peptides is a synthetic peptide that includes sequence of, or derived from, at least one CGI peptide, fused to a signal peptide functional in the cell in which the synthetic peptide is to be expressed. Many signal peptide sequences have been described, for example, the Tat (Twin-arginine translocation) signal sequence is typically an N-terminal peptide sequence containing a consensus SRRxFLK “twin-arginine” motif, which serves to translocate a folded protein containing such a Tat signal peptide across a lipid bilayer. See also, e.g., the Signal Peptide Database publicly available at www[dot]signalpeptide[dot]de. For example, a synthetic peptide including a CGI peptide sequence fused to a bacterial signal peptide functional in a plant cell can be expressed in a plant cell or plant. See, e.g., the bacterial signal peptide with the sequence MVKVKCYVLFTALLSSLCAYG (SEQ ID NO: 2195), Moeller (2019) J. Exp. Bot., 60:3337-3335; doi:10.1093/jxb/erp167. Signal peptides are also useful for directing a protein to specific organelles; see, e.g., the experimentally determined and computationally predicted signal peptides disclosed in the Spdb signal peptide database, publicly available at proline[dot]bic[dot]nus[dot]edu[dot]sg/spdb.

Another embodiment of these synthetic peptides is a synthetic peptide that includes sequence of, or derived from, at least one CGI peptide, fused to a cell-penetrating peptide (CPP). Hundreds of CPP sequences have been described; see, e.g., the database of cell-penetrating peptides, CPPsite, publicly available at crdd[dot]osdd[dot]net/raghava/cppsite/. An example of a commonly used CPP sequence is a poly-arginine sequence, e.g., octoarginine or nonoarginine, which can be fused to the C-terminus of the CGI peptide.

Another embodiment of these synthetic peptides is a synthetic peptide that includes sequence of, or derived from, at least one CGI peptide, fused or complexed to a self-assembling peptide. In some embodiments, the self-assembling peptide is (M)(YEYK)ₙYEY (SEQ ID NO: 2194), where n=3 or n is between 3-10, and where methionine is the terminal and optional amino acid, and wherein the self-assembling peptide is covalently or non-covalently linked to one or more CGI peptides; see, e.g. Miki et al. (2021) Nature Communications, 21:3412, DOI: 10.1038/s41467-021-23794-6. In embodiments, a self-assembling peptide is fused to the CGI peptide's N-terminus, or to an internal site (e.g., at a linker peptide sequence connecting two CGI peptides). In embodiments, self-assembling peptide/CGI fusions or complexes form particles or structures (e.g., gels or sheets or fibres) having antifungal properties, and can be formulated (e.g., as paints, coatings, films, fabrics, etc.) and applied to protect a living or non-living substrate or matrix (e.g., a plant tissue surface, a seed, harvested plant parts, foodstuff, wood, plaster, inorganic building material) from fungal infestation or growth or damage.

Combinations of the above embodiments are also envisioned. For example, a synthetic peptide inhibitor of fungal growth or viability can include copies of two or more different CGI peptides (e.g., alpha pheromone peptides from different source organisms or encoded in different source genomes, or derivatives of these alpha pheromone peptides), wherein the CGI peptides are linked with an amino acid linking sequence (e.g., glycine residues) and wherein a poly-arginine CPP sequence is fused to the C-terminus of the synthetic peptide. Embodiments of these synthetic peptides optionally further include additional amino acids, such as amino acids forming a spacer or linker sequence, or that are located adjacent to the alpha pheromone sequence (e.g., a “cap” of one or more amino acids at the N-terminus or C-terminus of an individual alpha pheromone sequence or of the peptide molecule as a whole).

Non-limiting examples of these synthetic peptides are provided in Table 4.

TABLE 4
Examples of synthetic peptides.

| Amino acid sequence | Description |
|---|---|
| WHWLQLKPGQPMYWCTWKGQPCW (SEQ ID NO: | Saccharomyces cerevisiae |
| 2231) | alpha pheromone and |
| | Fusarium graminearum alpha |
| | pheromone, fused in tandem |
| | with no linker |
| WHWLQLKPGQPMYGGGGWCTWKGQPCW (SEQ ID | Saccharomyces cerevisiae |
| NO: 2232) | alpha pheromone and |
| | Fusarium graminearum alpha |
| | pheromone, fused in tandem |
| | with a four-glycine linker |
| WCTWKGQPCWWHWLQLKPGQPMY (SEQ ID NO: | Fusarium graminearum alpha |
| 2233) | pheromone and |
| | Saccharomyces cerevisiae |
| | alpha pheromone, fused in |
| | tandem with no linker |
| WCTWKGQPCWGGGGWHWLQLKPGQPMY (SEQ ID | Fusarium graminearum alpha |
| NO: 2234) | pheromone and |
| | Saccharomyces cerevisiae |
| | alpha pheromone, fused in |
| | tandem with a four-glycine |
| | linker |
| WHWLQLKPGQPMYWCGRPGQPC (SEQ ID NO: 2235) | Saccharomyces cerevisiae |
| | alpha pheromone and Botrytis |
| | cinerea alpha pheromone, |
| | fused in tandem with no linker |
| WHWLQLKPGQPMYGGGGWCGRPGQPC (SEQ ID NO: | Saccharomyces cerevisiae |
| 2236) | alpha pheromone and Botrytis |
| | cinerea alpha pheromone, |
| | fused in tandem with a four- |
| | glycine linker |
| WCTWKGQPCWWCGRPGQPC (SEQ ID NO: 2237) | Fusarium graminearum alpha |
| | pheromone and Botrytis |
| | cinerea alpha pheromone, |
| | fused in tandem with no linker |
| WCTWKGQPCWGGGGWCGRPGQPC (SEQ ID NO: 2238) | Fusarium graminearum alpha |
| | pheromone and Botrytis |
| | cinerea alpha pheromone, |
| | fused in tandem with a four- |
| | glycine linker |
| MVKVKCYVLFTALLSSLCAYGWHWLQLKPGQPMY | E. coli signal peptide |
| (SEQ ID NO: 2239) | (functional in plant cells) |
| | fused to N-terminus of |
| | Saccharomyces cerevisiae |
| | alpha pheromone |
| WHWLQLKPGQPMYRRRRRRRR (SEQ ID NO: 2240) | Saccharomyces cerevisiae |
| | alpha pheromone fused at its |
| | C-terminus to an octoarginine |
| | CPP sequence |
| YEYKYEYKYEYKYEYWHWLQLKPGQPMY (SEQ ID | Y15 self-assembling peptide |
| NO: 2241) | fused to N-terminus of |
| | Saccharomyces cerevisiae |
| | alpha pheromone |
| MYEYKYEYKYEYKYEYWHWLQLKPGQPMY (SEQ ID | Y15 self-assembling peptide |
| NO: 2242) | (including optional N-terminal |
| | methionine) fused to N- |
| | terminus of Saccharomyces |
| | cerevisiae alpha pheromone |
| WHWLQLKPGQPMYGGGGWCTWKGQPCWRRRRRRRR | Saccharomyces cerevisiae |
| (SEQ ID NO: 2243) | alpha pheromone and |
| | Fusarium graminearum alpha |
| | pheromone, fused in tandem |
| | with a four-glycine linker, and |
| | additionally having an |
| | octoarginine CPP sequence at |
| | the C-terminus |

### Example 11

This example describes novel synthetic fusion peptides which include at least one signal peptide and at least one CGI peptide. In embodiments, one or more amino acid linkers is included in the fusion peptide, e.g., linking a signal peptide to an adjacent CGI peptide, or in fusion peptides containing more than one CGI peptide, linking one CGI peptide to another. For economy, the amino acid linkers are relatively short, e.g., ten amino acids or fewer. In embodiments, the signal peptide is cleaved from the CGI peptide in vivo. These fusion peptide sequences are useful, e.g., for expression of the fusion peptide or of the CGI peptide in a eukaryotic cell, such as a plant cell. Related embodiments include eukaryotic cells and eukaryotic organisms (e.g., plant cells or tissues or intact plants) that transiently or stably express one or more CGI peptides or CGI peptide derivatives, such as these synthetic fusion peptides. These synthetic fusion peptides are useful for inhibiting conidial germination, and/or to decrease cell viability, of fungi.

Table 5 provides non-limiting examples of synthetic fusion peptides including at least one signal peptide and at least one CGI peptide, optionally including one or more amino acid linkers. These fusion peptides include various combinations of at least one CGI peptide with the PRI signal sequence MGFVLFSQLPSFLLVSTLLLFLVISHSCRA (SEQ ID NO: 5699) or with the GRP signal sequence MATTKHLALAILVLLSIGMTTSA (SEQ ID NO: 5700), and optionally, further include amino acid linkers of between 1 to 9 amino acids (shown as underlined text in Table 5).

TABLE 5
Examples of synthetic fusion peptides including at least one signal
peptide and at least one CGI peptide.

| SEQ ID | | | |
|---|---|---|---|
| NO: | Description | Linker Sequence | |
| | PR1 signal peptide | QNSQQD | MGFVLFSQLPSFLLVSTLLLFLVISHSC |
| | fused to | (SEQ ID NO: | RAQNSQQDWHWLQLKPGQPMY |
| | S. cerevisiae | 5701) | (SEQ ID NO: 5707) |
| | alpha | | |
| | pheromone | | |
| | | QNSQQ (SEQ ID NO: | MGFVLFSQLPSFLLVSTLLLFLVISHSC |
| | | 5702) | RAQNSQQWHWLQLKPGQPMY (SEQ |
| | | | ID NO: 5708) |
| | | QNSQ (SEQ ID NO: | MGFVLFSQLPSFLLVSTLLLFLVISHSC |
| | | 5703) | RAQNSQWHWLQLKPGQPMY (SEQ ID |
| | | | NO: 5709) |
| | | | MGFVLFSQLPSFLLVSTLLLFLVISHSC |
| | | | RAWHWLQLKPGQPMY (SEQ ID NO: |
| | | | 5710) |
| | PR1 signal peptide | QNS | MGFVLFSQLPSFLLVSTLLLFLVISHSC |
| | fused to | | RAQNSWHWLQLKPGQPMYWHWLQ |
| | S. cerevisiae | | LKPGQPMY (SEQ ID NO: 5711) |
| | alpha | | |
| | pheromone | | |
| | heterodimer (no | | |
| | linker) | | |
| | | QN | MGFVLFSQLPSFLLVSTLLLFLVISHSC |
| | | | RAQNWHWLQLKPGQPMYWHWLQL |
| | | | KPGQPMY (SEQ ID NO: 5712) |
| | | Q | MGFVLFSQLPSFLLVSTLLLFLVISHSC |
| | | | RAQWHWLQLKPGQPMYWHWLQLK |
| | | | PGQPMY (SEQ ID NO: 5713) |
| | | | MGFVLFSQLPSFLLVSTLLLFLVISHSC |
| | | | RAWHWLQLKPGQPMYWHWLQLKP |
| | | | GQPMY (SEQ ID NO: 5714) |
| | PR1 signal peptide | Q | MGFVLFSQLPSFLLVSTLLLFLVISHSC |
| | fused to | | RAQWHWLQLKPGQPMYGGGGSWH |
| | S. cerevisiae | | WLQLKPGQPMY (SEQ ID NO: 5715) |
| | alpha | | |
| | pheromone | | |
| | heterodimer | | |
| | (glycine linker) | | |
| | | | MGFVLFSQLPSFLLVSTLLLFLVISHSC |
| | | | RAWHWLQLKPGQPMYGGGGSWHW |
| | | | LQLKPGQPMY (SEQ ID NO: 5716) |
| | PR1 signal peptide | QNSQQDYLD (SEQ ID | MGFVLFSQLPSFLLVSTLLLFLVISHSC |
| | fused to | NO: 5704) | RAQNSQQDYLDWHWLQLKPGQPMY |
| | S. cerevisiae alpha | | RRRRRRRR (SEQ ID NO: 5717) |
| | pheromone fused to | | |
| | octoarginine | | |
| | | QNSQQDY (SEQ ID | MGFVLFSQLPSFLLVSTLLLFLVISHSC |
| | | NO: 5705) | RAQNSQQDYWHWLQLKPGQPMYRR |
| | | | RRRRRR (SEQ ID NO: 5718) |
| | | QNSQQD (SEQ ID NO: | MGFVLFSQLPSFLLVSTLLLFLVISHSC |
| | | 5701) | RAQNSQQDWHWLQLKPGQPMYRRR |
| | | | RRRRR (SEQ ID NO: 5719) |
| | | | MGFVLFSQLPSFLLVSTLLLFLVISHSC |
| | | | RAWHWLQLKPGQPMYRRRRRRRR |
| | | | (SEQ ID NO: 5720) |
| | GRP signal peptide | RTLLD (SEQ ID NO: | MATTKHLALAILVLLSIGMTTSARTLL |
| | fused to | 5706) | DWHWLQLKPGQPMY (SEQ ID NO: |
| | S. cerevisiae alpha | | 5721) |
| | pheromone | | |
| | | RT | MATTKHLALAILVLLSIGMTTSARTW |
| | | | HWLQLKPGQPMY (SEQ ID NO: 5722) |
| | | R | MATTKHLALAILVLLSIGMTTSARWH |
| | | | WLQLKPGQPMY (SEQ ID NO: 5723) |
| | | | MATTKHLALAILVLLSIGMTTSAWH |
| | | | WLQLKPGQPMY (SEQ ID NO: 5724) |
| | GRP signal peptide | R | MATTKHLALAILVLLSIGMTTSARWH |
| | fused to S. | | WLQLKPGQPMYWHWLQLKPGQPMY |
| | cerevisiae alpha | | (SEQ ID NO: 5725) |
| | pheromone | | |
| | heterodimer (no | | |
| | linker) | | |
| | | | MATTKHLALAILVLLSIGMTTSAWH |
| | | | WLQLKPGQPMYWHWLQLKPGQPMY |
| | | | (SEQ ID NO: 5726) |
| | GRP signal peptide | R | MATTKHLALAILVLLSIGMTTSARWH |
| | fused to S. | | WLQLKPGQPMYGGGGSWHWLQLKP |
| | cerevisiae alpha | | GQPMY (SEQ ID NO: 5727) |
| | pheromone | | |
| | heterodimer | | |
| | (glycine linker) | | |
| | | | MATTKHLALAILVLLSIGMTTSAWH |
| | | | WLQLKPGQPMYGGGGSWHWLQLKP |
| | | | GQPMY (SEQ ID NO: 5728) |
| | GRP signal peptide | RT | MATTKHLALAILVLLSIGMTTSARTW |
| | fused to S. | | HWLQLKPGQPMYRRRRRRRR (SEQ |
| | cerevisiae alpha | | ID NO: 5729) |
| | pheromone fused to | | |
| | octoarginine | | |
| | | R | MATTKHLALAILVLLSIGMTTSARWH |
| | | | WLQLKPGQPMYRRRRRRRR (SEQ ID |
| | | | NO: 5730) |
| | | | MATTKHLALAILVLLSIGMTTSAWH |
| | | | WLQLKPGQPMYRRRRRRRR (SEQ ID |
| | | | NO: 5731) |

### Example 12

This example describes identification of conidial germination-inhibiting (CGI) factor sequences.

Additional conidial germination-inhibiting (CGI) factor sequences were identified from ascomycete fungi and the CGI factor amino acid sequences are provided here as SEQ ID NOs: 2457-3361. In addition, at least one corresponding genomic DNA sequence was identified for each of the CGI factors, and are provided as SEQ ID NOs: 3362-5698. The ascomycete fungal genomes included genomes of fungal species known to be pathogenic to animals, e.g., invertebrates (such as arthropods, nematodes, annelids, helminths, molluscs) and vertebrates (such as mammals, birds, cartilaginous or bony fish, reptiles, or amphibians). In some embodiments, the fungal genome is of a fungal species that is pathogenic in birds, for example, domesticated poultry and waterfowl. In some embodiments, the fungal genome is of a fungal species that is pathogenic in mammals, for example, domestic mammals (dogs, cats, domestic ungulates, and lagomorphs), non-human primates, or humans. In certain embodiments, the fungal genome is of a fungal species that is pathogenic in humans, including adults and non-adults (infants and children).

TABLE 6
Additional CGI factor sequences

| | | Amino acid | Genomic DNA SEQ IN NO | | | | | | | | | | | | | | | |
|---|---|---|---|---|---|---|---|---|---|---|---|---|---|---|---|---|---|---|
| Organism Name | GenBank ID | SEQ ID | 1 | 2 | 3 | 4 | 5 | 6 | 7 | 8 | 9 | 10 | 11 | 12 | 13 | 14 | 15 | 16 |
| Aaosphaeria arxii CBS | KAF2015995.1 | 2253 | 3362 | | | | | | | | | | | | | | | |
| 175.79 | | | | | | | | | | | | | | | | | | |
| Aaosphaeria arxii CBS | KAF2015995.1 | 2254 | 3363 | | | | | | | | | | | | | | | |
| 175.79 | | | | | | | | | | | | | | | | | | |
| Aaosphaeria arxii CBS | KAF2015995.1 | 2255 | 3364 | 4693 | | | | | | | | | | | | | | |
| 175.79 | | | | | | | | | | | | | | | | | | |
| Aaosphaeria arxii CBS | KAF2015995.1 | 2256 | 3365 | 4694 | | | | | | | | | | | | | | |
| 175.79 | | | | | | | | | | | | | | | | | | |
| Aaosphaeria arxii CBS | KAF2015995.1 | 2257 | 3366 | 4695 | | | | | | | | | | | | | | |
| 175.79 | | | | | | | | | | | | | | | | | | |
| Aaosphaeria arxii CBS | KAF2015995.1 | 2258 | 3367 | | | | | | | | | | | | | | | |
| 175.79 | | | | | | | | | | | | | | | | | | |
| Acremonium chrysogenum | KFH48810.1 | 2259 | 3368 | | | | | | | | | | | | | | | |
| ATCC 11550 | | | | | | | | | | | | | | | | | | |
| Acremonium chrysogenum | KFH48810.1 | 2260 | 3369 | | | | | | | | | | | | | | | |
| ATCC 11550 | | | | | | | | | | | | | | | | | | |
| Akanthomyces lecanii | OAA76807.1 | 2261 | 3370 | | | | | | | | | | | | | | | |
| RCEF 1005 | | | | | | | | | | | | | | | | | | |
| Akanthomyces lecanii | OAA76807.1 | 2262 | 3371 | | | | | | | | | | | | | | | |
| RCEF 1005 | | | | | | | | | | | | | | | | | | |
| Amniculicola lignicola CBS | KAF1998093.1 | 2263 | 3372 | | | | | | | | | | | | | | | |
| 123094 | | | | | | | | | | | | | | | | | | |
| Amniculicola lignicola CBS | KAF1998093.1 | 2264 | 3373 | | | | | | | | | | | | | | | |
| 123094 | | | | | | | | | | | | | | | | | | |
| Amniculicola lignicola CBS | KAF1998093.1 | 2265 | 3374 | 4651 | | | | | | | | | | | | | | |
| 123094 | | | | | | | | | | | | | | | | | | |
| Amorphotheca resinae | PSS27704.1 | 2266 | 3375 | 4707 | 5040 | 5328 | | | | | | | | | | | | |
| ATCC 22711 | | | | | | | | | | | | | | | | | | |
| Amorphotheca resinae | PSS27704.1 | 2267 | 3376 | | | | | | | | | | | | | | | |
| ATCC 22711 | | | | | | | | | | | | | | | | | | |
| Amorphotheca resinae | PSS27704.1 | 2268 | 3377 | | | | | | | | | | | | | | | |
| ATCC 22711 | | | | | | | | | | | | | | | | | | |
| Amorphotheca resinae | PSS27704.1 | 2269 | 3378 | | | | | | | | | | | | | | | |
| ATCC 22711 | | | | | | | | | | | | | | | | | | |
| Aplosporella prunicola CBS | KAF2141764.1 | 2270 | 3379 | 4728 | | | | | | | | | | | | | | |
| 121167 | | | | | | | | | | | | | | | | | | |
| Aplosporella prunicola CBS | KAF2141764.1 | 2271 | 3380 | | | | | | | | | | | | | | | |
| 121167 | | | | | | | | | | | | | | | | | | |
| Aplosporella prunicola CBS | KAF2141764.1 | 2272 | 3381 | 4730 | | | | | | | | | | | | | | |
| 121167 | | | | | | | | | | | | | | | | | | |
| Aspergillus aculeatinus | RAH72722.1 | 2273 | 3382 | | | | | | | | | | | | | | | |
| CBS 121060 | | | | | | | | | | | | | | | | | | |
| Aspergillus aculeatinus | RAH72722.1 | 2274 | 3383 | | | | | | | | | | | | | | | |
| CBS 121060 | | | | | | | | | | | | | | | | | | |
| Aspergillus aculeatus | OJK00586.1 | 2275 | 3384 | | | | | | | | | | | | | | | |
| ATCC 16872 | | | | | | | | | | | | | | | | | | |
| Aspergillus aculeatus | OJK00586.1 | 2276 | 3385 | | | | | | | | | | | | | | | |
| ATCC 16872 | | | | | | | | | | | | | | | | | | |
| Aspergillus alliaceus | KAB8230652.1 | 2277 | 3386 | | | | | | | | | | | | | | | |
| Aspergillus alliaceus | KAB8230652.1 | 2278 | 3387 | | | | | | | | | | | | | | | |
| Aspergillus alliaceus | KAE8389964.1 | 2279 | 3388 | | | | | | | | | | | | | | | |
| Aspergillus alliaceus | KAE8389964.1 | 2280 | 3389 | | | | | | | | | | | | | | | |
| Aspergillus arachidicola | PIG89612.1 | 2281 | 3390 | | | | | | | | | | | | | | | |
| Aspergillus arachidicola | PIG89612.1 | 2282 | 3391 | | | | | | | | | | | | | | | |
| Aspergillus avenaceus | KAE8144608.1 | 2283 | 3392 | | | | | | | | | | | | | | | |
| Aspergillus avenaceus | KAE8144608.1 | 2284 | 3393 | | | | | | | | | | | | | | | |
| Aspergillus awamori | GCB22304.1 | 2285 | 3394 | | | | | | | | | | | | | | | |
| Aspergillus awamori | GCB22304.1 | 2286 | 3395 | | | | | | | | | | | | | | | |
| Aspergillus bertholletiae | KAE8376855.1 | 2287 | 3396 | | | | | | | | | | | | | | | |
| Aspergillus bertholletiae | KAE8376855.1 | 2288 | 3397 | | | | | | | | | | | | | | | |
| Aspergillus bombycis | OGM40854.1 | 2289 | 3398 | | | | | | | | | | | | | | | |
| Aspergillus bombycis | OGM40854.1 | 2290 | 3399 | | | | | | | | | | | | | | | |
| Aspergillus brasiliensis | OJJ70798.1 | 2291 | 3400 | | | | | | | | | | | | | | | |
| CBS 101740 | | | | | | | | | | | | | | | | | | |
| Aspergillus brasiliensis | OJJ70798.1 | 2292 | 3401 | | | | | | | | | | | | | | | |
| CBS 101740 | | | | | | | | | | | | | | | | | | |
| Aspergillus brunneoviolaceus CBS | RAH46433.1 | 2293 | 3402 | | | | | | | | | | | | | | | |
| 621.78 | | | | | | | | | | | | | | | | | | |
| Aspergillus brunneoviolaceus CBS | RAH46433.1 | 2294 | 3403 | | | | | | | | | | | | | | | |
| 621.78 | | | | | | | | | | | | | | | | | | |
| Aspergillus campestris IBT | PKY03999.1 | 2295 | 3404 | | | | | | | | | | | | | | | |
| 28561 | | | | | | | | | | | | | | | | | | |
| Aspergillus campestris IBT | PKY03999.1 | 2296 | 3405 | | | | | | | | | | | | | | | |
| 28561 | | | | | | | | | | | | | | | | | | |
| Aspergillus candidus | PLB36309.1 | 2297 | 3406 | | | | | | | | | | | | | | | |
| Aspergillus candidus | PLB36309.1 | 2298 | 3407 | | | | | | | | | | | | | | | |
| Aspergillus carbonarius | OOF96912.1 | 2299 | 3408 | | | | | | | | | | | | | | | |
| ITEM 5010 | | | | | | | | | | | | | | | | | | |
| Aspergillus carbonarius | OOF96912.1 | 2300 | 3409 | | | | | | | | | | | | | | | |
| ITEM 5010 | | | | | | | | | | | | | | | | | | |
| Aspergillus carbonarius | OOF96947.1 | 2301 | 3410 | | | | | | | | | | | | | | | |
| ITEM 5010 | | | | | | | | | | | | | | | | | | |
| Aspergillus carbonarius | OOF96947.1 | 2302 | 3411 | | | | | | | | | | | | | | | |
| ITEM 5010 | | | | | | | | | | | | | | | | | | |
| Aspergillus clavatus NRRL 1 | EAW11125.1 | 2303 | 3412 | | | | | | | | | | | | | | | |
| Aspergillus clavatus NRRL 1 | EAW11125.1 | 2304 | 3413 | | | | | | | | | | | | | | | |
| Aspergillus coremiiformis | KAE8348912.1 | 2305 | 3414 | | | | | | | | | | | | | | | |
| Aspergillus coremiiformis | KAE8348912.1 | 2306 | 3415 | | | | | | | | | | | | | | | |
| Aspergillus costaricaensis | RAK83224.1 | 2307 | 3416 | | | | | | | | | | | | | | | |
| CBS 115574 | | | | | | | | | | | | | | | | | | |
| Aspergillus costaricaensis | RAK83224.1 | 2308 | 3417 | | | | | | | | | | | | | | | |
| CBS 115574 | | | | | | | | | | | | | | | | | | |
| Aspergillus ellipticus CBS | PYH94413.1 | 2309 | 3418 | | | | | | | | | | | | | | | |
| 707.79 | | | | | | | | | | | | | | | | | | |
| Aspergillus ellipticus CBS | PYH94413.1 | 2310 | 3419 | | | | | | | | | | | | | | | |
| 707.79 | | | | | | | | | | | | | | | | | | |
| Aspergillus eucalypticola | PWY62495.1 | 2311 | 3420 | | | | | | | | | | | | | | | |
| CBS 122712 | | | | | | | | | | | | | | | | | | |
| Aspergillus eucalypticola | PWY62495.1 | 2312 | 3421 | | | | | | | | | | | | | | | |
| CBS 122712 | | | | | | | | | | | | | | | | | | |
| Aspergillus fijiensis CBS | RAK82478.1 | 2313 | 3422 | | | | | | | | | | | | | | | |
| 313.89 | | | | | | | | | | | | | | | | | | |
| Aspergillus fijiensis CBS | RAK82478.1 | 2314 | 3423 | | | | | | | | | | | | | | | |
| 313.89 | | | | | | | | | | | | | | | | | | |
| Aspergillus fischeri NRRL | EAW15854.1 | 2315 | 3424 | | | | | | | | | | | | | | | |
| 181 | | | | | | | | | | | | | | | | | | |
| Aspergillus fischeri NRRL | EAW15854.1 | 2316 | 3425 | | | | | | | | | | | | | | | |
| 181 | | | | | | | | | | | | | | | | | | |
| Aspergillus flavus | RAQ43823.1 | 2317 | 3426 | | | | | | | | | | | | | | | |
| Aspergillus flavus | RAQ43823.1 | 2318 | 3427 | | | | | | | | | | | | | | | |
| Aspergillus flavus | RAQ67862.1 | 2319 | 3428 | | | | | | | | | | | | | | | |
| Aspergillus flavus | RAQ67862.1 | 2320 | 3429 | | | | | | | | | | | | | | | |
| Aspergillus flavus | RAQ79703.1 | 2321 | 3430 | | | | | | | | | | | | | | | |
| Aspergillus flavus | RAQ79703.1 | 2322 | 3431 | | | | | | | | | | | | | | | |
| Aspergillus flavus | RMZ41536.1 | 2323 | 3432 | | | | | | | | | | | | | | | |
| Aspergillus flavus | RMZ41536.1 | 2324 | 3433 | | | | | | | | | | | | | | | |
| Aspergillus flavus AF70 | KOC09807.1 | 2325 | 3434 | | | | | | | | | | | | | | | |
| Aspergillus flavus AF70 | KOC09807.1 | 2326 | 3435 | | | | | | | | | | | | | | | |
| Aspergillus flavus | EED56518.1 | 2327 | 3436 | | | | | | | | | | | | | | | |
| NRRL3357 | | | | | | | | | | | | | | | | | | |
| Aspergillus flavus | EED56518.1 | 2328 | 3437 | | | | | | | | | | | | | | | |
| NRRL3357 | | | | | | | | | | | | | | | | | | |
| Aspergillus fumigatiaffinis | KAF4221143.1 | 2329 | 3438 | 4602 | 5023 | 5260 | 5401 | 5505 | | | | | | | | | | |
| Aspergillus fumigatiaffinis | KAF4221143.1 | 2330 | 3439 | 4603 | 5024 | 5261 | 5434 | | | | | | | | | | | |
| Aspergillus fumigatiaffinis | KAF4231642.1 | 2331 | 3440 | | | | | | | | | | | | | | | |
| Aspergillus fumigatiaffinis | KAF4231642.1 | 2332 | 3441 | | | | | | | | | | | | | | | |
| Aspergillus fumigatiaffinis | KAF4243273.1 | 2333 | 3442 | | | | | | | | | | | | | | | |
| Aspergillus fumigatiaffinis | KAF4243273.1 | 2334 | 3443 | | | | | | | | | | | | | | | |
| Aspergillus fumigatiaffinis | KAF4251015.1 | 2335 | 3444 | 4777 | 5084 | | | | | | | | | | | | | |
| Aspergillus fumigatiaffinis | KAF4251015.1 | 2336 | 3445 | 4778 | 5085 | | | | | | | | | | | | | |
| Aspergillus fumigatus | KAF4253266.1 | 2337 | 3446 | | | | | | | | | | | | | | | |
| Aspergillus fumigatus | KAF4253266.1 | 2338 | 3447 | | | | | | | | | | | | | | | |
| Aspergillus fumigatus | KAF4258265.1 | 2339 | 3448 | | | | | | | | | | | | | | | |
| Aspergillus fumigatus | KAF4258265.1 | 2340 | 3449 | | | | | | | | | | | | | | | |
| Aspergillus fumigatus | KAF4260528.1 | 2341 | 3450 | | | | | | | | | | | | | | | |
| Aspergillus fumigatus | KAF4260528.1 | 2342 | 3451 | | | | | | | | | | | | | | | |
| Aspergillus fumigatus | KAF4280373.1 | 2343 | 3452 | | | | | | | | | | | | | | | |
| Aspergillus fumigatus | KAF4280373.1 | 2344 | 3453 | | | | | | | | | | | | | | | |
| Aspergillus fumigatus | KAF4289752.1 | 2345 | 3454 | | | | | | | | | | | | | | | |
| Aspergillus fumigatus | KAF4289752.1 | 2346 | 3455 | | | | | | | | | | | | | | | |
| Aspergillus fumigatus | OXN06038.1 | 2347 | 3456 | | | | | | | | | | | | | | | |
| Aspergillus fumigatus | OXN06038.1 | 2348 | 3457 | | | | | | | | | | | | | | | |
| Aspergillus fumigatus | OXN25660.1 | 2349 | 3458 | | | | | | | | | | | | | | | |
| Aspergillus fumigatus | OXN25660.1 | 2350 | 3459 | | | | | | | | | | | | | | | |
| Aspergillus fumigatus | EDP49202.1 | 2351 | 3460 | | | | | | | | | | | | | | | |
| A1163 | | | | | | | | | | | | | | | | | | |
| Aspergillus fumigatus | EDP49202.1 | 2352 | 3461 | | | | | | | | | | | | | | | |
| A1163 | | | | | | | | | | | | | | | | | | |
| Aspergillus fumigatus | EAL88490.1 | 2353 | 3462 | | | | | | | | | | | | | | | |
| Af293 | | | | | | | | | | | | | | | | | | |
| Aspergillus fumigatus | EAL88490.1 | 2354 | 3463 | | | | | | | | | | | | | | | |
| Af293 | | | | | | | | | | | | | | | | | | |
| Aspergillus fumigatus Z5 | KMK62965.1 | 2355 | 3464 | | | | | | | | | | | | | | | |
| Aspergillus fumigatus Z5 | KMK62965.1 | 2356 | 3465 | | | | | | | | | | | | | | | |
| Aspergillus fumigatus var. | KEY83811.1 | 2357 | 3466 | | | | | | | | | | | | | | | |
| RP-2014 | | | | | | | | | | | | | | | | | | |
| Aspergillus fumigatus var. | KEY83811.1 | 2358 | 3467 | | | | | | | | | | | | | | | |
| RP-2014 | | | | | | | | | | | | | | | | | | |
| Aspergillus heteromorphus | PWY76160.1 | 2359 | 3468 | | | | | | | | | | | | | | | |
| CBS 117.55 | | | | | | | | | | | | | | | | | | |
| Aspergillus heteromorphus | PWY76160.1 | 2360 | 3469 | | | | | | | | | | | | | | | |
| CBS 117.55 | | | | | | | | | | | | | | | | | | |
| Aspergillus homomorphus | RAL08699.1 | 2361 | 3470 | | | | | | | | | | | | | | | |
| CBS 101889 | | | | | | | | | | | | | | | | | | |
| Aspergillus homomorphus | RAL08699.1 | 2362 | 3471 | | | | | | | | | | | | | | | |
| CBS 101889 | | | | | | | | | | | | | | | | | | |
| Aspergillus ibericus CBS | RAK98322.1 | 2363 | 3472 | | | | | | | | | | | | | | | |
| 121593 | | | | | | | | | | | | | | | | | | |
| Aspergillus ibericus CBS | RAK98322.1 | 2364 | 3473 | | | | | | | | | | | | | | | |
| 121593 | | | | | | | | | | | | | | | | | | |
| Aspergillus indologenus | PYI25318.1 | 2365 | 3474 | | | | | | | | | | | | | | | |
| CBS 114.80 | | | | | | | | | | | | | | | | | | |
| Aspergillus indologenus | PYI25318.1 | 2366 | 3475 | | | | | | | | | | | | | | | |
| CBS 114.80 | | | | | | | | | | | | | | | | | | |
| Aspergillus japonicus CBS | RAH83413.1 | 2367 | 3476 | | | | | | | | | | | | | | | |
| 114.51 | | | | | | | | | | | | | | | | | | |
| Aspergillus japonicus CBS | RAH83413.1 | 2368 | 3477 | | | | | | | | | | | | | | | |
| 114.51 | | | | | | | | | | | | | | | | | | |
| Aspergillus lentulus | GAQ03497.1 | 2369 | 3478 | | | | | | | | | | | | | | | |
| Aspergillus lentulus | GAQ03497.1 | 2370 | 3479 | | | | | | | | | | | | | | | |
| Aspergillus lentulus | GFF27988.1 | 2371 | 3480 | | | | | | | | | | | | | | | |
| Aspergillus lentulus | GFF27988.1 | 2372 | 3481 | | | | | | | | | | | | | | | |
| Aspergillus lentulus | GFF47840.1 | 2373 | 3482 | | | | | | | | | | | | | | | |
| Aspergillus lentulus | GFF47840.1 | 2374 | 3483 | | | | | | | | | | | | | | | |
| Aspergillus lentulus | GFF76341.1 | 2375 | 3484 | | | | | | | | | | | | | | | |
| Aspergillus lentulus | GFF76341.1 | 2376 | 3485 | | | | | | | | | | | | | | | |
| Aspergillus lentulus | GFF92029.1 | 2377 | 3486 | | | | | | | | | | | | | | | |
| Aspergillus lentulus | GFF92029.1 | 2378 | 3487 | | | | | | | | | | | | | | | |
| Aspergillus lentulus | GFG02708.1 | 2379 | 3488 | | | | | | | | | | | | | | | |
| Aspergillus lentulus | GFG02708.1 | 2380 | 3489 | | | | | | | | | | | | | | | |
| Aspergillus lentulus | KAF4156140.1 | 2381 | 3490 | | | | | | | | | | | | | | | |
| Aspergillus lentulus | KAF4156140.1 | 2382 | 3491 | | | | | | | | | | | | | | | |
| Aspergillus lentulus | KAF4163322.1 | 2383 | 3492 | | | | | | | | | | | | | | | |
| Aspergillus lentulus | KAF4163322.1 | 2384 | 3493 | | | | | | | | | | | | | | | |
| Aspergillus lentulus | KAF4181253.1 | 2385 | 3494 | 4672 | 5052 | 5262 | | | | | | | | | | | | |
| Aspergillus lentulus | KAF4181253.1 | 2386 | 3495 | 4673 | 5054 | | | | | | | | | | | | | |
| Aspergillus lentulus | KAF4186434.1 | 2387 | 3496 | | | | | | | | | | | | | | | |
| Aspergillus lentulus | KAF4186434.1 | 2388 | 3497 | | | | | | | | | | | | | | | |
| Aspergillus lentulus | KAF4198353.1 | 2389 | 3498 | 4674 | 5053 | 5263 | | | | | | | | | | | | |
| Aspergillus lentulus | KAF4198353.1 | 2390 | 3499 | 4675 | 5055 | | | | | | | | | | | | | |
| Aspergillus lentulus | KAF4205243.1 | 2391 | 3500 | | | | | | | | | | | | | | | |
| Aspergillus lentulus | KAF4205243.1 | 2392 | 3501 | | | | | | | | | | | | | | | |
| Aspergillus luchuensis | GAT21750.1 | 2393 | 3502 | | | | | | | | | | | | | | | |
| Aspergillus luchuensis | GAT21750.1 | 2394 | 3503 | | | | | | | | | | | | | | | |
| Aspergillus luchuensis CBS | OJZ84143.1 | 2395 | 3504 | | | | | | | | | | | | | | | |
| 106.47 | | | | | | | | | | | | | | | | | | |
| Aspergillus luchuensis CBS | OJZ84143.1 | 2396 | 3505 | | | | | | | | | | | | | | | |
| 106.47 | | | | | | | | | | | | | | | | | | |
| Aspergillus luchuensis IFO | GAA90114.1 | 2397 | 3506 | | | | | | | | | | | | | | | |
| 4308 | | | | | | | | | | | | | | | | | | |
| Aspergillus luchuensis IFO | GAA90114.1 | 2398 | 3507 | | | | | | | | | | | | | | | |
| 4308 | | | | | | | | | | | | | | | | | | |
| Aspergillus mulundensis | RDW57407.1 | 2399 | 3508 | | | | | | | | | | | | | | | |
| Aspergillus mulundensis | RDW57407.1 | 2400 | 3509 | 4492 | 4910 | | | | | | | | | | | | | |
| Aspergillus neoniger CBS | PYH29563.1 | 2401 | 3510 | | | | | | | | | | | | | | | |
| 115656 | | | | | | | | | | | | | | | | | | |
| Aspergillus neoniger CBS | PYH29563.1 | 2402 | 3511 | | | | | | | | | | | | | | | |
| 115656 | | | | | | | | | | | | | | | | | | |
| Aspergillus niger | CAK43384.1 | 2403 | 3512 | | | | | | | | | | | | | | | |
| Aspergillus niger | CAK43384.1 | 2404 | 3513 | | | | | | | | | | | | | | | |
| Aspergillus niger | GAQ44527.1 | 2405 | 3514 | | | | | | | | | | | | | | | |
| Aspergillus niger | GAQ44527.1 | 2406 | 3515 | | | | | | | | | | | | | | | |
| Aspergillus niger | SPB53044.1 | 2407 | 3516 | | | | | | | | | | | | | | | |
| Aspergillus niger | SPB53044.1 | 2408 | 3517 | | | | | | | | | | | | | | | |
| Aspergillus niger | TPR01726.1 | 2409 | 3518 | | | | | | | | | | | | | | | |
| Aspergillus niger | TPR01726.1 | 2410 | 3519 | | | | | | | | | | | | | | | |
| Aspergillus niger ATCC | EHA19251.1 | 2411 | 3520 | | | | | | | | | | | | | | | |
| 1015 | | | | | | | | | | | | | | | | | | |
| Aspergillus niger ATCC | EHA19251.1 | 2412 | 3521 | | | | | | | | | | | | | | | |
| 1015 | | | | | | | | | | | | | | | | | | |
| Aspergillus niger ATCC | RDH23413.1 | 2413 | 3522 | | | | | | | | | | | | | | | |
| 13496 | | | | | | | | | | | | | | | | | | |
| Aspergillus niger ATCC | RDH23413.1 | 2414 | 3523 | | | | | | | | | | | | | | | |
| 13496 | | | | | | | | | | | | | | | | | | |
| Aspergillus niger CBS | PYH56423.1 | 2415 | 3524 | | | | | | | | | | | | | | | |
| 101883 | | | | | | | | | | | | | | | | | | |
| Aspergillus niger CBS | PYH56423.1 | 2416 | 3525 | | | | | | | | | | | | | | | |
| 101883 | | | | | | | | | | | | | | | | | | |
| Aspergillus nomiae NRRL | KNG86060.1 | 2417 | 3526 | | | | | | | | | | | | | | | |
| 13137 | | | | | | | | | | | | | | | | | | |
| Aspergillus nomiae NRRL | KNG86060.1 | 2418 | 3527 | | | | | | | | | | | | | | | |
| 13137 | | | | | | | | | | | | | | | | | | |
| Aspergillus novofumigatus | PKX95749.1 | 2419 | 3528 | | | | | | | | | | | | | | | |
| IBT 16806 | | | | | | | | | | | | | | | | | | |
| Aspergillus novofumigatus | PKX95749.1 | 2420 | 3529 | | | | | | | | | | | | | | | |
| IBT 16806 | | | | | | | | | | | | | | | | | | |
| Aspergillus phoenicis | RDK36520.1 | 2421 | 3530 | | | | | | | | | | | | | | | |
| ATCC 13157 | | | | | | | | | | | | | | | | | | |
| Aspergillus phoenicis | RDK36520.1 | 2422 | 3531 | | | | | | | | | | | | | | | |
| ATCC 13157 | | | | | | | | | | | | | | | | | | |
| Aspergillus piperis CBS | RAH51938.1 | 2423 | 3532 | | | | | | | | | | | | | | | |
| 112811 | | | | | | | | | | | | | | | | | | |
| Aspergillus piperis CBS | RAH51938.1 | 2424 | 3533 | | | | | | | | | | | | | | | |
| 112811 | | | | | | | | | | | | | | | | | | |
| Aspergillus saccharolyticus | PYH40521.1 | 2425 | 3534 | | | | | | | | | | | | | | | |
| JOP 1030-1 | | | | | | | | | | | | | | | | | | |
| Aspergillus saccharolyticus | PYH40521.1 | 2426 | 3535 | | | | | | | | | | | | | | | |
| JOP 1030-1 | | | | | | | | | | | | | | | | | | |
| Aspergillus sclerotialis | RJE26598.1 | 2427 | 3536 | | | | | | | | | | | | | | | |
| Aspergillus sclerotialis | RJE26598.1 | 2428 | 3537 | | | | | | | | | | | | | | | |
| Aspergillus sclerotiicarbonarius CBS | PYI09055.1 | 2429 | 3538 | | | | | | | | | | | | | | | |
| 121057 | | | | | | | | | | | | | | | | | | |
| Aspergillus sclerotiicarbonarius CBS | PYI09055.1 | 2430 | 3539 | | | | | | | | | | | | | | | |
| 121057 | | | | | | | | | | | | | | | | | | |
| Aspergillus taichungensis | PLN84247.1 | 2431 | 3540 | | | | | | | | | | | | | | | |
| Aspergillus taichungensis | PLN84247.1 | 2432 | 3541 | | | | | | | | | | | | | | | |
| Aspergillus terreus | GFF18647.1 | 2433 | 3542 | | | | | | | | | | | | | | | |
| Aspergillus terreus | GFF18647.1 | 2434 | 3543 | | | | | | | | | | | | | | | |
| Aspergillus terreus | EAU31669.1 | 2435 | 3544 | | | | | | | | | | | | | | | |
| NIH2624 | | | | | | | | | | | | | | | | | | |
| Aspergillus terreus | EAU31669.1 | 2436 | 3545 | | | | | | | | | | | | | | | |
| NIH2624 | | | | | | | | | | | | | | | | | | |
| Aspergillus thermomutatus | RHZ62685.1 | 2437 | 3546 | | | | | | | | | | | | | | | |
| Aspergillus thermomutatus | RHZ62685.1 | 2438 | 3547 | | | | | | | | | | | | | | | |
| Aspergillus tubingensis | GFN10216.1 | 2439 | 3548 | | | | | | | | | | | | | | | |
| Aspergillus tubingensis | GFN10216.1 | 2440 | 3549 | | | | | | | | | | | | | | | |
| Aspergillus tubingensis | OJI90517.1 | 2441 | 3550 | | | | | | | | | | | | | | | |
| CBS 134.48 | | | | | | | | | | | | | | | | | | |
| Aspergillus tubingensis | OJI90517.1 | 2442 | 3551 | | | | | | | | | | | | | | | |
| CBS 134.48 | | | | | | | | | | | | | | | | | | |
| Aspergillus turcosus | RHZ68865.1 | 2443 | 3552 | | | | | | | | | | | | | | | |
| Aspergillus turcosus | RHZ68865.1 | 2444 | 3553 | | | | | | | | | | | | | | | |
| Aspergillus turcosus | RLL97228.1 | 2445 | 3554 | | | | | | | | | | | | | | | |
| Aspergillus turcosus | RLL97228.1 | 2446 | 3555 | | | | | | | | | | | | | | | |
| Aspergillus udagawae | GAO87704.1 | 2447 | 3556 | | | | | | | | | | | | | | | |
| Aspergillus udagawae | GAO87704.1 | 2448 | 3557 | | | | | | | | | | | | | | | |
| Aspergillus udagawae | GFF35348.1 | 2449 | 3558 | | | | | | | | | | | | | | | |
| Aspergillus udagawae | GFF35348.1 | 2450 | 3559 | | | | | | | | | | | | | | | |
| Aspergillus udagawae | GFF49395.1 | 2451 | 3560 | | | | | | | | | | | | | | | |
| Aspergillus udagawae | GFF49395.1 | 2452 | 3561 | | | | | | | | | | | | | | | |
| Aspergillus udagawae | GFF89968.1 | 2453 | 3562 | | | | | | | | | | | | | | | |
| Aspergillus udagawae | GFF89968.1 | 2454 | 3563 | | | | | | | | | | | | | | | |
| Aspergillus udagawae | GFG13593.1 | 2455 | 3564 | | | | | | | | | | | | | | | |
| Aspergillus udagawae | GFG13593.1 | 2456 | 3565 | | | | | | | | | | | | | | | |
| Aspergillus uvarum CBS | PYH76147.1 | 2457 | 3566 | | | | | | | | | | | | | | | |
| 121591 | | | | | | | | | | | | | | | | | | |
| Aspergillus uvarum CBS | PYH76147.1 | 2458 | 3567 | | | | | | | | | | | | | | | |
| 121591 | | | | | | | | | | | | | | | | | | |
| Aspergillus vadensis CBS | PYH71369.1 | 2459 | 3568 | | | | | | | | | | | | | | | |
| 113365 | | | | | | | | | | | | | | | | | | |
| Aspergillus vadensis CBS | PYH71369.1 | 2460 | 3569 | | | | | | | | | | | | | | | |
| 113365 | | | | | | | | | | | | | | | | | | |
| Aspergillus violaceofuscus | PYI23329.1 | 2461 | 3570 | | | | | | | | | | | | | | | |
| CBS 115571 | | | | | | | | | | | | | | | | | | |
| Aspergillus violaceofuscus | PYI23329.1 | 2462 | 3571 | | | | | | | | | | | | | | | |
| CBS 115571 | | | | | | | | | | | | | | | | | | |
| Aspergillus welwitschiae | RDH32041.1 | 2463 | 3572 | | | | | | | | | | | | | | | |
| Aspergillus welwitschiae | RDH32041.1 | 2464 | 3573 | | | | | | | | | | | | | | | |
| Aulographum hederae CBS | KAF1987766.1 | 2465 | 3574 | | | | | | | | | | | | | | | |
| 113979 | | | | | | | | | | | | | | | | | | |
| Aulographum hederae CBS | KAF1987766.1 | 2466 | 3575 | | | | | | | | | | | | | | | |
| 113979 | | | | | | | | | | | | | | | | | | |
| Aulographum hederae CBS | KAF1987766.1 | 2467 | 3576 | | | | | | | | | | | | | | | |
| 113979 | | | | | | | | | | | | | | | | | | |
| Aulographum hederae CBS | KAF1987766.1 | 2468 | 3577 | | | | | | | | | | | | | | | |
| 113979 | | | | | | | | | | | | | | | | | | |
| Aulographum hederae CBS | KAF1987766.1 | 2469 | 3578 | | | | | | | | | | | | | | | |
| 113979 | | | | | | | | | | | | | | | | | | |
| Beauveria bassiana | KAF1729757.1 | 2470 | 3579 | 4769 | | | | | | | | | | | | | | |
| Beauveria bassiana | KAF1729757.1 | 2471 | 3580 | 4770 | | | | | | | | | | | | | | |
| Beauveria bassiana | KAF1729757.1 | 2472 | 3581 | 4765 | 5080 | | | | | | | | | | | | | |
| Beauveria bassiana | PMB64760.1 | 2473 | 3582 | 4771 | | | | | | | | | | | | | | |
| Beauveria bassiana | PMB64760.1 | 2474 | 3583 | 4772 | | | | | | | | | | | | | | |
| Beauveria bassiana | PMB64760.1 | 2475 | 3584 | 4766 | 5081 | | | | | | | | | | | | | |
| Beauveria bassiana ARSEF | EJP68112.1 | 2476 | 3585 | 4773 | | | | | | | | | | | | | | |
| 2860 | | | | | | | | | | | | | | | | | | |
| Beauveria bassiana ARSEF | EJP68112.1 | 2477 | 3586 | 4774 | | | | | | | | | | | | | | |
| 2860 | | | | | | | | | | | | | | | | | | |
| Beauveria bassiana ARSEF | EJP68112.1 | 2478 | 3587 | 4767 | 5082 | | | | | | | | | | | | | |
| 2860 | | | | | | | | | | | | | | | | | | |
| Beauveria bassiana D1-5 | KGQ07814.1 | 2479 | 3588 | 4775 | | | | | | | | | | | | | | |
| Beauveria bassiana D1-5 | KGQ07814.1 | 2480 | 3589 | 4776 | | | | | | | | | | | | | | |
| Beauveria bassiana D1-5 | KGQ07814.1 | 2481 | 3590 | 4768 | 5083 | | | | | | | | | | | | | |
| Beauveria brongniartii | OAA52838.1 | 2482 | 3591 | 4857 | | | | | | | | | | | | | | |
| RCEF 3172 | | | | | | | | | | | | | | | | | | |
| Beauveria brongniartii | OAA52838.1 | 2483 | 3592 | 4858 | | | | | | | | | | | | | | |
| RCEF 3172 | | | | | | | | | | | | | | | | | | |
| Beauveria brongniartii | OAA52838.1 | 2484 | 3593 | | | | | | | | | | | | | | | |
| RCEF 3172 | | | | | | | | | | | | | | | | | | |
| Botryosphaeria dothidea | KAF4309375.1 | 2485 | 3594 | | | | | | | | | | | | | | | |
| Botryosphaeria dothidea | KAF4309375.1 | 2486 | 3595 | 4659 | 5018 | | | | | | | | | | | | | |
| Botryosphaeria dothidea | KAF4309375.1 | 2487 | 3596 | 4664 | | | | | | | | | | | | | | |
| Botryosphaeria dothidea | KAF4309375.1 | 2488 | 3597 | | | | | | | | | | | | | | | |
| Botryosphaeria dothidea | KAF4312262.1 | 2489 | 3598 | | | | | | | | | | | | | | | |
| Botryosphaeria dothidea | KAF4312262.1 | 2490 | 3599 | 4660 | 5019 | | | | | | | | | | | | | |
| Botryosphaeria dothidea | KAF4312262.1 | 2491 | 3600 | | | | | | | | | | | | | | | |
| Botryosphaeria dothidea | KAF4312262.1 | 2492 | 3601 | | | | | | | | | | | | | | | |
| Botryotinia calthae | TEY43656.1 | 2493 | 3602 | | | | | | | | | | | | | | | |
| Botryotinia calthae | TEY43656.1 | 2494 | 3603 | | | | | | | | | | | | | | | |
| Botryotinia calthae | TEY43656.1 | 2495 | 3604 | 4833 | 5126 | 5319 | 5431 | 5503 | 5559 | | | | | | | | | |
| Botryotinia calthae | TEY43656.1 | 2496 | 3605 | 4828 | 5127 | 5320 | 5432 | 5504 | 5560 | | | | | | | | | |
| Botryotinia calthae | TEY43656.1 | 2497 | 3606 | 4829 | 5128 | 5321 | | | | | | | | | | | | |
| Botryotinia calthae | TEY43656.1 | 2498 | 3607 | | | | | | | | | | | | | | | |
| Botryotinia calthae | TEY43656.1 | 2499 | 3608 | | | | | | | | | | | | | | | |
| Botryotinia calthae | TEY43656.1 | 2500 | 3609 | | | | | | | | | | | | | | | |
| Botryotinia calthae | TEY43656.1 | 2501 | 3610 | | | | | | | | | | | | | | | |
| Botryotinia convoluta | TGO65046.1 | 2502 | 3611 | | | | | | | | | | | | | | | |
| Botryotinia convoluta | TGO65046.1 | 2503 | 3612 | 4823 | 5115 | 5294 | 5413 | 5483 | 5557 | | | | | | | | | |
| Botryotinia convoluta | TGO65046.1 | 2504 | 3613 | 4821 | 5116 | 5295 | 5414 | 5484 | 5558 | | | | | | | | | |
| Botryotinia convoluta | TGO65046.1 | 2505 | 3614 | 4861 | | | | | | | | | | | | | | |
| Botryotinia convoluta | TGO65046.1 | 2506 | 3615 | | | | | | | | | | | | | | | |
| Botryotinia convoluta | TGO65046.1 | 2507 | 3616 | | | | | | | | | | | | | | | |
| Botryotinia narcissicola | TGO66426.1 | 2508 | 3617 | | | | | | | | | | | | | | | |
| Botryotinia narcissicola | TGO66426.1 | 2509 | 3618 | 4557 | 4952 | 5209 | 5372 | 5465 | 5565 | 5602 | 5632 | | | | | | | |
| Botryotinia narcissicola | TGO66426.1 | 2510 | 3619 | 4517 | 4972 | 5218 | 5378 | 5471 | 5537 | 5585 | 2629 | | | | | | | |
| Botryotinia narcissicola | TGO66426.1 | 2511 | 3620 | | | | | | | | | | | | | | | |
| Botryotinia narcissicola | TGO66426.1 | 2512 | 3621 | | | | | | | | | | | | | | | |
| Botryotinia narcissicola | TGO66426.1 | 2513 | 3622 | | | | | | | | | | | | | | | |
| Botrytis cinerea B05.10 | ATZ46079.1 | 2514 | 3623 | | | | | | | | | | | | | | | |
| Botrytis cinerea B05.10 | ATZ46079.1 | 2515 | 3624 | 4553 | 4946 | 5206 | 5369 | 5461 | 5526 | 5598 | 5626 | 5652 | | | | | | |
| Botrytis cinerea B05.10 | ATZ46079.1 | 2516 | 3625 | 4488 | 4900 | 5169 | 5343 | 5455 | 5532 | 5580 | 5614 | 5639 | 5663 | | | | | |
| Botrytis cinerea B05.10 | ATZ46079.1 | 2517 | 3626 | 4518 | 4917 | 5174 | 5347 | 5445 | 5518 | | | | | | | | | |
| Botrytis cinerea B05.10 | ATZ46079.1 | 2518 | 3627 | 4522 | 4921 | 5177 | 5364 | 5468 | 5540 | | | | | | | | | |
| Botrytis cinerea B05.10 | ATZ46079.1 | 2519 | 3628 | | | | | | | | | | | | | | | |
| Botrytis cinerea B05.10 | ATZ46079.1 | 2520 | 3629 | | | | | | | | | | | | | | | |
| Botrytis cinerea B05.10 | ATZ46079.1 | 2521 | 3630 | | | | | | | | | | | | | | | |
| Botrytis cinerea BcDW1 | EMR84438.1 | 2522 | 3631 | | | | | | | | | | | | | | | |
| Botrytis cinerea BcDW1 | EMR84438.1 | 2523 | 3632 | 4554 | 4949 | 5208 | 5371 | 5463 | 5528 | 5600 | 5628 | 5654 | | | | | | |
| Botrytis cinerea BcDW1 | EMR84438.1 | 2524 | 3633 | 4489 | 4901 | 5170 | 5344 | 5456 | 5533 | 5581 | 5615 | 5640 | | | | | | |
| Botrytis cinerea BcDW1 | EMR84438.1 | 2525 | 3634 | 4519 | 4918 | 5175 | 5348 | 5446 | 5519 | | | | | | | | | |
| Botrytis cinerea BcDW1 | EMR84438.1 | 2526 | 3635 | 4523 | 4923 | 5179 | 5366 | 5470 | 5542 | | | | | | | | | |
| Botrytis cinerea BcDW1 | EMR84438.1 | 2527 | 3636 | | | | | | | | | | | | | | | |
| Botrytis cinerea BcDW1 | EMR84438.1 | 2528 | 3637 | | | | | | | | | | | | | | | |
| Botrytis cinerea BcDW1 | EMR84438.1 | 2529 | 3638 | | | | | | | | | | | | | | | |
| Botrytis cinerea T4 | CCD45798.1 | 2530 | 3639 | | | | | | | | | | | | | | | |
| Botrytis cinerea T4 | CCD45798.1 | 2531 | 3640 | 4555 | 4947 | 5207 | 5370 | 5462 | 5527 | 5599 | 5627 | 2653 | 5664 | | | | | |
| Botrytis cinerea T4 | CCD45798.1 | 2532 | 3641 | 4490 | 4902 | 5171 | 5345 | 5457 | 5534 | 5582 | 5616 | 5641 | | | | | | |
| Botrytis cinerea T4 | CCD45798.1 | 2533 | 3642 | 4520 | 4919 | 5176 | 5349 | 5447 | 5520 | | | | | | | | | |
| Botrytis cinerea T4 | CCD45798.1 | 2534 | 3643 | 4524 | 4922 | 5178 | 5365 | 5469 | 5541 | | | | | | | | | |
| Botrytis cinerea T4 | CCD45798.1 | 2535 | 3644 | | | | | | | | | | | | | | | |
| Botrytis cinerea T4 | CCD45798.1 | 2536 | 3645 | | | | | | | | | | | | | | | |
| Botrytis cinerea T4 | CCD45798.1 | 2537 | 3646 | | | | | | | | | | | | | | | |
| Botrytis elliptica | TGO79273.1 | 2538 | 3647 | | | | | | | | | | | | | | | |
| Botrytis elliptica | TGO79273.1 | 2539 | 3648 | | | | | | | | | | | | | | | |
| Botrytis elliptica | TGO79273.1 | 2540 | 3649 | 4818 | 5114 | 5293 | 5412 | 5481 | 5553 | 5594 | 5622 | | | | | | | |
| Botrytis elliptica | TGO79273.1 | 2541 | 3650 | | | | | | | | | | | | | | | |
| Botrytis elliptica | TGO79273.1 | 2542 | 3651 | | | | | | | | | | | | | | | |
| Botrytis fragariae | KAF5878228.1 | 2543 | 3652 | 4681 | | | | | | | | | | | | | | |
| Botrytis fragariae | KAF5878228.1 | 2544 | 3653 | 4516 | 4945 | 5190 | 5363 | 5458 | 5523 | 5573 | 5607 | 5650 | 5671 | 5680 | 5688 | | | |
| Botrytis fragariae | KAF5878228.1 | 2545 | 3654 | 4471 | 4898 | 5167 | 5340 | 5442 | 5512 | 5568 | 5605 | 5635 | 5658 | 5675 | 5683 | 5691 | 5695 | |
| Botrytis fragariae | KAF5878228.1 | 2546 | 3655 | 4556 | 4948 | 5205 | 5367 | 5459 | 5524 | 5583 | 5617 | 5651 | 5672 | 5681 | 5689 | | | |
| Botrytis fragariae | KAF5878228.1 | 2547 | 3656 | 4472 | 4896 | 5166 | 5341 | 5443 | 5513 | 5572 | 5612 | 5367 | 5660 | 5677 | 5685 | 5692 | 5696 | 5698 |
| Botrytis fragariae | KAF5878228.1 | 2548 | 3657 | 4485 | 4920 | 5180 | 5350 | 5448 | 5514 | 5571 | 5604 | 5634 | 5659 | 5676 | 5684 | 5693 | 5697 | |
| Botrytis galanthina | THV53187.1 | 2549 | 3658 | | | | | | | | | | | | | | | |
| Botrytis galanthina | THV53187.1 | 2550 | 3659 | 4558 | 4950 | 5191 | 5368 | 5460 | 5525 | 5601 | 5631 | 5656 | 5673 | | | | | |
| Botrytis galanthina | THV53187.1 | 2551 | 3660 | 4473 | 4913 | 5172 | 5346 | 5444 | 5521 | 5577 | 5613 | 5638 | 5662 | | | | | |
| Botrytis galanthina | THV53187.1 | 2552 | 3661 | | | | | | | | | | | | | | | |
| Botrytis galanthina | THV53187.1 | 2553 | 3662 | | | | | | | | | | | | | | | |
| Botrytis galanthina | THV53187.1 | 2554 | 3663 | | | | | | | | | | | | | | | |
| Botrytis hyacinthi | TGO34871.1 | 2555 | 3664 | | | | | | | | | | | | | | | |
| Botrytis hyacinthi | TGO34871.1 | 2556 | 3665 | 4824 | 5121 | 5299 | 5415 | 5490 | 5551 | 5591 | 5620 | 5647 | 5669 | | | | | |
| Botrytis hyacinthi | TGO34871.1 | 2557 | 3666 | 4822 | 5122 | 5302 | 5416 | 5491 | 5552 | 5592 | 5621 | 5648 | 5670 | | | | | |
| Botrytis hyacinthi | TGO34871.1 | 2558 | 3667 | | | | | | | | | | | | | | | |
| Botrytis hyacinthi | TGO34871.1 | 2559 | 3668 | | | | | | | | | | | | | | | |
| Botrytis hyacinthi | TGO34871.1 | 2560 | 3669 | 4820 | 5120 | 5297 | 5426 | 5500 | 5554 | 5593 | 5623 | 5649 | | | | | | |
| Botrytis paeoniae | TGO31321.1 | 2561 | 3670 | | | | | | | | | | | | | | | |
| Botrytis paeoniae | TGO31321.1 | 2562 | 3671 | 4830 | 5124 | 5300 | 5420 | 5501 | 5561 | | | | | | | | | |
| Botrytis paeoniae | TGO31321.1 | 2563 | 3672 | 4831 | 5123 | 5303 | 5421 | 5502 | 5562 | | | | | | | | | |
| Botrytis paeoniae | TGO31321.1 | 2564 | 3673 | | | | | | | | | | | | | | | |
| Botrytis paeoniae | TGO31321.1 | 2565 | 3674 | | | | | | | | | | | | | | | |
| Botrytis paeoniae | TGO31321.1 | 2566 | 3675 | | | | | | | | | | | | | | | |
| Botrytis paeoniae | TGO31321.1 | 2567 | 3676 | | | | | | | | | | | | | | | |
| Botrytis porri | TGO85790.1 | 2568 | 3677 | 4832 | 5147 | | | | | | | | | | | | | |
| Botrytis porri | TGO85790.1 | 2569 | 3678 | | | | | | | | | | | | | | | |
| Botrytis porri | TGO85790.1 | 2570 | 3679 | | | | | | | | | | | | | | | |
| Botrytis tulipae | TGO18793.1 | 2571 | 3680 | | | | | | | | | | | | | | | |
| Botrytis tulipae | TGO18793.1 | 2572 | 3681 | 4559 | 4951 | 5192 | 5362 | 5464 | 5529 | 5597 | 5630 | 5655 | | | | | | |
| Botrytis tulipae | TGO18793.1 | 2573 | 3682 | 4474 | 4899 | 5168 | 5342 | | 5522 | 5578 | 5611 | 5645 | | | | | | |
| Botrytis tulipae | TGO18793.1 | 2574 | 3683 | | | | | | | | | | | | | | | |
| Botrytis tulipae | TGO18793.1 | 2575 | 3684 | | | | | | | | | | | | | | | |
| Botrytis tulipae | TGO18793.1 | 2576 | 3685 | | | | | | | | | | | | | | | |
| Cadophora sp. DSE1049 | PVH85678.1 | 2577 | 3686 | 4705 | 5041 | 5324 | 5435 | 5508 | 5564 | 5596 | | | | | | | | |
| Cadophora sp. DSE1049 | PVH85678.1 | 2578 | 3687 | 4706 | 5042 | 5325 | 5436 | 5507 | 5563 | 5595 | 5625 | | | | | | | |
| Cenococcum geophilum | OCK98678.1 | 2579 | 3688 | | | | | | | | | | | | | | | |
| 1.58 | | | | | | | | | | | | | | | | | | |
| Cenococcum geophilum | OCK98678.1 | 2580 | 3689 | | | | | | | | | | | | | | | |
| 1.58 | | | | | | | | | | | | | | | | | | |
| Cenococcum geophilum | OCK98678.1 | 2581 | 3690 | 4654 | 5015 | | | | | | | | | | | | | |
| 1.58 | | | | | | | | | | | | | | | | | | |
| Cenococcum geophilum | OCK98678.1 | 2581 | 3691 | | | | | | | | | | | | | | | |
| 1.58 | | | | | | | | | | | | | | | | | | |
| Chaetomium thermophilum | EGS16968.1 | 2583 | 3692 | | | | | | | | | | | | | | | |
| var. thermophilum DSM | | | | | | | | | | | | | | | | | | |
| 1495 | | | | | | | | | | | | | | | | | | |
| Chaetomium thermophilum | EGS16968.1 | 2584 | 3693 | | | | | | | | | | | | | | | |
| var. thermophilum DSM | | | | | | | | | | | | | | | | | | |
| 1495 | | | | | | | | | | | | | | | | | | |
| Chaetomium thermophilum | EGS16968.1 | 2585 | 3694 | | | | | | | | | | | | | | | |
| var. thermophilum DSM | | | | | | | | | | | | | | | | | | |
| 1495 | | | | | | | | | | | | | | | | | | |
| Chaetomium thermophilum | EGS16968.1 | 2586 | 3695 | | | | | | | | | | | | | | | |
| var. thermophilum DSM | | | | | | | | | | | | | | | | | | |
| 1495 | | | | | | | | | | | | | | | | | | |
| Chaetomium thermophilum | EGS16968.1 | 2587 | 3696 | | | | | | | | | | | | | | | |
| var. thermophilum DSM | | | | | | | | | | | | | | | | | | |
| 1495 | | | | | | | | | | | | | | | | | | |
| Chaetomium thermophilum | EGS16968.1 | 2588 | 3697 | 4743 | 5139 | 5313 | | | | | | | | | | | | |
| var. thermophilum DSM | | | | | | | | | | | | | | | | | | |
| 1495 | | | | | | | | | | | | | | | | | | |
| Chalara longipes BDJ | KAE9381296.1 | 2589 | 3698 | 4867 | 5154 | 5329 | | | | | | | | | | | | |
| Chalara longipes BDJ | KAE9381296.1 | 2590 | 3699 | | | | | | | | | | | | | | | |
| Chlorociboria aeruginascens | TAQ88760.1 | 2591 | 3700 | 4870 | | | | | | | | | | | | | | |
| Chlorociboria aeruginascens | TAQ88760.1 | 2592 | 3701 | 4868 | | | | | | | | | | | | | | |
| Chlorociboria aeruginascens | TAQ88760.1 | 2593 | 3702 | 4869 | 5153 | | | | | | | | | | | | | |
| Chlorociboria aeruginascens | TAQ88760.1 | 2594 | 3703 | | | | | | | | | | | | | | | |
| Chlorociboria aeruginascens | TAQ88760.1 | 2595 | 3704 | | | | | | | | | | | | | | | |
| Chlorociboria aeruginascens | TAQ88760.1 | 2596 | 3705 | | | | | | | | | | | | | | | |
| Claviceps purpurea 20.1 | CCE34786.1 | 2597 | 3706 | | | | | | | | | | | | | | | |
| Claviceps purpurea 20.1 | CCE34786.1 | 2598 | 3707 | | | | | | | | | | | | | | | |
| Coleophoma cylindrospora | RDW77053.1 | 2599 | 3708 | | | | | | | | | | | | | | | |
| Coleophoma cylindrospora | RDW77053.1 | 2600 | 3709 | 4680 | 5028 | | | | | | | | | | | | | |
| Colletotrichum aenigma | KAF5528507.1 | 2601 | 3710 | 4741 | 5063 | 5268 | 5403 | 5496 | | | | | | | | | | |
| Colletotrichum asianum | KAF0331997.1 | 2602 | 3711 | | | | | | | | | | | | | | | |
| Colletotrichum asianum | KAF0331997.1 | 2603 | 3712 | | | | | | | | | | | | | | | |
| Colletotrichum asianum | KAF0331997.1 | 2604 | 3713 | 4749 | 5067 | | | | | | | | | | | | | |
| Colletotrichum asianum | KAF0331997.1 | 2605 | 3714 | | | | | | | | | | | | | | | |
| Colletotrichum fructicola | KAE9574638.1 | 2606 | 3715 | | | | | | | | | | | | | | | |
| Colletotrichum fructicola | KAE9574638.1 | 2607 | 3716 | 4750 | 5068 | 5269 | | | | | | | | | | | | |
| Colletotrichum fructicola | KAF4433056.1 | 2608 | 3717 | | | | | | | | | | | | | | | |
| Colletotrichum fructicola | KAF4433056.1 | 2609 | 3718 | 4543 | 4980 | 5227 | | | | | | | | | | | | |
| Colletotrichum fructicola | KAF4899184.1 | 2610 | 3719 | | | | | | | | | | | | | | | |
| Colletotrichum fructicola | KAF4899184.1 | 2611 | 3720 | 4544 | 4981 | 5228 | | | | | | | | | | | | |
| Colletotrichum fructicola | KAF4906926.1 | 2612 | 3721 | | | | | | | | | | | | | | | |
| Colletotrichum fructicola | KAF4906926.1 | 2613 | 3722 | 4751 | 5069 | 5270 | | | | | | | | | | | | |
| Colletotrichum fructicola | KAF4936062.1 | 2614 | 3723 | | | | | | | | | | | | | | | |
| Colletotrichum fructicola | KAF4936062.1 | 2615 | 3724 | 4545 | 4982 | 5229 | | | | | | | | | | | | |
| Colletotrichum fructicola | KAF5513975.1 | 2616 | 3725 | | | | | | | | | | | | | | | |
| Colletotrichum fructicola | KAF5513975.1 | 2617 | 3726 | 4752 | 5070 | 5271 | | | | | | | | | | | | |
| Colletotrichum fructicola | KAF4491684.1 | 2618 | 3727 | | | | | | | | | | | | | | | |
| Nara gc5 | | | | | | | | | | | | | | | | | | |
| Colletotrichum fructicola | KAF4491684.1 | 2619 | 3728 | 4753 | 5071 | 5272 | | | | | | | | | | | | |
| Nara gc5 | | | | | | | | | | | | | | | | | | |
| Colletotrichum gloeosporioides | KAF3803768.1 | 2620 | 3729 | 4480 | 4933 | 5219 | 5373 | 5473 | | | | | | | | | | |
| Colletotrichum higginsianum | TIC93712.1 | 2621 | 3730 | | | | | | | | | | | | | | | |
| Colletotrichum higginsianum | TIC93712.1 | 2622 | 3731 | | | | | | | | | | | | | | | |
| Colletotrichum | OBR04629.1 | 2623 | 3732 | | | | | | | | | | | | | | | |
| higginsianum IMI 349063 | | | | | | | | | | | | | | | | | | |
| Colletotrichum | OBR04629.1 | 2624 | 3733 | | | | | | | | | | | | | | | |
| higginsianum IMI 349063 | | | | | | | | | | | | | | | | | | |
| Colletotrichum orchidophilum | OHF03557.1 | 2625 | 3734 | | | | | | | | | | | | | | | |
| Colletotrichum orchidophilum | OHF03557.1 | 2626 | 3735 | | | | | | | | | | | | | | | |
| Colletotrichum orchidophilum | OHF03557.1 | 2627 | 3736 | | | | | | | | | | | | | | | |
| Colletotrichum salicis | KXH61308.1 | 2628 | 3737 | | | | | | | | | | | | | | | |
| Colletotrichum salicis | KXH61308.1 | 2629 | 3738 | | | | | | | | | | | | | | | |
| Colletotrichum salicis | KXH61308.1 | 2630 | 3739 | | | | | | | | | | | | | | | |
| Colletotrichum shisoi | TQN69737.1 | 2631 | 3740 | | | | | | | | | | | | | | | |
| Colletotrichum shisoi | TQN69737.1 | 2632 | 3741 | | | | | | | | | | | | | | | |
| Colletotrichum shisoi | TQN69737.1 | 2633 | 3742 | | | | | | | | | | | | | | | |
| Colletotrichum siamense | KAF4825665.1 | 2634 | 3743 | 4546 | 4979 | 5221 | 5383 | | | | | | | | | | | |
| Colletotrichum siamense | KAF4843307.1 | 2635 | 3744 | 4605 | 4987 | 5224 | 5381 | | | | | | | | | | | |
| Colletotrichum siamense | KAF4858997.1 | 2636 | 3745 | 4606 | 4988 | 5225 | 5382 | | | | | | | | | | | |
| Colletotrichum siamense | KAF4872975.1 | 2637 | 3746 | | | | | | | | | | | | | | | |
| Colletotrichum siamense | KAF4872975.1 | 2638 | 3747 | 4547 | 4986 | 5243 | | | | | | | | | | | | |
| Colletotrichum siamense | KAF4817053.1 | 2639 | 3748 | 4754 | 5072 | 5273 | 5430 | 5497 | | | | | | | | | | |
| Colletotrichum tanaceti | TKW50855.1 | 2640 | 3749 | | | | | | | | | | | | | | | |
| Colletotrichum tanaceti | TKW50855.1 | 2641 | 3750 | | | | | | | | | | | | | | | |
| Colletotrichum tanaceti | TKW50855.1 | 2642 | 3751 | | | | | | | | | | | | | | | |
| Colletotrichum tropicale | KAF4835861.1 | 2643 | 3752 | 4742 | 5062 | 5267 | 5402 | | | | | | | | | | | |
| Colletotrichum viniferum | KAF4917890.1 | 2644 | 3753 | | | | | | | | | | | | | | | |
| Colletotrichum viniferum | KAF4917890.1 | 2645 | 3754 | 4607 | 4991 | | | | | | | | | | | | | |
| Coniella lustricola | PSR86972.1 | 2646 | 3755 | 4808 | 5111 | 5290 | 5410 | 5494 | 5548 | 5590 | | | | | | | | |
| Coniella lustricola | PSR86972.1 | 2647 | 3756 | 4809 | 5112 | 5291 | 5411 | 5495 | 5549 | 5589 | 5619 | | | | | | | |
| Coniella lustricola | PSR86972.1 | 2648 | 3757 | 4815 | 5113 | 5292 | 5429 | 5499 | 5550 | | | | | | | | | |
| Coniochaeta ligniaria | OIW34577.1 | 2649 | 3758 | 4732 | 5057 | | | | | | | | | | | | | |
| NRRL 30616 | | | | | | | | | | | | | | | | | | |
| Coniochaeta pulveracea | RKU46130.1 | 2650 | 3759 | | | | | | | | | | | | | | | |
| Coniochaeta pulveracea | RKU46130.1 | 2651 | 3760 | 4491 | | | | | | | | | | | | | | |
| Coniochaeta pulveracea | RKU46130.1 | 2652 | 3761 | | | | | | | | | | | | | | | |
| Coniochaeta pulveracea | RKU46130.1 | 2653 | 3762 | | | | | | | | | | | | | | | |
| Coniochaeta pulveracea | RKU46130.1 | 2654 | 3763 | 4542 | | | | | | | | | | | | | | |
| Coniochaeta pulveracea | RKU46130.1 | 2655 | 3764 | 4610 | | | | | | | | | | | | | | |
| Coniochaeta pulveracea | RKU46130.1 | 2656 | 2765 | | | | | | | | | | | | | | | |
| Coniochaeta pulveracea | RKU46130.1 | 2657 | 3766 | | | | | | | | | | | | | | | |
| Coniochaeta sp. 2T2.1 | KAB5551105.1 | 2658 | 3767 | | | | | | | | | | | | | | | |
| Coniochaeta sp. 2T2.1 | KAB5551105.1 | 2659 | 3768 | | | | | | | | | | | | | | | |
| Coniochaeta sp. 2T2.1 | KAB5551105.1 | 2660 | 3769 | | | | | | | | | | | | | | | |
| Coniochaeta sp. 2T2.1 | KAB5551105.1 | 2661 | 3770 | | | | | | | | | | | | | | | |
| Coniochaeta sp. 2T2.1 | KAB5551105.1 | 2662 | 3771 | | | | | | | | | | | | | | | |
| Coniochaeta sp. 2T2.1 | KAB5580195.1 | 2663 | 3772 | | | | | | | | | | | | | | | |
| Coniochaeta sp. 2T2.1 | KAB5580195.1 | 2664 | 3773 | 4849 | 5148 | | | | | | | | | | | | | |
| Coniochaeta sp. 2T2.1 | KAB5580195.1 | 2665 | 3774 | | | | | | | | | | | | | | | |
| Coniochaeta sp. 2T2.1 | KAB5580195.1 | 2666 | 3775 | | | | | | | | | | | | | | | |
| Coniochaeta sp. 2T2.1 | KAB5580195.1 | 2667 | 3776 | 4863 | | | | | | | | | | | | | | |
| Coniosporium apollinis | EON69158.1 | 2668 | 3777 | | | | | | | | | | | | | | | |
| CBS 100218 | | | | | | | | | | | | | | | | | | |
| Coniosporium apollinis | EON69158.1 | 2669 | 3778 | | | | | | | | | | | | | | | |
| CBS 100218 | | | | | | | | | | | | | | | | | | |
| Coniosporium apollinis | EON69158.1 | 2670 | 3779 | | | | | | | | | | | | | | | |
| CBS 100218 | | | | | | | | | | | | | | | | | | |
| Coniosporium apollinis | EON69158.1 | 2671 | 3780 | | | | | | | | | | | | | | | |
| CBS 100218 | | | | | | | | | | | | | | | | | | |
| Cordyceps fumosorosea | OAA60883.1 | 2672 | 3781 | 4628 | | | | | | | | | | | | | | |
| ARSEF 2679 | | | | | | | | | | | | | | | | | | |
| Cordyceps fumosorosea | OAA60883.1 | 2673 | 3782 | 4637 | | | | | | | | | | | | | | |
| ARSEF 2679 | | | | | | | | | | | | | | | | | | |
| Cordyceps fumosorosea | OAA60883.1 | 2674 | 3783 | 4608 | | | | | | | | | | | | | | |
| ARSEF 2679 | | | | | | | | | | | | | | | | | | |
| Cordyceps militaris | ATY63712.1 | 2675 | 3784 | | | | | | | | | | | | | | | |
| Cordyceps militaris | ATY63712.1 | 2676 | 3785 | | | | | | | | | | | | | | | |
| Cordyceps militaris | ATY63712.1 | 2677 | 3786 | | | | | | | | | | | | | | | |
| Cryomyces minteri | TKA66690.1 | 2678 | 3787 | | | | | | | | | | | | | | | |
| Cryomyces minteri | TKA66690.1 | 2679 | 3788 | 4623 | | | | | | | | | | | | | | |
| Cryphonectria parasitica | KAF3762650.1 | 2680 | 3789 | | | | | | | | | | | | | | | |
| EP155 | | | | | | | | | | | | | | | | | | |
| Cryphonectria parasitica | KAF3762650.1 | 2681 | 3790 | 4560 | 4954 | 5200 | 5359 | 5454 | 5536 | | | | | | | | | |
| EP155 | | | | | | | | | | | | | | | | | | |
| Cryphonectria parasitica | KAF3762650.1 | 2682 | 3791 | 4541 | 4953 | 5196 | 5358 | 5466 | 5535 | 5579 | | | | | | | | |
| EP155 | | | | | | | | | | | | | | | | | | |
| Cryphonectria parasitica | KAF3762650.1 | 2683 | 3792 | 4565 | 4960 | 5201 | 5360 | 5467 | | | | | | | | | | |
| EP155 | | | | | | | | | | | | | | | | | | |
| Cytospora leucostoma | ROW02039.1 | 2684 | 3793 | | | | | | | | | | | | | | | |
| Cytospora leucostoma | ROW02039.1 | 2685 | 3794 | 4807 | 5105 | 5285 | 5405 | | | | | | | | | | | |
| Delitschia confertaspora | KAF2197766.1 | 2686 | 3795 | | | | | | | | | | | | | | | |
| ATCC 74209 | | | | | | | | | | | | | | | | | | |
| Delitschia confertaspora | KAF2197766.1 | 2687 | 3796 | 4640 | | | | | | | | | | | | | | |
| ATCC 74209 | | | | | | | | | | | | | | | | | | |
| Delitschia confertaspora | KAF2197766.1 | 2688 | 3797 | 4526 | 4993 | | | | | | | | | | | | | |
| ATCC 74209 | | | | | | | | | | | | | | | | | | |
| Diaporthe ampelina | KKY30715.1 | 2689 | 3798 | | | | | | | | | | | | | | | |
| Diaporthe ampelina | KKY30715.1 | 2690 | 3799 | | | | | | | | | | | | | | | |
| Diaporthe ampelina | KKY30715.1 | 2691 | 3800 | 4859 | 5137 | 5311 | 5427 | 5493 | | | | | | | | | | |
| Diaporthe ampelina | KKY30715.1 | 2692 | 3801 | 4860 | 5138 | 5312 | 5428 | | | | | | | | | | | |
| Diaporthe helianthi | POS74470.1 | 2693 | 3802 | | | | | | | | | | | | | | | |
| Diaporthe helianthi | POS74470.1 | 2694 | 3803 | | | | | | | | | | | | | | | |
| Diaporthe helianthi | POS74470.1 | 2695 | 3804 | 4563 | 4958 | 5197 | 5357 | | | | | | | | | | | |
| Diaporthe helianthi | POS74470.1 | 2696 | 3805 | 4479 | 4932 | 5198 | 5374 | | | | | | | | | | | |
| Diplocarpon rosae | PBP22901.1 | 2697 | 3806 | 4704 | 5039 | 5257 | 5399 | 5506 | | | | | | | | | | |
| Diplocarpon rosae | PBP22901.1 | 2698 | 3807 | | | | | | | | | | | | | | | |
| Diplocarpon rosae | PBP22901.1 | 2699 | 3808 | | | | | | | | | | | | | | | |
| Diplocarpon rosae | PBP22837.1 | 2700 | 3809 | 4703 | 5038 | 5256 | 5400 | | | | | | | | | | | |
| Diplodia corticola | OJD32371.1 | 2701 | 3810 | | | | | | | | | | | | | | | |
| Diplodia corticola | OJD32371.1 | 2702 | 3811 | 4877 | 5159 | 5339 | | | | | | | | | | | | |
| Diplodia corticola | OJD32371.1 | 2703 | 3812 | 4876 | 5158 | 5338 | | | | | | | | | | | | |
| Dothistroma septosporum | EME39369.1 | 2704 | 3813 | | | | | | | | | | | | | | | |
| NZE10 | | | | | | | | | | | | | | | | | | |
| Dothistroma septosporum | EME39369.1 | 2705 | 3814 | | | | | | | | | | | | | | | |
| NZE10 | | | | | | | | | | | | | | | | | | |
| Dothistroma septosporum | EME39369.1 | 2706 | 3815 | 4684 | 5033 | 5254 | 5397 | 5479 | 5543 | 5586 | 5618 | 5646 | 5665 | 5679 | 5687 | 5694 | | |
| NZE10 | | | | | | | | | | | | | | | | | | |
| Dothistroma septosporum | EME39369.1 | 2707 | 3816 | | | | | | | | | | | | | | | |
| NZE10 | | | | | | | | | | | | | | | | | | |
| Dothistroma septosporum | EME39369.1 | 2708 | 3817 | | | | | | | | | | | | | | | |
| NZE10 | | | | | | | | | | | | | | | | | | |
| Dothistroma septosporum | EME39369.1 | 2709 | 3818 | 4259 | 4944 | 5188 | 5356 | 5453 | 5517 | 5570 | 5603 | 5633 | 5657 | 5674 | 5682 | 5690 | | |
| NZE10 | | | | | | | | | | | | | | | | | | |
| Drechmeria coniospora | KYK56087.1 | 2710 | 3819 | 4763 | | | | | | | | | | | | | | |
| Drechmeria coniospora | KYK56087.1 | 2711 | 3820 | 4758 | 5075 | | | | | | | | | | | | | |
| Drechmeria coniospora | KYK56087.1 | 2712 | 3821 | 4759 | 5076 | | | | | | | | | | | | | |
| Drechmeria coniospora | ODA77761.1 | 2713 | 3822 | 4611 | | | | | | | | | | | | | | |
| Drechmeria coniospora | ODA77761.1 | 2714 | 3823 | 4574 | 5012 | | | | | | | | | | | | | |
| Drechmeria coniospora | ODA77761.1 | 2715 | 3824 | 4582 | 5027 | | | | | | | | | | | | | |
| Drepanopeziza brunnea f. | EKD17274.1 | 2716 | 3825 | 4708 | 5152 | 5327 | | | | | | | | | | | | |
| sp. ‘multigermtubi’ MB_m1 | | | | | | | | | | | | | | | | | | |
| Escovopsis weberi | KOS21127.1 | 2717 | 3826 | | | | | | | | | | | | | | | |
| Escovopsis weberi | KOS21127.1 | 2718 | 3827 | | | | | | | | | | | | | | | |
| Escovopsis weberi | KOS21127.1 | 2719 | 3828 | | | | | | | | | | | | | | | |
| Escovopsis weberi | KOS21127.1 | 2720 | 3829 | 4736 | 5061 | | | | | | | | | | | | | |
| Escovopsis weberi | KOS21127.1 | 2721 | 3830 | | | | | | | | | | | | | | | |
| Escovopsis weberi | KOS21127.1 | 2722 | 3831 | 4737 | | | | | | | | | | | | | | |
| Escovopsis weberi | KOS21127.1 | 2723 | 3832 | | | | | | | | | | | | | | | |
| Gaeumannomyces tritici | EJT72500.1 | 2724 | 3833 | | | | | | | | | | | | | | | |
| R3-111a-1 | | | | | | | | | | | | | | | | | | |
| Gaeumannomyces tritici | EJT72500.1 | 2725 | 3834 | | | | | | | | | | | | | | | |
| R3-111a-1 | | | | | | | | | | | | | | | | | | |
| Gaeumannomyces tritici | EJT72500.1 | 2726 | 3835 | | | | | | | | | | | | | | | |
| R3-111a-1 | | | | | | | | | | | | | | | | | | |
| Gaeumannomyces tritici | EJT72500.1 | 2727 | 3836 | | | | | | | | | | | | | | | |
| R3-111a-1 | | | | | | | | | | | | | | | | | | |
| Gaeumannomyces tritici | EJT72500.1 | 2728 | 3837 | | | | | | | | | | | | | | | |
| R3-111a-1 | | | | | | | | | | | | | | | | | | |
| Gaeumannomyces tritici | EJT72500.1 | 2729 | 3838 | | | | | | | | | | | | | | | |
| R3-111a-1 | | | | | | | | | | | | | | | | | | |
| Gaeumannomyces tritici | EJT72500.1 | 2730 | 3839 | | | | | | | | | | | | | | | |
| R3-111a-1 | | | | | | | | | | | | | | | | | | |
| Gaeumannomyces tritici | EJT72500.1 | 2731 | 3840 | | | | | | | | | | | | | | | |
| R3-111a-1 | | | | | | | | | | | | | | | | | | |
| Glarea lozoyensis 74030 | EHL02023.1 | 2732 | 3841 | 4887 | | | | | | | | | | | | | | |
| Glarea lozoyensis 74030 | EHL02023.1 | 2733 | 3842 | 4884 | | | | | | | | | | | | | | |
| Glarea lozoyensis 74030 | EHL02023.1 | 2734 | 3843 | 4885 | | | | | | | | | | | | | | |
| Glarea lozoyensis ATCC | EPE28052.1 | 2735 | 3844 | 4888 | | | | | | | | | | | | | | |
| 20868 | | | | | | | | | | | | | | | | | | |
| Glarea lozoyensis ATCC | EPE28052.1 | 2736 | 3845 | 4883 | 5160 | | | | | | | | | | | | | |
| 20868 | | | | | | | | | | | | | | | | | | |
| Glarea lozoyensis ATCC | EPE28052.1 | 2737 | 3846 | 4886 | | | | | | | | | | | | | | |
| 20868 | | | | | | | | | | | | | | | | | | |
| Glonium stellatum | OCL15332.1 | 2738 | 3847 | | | | | | | | | | | | | | | |
| Glonium stellatum | OCL15332.1 | 2739 | 3848 | 4686 | 5035 | | | | | | | | | | | | | |
| Grosmannia clavigera | EFW99502.1 | 2740 | 3849 | | | | | | | | | | | | | | | |
| kw1407 | | | | | | | | | | | | | | | | | | |
| Grosmannia clavigera | EFW99502.1 | 2741 | 3850 | | | | | | | | | | | | | | | |
| kw1407 | | | | | | | | | | | | | | | | | | |
| Helotiales sp. | KAE8446876.1 | 2742 | 3851 | 4503 | 4904 | | | | | | | | | | | | | |
| DMI_Dod_QoI | | | | | | | | | | | | | | | | | | |
| Helotiales sp. | KAE8446876.1 | 2743 | 3852 | | | | | | | | | | | | | | | |
| DMI_Dod_QoI | | | | | | | | | | | | | | | | | | |
| Helotiales sp. | KAE8446876.1 | 2744 | 3853 | 4585 | 4996 | 5332 | | | | | | | | | | | | |
| DMI_Dod_QoI | | | | | | | | | | | | | | | | | | |
| Hyaloscypha bicolor E | PMD52247.1 | 2745 | 3854 | 4866 | 5151 | 5326 | 5437 | | | | | | | | | | | |
| Hyaloscypha bicolor E | PMD52247.1 | 2746 | 3855 | | | | | | | | | | | | | | | |
| Hyaloscypha hepaticicola | PMD19774.1 | 2747 | 3856 | | | | | | | | | | | | | | | |
| Hyaloscypha hepaticicola | PMD19774.1 | 2748 | 3857 | | | | | | | | | | | | | | | |
| Hyaloscypha hepaticicola | PMD19774.1 | 2749 | 3858 | | | | | | | | | | | | | | | |
| Lachnellula hyalina | TVY28938.1 | 2750 | 3859 | 4676 | | | | | | | | | | | | | | |
| Lachnellula hyalina | TVY28938.1 | 2751 | 3860 | 4668 | | | | | | | | | | | | | | |
| Lachnellula hyalina | TVY28938.1 | 2752 | 3861 | | | | | | | | | | | | | | | |
| Lachnellula hyalina | TVY28938.1 | 2753 | 3862 | | | | | | | | | | | | | | | |
| Lachnellula hyalina | TVY28938.1 | 2754 | 3863 | | | | | | | | | | | | | | | |
| Lachnellula occidentalis | TVY33705.1 | 2755 | 3864 | | | | | | | | | | | | | | | |
| Lachnellula occidentalis | TVY33705.1 | 2756 | 3865 | 4615 | | | | | | | | | | | | | | |
| Lachnellula occidentalis | TVY33705.1 | 2757 | 3866 | 4666 | | | | | | | | | | | | | | |
| Lachnellula subtilissima | TVY31325.1 | 2758 | 3867 | 4617 | 5026 | | | | | | | | | | | | | |
| Lachnellula subtilissima | TVY31325.1 | 2759 | 3868 | | | | | | | | | | | | | | | |
| Lachnellula subtilissima | TVY31325.1 | 2760 | 3869 | | | | | | | | | | | | | | | |
| Lachnellula subtilissima | TVY31325.1 | 2761 | 3870 | 4667 | | | | | | | | | | | | | | |
| Lachnellula suecica | TVY60901.1 | 2762 | 3871 | 4871 | | | | | | | | | | | | | | |
| Lachnellula suecica | TVY60901.1 | 2763 | 3872 | 4872 | | | | | | | | | | | | | | |
| Lachnellula suecica | TVY60901.1 | 2764 | 3873 | 4873 | | | | | | | | | | | | | | |
| Lachnellula suecica | TVY60901.1 | 2765 | 3874 | 4655 | | | | | | | | | | | | | | |
| Lachnellula willkommii | TVY86521.1 | 2766 | 3875 | 4616 | 5025 | | | | | | | | | | | | | |
| Lachnellula willkommii | TVY86521.1 | 2767 | 3876 | 4630 | | | | | | | | | | | | | | |
| Lachnellula willkommii | TVY86521.1 | 2768 | 3877 | | | | | | | | | | | | | | | |
| Lachnellula willkommii | TVY86521.1 | 2769 | 3878 | | | | | | | | | | | | | | | |
| Lasallia pustulata | KAA64153671 | 2770 | 3879 | 4639 | | | | | | | | | | | | | | |
| Lasallia pustulata | KAA6415367.1 | 2771 | 3880 | | | | | | | | | | | | | | | |
| Lasallia pustulata | KAA6415367.1 | 2772 | 3881 | | | | | | | | | | | | | | | |
| Lasallia pustulata | SLM33497.1 | 2773 | 3882 | | | | | | | | | | | | | | | |
| Lasallia pustulata | SLM33497.1 | 2774 | 3883 | | | | | | | | | | | | | | | |
| Lasallia pustulata | SLM33497.1 | 2775 | 3884 | | | | | | | | | | | | | | | |
| Lasallia pustulata | SLM33497.1 | 2776 | 3885 | | | | | | | | | | | | | | | |
| Lasiodiplodia theobromae | KAB2570072.1 | 2777 | 3886 | | | | | | | | | | | | | | | |
| Lasiodiplodia theobromae | KAB2570072.1 | 2778 | 3887 | 4483 | 4894 | 5213 | 5385 | | | | | | | | | | | |
| Lasiodiplodia theobromae | KAB2570072.1 | 2779 | 3888 | 4598 | 4976 | 5211 | 5440 | | | | | | | | | | | |
| Lasiodiplodia theobromae | KAF4536744.1 | 2780 | 3889 | | | | | | | | | | | | | | | |
| Lasiodiplodia theobromae | KAF4536744.1 | 2781 | 3890 | 4484 | 4895 | 5214 | 5386 | | | | | | | | | | | |
| Lasiodiplodia theobromae | KAF4536744.1 | 2782 | 3891 | 4599 | 4977 | 5212 | 5441 | | | | | | | | | | | |
| Letharia columbiana | KAF6235908.1 | 2783 | 3892 | | | | | | | | | | | | | | | |
| Letharia columbiana | KAF6235908.1 | 2784 | 3893 | | | | | | | | | | | | | | | |
| Letharia columbiana | KAF6235908.1 | 2785 | 3894 | | | | | | | | | | | | | | | |
| Letharia columbiana | KAF6235908.1 | 2786 | 3895 | | | | | | | | | | | | | | | |
| Letharia columbiana | KAF6235908.1 | 2787 | 3896 | | | | | | | | | | | | | | | |
| Lindgomyces ingoldianus | KAF2474749.1 | 2788 | 3897 | | | | | | | | | | | | | | | |
| Lindgomyces ingoldianus | KAF2474749.1 | 2789 | 3898 | 4527 | 4974 | 5210 | | | | | | | | | | | | |
| Lindgomyces ingoldianus | KAF2474749.1 | 2790 | 3899 | 4528 | | | | | | | | | | | | | | |
| Lindgomyces ingoldianus | KAF2474749.1 | 2791 | 3900 | 4493 | 4915 | 5193 | | | | | | | | | | | | |
| Lindgomyces ingoldianus | KAF2474749.1 | 2792 | 3901 | 4494 | 4916 | 5194 | | | | | | | | | | | | |
| Lindgomyces ingoldianus | KAF2474749.1 | 2793 | 3902 | | | | | | | | | | | | | | | |
| Lineolata rhizophorae | KAF2456623.1 | 2794 | 3903 | | | | | | | | | | | | | | | |
| Lineolata rhizophorae | KAF2456623.1 | 2795 | 3904 | | | | | | | | | | | | | | | |
| Lineolata rhizophorae | KAF2456623.1 | 2796 | 3905 | | | | | | | | | | | | | | | |
| Lineolata rhizophorae | KAF2456623.1 | 2797 | 3906 | | | | | | | | | | | | | | | |
| Lineolata rhizophorae | KAF2456623.1 | 2798 | 3907 | | | | | | | | | | | | | | | |
| Lophiostoma macrostomum | KAF2657304.1 | 2799 | 3908 | 4509 | 4909 | 5234 | 5379 | 5472 | | | | | | | | | | |
| CBS 122681 | | | | | | | | | | | | | | | | | | |
| Lophiotrema nucula | KAF2109031.1 | 2800 | 3909 | | | | | | | | | | | | | | | |
| Lophiotrema nucula | KAF2109031.1 | 2801 | 3910 | | | | | | | | | | | | | | | |
| Lophiotrema nucula | KAF2109031.1 | 2802 | 3911 | | | | | | | | | | | | | | | |
| Lophiotrema nucula | KAF2109031.1 | 2803 | 3912 | 4698 | | | | | | | | | | | | | | |
| Lophiotrema nucula | KAF2109031.1 | 2804 | 3913 | 4699 | 5037 | | | | | | | | | | | | | |
| Lophium mytilinum | KAF2499287.1 | 2805 | 3914 | 4649 | 5010 | 5241 | 5388 | 5474 | 5538 | 5584 | | | | | | | | |
| Lophium mytilinum | KAF2499287.1 | 2806 | 3915 | 4515 | 4912 | 5242 | 5380 | | | | | | | | | | | |
| Lophium mytilinum | KAF2499287.1 | 2807 | 3916 | | | | | | | | | | | | | | | |
| Lophium mytilinum | KAF2499287.1 | 2808 | 3917 | 4650 | 5011 | | | | | | | | | | | | | |
| Madurella mycetomatis | KXX80798.1 | 2809 | 3918 | | | | | | | | | | | | | | | |
| Madurella mycetomatis | KXX80798.1 | 2810 | 3919 | | | | | | | | | | | | | | | |
| Madurella mycetomatis | KXX80798.1 | 2811 | 3920 | | | | | | | | | | | | | | | |
| Madurella mycetomatis | KXX80798.1 | 2812 | 3921 | | | | | | | | | | | | | | | |
| Madurella mycetomatis | KXX80798.1 | 2813 | 3922 | | | | | | | | | | | | | | | |
| Madurella mycetomatis | KXX80798.1 | 2814 | 3923 | | | | | | | | | | | | | | | |
| Madurella mycetomatis | KXX80798.1 | 2815 | 3924 | 4764 | | | | | | | | | | | | | | |
| Madurella mycetomatis | KXX80798.1 | 2816 | 3925 | 4744 | 5064 | 5274 | | | | | | | | | | | | |
| Madurella mycetomatis | KXX80798.1 | 2817 | 3926 | 4745 | 5079 | | | | | | | | | | | | | |
| Madurella mycetomatis | KXX80798.1 | 2818 | 3927 | 4746 | 5078 | 5275 | | | | | | | | | | | | |
| Marssonina coronariae | OWP04968.1 | 2819 | 3928 | 4504 | 4903 | | | | | | | | | | | | | |
| Marssonina coronariae | OWP04968.1 | 2820 | 3929 | 4627 | | | | | | | | | | | | | | |
| Marssonina coronariae | OWP04968.1 | 2821 | 3930 | | | | | | | | | | | | | | | |
| Massarina eburnea CBS | KAF2645689.1 | 2822 | 3931 | 4600 | | | | | | | | | | | | | | |
| 473.64 | | | | | | | | | | | | | | | | | | |
| Massarina eburnea CBS | KAF2645689.1 | 2823 | 3932 | 4658 | | | | | | | | | | | | | | |
| 473.64 | | | | | | | | | | | | | | | | | | |
| Massarina eburnea CBS | KAF2645689.1 | 2824 | 3933 | 4657 | | | | | | | | | | | | | | |
| 473.64 | | | | | | | | | | | | | | | | | | |
| Melanomma pulvis-pyrius | KAF2795620.1 | 2825 | 3934 | 4700 | | | | | | | | | | | | | | |
| CBS 109.77 | | | | | | | | | | | | | | | | | | |
| Melanomma pulvis-pyrius | KAF2795620.1 | 2826 | 3935 | 4696 | | | | | | | | | | | | | | |
| CBS 109.77 | | | | | | | | | | | | | | | | | | |
| Melanomma pulvis-pyrius | KAF2795620.1 | 2827 | 3936 | 4701 | | | | | | | | | | | | | | |
| CBS 109.77 | | | | | | | | | | | | | | | | | | |
| Melanomma pulvis-pyrius | KAF2795620.1 | 2828 | 3937 | 4702 | | | | | | | | | | | | | | |
| CBS 109.77 | | | | | | | | | | | | | | | | | | |
| Metarhizium acridum | EFY84452.1 | 2829 | 3938 | | | | | | | | | | | | | | | |
| CQMa 102 | | | | | | | | | | | | | | | | | | |
| Metarhizium acridum | EFY84452.1 | 2830 | 3939 | 4851 | | | | | | | | | | | | | | |
| CQMa 102 | | | | | | | | | | | | | | | | | | |
| Metarhizium acridum | EFY84452.1 | 2831 | 3940 | 4850 | | | | | | | | | | | | | | |
| CQMa 102 | | | | | | | | | | | | | | | | | | |
| Metarhizium acridum | EFY84452.1 | 2832 | 3941 | | | | | | | | | | | | | | | |
| CQMa 102 | | | | | | | | | | | | | | | | | | |
| Metarhizium acridum | EFY84452.1 | 2833 | 3942 | | | | | | | | | | | | | | | |
| CQMa 102 | | | | | | | | | | | | | | | | | | |
| Metarhizium album ARSEF | KHN97959.1 | 2834 | 3943 | | | | | | | | | | | | | | | |
| 1941 | | | | | | | | | | | | | | | | | | |
| Metarhizium album ARSEF | KHN97959.1 | 2835 | 3944 | | | | | | | | | | | | | | | |
| 1941 | | | | | | | | | | | | | | | | | | |
| Metarhizium album ARSEF | KHN97959.1 | 2836 | 3945 | | | | | | | | | | | | | | | |
| 1941 | | | | | | | | | | | | | | | | | | |
| Metarhizium album ARSEF | KHN97959.1 | 2837 | 3946 | | | | | | | | | | | | | | | |
| 1941 | | | | | | | | | | | | | | | | | | |
| Metarhizium anisopliae | KAF5133551.1 | 2838 | 3947 | 4511 | 4940 | 5189 | 5392 | | | | | | | | | | | |
| Metarhizium anisopliae | KAF5133551.1 | 2839 | 3948 | 4512 | 4941 | 5250 | 5395 | 5498 | | | | | | | | | | |
| Metarhizium anisopliae | KAF5133551.1 | 2840 | 3949 | 4513 | 4942 | 5216 | 5393 | | | | | | | | | | | |
| Metarhizium anisopliae | KAF5133551.1 | 2841 | 3950 | 4514 | 4943 | 5217 | 5394 | | | | | | | | | | | |
| Metarhizium anisopliae | KFG78352.1 | 2842 | 3951 | | | | | | | | | | | | | | | |
| Metarhizium anisopliae | KFG78352.1 | 2843 | 3952 | | | | | | | | | | | | | | | |
| Metarhizium anisopliae | KFG78352.1 | 2844 | 3953 | | | | | | | | | | | | | | | |
| Metarhizium anisopliae | KFG78352.1 | 2845 | 3954 | | | | | | | | | | | | | | | |
| Metarhizium anisopliae | KID61826.1 | 2846 | 3955 | | | | | | | | | | | | | | | |
| ARSEF 549 | | | | | | | | | | | | | | | | | | |
| Metarhizium anisopliae | KID61826.1 | 2847 | 3956 | | | | | | | | | | | | | | | |
| ARSEF 549 | | | | | | | | | | | | | | | | | | |
| Metarhizium anisopliae | KID61826.1 | 2848 | 3957 | | | | | | | | | | | | | | | |
| ARSEF 549 | | | | | | | | | | | | | | | | | | |
| Metarhizium anisopliae | KID61826.1 | 2849 | 3958 | | | | | | | | | | | | | | | |
| ARSEF 549 | | | | | | | | | | | | | | | | | | |
| Metarhizium anisopliae | KJK92374.1 | 2850 | 3959 | | | | | | | | | | | | | | | |
| BRIP 53284 | | | | | | | | | | | | | | | | | | |
| Metarhizium anisopliae | KJK92374.1 | 2851 | 3960 | | | | | | | | | | | | | | | |
| BRIP 53284 | | | | | | | | | | | | | | | | | | |
| Metarhizium anisopliae | KJK92374.1 | 2852 | 3961 | | | | | | | | | | | | | | | |
| BRIP 53284 | | | | | | | | | | | | | | | | | | |
| Metarhizium anisopliae | KJK92374.1 | 2853 | 3962 | | | | | | | | | | | | | | | |
| BRIP 53284 | | | | | | | | | | | | | | | | | | |
| Metarhizium anisopliae | KJK92374.1 | 2854 | 3963 | | | | | | | | | | | | | | | |
| BRIP 53284 | | | | | | | | | | | | | | | | | | |
| Metarhizium anisopliae | KJK83512.1 | 2855 | 3964 | | | | | | | | | | | | | | | |
| BRIP 53293 | | | | | | | | | | | | | | | | | | |
| Metarhizium anisopliae | KJK83512.1 | 2856 | 3965 | | | | | | | | | | | | | | | |
| BRIP 53293 | | | | | | | | | | | | | | | | | | |
| Metarhizium anisopliae | KJK83512.1 | 2857 | 3966 | | | | | | | | | | | | | | | |
| BRIP 53293 | | | | | | | | | | | | | | | | | | |
| Metarhizium anisopliae | KJK83512.1 | 2858 | 3967 | | | | | | | | | | | | | | | |
| BRIP 53293 | | | | | | | | | | | | | | | | | | |
| Metarhizium anisopliae | KJK83512.1 | 2859 | 3968 | | | | | | | | | | | | | | | |
| BRIP 53293 | | | | | | | | | | | | | | | | | | |
| Metarhizium brunneum | QLI64013.1 | 2860 | 3969 | | | | | | | | | | | | | | | |
| Metarhizium brunneum | QLI64013.1 | 2861 | 3970 | | | | | | | | | | | | | | | |
| Metarhizium brunneum | QLI64013.1 | 2862 | 3971 | | | | | | | | | | | | | | | |
| Metarhizium brunneum | QLI64013.1 | 2863 | 3972 | | | | | | | | | | | | | | | |
| Metarhizium brunneum | QLI64013.1 | 2864 | 3973 | | | | | | | | | | | | | | | |
| Metarhizium brunneum | KID80709.1 | 2865 | 3974 | | | | | | | | | | | | | | | |
| ARSEF 3297 | | | | | | | | | | | | | | | | | | |
| Metarhizium brunneum | KID80709.1 | 2866 | 3975 | | | | | | | | | | | | | | | |
| ARSEF 3297 | | | | | | | | | | | | | | | | | | |
| Metarhizium brunneum | KID80709.1 | 2867 | 3976 | | | | | | | | | | | | | | | |
| ARSEF 3297 | | | | | | | | | | | | | | | | | | |
| Metarhizium brunneum | KID80709.1 | 2868 | 3977 | | | | | | | | | | | | | | | |
| ARSEF 3297 | | | | | | | | | | | | | | | | | | |
| Metarhizium brunneum | KID80709.1 | 2869 | 3978 | | | | | | | | | | | | | | | |
| ARSEF 3297 | | | | | | | | | | | | | | | | | | |
| Metarhizium guizhouense | KID90822.1 | 2870 | 3979 | | | | | | | | | | | | | | | |
| ARSEF 977 | | | | | | | | | | | | | | | | | | |
| Metarhizium guizhouense | KID90822.1 | 2871 | 3980 | | | | | | | | | | | | | | | |
| ARSEF 977 | | | | | | | | | | | | | | | | | | |
| Metarhizium guizhouense | KID90822.1 | 2872 | 3981 | | | | | | | | | | | | | | | |
| ARSEF 977 | | | | | | | | | | | | | | | | | | |
| Metarhizium guizhouense | KID90822.1 | 2873 | 3982 | | | | | | | | | | | | | | | |
| ARSEF 977 | | | | | | | | | | | | | | | | | | |
| Metarhizium majus ARSEF | KIE00973.1 | 2874 | 3983 | | | | | | | | | | | | | | | |
| 297 | | | | | | | | | | | | | | | | | | |
| Metarhizium majus ARSEF | KIE00973.1 | 2875 | 3984 | | | | | | | | | | | | | | | |
| 297 | | | | | | | | | | | | | | | | | | |
| Metarhizium majus ARSEF | KIE00973.1 | 2876 | 3985 | | | | | | | | | | | | | | | |
| 297 | | | | | | | | | | | | | | | | | | |
| Metarhizium rileyi | TWU78575.1 | 2877 | 3986 | | | | | | | | | | | | | | | |
| Metarhizium rileyi | TWU78575.1 | 2878 | 3987 | 4652 | | | | | | | | | | | | | | |
| Metarhizium rileyi | TWU78575.1 | 2879 | 3988 | 4561 | 5021 | | | | | | | | | | | | | |
| Metarhizium rileyi | TWU78575.1 | 2880 | 3989 | 4677 | | | | | | | | | | | | | | |
| Metarhizium rileyi RCEF | OAA43713.1 | 2881 | 3990 | | | | | | | | | | | | | | | |
| 4871 | | | | | | | | | | | | | | | | | | |
| Metarhizium rileyi RCEF | OAA43713.1 | 2882 | 3991 | 4653 | | | | | | | | | | | | | | |
| 4871 | | | | | | | | | | | | | | | | | | |
| Metarhizium rileyi RCEF | OAA43713.1 | 2883 | 3992 | 4562 | 5022 | | | | | | | | | | | | | |
| 4871 | | | | | | | | | | | | | | | | | | |
| Metarhizium rileyi RCEF | OAA43713.1 | 2884 | 3993 | 4678 | | | | | | | | | | | | | | |
| 4871 | | | | | | | | | | | | | | | | | | |
| Metarhizium robertsii | EXV00913.1 | 2885 | 3994 | | | | | | | | | | | | | | | |
| Metarhizium robertsii | EXV00913.1 | 2886 | 3995 | | | | | | | | | | | | | | | |
| Metarhizium robertsii | EXV00913.1 | 2887 | 3996 | | | | | | | | | | | | | | | |
| Metarhizium robertsii | EXV00913.1 | 2888 | 3997 | | | | | | | | | | | | | | | |
| Metarhizium robertsii | EFZ04110.1 | 2889 | 3998 | | | | | | | | | | | | | | | |
| ARSEF 23 | | | | | | | | | | | | | | | | | | |
| Metarhizium robertsii | EFZ04110.1 | 2890 | 3999 | | | | | | | | | | | | | | | |
| ARSEF 23 | | | | | | | | | | | | | | | | | | |
| Metarhizium robertsii | EFZ04110.1 | 2891 | 4000 | | | | | | | | | | | | | | | |
| ARSEF 23 | | | | | | | | | | | | | | | | | | |
| Metarhizium robertsii | EFZ04110.1 | 2892 | 4001 | | | | | | | | | | | | | | | |
| ARSEF 23 | | | | | | | | | | | | | | | | | | |
| Moelleriella libera RCEF | KZZ94271.1 | 2893 | 4002 | | | | | | | | | | | | | | | |
| 2490 | | | | | | | | | | | | | | | | | | |
| Moelleriella libera RCEF | KZZ94271.1 | 2894 | 4003 | | | | | | | | | | | | | | | |
| 2490 | | | | | | | | | | | | | | | | | | |
| Moelleriella libera RCEF | KZZ94271.1 | 2895 | 4004 | | | | | | | | | | | | | | | |
| 2490 | | | | | | | | | | | | | | | | | | |
| Mollisia scopiformis | KUJ22789.1 | 2896 | 4005 | 4505 | 4911 | | | | | | | | | | | | | |
| Mollisia scopiformis | KUJ22789.1 | 2897 | 4006 | | | | | | | | | | | | | | | |
| Mollisia scopiformis | KUJ22789.1 | 2898 | 4007 | 4486 | 4971 | | | | | | | | | | | | | |
| Monilinia fructicola | KAA8576226.1 | 2899 | 4008 | | | | | | | | | | | | | | | |
| Monilinia fructicola | KAA8576226.1 | 2900 | 4009 | 4525 | 4939 | 5232 | 5377 | 5478 | | | | | | | | | | |
| Monilinia fructicola | KAA8576226.1 | 2901 | 4010 | | | | | | | | | | | | | | | |
| Monilinia fructicola | KAA8576226.1 | 2902 | 4011 | 4588 | 5161 | | | | | | | | | | | | | |
| Monilinia fructicola | KAA8576226.1 | 2903 | 4012 | | | | | | | | | | | | | | | |
| Monilinia fructigena | RAL62545.1 | 2904 | 4013 | | | | | | | | | | | | | | | |
| Monilinia fructigena | RAL62545.1 | 2905 | 4014 | 4487 | 4897 | 5173 | 5355 | 5477 | | | | | | | | | | |
| Monilinia fructigena | RAL62545.1 | 2906 | 4015 | | | | | | | | | | | | | | | |
| Monilinia fructigena | RAL62545.1 | 2907 | 4016 | | | | | | | | | | | | | | | |
| Monilinia laxa | KAB8302934.1 | 2908 | 4017 | | | | | | | | | | | | | | | |
| Monilinia laxa | KAB8302934.1 | 2909 | 4018 | 4827 | 5119 | 5296 | 5417 | 5482 | | | | | | | | | | |
| Monilinia laxa | KAB8302934.1 | 2910 | 4019 | | | | | | | | | | | | | | | |
| Monilinia laxa | KAB8302934.1 | 2911 | 4020 | | | | | | | | | | | | | | | |
| Mytilinidion resinicola | KAF2810334.1 | 2912 | 4021 | 4685 | 5034 | 5255 | 5398 | 5480 | 5544 | 5587 | 5624 | | | | | | | |
| Mytilinidion resinicola | KAF2810334.1 | 2913 | 4022 | 4731 | 5056 | | | | | | | | | | | | | |
| Mytilinidion resinicola | KAF2810334.1 | 2914 | 4023 | | | | | | | | | | | | | | | |
| Mytilinidion resinicola | KAF2810334.1 | 2915 | 4024 | | | | | | | | | | | | | | | |
| Mytilinidion resinicola | KAF2810334.1 | 2916 | 4025 | 4661 | 5016 | 5245 | 5389 | | | | | | | | | | | |
| Mytilinidion resinicola | KAF2810334.1 | 2917 | 4026 | | | | | | | | | | | | | | | |
| Neofusicoccum parvum | EOD48838.1 | 2918 | 4027 | | | | | | | | | | | | | | | |
| UCRNP2 | | | | | | | | | | | | | | | | | | |
| Neofusicoccum parvum | EOD48838.1 | 2919 | 4028 | 4638 | 5005 | 5237 | | | | | | | | | | | | |
| UCRNP2 | | | | | | | | | | | | | | | | | | |
| Neofusicoccum parvum | EOD48838.1 | 2920 | 4029 | 4889 | | | | | | | | | | | | | | |
| UCRNP2 | | | | | | | | | | | | | | | | | | |
| Neofusicoccum parvum | EOD48838.1 | 2921 | 4030 | 4629 | | | | | | | | | | | | | | |
| UCRNP2 | | | | | | | | | | | | | | | | | | |
| Neonectria ditissima | KPM43150.1 | 2922 | 4031 | | | | | | | | | | | | | | | |
| Neonectria ditissima | KPM43150.1 | 2923 | 4032 | | | | | | | | | | | | | | | |
| Neonectria ditissima | KPM43150.1 | 2924 | 4033 | | | | | | | | | | | | | | | |
| Neonectria ditissima | KPM43150.1 | 2925 | 4034 | | | | | | | | | | | | | | | |
| Neonectria ditissima | KPM43150.1 | 2926 | 4035 | | | | | | | | | | | | | | | |
| Neurospora crassa | EAA35858.1 | 2927 | 4036 | 4569 | 4963 | 5235 | | | | | | | | | | | | |
| Neurospora crassa | EAA35858.1 | 2928 | 4037 | 4481 | 4961 | | | | | | | | | | | | | |
| Neurospora crassa | EAA35858.1 | 2929 | 4038 | 4576 | 4965 | | | | | | | | | | | | | |
| Neurospora crassa | EAA35858.1 | 2930 | 4039 | 4566 | 4955 | 5202 | | | | | | | | | | | | |
| Neurospora crassa | KHE89926.1 | 2931 | 4040 | 4570 | 4964 | 5236 | | | | | | | | | | | | |
| Neurospora crassa | KHE89926.1 | 2932 | 4041 | 4482 | 4962 | | | | | | | | | | | | | |
| Neurospora crassa | KHE89926.1 | 2933 | 4042 | 4577 | 4966 | | | | | | | | | | | | | |
| Neurospora crassa | KHE89926.1 | 2934 | 4043 | 4567 | 4956 | 5203 | | | | | | | | | | | | |
| Neurospora tetrasperma | EGO53172.1 | 2935 | 4044 | | | | | | | | | | | | | | | |
| FGSC 2508 | | | | | | | | | | | | | | | | | | |
| Neurospora tetrasperma | EGO53172.1 | 2936 | 4045 | 4792 | 5097 | 5283 | | | | | | | | | | | | |
| FGSC 2508 | | | | | | | | | | | | | | | | | | |
| Neurospora tetrasperma | EGO53172.1 | 2937 | 4046 | 4795 | 5099 | | | | | | | | | | | | | |
| FGSC 2508 | | | | | | | | | | | | | | | | | | |
| Neurospora tetrasperma | EGO53172.1 | 2938 | 4047 | 4796 | 5100 | | | | | | | | | | | | | |
| FGSC 2508 | | | | | | | | | | | | | | | | | | |
| Neurospora tetrasperma | EGO53172.1 | 2939 | 4048 | 4794 | 5098 | 5284 | | | | | | | | | | | | |
| FGSC 2508 | | | | | | | | | | | | | | | | | | |
| Neurospora tetrasperma | EGZ78015.1 | 2940 | 4049 | | | | | | | | | | | | | | | |
| FGSC 2509 | | | | | | | | | | | | | | | | | | |
| Neurospora tetrasperma | EGZ78015.1 | 2941 | 4050 | 4790 | 5095 | 5282 | 5404 | | | | | | | | | | | |
| FGSC 2509 | | | | | | | | | | | | | | | | | | |
| Neurospora tetrasperma | EGZ78015.1 | 2942 | 4051 | 4791 | | | | | | | | | | | | | | |
| FGSC 2509 | | | | | | | | | | | | | | | | | | |
| Neurospora tetrasperma | EGZ78015.1 | 2943 | 4052 | 4793 | 5096 | | | | | | | | | | | | | |
| FGSC 2509 | | | | | | | | | | | | | | | | | | |
| Neurospora tetrasperma | EGZ78015.1 | 2944 | 4053 | | | | | | | | | | | | | | | |
| FGSC 2509 | | | | | | | | | | | | | | | | | | |
| Ophiocordyceps camponoti- | KAF4594858.1 | 2945 | 4054 | | | | | | | | | | | | | | | |
| rufipedis | | | | | | | | | | | | | | | | | | |
| Ophiocordyceps camponoti- | KAF4594858.1 | 2946 | 4055 | 4710 | | | | | | | | | | | | | | |
| rufipedis | | | | | | | | | | | | | | | | | | |
| Ophiocordyceps camponoti- | KAF4594858.1 | 2947 | 4056 | 4549 | 4935 | 5184 | 5352 | 5450 | 5530 | 5575 | 5609 | 5643 | 5667 | | | | | |
| rufipedis | | | | | | | | | | | | | | | | | | |
| Ophiocordyceps camponoti- | KAF4594858.1 | 2948 | 4057 | 4550 | 4936 | 5185 | 5353 | 5451 | 5531 | 5576 | 5610 | 5644 | 5666 | | | | | |
| rufipedis | | | | | | | | | | | | | | | | | | |
| Ophiocordyceps camponoti- | KAF4594858.1 | 2949 | 4058 | 4551 | 4937 | 5186 | 5354 | 5452 | 5516 | 5574 | 5608 | 5642 | 5668 | | | | | |
| rufipedis | | | | | | | | | | | | | | | | | | |
| Ophiocordyceps camponoti- | KAF4594858.1 | 2950 | 4059 | 4597 | 4975 | 5215 | 5387 | 5475 | 5539 | 5588 | | | | | | | | |
| rufipedis | | | | | | | | | | | | | | | | | | |
| Ophiocordyceps camponoti- | KAF4594858.1 | 2951 | 4060 | 4548 | 4934 | 5183 | 5351 | 5449 | 5515 | 5569 | 5606 | 5636 | 5661 | 5678 | 5686 | | | |
| rufipedis | | | | | | | | | | | | | | | | | | |
| Ophiocordyceps camponoti- | PHH73426.1 | 2952 | 4061 | | | | | | | | | | | | | | | |
| rufipedis | | | | | | | | | | | | | | | | | | |
| Ophiocordyceps camponoti- | PHH73426.1 | 2953 | 4062 | | | | | | | | | | | | | | | |
| rufipedis | | | | | | | | | | | | | | | | | | |
| Ophiocordyceps camponoti- | PHH73426.1 | 2954 | 4063 | | | | | | | | | | | | | | | |
| rufipedis | | | | | | | | | | | | | | | | | | |
| Ophiocordyceps camponoti- | PHH73426.1 | 2955 | 4064 | 4552 | 4938 | 5187 | 5376 | | | | | | | | | | | |
| rufipedis | | | | | | | | | | | | | | | | | | |
| Ophiocordyceps camponoti- | PHH73426.1 | 2956 | 4065 | | | | | | | | | | | | | | | |
| rufipedis | | | | | | | | | | | | | | | | | | |
| Ophiocordyceps camponoti- | PHH73426.1 | 2957 | 4066 | 4656 | | | | | | | | | | | | | | |
| rufipedis | | | | | | | | | | | | | | | | | | |
| Ophiostoma piceae UAMH | EPE06372.1 | 2958 | 4067 | | | | | | | | | | | | | | | |
| 11346 | | | | | | | | | | | | | | | | | | |
| Ophiostoma piceae UAMH | EPE06372.1 | 2959 | 4068 | | | | | | | | | | | | | | | |
| 11346 | | | | | | | | | | | | | | | | | | |
| Ophiostoma piceae UAMH | EPE06372.1 | 2960 | 4069 | 4618 | 4995 | | | | | | | | | | | | | |
| 11346 | | | | | | | | | | | | | | | | | | |
| Penicilliopsis zonata CBS | OJJ47425.1 | 2961 | 4070 | | | | | | | | | | | | | | | |
| 506.65 | | | | | | | | | | | | | | | | | | |
| Penicilliopsis zonata CBS | OJJ47425.1 | 2962 | 4071 | | | | | | | | | | | | | | | |
| 506.65 | | | | | | | | | | | | | | | | | | |
| Penicillium antarcticum | OQD82645.1 | 2963 | 4072 | | | | | | | | | | | | | | | |
| Penicillium antarcticum | OQD82645.1 | 2964 | 4073 | | | | | | | | | | | | | | | |
| Penicillium arizonense | OGE48721.1 | 2965 | 4074 | | | | | | | | | | | | | | | |
| Penicillium arizonense | OGE48721.1 | 2966 | 4075 | | | | | | | | | | | | | | | |
| Penicillium brasilianum | CEJ54559.1 | 2967 | 4076 | | | | | | | | | | | | | | | |
| Penicillium brasilianum | CEJ54559.1 | 2968 | 4077 | | | | | | | | | | | | | | | |
| Penicillium brasilianum | CEJ54559.1 | 2969 | 4078 | | | | | | | | | | | | | | | |
| Penicillium brasilianum | OOQ86087.1 | 2970 | 4079 | | | | | | | | | | | | | | | |
| Penicillium brasilianum | OOQ86087.1 | 2971 | 4080 | | | | | | | | | | | | | | | |
| Penicillium brasilianum | OOQ86087.1 | 2972 | 4081 | | | | | | | | | | | | | | | |
| Penicillium chrysogenum | KZN90002.1 | 2973 | 4082 | | | | | | | | | | | | | | | |
| Penicillium chrysogenum | KZN90002.1 | 2974 | 4083 | | | | | | | | | | | | | | | |
| Penicillium decumbens | OQD70082.1 | 2975 | 4084 | | | | | | | | | | | | | | | |
| Penicillium decumbens | OQD70082.1 | 2976 | 4085 | | | | | | | | | | | | | | | |
| Penicillium digitatum | EKV10174.1 | 2977 | 4086 | | | | | | | | | | | | | | | |
| PHI26 | | | | | | | | | | | | | | | | | | |
| Penicillium digitatum | EKV10174.1 | 2978 | 4087 | | | | | | | | | | | | | | | |
| PHI26 | | | | | | | | | | | | | | | | | | |
| Penicillium digitatum Pd1 | EKV08521.1 | 2979 | 4088 | | | | | | | | | | | | | | | |
| Penicillium digitatum Pd1 | EKV08521.1 | 2980 | 4089 | | | | | | | | | | | | | | | |
| Penicillium rolfsii | KAF3394705.1 | 2981 | 4090 | | | | | | | | | | | | | | | |
| Penicillium rolfsii | KAF3394705.1 | 2982 | 4091 | | | | | | | | | | | | | | | |
| Penicillium roqueforti | CDM34959.1 | 2983 | 4092 | | | | | | | | | | | | | | | |
| FM164 | | | | | | | | | | | | | | | | | | |
| Penicillium roqueforti | CDM34959.1 | 2984 | 4093 | | | | | | | | | | | | | | | |
| FM164 | | | | | | | | | | | | | | | | | | |
| Penicillium steckii | OQE14207.1 | 2985 | 4094 | | | | | | | | | | | | | | | |
| Penicillium steckii | OQE14207.1 | 2986 | 4095 | | | | | | | | | | | | | | | |
| Penicillium subrubescens | OKP08594.1 | 2987 | 4096 | | | | | | | | | | | | | | | |
| Penicillium subrubescens | OKP08594.1 | 2988 | 4097 | | | | | | | | | | | | | | | |
| Penicillium subrubescens | OKP08594.1 | 2989 | 4098 | | | | | | | | | | | | | | | |
| Phaeoacremonium | EOO02638.1 | 2990 | 4099 | 4757 | | | | | | | | | | | | | | |
| minimum UCRPA7 | | | | | | | | | | | | | | | | | | |
| Phaeoacremonium | EOO02638.1 | 2991 | 4100 | 4740 | | | | | | | | | | | | | | |
| minimum UCRPA7 | | | | | | | | | | | | | | | | | | |
| Phaeoacremonium | EOO02638.1 | 2992 | 4101 | 4738 | 5074 | | | | | | | | | | | | | |
| minimum UCRPA7 | | | | | | | | | | | | | | | | | | |
| Phaeoacremonium | EOO02638.1 | 2993 | 4102 | 4739 | 5073 | | | | | | | | | | | | | |
| minimum UCRPA7 | | | | | | | | | | | | | | | | | | |
| Phialemoniopsis curvata | TPX15536.1 | 2994 | 4103 | 4787 | | | | | | | | | | | | | | |
| Phialemoniopsis curvata | TPX15536.1 | 2995 | 4104 | 4785 | 5086 | | | | | | | | | | | | | |
| Phialemoniopsis curvata | TPX15536.1 | 2996 | 4105 | 4786 | | | | | | | | | | | | | | |
| Phialocephala subalpina | CZR51131.1 | 2997 | 4106 | | | | | | | | | | | | | | | |
| Phialocephala subalpina | CZR51131.1 | 2998 | 4107 | | | | | | | | | | | | | | | |
| Phialocephala subalpina | CZR51131.1 | 2999 | 4108 | | | | | | | | | | | | | | | |
| Phialocephala subalpina | CZR51131.1 | 3000 | 4109 | | | | | | | | | | | | | | | |
| Phialocephala subalpina | CZR51131.1 | 3001 | 4110 | | | | | | | | | | | | | | | |
| Phialocephala subalpina | CZR51131.1 | 3002 | 4111 | | | | | | | | | | | | | | | |
| Pleomassaria siparia CBS | KAF2710460.1 | 3003 | 4112 | | | | | | | | | | | | | | | |
| 279.74 | | | | | | | | | | | | | | | | | | |
| Pleomassaria siparia CBS | KAF2710460.1 | 3004 | 4113 | | | | | | | | | | | | | | | |
| 279.74 | | | | | | | | | | | | | | | | | | |
| Pleomassaria siparia CBS | KAF2710460.1 | 3005 | 4114 | | | | | | | | | | | | | | | |
| 279.74 | | | | | | | | | | | | | | | | | | |
| Pleomassaria siparia CBS | KAF2710460.1 | 3006 | 4115 | | | | | | | | | | | | | | | |
| 279.74 | | | | | | | | | | | | | | | | | | |
| Pochonia chlamydosporia | RZR62407.1 | 3007 | 4116 | | | | | | | | | | | | | | | |
| 123 | | | | | | | | | | | | | | | | | | |
| Pochonia chlamydosporia | RZR62407.1 | 3008 | 4117 | | | | | | | | | | | | | | | |
| 123 | | | | | | | | | | | | | | | | | | |
| Pochonia chlamydosporia | RZR62407.1 | 3009 | 4118 | | | | | | | | | | | | | | | |
| 123 | | | | | | | | | | | | | | | | | | |
| Pochonia chlamydosporia | RZR62407.1 | 3010 | 4119 | 4853 | 5150 | | | | | | | | | | | | | |
| 123 | | | | | | | | | | | | | | | | | | |
| Pochonia chlamydosporia | RZR62407.1 | 3011 | 4120 | 4854 | 5149 | | | | | | | | | | | | | |
| 123 | | | | | | | | | | | | | | | | | | |
| Pochonia chlamydosporia | RZR62407.1 | 3012 | 4121 | | | | | | | | | | | | | | | |
| 123 | | | | | | | | | | | | | | | | | | |
| Pochonia chlamydosporia | OAQ71329.1 | 3013 | 4122 | | | | | | | | | | | | | | | |
| 170 | | | | | | | | | | | | | | | | | | |
| Pochonia chlamydosporia | OAQ71329.1 | 3014 | 4123 | | | | | | | | | | | | | | | |
| 170 | | | | | | | | | | | | | | | | | | |
| Pochonia chlamydosporia | OAQ71329.1 | 3015 | 4124 | | | | | | | | | | | | | | | |
| 170 | | | | | | | | | | | | | | | | | | |
| Pochonia chlamydosporia | OAQ71329.1 | 3016 | 4125 | 4865 | | | | | | | | | | | | | | |
| 170 | | | | | | | | | | | | | | | | | | |
| Pochonia chlamydosporia | OAQ71329.1 | 3017 | 4126 | 4864 | | | | | | | | | | | | | | |
| 170 | | | | | | | | | | | | | | | | | | |
| Pochonia chlamydosporia | OAQ71329.1 | 3018 | 4127 | 4855 | | | | | | | | | | | | | | |
| 170 | | | | | | | | | | | | | | | | | | |
| Pochonia chlamydosporia | OAQ71329.1 | 3019 | 4128 | 4856 | | | | | | | | | | | | | | |
| 170 | | | | | | | | | | | | | | | | | | |
| Podospora comata | VBB72273.1 | 3020 | 4129 | | | | | | | | | | | | | | | |
| Podospora comata | VBB72273.1 | 3021 | 4130 | | | | | | | | | | | | | | | |
| Podospora comata | VBB72273.1 | 3022 | 4131 | | | | | | | | | | | | | | | |
| Polychaeton citri CBS | KAF2725433.1 | 3023 | 4132 | | | | | | | | | | | | | | | |
| 116435 | | | | | | | | | | | | | | | | | | |
| Polyplosphaeria fusca | KAF2740445.1 | 3024 | 4133 | | | | | | | | | | | | | | | |
| Polyplosphaeria fusca | KAF2740445.1 | 3025 | 4134 | | | | | | | | | | | | | | | |
| Polyplosphaeria fusca | KAF2740445.1 | 3026 | 4135 | 4697 | 5036 | 5323 | | | | | | | | | | | | |
| Polyplosphaeria fusca | KAF2740445.1 | 3027 | 4136 | | | | | | | | | | | | | | | |
| Polyplosphaeria fusca | KAF2740445.1 | 3028 | 4137 | | | | | | | | | | | | | | | |
| Polyplosphaeria fusca | KAF2740445.1 | 3029 | 4138 | | | | | | | | | | | | | | | |
| Pseudogymnoascus destructans | OAF56923.1 | 3030 | 4139 | | | | | | | | | | | | | | | |
| Pseudogymnoascus destructans | OAF56923.1 | 3031 | 4140 | | | | | | | | | | | | | | | |
| Pseudogymnoascus destructans | OAF56923.1 | 3032 | 4141 | | | | | | | | | | | | | | | |
| Pseudogymnoascus destructans | OAF56923.1 | 3033 | 4142 | | | | | | | | | | | | | | | |
| Pseudogymnoascus destructans | OAF56923.1 | 3034 | 4143 | | | | | | | | | | | | | | | |
| Pseudogymnoascus | ELR03405.1 | 3035 | 4144 | | | | | | | | | | | | | | | |
| destructans 20631-21 | | | | | | | | | | | | | | | | | | |
| Pseudogymnoascus | ELR03405.1 | 3036 | 4145 | | | | | | | | | | | | | | | |
| destructans 20631-21 | | | | | | | | | | | | | | | | | | |
| Pseudogymnoascus | ELR03405.1 | 3037 | 4146 | | | | | | | | | | | | | | | |
| destructans 20631-21 | | | | | | | | | | | | | | | | | | |
| Pseudogymnoascus | ELR03405.1 | 3038 | 4147 | | | | | | | | | | | | | | | |
| destructans 20631-21 | | | | | | | | | | | | | | | | | | |
| Pseudogymnoascus | ELR03405.1 | 3039 | 4148 | | | | | | | | | | | | | | | |
| destructans 20631-21 | | | | | | | | | | | | | | | | | | |
| Pseudogymnoascus sp. | OBT90158.1 | 3040 | 4149 | 4723 | 5155 | 5331 | | | | | | | | | | | | |
| 03VT05 | | | | | | | | | | | | | | | | | | |
| Pseudogymnoascus sp. | OBT90158.1 | 3041 | 4150 | 4714 | 5046 | 5330 | | | | | | | | | | | | |
| 03VT05 | | | | | | | | | | | | | | | | | | |
| Pseudogymnoascus sp. | OBT90158.1 | 3042 | 4151 | 4535 | 4930 | | | | | | | | | | | | | |
| 03VT05 | | | | | | | | | | | | | | | | | | |
| Pseudogymnoascus sp. | OBT77860.1 | 3043 | 4152 | 4724 | 5156 | | | | | | | | | | | | | |
| 05NY08 | | | | | | | | | | | | | | | | | | |
| Pseudogymnoascus sp. | OBT77860.1 | 3044 | 4153 | 4716 | 5048 | | | | | | | | | | | | | |
| 05NY08 | | | | | | | | | | | | | | | | | | |
| Pseudogymnoascus sp. | OBT77860.1 | 3045 | 4154 | 4536 | 4928 | | | | | | | | | | | | | |
| 05NY08 | | | | | | | | | | | | | | | | | | |
| Pseudogymnoascus sp. | OBT51310.1 | 3046 | 4155 | 4619 | 4998 | | | | | | | | | | | | | |
| 24MN13 | | | | | | | | | | | | | | | | | | |
| Pseudogymnoascus sp. | OBT51310.1 | 3047 | 4156 | 4478 | 5009 | | | | | | | | | | | | | |
| 24MN13 | | | | | | | | | | | | | | | | | | |
| Pseudogymnoascus sp. | OBT51310.1 | 3048 | 4157 | 4477 | 5006 | | | | | | | | | | | | | |
| 24MN13 | | | | | | | | | | | | | | | | | | |
| Pseudogymnoascus sp. | OBT70084.1 | 3049 | 4158 | 4502 | 4978 | | | | | | | | | | | | | |
| 23342-1-I1 | | | | | | | | | | | | | | | | | | |
| Pseudogymnoascus sp. | OBT70084.1 | 3050 | 4159 | 4626 | | | | | | | | | | | | | | |
| 23342-1-I1 | | | | | | | | | | | | | | | | | | |
| Pseudogymnoascus sp. | KFY71191.1 | 3051 | 4160 | 4720 | 5050 | | | | | | | | | | | | | |
| VKM F-103 | | | | | | | | | | | | | | | | | | |
| Pseudogymnoascus sp. | KFY71191.1 | 3052 | 4161 | 4713 | 5045 | | | | | | | | | | | | | |
| VKM F-103 | | | | | | | | | | | | | | | | | | |
| Pseudogymnoascus sp. | KFY71191.1 | 3053 | 4162 | 4531 | 4927 | | | | | | | | | | | | | |
| VKM F-103 | | | | | | | | | | | | | | | | | | |
| Pseudogymnoascus sp. | KFX87612.1 | 3054 | 4163 | 4717 | 5043 | 5258 | | | | | | | | | | | | |
| VKM F-3557 | | | | | | | | | | | | | | | | | | |
| Pseudogymnoascus sp. | KFX87612.1 | 3055 | 4164 | 4711 | | | | | | | | | | | | | | |
| VKM F-3557 | | | | | | | | | | | | | | | | | | |
| Pseudogymnoascus sp. | KFX87612.1 | 3056 | 4165 | 4532 | 4924 | 5181 | | | | | | | | | | | | |
| VKM F-3557 | | | | | | | | | | | | | | | | | | |
| Pseudogymnoascus sp. | KFY06024.1 | 3057 | 4166 | 4510 | | | | | | | | | | | | | | |
| VKM F-3775 | | | | | | | | | | | | | | | | | | |
| Pseudogymnoascus sp. | KFY06024.1 | 3058 | 4167 | | | | | | | | | | | | | | | |
| VKM F-3775 | | | | | | | | | | | | | | | | | | |
| Pseudogymnoascus sp. | KFY11495.1 | 3059 | 4168 | 4507 | 4907 | | | | | | | | | | | | | |
| VKM F-4246 | | | | | | | | | | | | | | | | | | |
| Pseudogymnoascus sp. | KFY11495.1 | 3060 | 4169 | 4620 | | | | | | | | | | | | | | |
| VKM F-4246 | | | | | | | | | | | | | | | | | | |
| Pseudogymnoascus sp. | KFY29023.1 | 3061 | 4170 | | | | | | | | | | | | | | | |
| VKM F-4281 (FW-2241) | | | | | | | | | | | | | | | | | | |
| Pseudogymnoascus sp. | KFY29023.1 | 3062 | 4171 | | | | | | | | | | | | | | | |
| VKM F-4281 (FW-2241) | | | | | | | | | | | | | | | | | | |
| Pseudogymnoascus sp. | KFY29023.1 | 3063 | 4172 | | | | | | | | | | | | | | | |
| VKM F-4281 (FW-2241) | | | | | | | | | | | | | | | | | | |
| Pseudogymnoascus sp. | KFY29023.1 | 3064 | 4173 | | | | | | | | | | | | | | | |
| VKM F-4281 (FW-2241) | | | | | | | | | | | | | | | | | | |
| Pseudogymnoascus sp. | KFY34189.1 | 3065 | 4174 | 4508 | 4908 | | | | | | | | | | | | | |
| VKM F-4513 (FW-928) | | | | | | | | | | | | | | | | | | |
| Pseudogymnoascus sp. | KFY34189.1 | 3066 | 4175 | 4621 | | | | | | | | | | | | | | |
| VKM F-4513 (FW-928) | | | | | | | | | | | | | | | | | | |
| Pseudogymnoascus sp. | KFY38045.1 | 3067 | 4176 | 4500 | 4905 | 5239 | | | | | | | | | | | | |
| VKM F-4514 (FW-929) | | | | | | | | | | | | | | | | | | |
| Pseudogymnoascus sp. | KFY38045.1 | 3068 | 4177 | 4624 | | | | | | | | | | | | | | |
| VKM F-4514 (FW-929) | | | | | | | | | | | | | | | | | | |
| Pseudogymnoascus sp. | KFY38045.1 | 3069 | 4178 | 4501 | 4906 | 5240 | | | | | | | | | | | | |
| VKM F-4514 (FW-929) | | | | | | | | | | | | | | | | | | |
| Pseudogymnoascus sp. | KFY64466.1 | 3070 | 4179 | 4663 | 5017 | | | | | | | | | | | | | |
| VKM F-4515 (FW-2607) | | | | | | | | | | | | | | | | | | |
| Pseudogymnoascus sp. | KFY64466.1 | 3071 | 4180 | 4662 | | | | | | | | | | | | | | |
| VKM F-4515 (FW-2607) | | | | | | | | | | | | | | | | | | |
| Pseudogymnoascus sp. | KFY56828.1 | 3072 | 4181 | 4718 | 5044 | 5259 | | | | | | | | | | | | |
| VKM F-4516 (FW-969) | | | | | | | | | | | | | | | | | | |
| Pseudogymnoascus sp. | KFY56828.1 | 3073 | 4182 | 4712 | | | | | | | | | | | | | | |
| VKM F-4516 (FW-969) | | | | | | | | | | | | | | | | | | |
| Pseudogymnoascus sp. | KFY56828.1 | 3074 | 4183 | 4533 | 4925 | 5182 | | | | | | | | | | | | |
| VKM F-4516 (FW-969) | | | | | | | | | | | | | | | | | | |
| Pseudogymnoascus sp. | KFY93521.1 | 3075 | 4184 | 4506 | 5014 | | | | | | | | | | | | | |
| VKM F-4517 (FW-2822) | | | | | | | | | | | | | | | | | | |
| Pseudogymnoascus sp. | KFY93521.1 | 3076 | 4185 | 4625 | | | | | | | | | | | | | | |
| VKM F-4517 (FW-2822) | | | | | | | | | | | | | | | | | | |
| Pseudogymnoascus sp. | KFY96313.1 | 3077 | 4186 | 4879 | | | | | | | | | | | | | | |
| VKM F-4518 (FW-2643) | | | | | | | | | | | | | | | | | | |
| Pseudogymnoascus sp. | KFY96313.1 | 3078 | 4187 | 4878 | | | | | | | | | | | | | | |
| VKM F-4518 (FW-2643) | | | | | | | | | | | | | | | | | | |
| Pseudogymnoascus sp. | KFZ06200.1 | 3079 | 4188 | 4719 | 5049 | | | | | | | | | | | | | |
| VKM F-4519 (FW-2642) | | | | | | | | | | | | | | | | | | |
| Pseudogymnoascus sp. | KFZ06200.1 | 3080 | 4189 | 4715 | 5047 | | | | | | | | | | | | | |
| VKM F-4519 (FW-2642) | | | | | | | | | | | | | | | | | | |
| Pseudogymnoascus sp. | KFZ06200.1 | 3081 | 4190 | 4530 | 4926 | | | | | | | | | | | | | |
| VKM F-4519 (FW-2642) | | | | | | | | | | | | | | | | | | |
| Pseudogymnoascus sp. | KFZ18892.1 | 3082 | 4191 | 4646 | | | | | | | | | | | | | | |
| VKM F-4520 (FW-2644) | | | | | | | | | | | | | | | | | | |
| Pseudogymnoascus sp. | KFZ18892.1 | 3083 | 4192 | 4622 | | | | | | | | | | | | | | |
| VKM F-4520 (FW-2644) | | | | | | | | | | | | | | | | | | |
| Pseudogymnoascus sp. | OBT48713.1 | 3084 | 4193 | | | | | | | | | | | | | | | |
| WSF 3629 | | | | | | | | | | | | | | | | | | |
| Pseudogymnoascus sp. | OBT48713.1 | 3085 | 4194 | 4722 | 5157 | | | | | | | | | | | | | |
| WSF 3629 | | | | | | | | | | | | | | | | | | |
| Pseudogymnoascus sp. | OBT48713.1 | 3086 | 4195 | 4721 | | | | | | | | | | | | | | |
| WSF 3629 | | | | | | | | | | | | | | | | | | |
| Pseudogymnoascus sp. | OBT48713.1 | 3087 | 4196 | 4534 | 4929 | | | | | | | | | | | | | |
| WSF 3629 | | | | | | | | | | | | | | | | | | |
| Pseudogymnoascus verrucosus | OBT98717.1 | 3088 | 4197 | 4475 | 5007 | | | | | | | | | | | | | |
| Pseudogymnoascus verrucosus | OBT98717.1 | 3089 | 4198 | 4601 | 4997 | | | | | | | | | | | | | |
| Pseudogymnoascus verrucosus | OBT98717.1 | 3090 | 4199 | 4476 | 5008 | | | | | | | | | | | | | |
| Purpureocillium lilacinum | OAQ77955.1 | 3091 | 4200 | 4733 | 5058 | 5266 | | | | | | | | | | | | |
| Purpureocillium lilacinum | OAQ77955.1 | 3092 | 4201 | | | | | | | | | | | | | | | |
| Purpureocillium lilacinum | OAQ77955.1 | 3093 | 4202 | 4760 | | | | | | | | | | | | | | |
| Purpureocillium lilacinum | OAQ77955.1 | 3094 | 4203 | | | | | | | | | | | | | | | |
| Purpureocillium lilacinum | OAQ87765.1 | 3095 | 4204 | 4734 | 5059 | 5264 | | | | | | | | | | | | |
| Purpureocillium lilacinum | OAQ87765.1 | 3096 | 4205 | | | | | | | | | | | | | | | |
| Purpureocillium lilacinum | OAQ87765.1 | 3097 | 4206 | 4761 | | | | | | | | | | | | | | |
| Purpureocillium lilacinum | OAQ87765.1 | 3098 | 4207 | | | | | | | | | | | | | | | |
| Purpureocillium lilacinum | PWI71843.1 | 3099 | 4208 | 4735 | 5060 | 5265 | | | | | | | | | | | | |
| Purpureocillium lilacinum | PWI71843.1 | 3100 | 4209 | | | | | | | | | | | | | | | |
| Purpureocillium lilacinum | PWI71843.1 | 3101 | 4210 | 4762 | 5140 | | | | | | | | | | | | | |
| Purpureocillium lilacinum | PWI71843.1 | 3102 | 4211 | | | | | | | | | | | | | | | |
| Pyricularia grisea | TLD05564.1 | 3103 | 4212 | | | | | | | | | | | | | | | |
| Pyricularia grisea | TLD05564.1 | 3104 | 4213 | 4580 | 4985 | 5220 | | | | | | | | | | | | |
| Pyricularia grisea | TLD05564.1 | 3105 | 4214 | 4572 | 4957 | 5195 | | | | | | | | | | | | |
| Pyricularia oryzae | QBZ59762.1 | 3106 | 4215 | | | | | | | | | | | | | | | |
| Pyricularia oryzae | QBZ59762.1 | 3107 | 4216 | 4779 | 5090 | 5279 | | | | | | | | | | | | |
| Pyricularia oryzae | QBZ59762.1 | 3108 | 4217 | 4782 | 5087 | 5276 | | | | | | | | | | | | |
| Pyricularia oryzae 70-15 | EHA53132.1 | 3109 | 4218 | | | | | | | | | | | | | | | |
| Pyricularia oryzae 70-15 | EHA53132.1 | 3110 | 4219 | 4780 | 5091 | 5280 | | | | | | | | | | | | |
| Pyricularia oryzae 70-15 | EHA53132.1 | 3111 | 4220 | 4783 | 5088 | 5277 | | | | | | | | | | | | |
| Pyricularia oryzae P131 | ELQ63379.1 | 3112 | 4221 | | | | | | | | | | | | | | | |
| Pyricularia oryzae P131 | ELQ63379.1 | 3113 | 4222 | 4579 | 4983 | 5223 | | | | | | | | | | | | |
| Pyricularia oryzae P131 | ELQ63379.1 | 3114 | 4223 | 4578 | 4968 | 5231 | | | | | | | | | | | | |
| Pyricularia oryzae Y34 | ELQ41287.1 | 3115 | 4224 | | | | | | | | | | | | | | | |
| Pyricularia oryzae Y34 | ELQ41287.1 | 3116 | 4225 | 4781 | 5092 | 5281 | | | | | | | | | | | | |
| Pyricularia oryzae Y34 | ELQ41287.1 | 3117 | 4226 | 4784 | 5089 | 5278 | | | | | | | | | | | | |
| Pyricularia pennisetigena | TLS30223.1 | 3118 | 4227 | 4789 | 5094 | 5315 | | | | | | | | | | | | |
| Pyricularia pennisetigena | TLS30223.1 | 3119 | 4228 | 4788 | 5093 | 5314 | | | | | | | | | | | | |
| Pyricularia sp. CBS 133598 | TLD31639.1 | 3120 | 4229 | | | | | | | | | | | | | | | |
| Pyricularia sp. CBS 133598 | TLD31639.1 | 3121 | 4230 | 4581 | 4990 | 5226 | | | | | | | | | | | | |
| Pyricularia sp. CBS 133598 | TLD31639.1 | 3122 | 4231 | 4571 | 4967 | 5204 | | | | | | | | | | | | |
| Ramularia collo-cygni | CZT18030.1 | 3123 | 4232 | | | | | | | | | | | | | | | |
| Ramularia collo-cygni | CZT18030.1 | 3124 | 4233 | | | | | | | | | | | | | | | |
| Ramularia collo-cygni | CZT18030.1 | 3125 | 4234 | | | | | | | | | | | | | | | |
| Ramularia collo-cygni | CZT18030.1 | 3126 | 4235 | 4880 | | | | | | | | | | | | | | |
| Ramularia collo-cygni | CZT18030.1 | 3127 | 4236 | | | | | | | | | | | | | | | |
| Ramularia collo-cygni | CZT18030.1 | 3128 | 4237 | 4875 | | | | | | | | | | | | | | |
| Rhizodiscina lignyota | KAF2099123.1 | 3129 | 4238 | | | | | | | | | | | | | | | |
| Rhizodiscina lignyota | KAF2099123.1 | 3130 | 4239 | | | | | | | | | | | | | | | |
| Rhizodiscina lignyota | KAF2099123.1 | 3131 | 4240 | | | | | | | | | | | | | | | |
| Rutstroemia sp. NJR-2017a | PQE12560.1 | 3132 | 4241 | | | | | | | | | | | | | | | |
| BBW | | | | | | | | | | | | | | | | | | |
| Rutstroemia sp. NJR-2017a | PQE12560.1 | 3133 | 4242 | 4839 | 5133 | 5308 | 5425 | 5492 | | | | | | | | | | |
| BBW | | | | | | | | | | | | | | | | | | |
| Rutstroemia sp. NJR-2017a | PQE12560.1 | 3134 | 4243 | 4819 | 5132 | 5307 | 5424 | 5487 | 5545 | | | | | | | | | |
| BBW | | | | | | | | | | | | | | | | | | |
| Rutstroemia sp. NJR-2017a | PQE12560.1 | 3135 | 4244 | | | | | | | | | | | | | | | |
| BBW | | | | | | | | | | | | | | | | | | |
| Rutstroemia sp. NJR-2017a | PQE08130.1 | 3136 | 4245 | | | | | | | | | | | | | | | |
| BVV2 | | | | | | | | | | | | | | | | | | |
| Rutstroemia sp. NJR-2017a | PQE08130.1 | 3137 | 4246 | 4837 | 5130 | 5306 | 5422 | 5488 | 5546 | | | | | | | | | |
| BVV2 | | | | | | | | | | | | | | | | | | |
| Rutstroemia sp. NJR-2017a | PQE08130.1 | 3138 | 4247 | 4838 | 5131 | 5305 | 5423 | 5489 | 5547 | | | | | | | | | |
| BVV2 | | | | | | | | | | | | | | | | | | |
| Rutstroemia sp. NJR-2017a | PQE08130.1 | 3139 | 4248 | | | | | | | | | | | | | | | |
| BVV2 | | | | | | | | | | | | | | | | | | |
| Rutstroemia sp. NJR-2017a | PQE08130.1 | 3140 | 4249 | 4836 | | | | | | | | | | | | | | |
| BVV2 | | | | | | | | | | | | | | | | | | |
| Rutstroemia sp. NJR-2017a | PQE28263.1 | 3141 | 4250 | | | | | | | | | | | | | | | |
| WRK4 | | | | | | | | | | | | | | | | | | |
| Rutstroemia sp. NJR-2017a | PQE28263.1 | 3142 | 4251 | 4586 | 4994 | 5333 | 5438 | | | | | | | | | | | |
| WRK4 | | | | | | | | | | | | | | | | | | |
| Rutstroemia sp. NJR-2017a | PQE28263.1 | 3143 | 4252 | 4631 | 5013 | 5244 | 5390 | 5510 | | | | | | | | | | |
| WRK4 | | | | | | | | | | | | | | | | | | |
| Rutstroemia sp. NJR-2017a | PQE28263.1 | 3144 | 4253 | | | | | | | | | | | | | | | |
| WRK4 | | | | | | | | | | | | | | | | | | |
| Saccharata proteae CBS | KAF2084183.1 | 3145 | 4254 | 4641 | | | | | | | | | | | | | | |
| 121410 | | | | | | | | | | | | | | | | | | |
| Saccharata proteae CBS | KAF2084183.1 | 3146 | 4255 | | | | | | | | | | | | | | | |
| 121410 | | | | | | | | | | | | | | | | | | |
| Saccharata proteae CBS | KAF2084183.1 | 3147 | 4256 | 4647 | | | | | | | | | | | | | | |
| 121410 | | | | | | | | | | | | | | | | | | |
| Sclerotinia borealis F-4128 | ESZ97974.1 | 3148 | 4257 | | | | | | | | | | | | | | | |
| Sclerotinia borealis F-4128 | ESZ97974.1 | 3149 | 4258 | | | | | | | | | | | | | | | |
| Sclerotinia borealis F-4128 | ESZ97974.1 | 3150 | 4259 | 4835 | 5129 | 5322 | | | | | | | | | | | | |
| Sclerotinia borealis F-4128 | ESZ97974.1 | 3151 | 4260 | 4834 | 5125 | 5304 | 5433 | | | | | | | | | | | |
| Sclerotinia borealis F-4128 | ESZ97974.1 | 3152 | 4261 | | | | | | | | | | | | | | | |
| Sclerotinia borealis F-4128 | ESZ97974.1 | 3153 | 4262 | 4862 | | | | | | | | | | | | | | |
| Sclerotinia sclerotiorum | APA07130.1 | 3154 | 4263 | | | | | | | | | | | | | | | |
| 1980 UF-70 | | | | | | | | | | | | | | | | | | |
| Sclerotinia sclerotiorum | APA07130.1 | 3155 | 4264 | 4587 | 4973 | 5230 | 5439 | 5511 | 5567 | | | | | | | | | |
| 1980 UF-70 | | | | | | | | | | | | | | | | | | |
| Sclerotinia sclerotiorum | APA07130.1 | 3156 | 4265 | 4521 | 4914 | 5233 | 5384 | 5476 | 5566 | | | | | | | | | |
| 1980 UF-70 | | | | | | | | | | | | | | | | | | |
| Sclerotinia sclerotiorum | APA07130.1 | 3157 | 4266 | | | | | | | | | | | | | | | |
| 1980 UF-70 | | | | | | | | | | | | | | | | | | |
| Sclerotinia sclerotiorum | APA07130.1 | 3158 | 4267 | | | | | | | | | | | | | | | |
| 1980 UF-70 | | | | | | | | | | | | | | | | | | |
| Sclerotinia sclerotiorum | EDO01680.1 | 3159 | 4268 | | | | | | | | | | | | | | | |
| 1980 UF-70 | | | | | | | | | | | | | | | | | | |
| Sclerotinia sclerotiorum | EDO01680.1 | 3160 | 4269 | 4825 | 5118 | 5298 | 5418 | 5485 | 5555 | | | | | | | | | |
| 1980 UF-70 | | | | | | | | | | | | | | | | | | |
| Sclerotinia sclerotiorum | EDO01680.1 | 3161 | 4270 | 4826 | 5117 | 5301 | 5419 | 5486 | 5556 | | | | | | | | | |
| 1980 UF-70 | | | | | | | | | | | | | | | | | | |
| Sclerotinia sclerotiorum | EDO01680.1 | 3162 | 4271 | | | | | | | | | | | | | | | |
| 1980 UF-70 | | | | | | | | | | | | | | | | | | |
| Sclerotinia sclerotiorum | EDO01680.1 | 3163 | 4272 | | | | | | | | | | | | | | | |
| 1980 UF-70 | | | | | | | | | | | | | | | | | | |
| Sodiomyces alkalinus F11 | ROT37183.1 | 3164 | 4273 | 4683 | 5032 | 5253 | | | | | | | | | | | | |
| Sodiomyces alkalinus F11 | ROT37183.1 | 3165 | 4274 | 4682 | 5031 | 5252 | 5396 | | | | | | | | | | | |
| Sordaria macrospora | KAA8633182.1 | 3166 | 4275 | 4799 | 5101 | | | | | | | | | | | | | |
| Sordaria macrospora | KAA8633182.1 | 3167 | 4276 | 4803 | 5143 | | | | | | | | | | | | | |
| Sordaria macrospora | KAA8633182.1 | 3168 | 4277 | 4805 | 5145 | | | | | | | | | | | | | |
| Sordaria macrospora | KAA8633182.1 | 3169 | 4278 | 4797 | 5141 | | | | | | | | | | | | | |
| Sordaria macrospora | KAA8633182.1 | 3170 | 4279 | 4801 | 5103 | 5316 | | | | | | | | | | | | |
| Sordaria macrospora k-hell | CCC08615.1 | 3171 | 4280 | 4800 | 5102 | | | | | | | | | | | | | |
| Sordaria macrospora k-hell | CCC08615.1 | 3172 | 4281 | 4804 | 5144 | | | | | | | | | | | | | |
| Sordaria macrospora k-hell | CCC08615.1 | 3173 | 4282 | 4806 | 5146 | | | | | | | | | | | | | |
| Sordaria macrospora k-hell | CCC08615.1 | 3174 | 4283 | 4798 | 5142 | | | | | | | | | | | | | |
| Sordaria macrospora k-hell | CCC08615.1 | 3175 | 4284 | 4802 | 5104 | 5317 | | | | | | | | | | | | |
| Source: UniProtKB/TrEMBL; | CBF81183.1 | 3176 | 4285 | | | | | | | | | | | | | | | |
| Acc: Q7SI74 | | | | | | | | | | | | | | | | | | |
| Source: UniProtKB/TrEMBL; | CBF81183.1 | 3177 | 4286 | | | | | | | | | | | | | | | |
| Acc: Q7SI74 | | | | | | | | | | | | | | | | | | |
| Sporothrix brasiliensis 5110 | KIH94007.1 | 3178 | 4287 | 4568 | 4989 | | | | | | | | | | | | | |
| Sporothrix brasiliensis 5110 | KIH94007.1 | 3179 | 4288 | 4648 | | | | | | | | | | | | | | |
| Sporothrix brasiliensis 5110 | KIH94007.1 | 3180 | 4289 | | | | | | | | | | | | | | | |
| Sporothrix brasiliensis 5110 | KIH94007.1 | 3181 | 4290 | | | | | | | | | | | | | | | |
| Sporothrix insectorum | OAA66999.1 | 3182 | 4291 | | | | | | | | | | | | | | | |
| RCEF 264 | | | | | | | | | | | | | | | | | | |
| Sporothrix insectorum | OAA66999.1 | 3183 | 4292 | 4874 | | | | | | | | | | | | | | |
| RCEF 264 | | | | | | | | | | | | | | | | | | |
| Sporothrix insectorum | OAA66999.1 | 3184 | 4293 | | | | | | | | | | | | | | | |
| RCEF 264 | | | | | | | | | | | | | | | | | | |
| Sporothrix schenckii 1099- | KJR83391.1 | 3185 | 4294 | 4665 | | | | | | | | | | | | | | |
| 18 | | | | | | | | | | | | | | | | | | |
| Sporothrix schenckii 1099- | KJR83391.1 | 3186 | 4295 | | | | | | | | | | | | | | | |
| 18 | | | | | | | | | | | | | | | | | | |
| Sporothrix schenckii 1099- | KJR83391.1 | 3187 | 4296 | | | | | | | | | | | | | | | |
| 18 | | | | | | | | | | | | | | | | | | |
| Sporothrix schenckii ATCC | ERS98966.1 | 3188 | 4297 | 4609 | | | | | | | | | | | | | | |
| 58251 | | | | | | | | | | | | | | | | | | |
| Sporothrix schenckii ATCC | ERS98966.1 | 3189 | 4298 | | | | | | | | | | | | | | | |
| 58251 | | | | | | | | | | | | | | | | | | |
| Sporothrix schenckii ATCC | ERS98966.1 | 3190 | 4299 | | | | | | | | | | | | | | | |
| 58251 | | | | | | | | | | | | | | | | | | |
| Stachybotrys chartarum IBT | KEY69261.1 | 3191 | 4300 | 4612 | 5003 | | | | | | | | | | | | | |
| 7711 | | | | | | | | | | | | | | | | | | |
| Stachybotrys chartarum IBT | KEY69261.1 | 3192 | 4301 | 4538 | | | | | | | | | | | | | | |
| 7711 | | | | | | | | | | | | | | | | | | |
| Stachybotrys chartarum IBT | KEY69261.1 | 3193 | 4302 | | | | | | | | | | | | | | | |
| 7711 | | | | | | | | | | | | | | | | | | |
| Stachybotrys chartarum IBT | KFA76124.1 | 3194 | 4303 | 4613 | 5004 | | | | | | | | | | | | | |
| 40288 | | | | | | | | | | | | | | | | | | |
| Stachybotrys chartarum IBT | KFA76124.1 | 3195 | 4304 | 4539 | | | | | | | | | | | | | | |
| 40288 | | | | | | | | | | | | | | | | | | |
| Stachybotrys chartarum IBT | KFA76124.1 | 3196 | 4305 | | | | | | | | | | | | | | | |
| 40288 | | | | | | | | | | | | | | | | | | |
| Stachybotrys chartarum IBT | KFA46039.1 | 3197 | 4306 | 4755 | 5077 | | | | | | | | | | | | | |
| 40293 | | | | | | | | | | | | | | | | | | |
| Stachybotrys chartarum IBT | KFA46039.1 | 3198 | 4307 | 4756 | | | | | | | | | | | | | | |
| 40293 | | | | | | | | | | | | | | | | | | |
| Stachybotrys chartarum IBT | KFA46039.1 | 3199 | 4308 | | | | | | | | | | | | | | | |
| 40293 | | | | | | | | | | | | | | | | | | |
| Stachybotrys | KFA67343.1 | 3200 | 4309 | 4540 | 4992 | 5238 | | | | | | | | | | | | |
| chlorohalonata IBT 40285 | | | | | | | | | | | | | | | | | | |
| Stachybotrys | KFA67343.1 | 3201 | 4310 | 4537 | 4931 | | | | | | | | | | | | | |
| chlorohalonata IBT 40285 | | | | | | | | | | | | | | | | | | |
| Stachybotrys | KFA67343.1 | 3202 | 4311 | | | | | | | | | | | | | | | |
| chlorohalonata IBT 40285 | | | | | | | | | | | | | | | | | | |
| Thermothielavioides | SPQ18545.1 | 3203 | 4312 | | | | | | | | | | | | | | | |
| terrestris | | | | | | | | | | | | | | | | | | |
| Thermothielavioides | SPQ18545.1 | 3204 | 4313 | | | | | | | | | | | | | | | |
| terrestris | | | | | | | | | | | | | | | | | | |
| Thermothielavioides | SPQ18545.1 | 3205 | 4314 | | | | | | | | | | | | | | | |
| terrestris | | | | | | | | | | | | | | | | | | |
| Thermothielavioides | SPQ18545.1 | 3206 | 4315 | 4747 | 5065 | | | | | | | | | | | | | |
| terrestris | | | | | | | | | | | | | | | | | | |
| Thermothielavioides | SPQ18545.1 | 3207 | 4316 | 4748 | 5066 | | | | | | | | | | | | | |
| terrestris | | | | | | | | | | | | | | | | | | |
| Tothia fuscella | KAF2433310.1 | 3208 | 4317 | 4614 | 5029 | | | | | | | | | | | | | |
| Tothia fuscella | KAF2433310.1 | 3209 | 4318 | | | | | | | | | | | | | | | |
| Tothia fuscella | KAF2433310.1 | 3210 | 4319 | | | | | | | | | | | | | | | |
| Trichoderma arundinaceum | RFU76614.1 | 3211 | 4320 | | | | | | | | | | | | | | | |
| Trichoderma arundinaceum | RFU76614.1 | 3212 | 4321 | | | | | | | | | | | | | | | |
| Trichoderma arundinaceum | RFU76614.1 | 3213 | 4322 | 4845 | | | | | | | | | | | | | | |
| Trichoderma asperellum | GFP57911.1 | 3214 | 4323 | 4498 | 4999 | 5246 | | | | | | | | | | | | |
| Trichoderma asperellum | PTB39383.1 | 3215 | 4324 | 4499 | 5000 | 5247 | | | | | | | | | | | | |
| CBS 433.97 | | | | | | | | | | | | | | | | | | |
| Trichoderma atroviride IMI | EHK50485.1 | 3216 | 4325 | 4632 | 5001 | 5249 | | | | | | | | | | | | |
| 206040 | | | | | | | | | | | | | | | | | | |
| Trichoderma citrinoviride | PTB66885.1 | 3217 | 4326 | | | | | | | | | | | | | | | |
| Trichoderma citrinoviride | PTB66885.1 | 3218 | 4327 | 4634 | | | | | | | | | | | | | | |
| Trichoderma citrinoviride | PTB66885.1 | 3219 | 4328 | | | | | | | | | | | | | | | |
| Trichoderma citrinoviride | PTB66885.1 | 3220 | 4329 | | | | | | | | | | | | | | | |
| Trichoderma gamsii | PNP46806.1 | 3221 | 4330 | 4852 | 5136 | 5310 | | | | | | | | | | | | |
| Trichoderma gamsii | PON27232.1 | 3222 | 4331 | 4633 | 5002 | 5248 | | | | | | | | | | | | |
| Trichoderma guizhouense | OPB47026.1 | 3223 | 4332 | | | | | | | | | | | | | | | |
| Trichoderma guizhouense | OPB47026.1 | 3224 | 4333 | | | | | | | | | | | | | | | |
| Trichoderma guizhouense | OPB47026.1 | 3225 | 4334 | | | | | | | | | | | | | | | |
| Trichoderma guizhouense | OPB47026.1 | 3226 | 4335 | | | | | | | | | | | | | | | |
| Trichoderma guizhouense | OPB47026.1 | 3227 | 4336 | 4669 | | | | | | | | | | | | | | |
| Trichoderma harzianum | KKP01241.1 | 3228 | 4337 | | | | | | | | | | | | | | | |
| Trichoderma harzianum | KKP01241.1 | 3229 | 4338 | | | | | | | | | | | | | | | |
| Trichoderma harzianum | KKP01241.1 | 3230 | 4339 | 4844 | | | | | | | | | | | | | | |
| Trichoderma harzianum | PKK54570.1 | 3231 | 4340 | | | | | | | | | | | | | | | |
| Trichoderma harzianum | PKK54570.1 | 3232 | 4341 | | | | | | | | | | | | | | | |
| Trichoderma harzianum | PKK54570.1 | 3233 | 4342 | | | | | | | | | | | | | | | |
| Trichoderma harzianum | PKK54570.1 | 3234 | 4343 | | | | | | | | | | | | | | | |
| Trichoderma harzianum | PNP51338.1 | 3235 | 4344 | | | | | | | | | | | | | | | |
| Trichoderma harzianum | PNP51338.1 | 3236 | 4345 | | | | | | | | | | | | | | | |
| Trichoderma harzianum | PNP51338.1 | 3237 | 4346 | 4670 | 5020 | | | | | | | | | | | | | |
| Trichoderma harzianum | PNP51338.1 | 3238 | 4347 | | | | | | | | | | | | | | | |
| Trichoderma harzianum | PTB53931.1 | 3239 | 4348 | | | | | | | | | | | | | | | |
| CBS 226.95 | | | | | | | | | | | | | | | | | | |
| Trichoderma harzianum | PTB53931.1 | 3240 | 4349 | | | | | | | | | | | | | | | |
| CBS 226.95 | | | | | | | | | | | | | | | | | | |
| Trichoderma harzianum | PTB53931.1 | 3241 | 4350 | | | | | | | | | | | | | | | |
| CBS 226.95 | | | | | | | | | | | | | | | | | | |
| Trichoderma lentiforme | KAF3076392.1 | 3242 | 4351 | | | | | | | | | | | | | | | |
| Trichoderma lentiforme | KAF3076392.1 | 3243 | 4352 | | | | | | | | | | | | | | | |
| Trichoderma lentiforme | KAF3076392.1 | 3244 | 4353 | | | | | | | | | | | | | | | |
| Trichoderma lentiforme | KAF3076392.1 | 3245 | 4354 | | | | | | | | | | | | | | | |
| Trichoderma longibrachiatum ATCC | PTB76312.1 | 3246 | 4355 | 4846 | 5135 | | | | | | | | | | | | | |
| 18648 | | | | | | | | | | | | | | | | | | |
| Trichoderma longibrachiatum ATCC | PTB76312.1 | 3247 | 4356 | | | | | | | | | | | | | | | |
| 18648 | | | | | | | | | | | | | | | | | | |
| Trichoderma longibrachiatum ATCC | PTB76312.1 | 3248 | 4357 | | | | | | | | | | | | | | | |
| 18648 | | | | | | | | | | | | | | | | | | |
| Trichoderma parareesei | OTA00537.1 | 3249 | 4358 | | | | | | | | | | | | | | | |
| Trichoderma parareesei | OTA00537.1 | 3250 | 4359 | 4635 | | | | | | | | | | | | | | |
| Trichoderma parareesei | OTA00537.1 | 3251 | 4360 | | | | | | | | | | | | | | | |
| Trichoderma reesei QM6a | EGR51418.1 | 3252 | 4361 | | | | | | | | | | | | | | | |
| Trichoderma reesei QM6a | EGR51418.1 | 3253 | 4362 | 4636 | | | | | | | | | | | | | | |
| Trichoderma reesei QM6a | EGR51418.1 | 3254 | 4363 | | | | | | | | | | | | | | | |
| Trichoderma reesei RUT C- | ETS04638.1 | 3255 | 4364 | | | | | | | | | | | | | | | |
| 30 | | | | | | | | | | | | | | | | | | |
| Trichoderma reesei RUT C- | ETS04638.1 | 3256 | 4365 | 4847 | | | | | | | | | | | | | | |
| 30 | | | | | | | | | | | | | | | | | | |
| Trichoderma reesei RUT C- | ETS04638.1 | 3257 | 4366 | | | | | | | | | | | | | | | |
| 30 | | | | | | | | | | | | | | | | | | |
| Trichoderma virens Gv29-8 | EHK18149.1 | 3258 | 4367 | | | | | | | | | | | | | | | |
| Trichoderma virens Gv29-8 | EHK18149.1 | 3259 | 4368 | | | | | | | | | | | | | | | |
| Trichoderma virens Gv29-8 | EHK18149.1 | 3260 | 4369 | 4848 | | | | | | | | | | | | | | |
| Trichoderma virens Gv29-8 | EHK18149.1 | 3261 | 4370 | | | | | | | | | | | | | | | |
| Ustilaginoidea virens | GAO19951.1 | 3262 | 4371 | 4817 | | | | | | | | | | | | | | |
| Ustilaginoidea virens | GAO19951.1 | 3263 | 4372 | 4816 | | | | | | | | | | | | | | |
| Ustilaginoidea virens | GAO19951.1 | 3264 | 4373 | | | | | | | | | | | | | | | |
| Ustilaginoidea virens | GAO19951.1 | 3265 | 4374 | | | | | | | | | | | | | | | |
| Ustilaginoidea virens | GAO19951.1 | 3266 | 4375 | | | | | | | | | | | | | | | |
| Ustilaginoidea virens | KDB15855.1 | 3267 | 4376 | 4671 | | | | | | | | | | | | | | |
| Ustilaginoidea virens | KDB15855.1 | 3268 | 4377 | 4679 | | | | | | | | | | | | | | |
| Ustilaginoidea virens | KDB15855.1 | 3269 | 4378 | | | | | | | | | | | | | | | |
| Ustilaginoidea virens | KDB15855.1 | 3270 | 4379 | | | | | | | | | | | | | | | |
| Ustilaginoidea virens | KDB15855.1 | 3271 | 4380 | | | | | | | | | | | | | | | |
| Valsa malicola | ROV90930.1 | 3272 | 4381 | | | | | | | | | | | | | | | |
| Valsa malicola | ROV90930.1 | 3273 | 4382 | 4812 | 5108 | 5318 | | | | | | | | | | | | |
| Valsa malicola | ROV90930.1 | 3274 | 4383 | | | | | | | | | | | | | | | |
| Valsa mali | KUI66006.1 | 3275 | 4384 | | | | | | | | | | | | | | | |
| Valsa mali | KUI66006.1 | 3276 | 4385 | 4573 | 4987 | 5222 | 5375 | | | | | | | | | | | |
| Valsa mali | KUI66006.1 | 3277 | 4386 | 4564 | 4959 | 5199 | 5361 | | | | | | | | | | | |
| Valsa mali var. pyri | KUI57582.1 | 3278 | 4387 | | | | | | | | | | | | | | | |
| Valsa mali var. pyri | KUI57582.1 | 3279 | 4388 | 4814 | 5109 | 5289 | 5407 | | | | | | | | | | | |
| Valsa mali var. pyri | KUI57582.1 | 3280 | 4389 | 4813 | 5110 | 5288 | 5406 | | | | | | | | | | | |
| Valsa sordida | ROV97266.1 | 3281 | 4390 | | | | | | | | | | | | | | | |
| Valsa sordida | ROV97266.1 | 3282 | 4391 | 4811 | 5107 | 5287 | 5408 | | | | | | | | | | | |
| Valsa sordida | ROV97266.1 | 3283 | 4392 | 4810 | 5106 | 5286 | 5409 | | | | | | | | | | | |
| Venustampulla echinocandica | RDL36895.1 | 3284 | 4393 | 4575 | 5030 | | | | | | | | | | | | | |
| Venustampulla echinocandica | RDL36895.1 | 3285 | 4394 | 4584 | 4970 | | | | | | | | | | | | | |
| Venustampulla echinocandica | RDL36895.1 | 3286 | 4395 | 4583 | 4969 | 5251 | 5391 | 5509 | | | | | | | | | | |
| Verticillium alfalfae | EEY18941.1 | 3287 | 4396 | 4881 | | | | | | | | | | | | | | |
| VaMs.102 | | | | | | | | | | | | | | | | | | |
| Verticillium alfalfae | EEY18941.1 | 3288 | 4397 | 4882 | | | | | | | | | | | | | | |
| VaMs.102 | | | | | | | | | | | | | | | | | | |
| Verticillium dahliae | PNH26582.1 | 3289 | 4398 | 4688 | | | | | | | | | | | | | | |
| Verticillium dahliae | PNH26582.1 | 3290 | 4399 | | | | | | | | | | | | | | | |
| Verticillium dahliae | PNH26582.1 | 3291 | 4400 | 4689 | | | | | | | | | | | | | | |
| Verticillium dahliae | PNH44414.1 | 3292 | 4401 | | | | | | | | | | | | | | | |
| Verticillium dahliae | PNH55381.1 | 3293 | 4402 | 4690 | | | | | | | | | | | | | | |
| Verticillium dahliae | PNH55381.1 | 3294 | 4403 | | | | | | | | | | | | | | | |
| Verticillium dahliae | PNH69252.1 | 3295 | 4404 | 4691 | | | | | | | | | | | | | | |
| Verticillium dahliae | PNH69252.1 | 3296 | 4405 | | | | | | | | | | | | | | | |
| Verticillium dahliae | PNH74357.1 | 3297 | 4406 | 4692 | | | | | | | | | | | | | | |
| Verticillium dahliae | PNH74357.1 | 3298 | 4407 | | | | | | | | | | | | | | | |
| Verticillium dahliae | RBQ66058.1 | 3299 | 4408 | | | | | | | | | | | | | | | |
| Verticillium dahliae | RBQ66058.1 | 3300 | 4409 | | | | | | | | | | | | | | | |
| Verticillium dahliae | RXG44844.1 | 3301 | 4410 | 4496 | | | | | | | | | | | | | | |
| Verticillium dahliae | RXG44844.1 | 3302 | 4411 | | | | | | | | | | | | | | | |
| Verticillium dahliae VDG1 | KAF3361077.1 | 3303 | 4412 | | | | | | | | | | | | | | | |
| Verticillium dahliae VDG1 | KAF3361077.1 | 3304 | 4413 | | | | | | | | | | | | | | | |
| Verticillium dahliae VDG2 | KAF3351572.1 | 3305 | 4414 | | | | | | | | | | | | | | | |
| Verticillium dahliae VDG2 | KAF3351572.1 | 3306 | 4415 | | | | | | | | | | | | | | | |
| Verticillium dahliae | EGY20680.1 | 3307 | 4416 | 4497 | | | | | | | | | | | | | | |
| VdLs.17 | | | | | | | | | | | | | | | | | | |
| Verticillium dahliae | EGY20680.1 | 3308 | 4417 | | | | | | | | | | | | | | | |
| VdLs.17 | | | | | | | | | | | | | | | | | | |
| Verticillium longisporum | CRK24293.1 | 3309 | 4418 | | | | | | | | | | | | | | | |
| Verticillium longisporum | CRK31202.1 | 3310 | 4419 | | | | | | | | | | | | | | | |
| Verticillium longisporum | CRK31202.1 | 3311 | 4420 | 4495 | | | | | | | | | | | | | | |
| Verticillium longisporum | CRK31202.1 | 3312 | 4421 | | | | | | | | | | | | | | | |
| Verticillium longisporum | CRK39339.1 | 3313 | 4422 | 4687 | | | | | | | | | | | | | | |
| Verticillium longisporum | CRK39339.1 | 3314 | 4423 | 4709 | | | | | | | | | | | | | | |
| Verticillium longisporum | CRK39339.1 | 3315 | 4424 | | | | | | | | | | | | | | | |
| Verticillium nonalfalfae | RNJ59748.1 | 3316 | 4425 | | | | | | | | | | | | | | | |
| Verticillium nonalfalfae | RNJ59748.1 | 3317 | 4426 | | | | | | | | | | | | | | | |
| Westerdykella ornata | KAF2280835.1 | 3318 | 4427 | 4726 | | | | | | | | | | | | | | |
| Westerdykella ornata | KAF2280835.1 | 3319 | 4428 | 4727 | | | | | | | | | | | | | | |
| Westerdykella ornata | KAF2280835.1 | 3320 | 4429 | | | | | | | | | | | | | | | |
| Westerdykella ornata | KAF2280835.1 | 3321 | 4430 | 4725 | | | | | | | | | | | | | | |
| Westerdykella ornata | KAF2280835.1 | 3322 | 4431 | | | | | | | | | | | | | | | |
| Xylona heveae TC161 | KZF24661.1 | 3323 | 4432 | 4604 | | | | | | | | | | | | | | |
| Xylona heveae TC161 | KZF24661.1 | 3324 | 4433 | | | | | | | | | | | | | | | |
| Xylona heveae TC161 | KZF24661.1 | 3325 | 4434 | | | | | | | | | | | | | | | |
| Zasmidium cellare ATCC | KAF2168898.1 | 3326 | 4435 | | | | | | | | | | | | | | | |
| 36951 | | | | | | | | | | | | | | | | | | |
| Zasmidium cellare ATCC | KAF2168898.1 | 3327 | 4436 | | | | | | | | | | | | | | | |
| 36951 | | | | | | | | | | | | | | | | | | |
| Zasmidium cellare ATCC | KAF2168898.1 | 3328 | 4437 | | | | | | | | | | | | | | | |
| 36951 | | | | | | | | | | | | | | | | | | |
| Zasmidium cellare ATCC | KAF2168898.1 | 3329 | 4438 | 4729 | 5051 | | | | | | | | | | | | | |
| 36951 | | | | | | | | | | | | | | | | | | |
| Zasmidium cellare ATCC | KAF2168898.1 | 3330 | 4439 | | | | | | | | | | | | | | | |
| 36951 | | | | | | | | | | | | | | | | | | |
| Zasmidium cellare ATCC | KAF2168898.1 | 3331 | 4440 | | | | | | | | | | | | | | | |
| 36951 | | | | | | | | | | | | | | | | | | |
| Zymoseptoria brevis | KJX95557.1 | 3332 | 4441 | 4841 | | | | | | | | | | | | | | |
| Zymoseptoria brevis | KJX95557.1 | 3333 | 4442 | 4842 | | | | | | | | | | | | | | |
| Zymoseptoria brevis | KJX95557.1 | 3334 | 4443 | 4840 | 5134 | 5309 | | | | | | | | | | | | |
| Zymoseptoria brevis | KJX95557.1 | 3335 | 4444 | 4843 | | | | | | | | | | | | | | |
| Zymoseptoria brevis | KJX95557.1 | 3336 | 4445 | | | | | | | | | | | | | | | |
| Zymoseptoria brevis | KJX95557.1 | 3337 | 4446 | | | | | | | | | | | | | | | |
| Zymoseptoria tritici IPO323 | EGP86263.1 | 3338 | 4447 | 4642 | | | | | | | | | | | | | | |
| Zymoseptoria tritici IPO323 | EGP86263.1 | 3339 | 4448 | 4589 | | | | | | | | | | | | | | |
| Zymoseptoria tritici IPO323 | EGP86263.1 | 3340 | 4449 | 4890 | 5162 | 5334 | | | | | | | | | | | | |
| Zymoseptoria tritici IPO323 | EGP86263.1 | 3341 | 4450 | 4590 | | | | | | | | | | | | | | |
| Zymoseptoria tritici IPO323 | EGP86263.1 | 3342 | 4451 | | | | | | | | | | | | | | | |
| Zymoseptoria tritici IPO323 | EGP86263.1 | 3343 | 4452 | | | | | | | | | | | | | | | |
| Zymoseptoria tritici | SMY25842.1 | 3344 | 4453 | 4643 | | | | | | | | | | | | | | |
| ST99CH_1A5 | | | | | | | | | | | | | | | | | | |
| Zymoseptoria tritici | SMY25842.1 | 3345 | 4454 | 4591 | | | | | | | | | | | | | | |
| ST99CH_1A5 | | | | | | | | | | | | | | | | | | |
| Zymoseptoria tritici | SMY25842.1 | 3346 | 4455 | 4891 | 5163 | 5335 | | | | | | | | | | | | |
| ST99CH_1A5 | | | | | | | | | | | | | | | | | | |
| Zymoseptoria tritici | SMY25842.1 | 3347 | 4456 | 4592 | | | | | | | | | | | | | | |
| ST99CH_1A5 | | | | | | | | | | | | | | | | | | |
| Zymoseptoria tritici | SMY25842.1 | 3348 | 4457 | | | | | | | | | | | | | | | |
| ST99CH_1A5 | | | | | | | | | | | | | | | | | | |
| Zymoseptoria tritici | SMY25842.1 | 3349 | 4458 | | | | | | | | | | | | | | | |
| ST99CH_1A5 | | | | | | | | | | | | | | | | | | |
| Zymoseptoria tritici | SMR55018.1 | 3350 | 4459 | 4644 | | | | | | | | | | | | | | |
| ST99CH_1E4 | | | | | | | | | | | | | | | | | | |
| Zymoseptoria tritici | SMR55018.1 | 3351 | 4460 | 4595 | | | | | | | | | | | | | | |
| ST99CH_1E4 | | | | | | | | | | | | | | | | | | |
| Zymoseptoria tritici | SMR55018.1 | 3352 | 4461 | 4892 | 5164 | 5336 | | | | | | | | | | | | |
| ST99CH_1E4 | | | | | | | | | | | | | | | | | | |
| Zymoseptoria tritici | SMR55018.1 | 3353 | 4462 | 4596 | | | | | | | | | | | | | | |
| ST99CH_1E4 | | | | | | | | | | | | | | | | | | |
| Zymoseptoria tritici | SMR55018.1 | 3354 | 4463 | | | | | | | | | | | | | | | |
| ST99CH_1E4 | | | | | | | | | | | | | | | | | | |
| Zymoseptoria tritici | SMR55018.1 | 3355 | 4464 | | | | | | | | | | | | | | | |
| ST99CH_1E4 | | | | | | | | | | | | | | | | | | |
| Zymoseptoria tritici | SMQ52196.1 | 3356 | 4465 | 4645 | | | | | | | | | | | | | | |
| ST99CH_3D7 | | | | | | | | | | | | | | | | | | |
| Zymoseptoria tritici | SMQ52196.1 | 3357 | 4466 | 4593 | | | | | | | | | | | | | | |
| ST99CH_3D7 | | | | | | | | | | | | | | | | | | |
| Zymoseptoria tritici | SMQ52196.1 | 3358 | 4467 | 4893 | 5165 | 5337 | | | | | | | | | | | | |
| ST99CH_3D7 | | | | | | | | | | | | | | | | | | |
| Zymoseptoria tritici | SMQ52196.1 | 3359 | 4468 | 4594 | | | | | | | | | | | | | | |
| ST99CH_3D7 | | | | | | | | | | | | | | | | | | |
| Zymoseptoria tritici | SMQ52196.1 | 3360 | 4469 | | | | | | | | | | | | | | | |
| ST99CH_3D7 | | | | | | | | | | | | | | | | | | |
| Zymoseptoria tritici | SMQ52196.1 | 3361 | 4470 | | | | | | | | | | | | | | | |
| ST99CH_3D7 | | | | | | | | | | | | | | | | | | |

### Example 13

This example describes expression of at least one conidial germination inhibiting (CGI) factor in plant cells. More specifically, this example illustrates bacterially mediated transfection of various DNA constructs encoding at least one CGI factor peptide into a plant, resulting in transient expression of the CGI factor peptide.

An expression cassette (construct “HD6”) was designed to include a Zea mays ubiquitin 10 promoter operably linked to and driving expression of coding sequence encoding a secretion signal peptide (“GRP”) fused to DNA encoding the synthetic CGI factor “HH38” (a dimer of the Saccharomyces cerevisiae alpha pheromone with a GGGG linker, with the sequence WHWLQLKPGQPMYGGGGSWHWLQLKPGQPMY, SEQ ID NO: 2189), operably linked to a NOS terminator. Variants of this expression cassette included additional elements. See Table 7. Construct “HD7” added a FLAG tag for immunogenic detection and the fluorophore mCherry. Construct “HD8” also included a FLAG tag and mCherry fluorophore, and further included the SV40 nuclear localization signal (“NLS”) in place of the GRP secretion signal peptide. Finally, construct “HD9” was a control plasmid similar to HD8 but lacking the DNA encoding the CGI factor HH38. See Table 7 for description of the plasmids.

TABLE 7

| | Plasmid ID | | | |
|---|---|---|---|---|
| | pHD6 | pHD7 | pHD8 | pHD9 |
| | Construct | | | |
| | HD6 | HD7 | HD8 | HD9 |
| | Construct SEQ ID NO: | | | |
| | 5732 | 5733 | 5734 | 5735 |
| Construct design | UBQ::GRP- | UBQ::GRP- | UBQ::NLS- | UBQ::NLS- |
| | HH38::NOS | HH38-FLAG- | HH38-FLAG- | FLAG- |
| | | mCherry | mCherry | mCherry-P2A- |
| | | | | HH38 |
| Ubiquitin 10 promoter | 13-1339 | 13-1339 | 13-1339 | 13-1339 |
| GRP secretion signal | 1367-1432 | 1367-1432 | — | — |
| SV40 NLS | — | — | 1367-1402 | 1367-1402 |
| HH38 | 1433-1525 | 1433-1525 | 1403-1495 | 2198-2290 |
| FLAG tag | — | 1526-1549 | 1496-1519 | 1403-1426 |
| mCherry | — | 1550-2257 | 1520-2227 | 1427-2131 |
| GSG linker | | | | 2132-2140 |
| P2A cleavage sequence | | | | 2141-2197 |
| Nos terminator | 1458-1710 | 2289-2538 | 2292-2544 | 2319-2571 |
| Total length (base pairs) | 1816 | 2548 | 2554 | 2581 |

Individual expression cassettes were inserted into a Ti plasmid pMP90, flanked by T-DNA right and left borders, to provide plasmids pHD6-pHD9, respectively. Competent Agrobacterium tumefaciens strain GV3101 was used for transient transformation of the resulting plasmids via leaf infiltration into tobacco, Nicotiana benthamiana. Leaf tissue from the transiently transformed plants was sampled 2 days after infiltration for protein extraction and analysis using Coomassie Brilliant Blue staining and Western blots using mouse or rabbit anti-FLAG antibodies. Coomassie staining indicated the presence of a peptide having the correct molecular weight in total protein extracted from leaves infiltrated with the HD6, HD7, HD8, and HD9 constructs, indicating possible protein expression in these leaves. To further characterize the protein band, Western blot analysis was performed with anti-FLAG antibodies, and positive bands of the correct size were observed as expected for total protein extracted from leaves infiltrated with the HD7, HD8, and HD9 constructs and also for protein extracted from apoplastic fluid from leaves infiltrated with the HD7 construct, which includes a GRP secretion signal, or with the HD9 construct, which includes a P2A cleavage sequence.

Leaf tissue from the transiently transformed plants was also sampled 2 days after infiltration for fluorescence imaging using an Olympus epifluorescence microscope (RFP/Cy3 channel) at 10× magnification. No fluorescence in the selected channel was observed in tissue from leaves infiltrated with the negative control construct, HD6, which lacks mCherry. Diffuse mCherry fluorescence was observed in tissue from leaves infiltrated with the HD7 construct, suggest that the mCherry-labelled HH38 CGI peptide was secreted. A punctate fluorescent signal indicating nuclear localization of mCherry was observed in tissue from leaves infiltrated with the HD9 construct, which included a nuclear localization signal fused to the mCherry reporter.

### Example 14

This example illustrates a method for controlling a fungal pathogen comprising applying to a locus that contains or will be exposed to a fungal pathogen, a composition including at least one conidial germination inhibiting (CGI) factor, a CGI factor precursor, a CGI factor fragment, or a CGI factor motif derived from a least one of the fungal pathogen, a fungus in the same genus as the fungal pathogen, a fungus in a different genus than the fungal pathogen, or a mixture thereof. This example further illustrates providing an organism with resistance to a fungal pathogen of the organism, including the step of contacting the organism with an antifungal composition that includes an effective amount of at least one CGI factor, optionally wherein the amino acid sequence of the CGI factor is not that of an alpha pheromone natively expressed by the fungal pathogen, or wherein the nucleotide sequence encoding the CGI factor does not occur in the genome of the fungal pathogen. More specifically, this example demonstrates that topical application of an antifungal CGI factor-containing composition onto leaves of a plant (Nicotiana benthamiana) effectively reduces or prevents symptoms of infection by a fungal pathogen (Botrytis cinerea). In this example, the antifungal CGI factor-containing composition included the synthetic CGI factor “HH38”, with the amino acid sequence WHWLQLKPGQPMYGGGGSWHWLQLKPGQPMY (SEQ ID NO:2189), which is a homodimer of the native Saccharomyces cerevisiae alpha pheromone WHWLQLKPGQPMY (SEQ ID NO: 2184) with an added GGGG linker, that is to say, the synthetic CGI factor “HH38” has an amino acid sequence that is different from the alpha pheromone WCGRPGQPC (SEQ ID NO: 2183) that is natively expressed by the fungal pathogen (Botrytis cinerea).

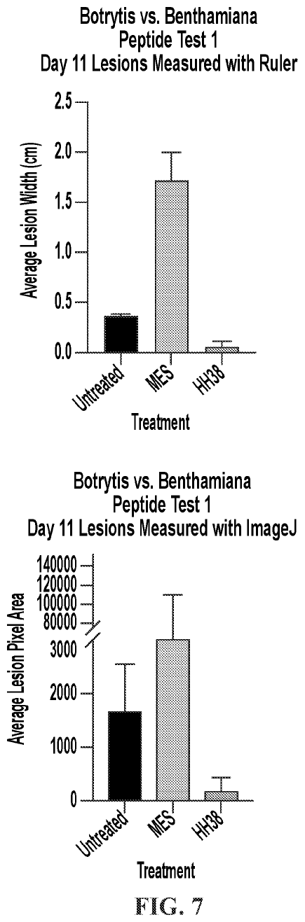

In one experiment, leaves of similar age were cut from healthy Nicotiana benthamiana plants and the petioles set into plates containing water agar. Leaves were visually divided into quadrants. Into each quadrant was placed one 10-microliter droplet of a Botrytis cinerea conidial suspension (3.2E+05 conidia/mL in 10% white grape juice in water), followed by 100 microliters of a given treatment solution (or no addition as a control); three leaves were used per treatment condition: 5 mM 2-(N-morpholino) ethanesulfonic acid (MES) buffer; 275 micromolar CGI factor “HH38” (SEQ ID NO: 2189) in 5 mM MES buffer; 275 micromolar Fenpiclonil (4-(2,3-dichlorophenyl)-1H-pyrrole-3-carbonitrile, a phenylpyrrole fungicide) in 50% ethanol; and 50% ethanol. The plates were covered and placed in a humidified secondary container and incubated in a growth chamber under a 14-hour light (20 degrees C.): 10-hour dark (18 degrees C.) cycle. After 11 days, fungal lesions on leaves were photographed and measured manually with a ruler. Leaves were then destained in 70% ethanol for 48 hours with a change of ethanol at 24 hours, rehydrated in water for at least 1 hour, photographed, and the area of the fungal lesions quantified using ImageJ software (see imagej[dot]nih[dot]gov/ij/). Under these experimental conditions, Fenpiclonil was observed to be phytotoxic, with tissue necrosis observed at the treatment site, and therefore this treatment and the corresponding ethanol control were discarded from the analysis. Results for the control, MES, and CGI factor peptide HH38 are provided in Table 8 and FIG. 7. Most (10/12) leaf quadrants inoculated with 10 microliters Botrytis cinerea inoculum developed small (about one-third cm diameter) fungal lesions at the inoculation site; the small volume of liquid applied to these quadrants presumably limited the lesion size but indicated that this tissue is generally susceptible to infection by B. cineria. The leaf quadrants inoculated with B. cinerea and treated with 100 microliters of treatment solution (MES buffer or HH38 in MES buffer) had comparatively larger areas wetted with liquid. All of the leaf quadrants treated with 5 mM MES buffer developed large (average 1.72 cm width) fungal lesions characterized by grey-black fungal growth. In contrast, only 1 out of 12 leaf quadrants treated with 375 micromolar CGI factor HH38 developed any measurable fungal lesion. These data indicate that topical application of the CGI factor HH38 (SEQ ID NO:2189) effectively inhibited Botrytis cinerea growth on Nicotiana benthamiana leaves. These results demonstrate that an antifungal composition that includes a CGI factor effectively inhibits growth of a fungal pathogen on or within an organism, wherein the amino acid sequence of the CGI factor is not that of an alpha pheromone natively expressed by the fungal pathogen, or wherein the nucleotide sequence encoding the CGI factor does not occur in the genome of the fungal pathogen.

TABLE 8

| | | | | | | | Treatment |
|---|---|---|---|---|---|---|---|
| | | Lesion 1 | Lesion 2 | Lesion 3 | Lesion 4 | Average | Average |
| Treatment | Replicate | (cm) | (cm) | (cm) | (cm) | (cm) | (cm) |
| Untreated | 1 | 0.5 | 0.5 | 0.3 | 0 | 0.32 | 0.36 |
| | 2 | 0.5 | 0.5 | 0.2 | 0.3 | 0.38 | |
| | 3 | 1 | 0.3 | 0.2 | 0 | 0.38 | |
| MES | 1 | 1 | 1.5 | 2.5 | 0.9 | 1.5 | 1.72 |
| | 2 | 1.5 | 3 | 2 | 1.6 | 2.0 | |
| | 3 | 1.6 | 2 | 1 | 2 | 1.6 | |
| HH38 | 1 | 0 | 0 | 0 | 0 | 0 | 0.042 |
| | 2 | 0 | 0 | 0 | 0 | 0 | |
| | 3 | 0 | 0.5 | 0 | 0 | 0.12 | |

| | | | | | | Replicate | Treatment |
|---|---|---|---|---|---|---|---|
| | | Lesion 1 | Lesion 2 | Lesion 3 | Lesion 4 | Averages | Average |
| Treatment | Replicate | (pixels) | (pixels) | (pixels) | (pixels) | (pixels) | (pixels) |
| Untreated | 1 | 1621 | 2258 | 284 | 0 | 1041 | 1651 |
| | 2 | 2226 | 1545 | 294 | 849 | 1228 | |
| | 3 | 7337 | 2093 | 1310 | 0 | 2685 | |
| 5 mM MES | 1 | 10310 | 19331 | 71887 | 14037 | 28891 | 71556 |
| | 2 | 120666 | 130012 | 74938 | 84692 | 102577 | |
| | 3 | 56849 | 124384 | 46621 | 104946 | 83200 | |
| HH38 | 1 | 0 | 0 | 0 | 0 | 0.00 | 157 |
| | 2 | 0 | 0 | 0 | 0 | 0.00 | |
| | 3 | 0 | 1889 | 0 | 0 | 472 | |

A second experiment to test the ability of the CGI factor “HH38” (SEQ ID NO:2189) to reduce or prevent symptoms of infection by Botrytis cinerea in Nicotiana benthamiana was carried out using similar methodology, except that the B. cinerea inoculum ((3.2E+05 conidia/mL in 10% white grape juice in water) was pre-mixed with an equal volume of a treatment solution selected from 10% diluted white grape juice in water (as untreated control), 5 mM MES buffer, 375 mM CGI factor HH38 in 2% DMSO (v/v) in 5 mM MES buffer, and 2% DMSO (v/v) in water. Twenty microliters of a given treatment mixture were pipetted onto a leaf quadrant (3 replicate leaves per treatment for a total of 12 quadrants). After 7 days incubation in a growth chamber, leaves were photographed, destained in 70% ethanol, photographed again, and the area of the fungal lesions quantified using ImageJ software. In this experiment, the fungal lesions in the leaf quadrants treated with the CGI factor HH38 peptide were 44% smaller than the untreated (diluted grape juice) control, a difference that was statistically significant using an unpaired t test. The fungal lesions in the HH38 treatment were also observed to be 39% smaller than in the 2% DMSO treatment, and 19% smaller than in the 5 mM MES treatment, although not to a statistically significant degree using an unpaired t test. These data indicate that topical application of the synthetic CGI factor HH38 (SEQ ID NO:2189) inhibited Botrytis cinerea growth on Nicotiana benthamiana leaves to a statistically significant degree.

### Example 15

This example illustrates an example of a synthetic antifungal peptide having an amino acid sequence derived from that of a naturally occurring CGI factor peptide and further including additional amino acids to provide a desired functionality. More specifically this example describes the antifungal activity of a synthetic CGI factor peptide having the amino acid sequence of a Saccharomyces cerevisiae alpha pheromone (WHWLQLKPGQPMY, SEQ ID NO: 2184) fused at its C-terminus to an octoarginine cell-penetrating peptide (CPP) sequence, WHWLQLKPGQPMYRRRRRRRR (SEQ ID NO: 2240). Other embodiments of synthetic antifungal peptides include peptides that have the amino acid sequence of at least one CGI peptide (e.g., a sequence selected from the group consisting of SEQ ID NOs: 961-1920, SEQ ID NOs: 1957-2189, SEQ ID NO: 2194-2210, SEQ ID NO: 2215-2243, SEQ ID NO: 2457-3361, and SEQ ID NO: 5707-5731), fused at its C-terminus to a polyarginine CPP sequence, a polylysine CPP sequence, a Tat basic domain sequence (RKKRRQRRR, SEQ ID NO: 5736), a BP100 CPP sequence (KKLFKKILKYL, SEQ ID NO:5737), a D-R9 CPP sequence (rrrrrrrrr (D form), SEQ ID NO:5738), a KLA10 CPP sequence (KALKKLLAKWLAAAKALL, SEQ ID NO:5739), a dhvar5 CPP sequence (LLLFLLKKRKKRKY, SEQ ID NO:5740), an HPV33L2-445/467 CPP sequence (SYFILRRRRKRFPYFFTDVRVAA, SEQ ID NO:5741), a Crot(27-29) derivative(2) CPP sequence (KMDCRWRWKCCKK, SEQ ID NO:5742), a CyLoP-1 CPP sequence (CRWRWKCCKK, SEQ ID NO: 5743), a M511 CPP sequence (FLGKKFKKYFLQLLK, SEQ ID NO:5744), a E162 CPP sequence (KTVLLRKLLKLLVRKI, SEQ ID NO:5745), an MG2d CPP sequence (GIGKFLHSAKKWGKAFVGQIMNC, SEQ ID NO:5746), or another CPP sequence (e.g., cell-penetrating peptide sequences publicly disclosed at CPPsite, crdd[dot]osdd[dot]net/raghava/cppsite/or disclosed in Numata et al. (2018) Scientific Reports, 8:10966, DOI:10.1038/s41598-018-29298-6).

Several CGI factor peptides were synthesized and screened against a panel of several fungal pathogens. The pathogens were grown in a modified RPMI medium (see Vicente et al. (2009) Mycol. Res., 113:754-757, DOI:10.1016/j.mycres.2009.02.011) at 25 degrees Celsius. Incubation times varied according to the species: 24 hours for Fusarium culmorum, 22 hours for Fusarium graminearum, 7 days for Phytophthora infestans, and 5 days for Zymoseptoria tritici. Peptides were dissolved in DMSO and diluted into the modified RPMI medium; precipitation was observed for a number of the peptides when an attempt was made to dissolve these in the fungal culture medium and these experiments were therefore disregarded. Nonetheless, a synthetic CGI factor peptide containing the octoarginine CPP sequence (SEQ ID NO: 2240) was observed to strongly inhibit (≥50% inhibition relative to a control) the important plant pathogens Fusarium culmorum (fungal causal agent of several “blights” and “rots” of various dicots and monocots including cereal crops; 24 hours incubation), Fusarium graminearum (anamorph of Gibberella zeae, and the fungal causative agent of fusarium head blight in wheat and barley, and of maize ear and stalk rots; 22 hours incubation), Phytophthora infestans (the oomycotal or “fungal” causative agent of tomato late blight and potato blight; 7 days incubation), and Zymoseptoria tritici (Septoria tritici, the fungal causative agent of Septoria leaf blotch in wheat; 5 days incubation). Thus, related embodiments include methods for treating or preventing infection or disease caused by Fusarium culmorum, Fusarium graminearum, Phytophthora infestans, and Zymoseptoria tritici, by providing to a plant infected with or at risk of infection by one or more of these pathogens, at least one CGI peptide or at least one synthetic polypeptide having a sequence that includes one or more CGI factor peptide sequences and a cell-penetrating peptide sequence; specific embodiments of the synthetic polypeptides include a polypeptide having the sequence of a CGI factor peptide selected from SEQ ID NOs: 961-1920, SEQ ID NOs: 1957-2189, SEQ ID NO: 2194-2210, SEQ ID NO: 2215-2243, SEQ ID NO: 2457-3361, and SEQ ID NO: 5707-5731 fused at its C-terminus to a polyarginine CPP sequence such as octoarginine or nonoarginine.

### Example 16

This example illustrates a method for reducing or inhibiting growth of a fungal pathogen by contacting a fungal pathogen with a composition including at least one conidial germination inhibiting (CGI) factor, a CGI factor precursor, a CGI factor fragment, or a CGI factor motif derived from a least one of the fungal pathogen, a fungus in the same genus as the fungal pathogen, a fungus in a different genus than the fungal pathogen, or a mixture thereof.

Experiments for testing the ability of CGI factors to inhibit the growth or to kill fungal pathogens were performed with the native Botrytis cinerea alpha pheromone WCGRPGQPC (SEQ ID NO: 2183), the native Saccharomyces cerevisiae alpha pheromone WHWLQLKPGQPMY (SEQ ID NO: 2184), and the synthetic CGI factor “HH38”, with the amino acid sequence WHWLQLKPGQPMYGGGGSWHWLQLKPGQPMY (SEQ ID NO:2189, a homodimer of the native Saccharomyces cerevisiae alpha pheromone with an added GGGG linker). The CGI factor peptides were synthesized and provided as lyophilized powder; for screening, the peptides were dissolved in DMSO and diluted to 9375 micromolar stocks in 50% DMSO.

The fungal species and strains selected for testing are listed in Table 9. These are either mycelium-forming fungi or grow as yeasts. Fungal stocks were first grown on an appropriate revival medium at 30 degrees Celsius before being grown on in the assay medium at 37 degrees Celsius.

TABLE 9

| | | | Revival | Assay |
|---|---|---|---|---|
| Species | Strain | Source | medium* | medium* |
| Aspergillus fumigatus | CBS 120.53 | Human lung, | PDA | AFT04 |
| | | bronchus, and lingula | | |
| Aspergillus flavus | DSM 1959 | Non-clinical isolate | PDA | AFT04 |
| | | (shoe sole) | | |
| Candida albicans | ATCC 10231 | Human with | TM186 | AFT04 |
| | | bronchomycosis | | |
| Candida parapsilosis | DSM 4237 | Clinical isolate | TM186 | AFT04 |
| Fusarium oxysporum | CBS 130311 | Lungs of patient | PDA | AFT04 |
| | | with lung disease | | |
| Mucor circinelloides f. | CBS 204.68 | Iguana, lung | PDA | AFT04 |
| janssenii | | | | |
| Rhizomucor miehei | CBS 147454 | Human lung tissue, | PDA | AFT04 |
| | | oncology patient | | |
| Malassezia furfur | DSM 6170 | Human skin | MLNA | (tbd) |
| Candida auris | DSM 21092 | Human ear, external | TM186 | AFT04 |
| | | canal | | |
| Cryptococcus neoformans | DSM 70219 | Human | TM186 | AFT04 |

*“PDA” = potato dextrose agar: 24.0 g potato dextrose broth (Difco 254920), 15.0 g agar (Roth 5210) in 1000 mL deionized water, pH adjusted to 7.0. “TM186” = universal medium for yeasts: yeast extract 3.0 g, malt extract 3.0 g, peptone from soybeans 5.0 g, glucose 10.0 g, agar 15.0 g, in 1000 mL distilled water “MLNA” = modified Leeming & Notman Agar: 5.0 g peptone (Bacto 211577), 5.0 g tryptone (Bacto 211705), 10.0 g glucose (Roth HN06), 11.5 g 87% glycerol, 2.0 g wheat extract (Merck 103753), 8.0 g ox bile (Fluka 70168), 0.5 g glycerol monostearate (Alfa Aesar 43883), 5.0 mL Tween 60, 20.0 mL olive oil, 15.0 g agar (Roth 5210) in 1000 mL deionized water, pH adjusted to 6.0 “AFT04”: 50.0 mL RPMI 1640 (10X, with glucose and phenol red, Sigma R1145), 0.15 g L-glutamine (Applichem A3734), 17.26 g MOPS (3-(N-Morpholino)-propansulfonic acid), 450 mL deionized water, pH adjusted to 7.0 and 0.5 mg folic acid (filter-sterilized stock, Sigma F8758) added.

The screening process was performed as a micro broth dilution assay in 96-well plates. The procedure was a modification of the guidelines provided by the Clinical and Laboratory Standards Institute (CLSI, clsi[dot]org) or in the Deutsches Institut für Normung e. V. (DIN, din[dot]de) standardized methods. Inoculation titres for the test plates were 0.5 to 5×10\hat{ }4 for fungal spores and 1 to 2.5×10\hat{ }3 for yeast cells. Mycelium formation was observed to often be unevenly distributed through the wells, and therefore fungal growth was further evaluated by inspection by microscope.

Preliminary results for the three peptides were as follows. No inhibition of growth of Aspergillus fumigatus, Aspergillus flavus, Fusarium oxysporum, Mucor circinelloides f. janssenii, or Rhizomucor miehei was observed after 3 days of incubation with either the Botrytis alpha pheromone (SEQ ID NO: 2183) or the Saccharomyces alpha pheromone (SEQ ID NO: 2184) at concentrations of 375 micromolar or 187.5 micromolar, but the synthetic CGI factor “HH38” (SEQ ID NO:2189) sometimes induced changes in growth pattern. No inhibition of growth of Candida albicans, Candida parapsilosis, or Candida auris was observed after 1 day of incubation with any of the three peptides at either 375 or 187.5 micromolar. Both the Botrytis alpha pheromone (SEQ ID NO: 2183) and the Saccharomyces alpha pheromone (SEQ ID NO: 2184) at concentrations of 375 micromolar reduced growth of Cryptococcus neoformans after 2 days of incubation, and the synthetic CGI factor “HH38” (SEQ ID NO: 2189) at 375 micromolar was observed to change this species' growth pattern. As these are preliminary results, the assays are being further refined to allow investigation of CGI peptide antifungal activity across a range of human or animal fungal pathogens.

### Example 17

This example illustrates a method for reducing or inhibiting growth of a fungal pathogen by contacting a fungal pathogen with a composition including at least one conidial germination inhibiting (CGI) factor, a CGI factor precursor, a CGI factor fragment, or a CGI factor motif derived from a least one of the fungal pathogen, a fungus in the same genus as the fungal pathogen, a fungus in a different genus than the fungal pathogen, or a mixture thereof. More specifically, this example illustrates use of a live-cell imaging system to investigate the effects of putative CGI factor peptides.

An oCelloScope™ (BioSense Solutions ApS, Farum, DK) optical detection system was employed to image 96-well plates containing conidia of Fusarium proliferatum, grown out to 22 hours in 30% diluted potato dextrose broth. Fungal growth was determined by using a “segmentation and extraction of surface areas” (“SESA”) algorithm; see Fredborg et al. (2013) J. Clinical Microbiol., 51:2047-2053; DOI:10.1128/JCM.00440-13. Ten CGI factor peptides were tested, including three naturally occurring alpha pheromone peptides and seven synthetic CGI factor having amino acid sequences derived from one or more naturally occurring alpha pheromone peptide sequences, including the following: (1) “HH1”, Fusarium graminearum alpha pheromone (WCTWKGQPCW, SEQ ID NO:2182); (2) “HH2” Botrytis cinerea alpha pheromone (WCGRPGQPC, SEQ ID NO: 2183); (3) “HH3” Saccharomyces cerevisiae alpha pheromone (WHWLQLKPGQPMY, SEQ ID NO: 2184); (4) “HH31” rearranged Saccharomyces cerevisiae alpha pheromone sequence (WKMGQYHQLPPLW, SEQ ID NO:2185); (5) “HH35” Saccharomyces cerevisiae alpha pheromone with C-terminal glycine (WHWLQLKPGQPMYG, SEQ ID NO:2186); (6) “HH36” Saccharomyces cerevisiae alpha pheromone with N-terminal glycine (GWHWLQLKPGQPMY, SEQ ID NO: 2187); (7) “HH37” Saccharomyces cerevisiae alpha pheromone homodimer with no linker (WHWLQLKPGQPMYWHWLQLKPGQPMY, SEQ ID NO:2188); (8) “HH38” Saccharomyces cerevisiae alpha pheromone homodimer including glycine linker (WHWLQLKPGQPMYGGGGSWHWLQLKPGQPMY, SEQ ID NO:2189); (9) “HH4” Saccharomyces cerevisiae alpha pheromone and Fusarium graminearum alpha pheromone heterodimer with no linker WHWLQLKPGQPMYWCTWKGQPCW (SEQ ID NO: 2231); and (10) “HH42” Fusarium graminearum alpha pheromone and Saccharomyces cerevisiae alpha pheromone heterodimer with no linker WCTWKGQPCWWHWLQLKPGQPMY (SEQ ID NO: 2233).

The CGI factor peptides were prepared in 30% potato dextrose broth and as some precipitation was observed, were filtered through a 1 mL syringe filter (PALL Acrodisc syringe filters fitted with 0.2 micron Ultipor nylon membrane); the CGI factor peptide concentrations given in the table are therefore treated as nominal (and minimal) concentrations. The results from this assay are provided in Table 10 as SESA values normalized against the untreated control.

TABLE 10

| | Concentration (micromolar) | | | | | | | |
|---|---|---|---|---|---|---|---|---|
| | 100 | | 178.5 | | 250 | | 375 | |
| Peptide | x̅ | σ | x̅ | σ | x̅ | σ | x̅ | σ |
| HH1 | na | na | na | na | 1.554 | 0.107 | 1.510 | 0.244 |
| HH2 | na | na | na | na | 1.540 | 0.218 | 1.743 | 0.218 |
| HH3 | na | na | na | na | 0.935 | 0.151 | 0.317 | 0.164 |
| HH31 | na | na | na | na | 0.850 | 0.556 | 0.439 | 0.155 |
| HH35 | na | na | na | na | 1.119 | 0.094 | 0.742 | 0.297 |
| HH36 | na | na | na | na | 0.519 | 0.439 | 0.367 | 0.251 |
| HH37 | 0.330 | 0.126 | 0.351 | 0.241 | na | na | na | na |
| HH38 | 0.268 | 0.306 | 0.813 | 0.256 | na | na | na | na |
| HH4 | 0.436 | 0.496 | 0.935 | 0.568 | na | na | na | na |
| HH42 | 0.303 | 0.303 | 0.430 | 0.526 | na | na | na | na |

Untreated

| | x̅ | σ |
|---|---|---|
| Untreated | 1.645 | 0.260 |
| Control | | |

na = not applicable; not tested at this concentration

Normalized values for Fusarium proliferatum treated with the HH3, HH31, HH35, and HH36 peptides at the nominal concentration of 375 micromolar were significantly lower than the untreated control; HH3, HH 35, and HH36 also displayed statistically significant antifungal activity at the nominal concentration of 250 micromolar. Four of the synthetic CGI factor peptides (HH37, HH38, HH4, and HH42) showed statistically significant antifungal activity at the lowest concentration tested, a nominal concentration of 100 micromolar; notably, these peptides are longer than the naturally occurring alpha pheromone peptides tested, being homo- or heterodimers with or without additional linking amino acids, indicating that antifungal activity might be improved by selecting a longer CGI peptide or by increasing the length of a CGI peptide sequence.

Although the foregoing disclosure describes aspects and embodiments of the invention in some detail by way of illustration and examples, the description and examples should not be construed as limiting the scope of the invention. Further embodiments are disclosed within the claims. The disclosures of all patent and scientific literature cited in this disclosure are expressly incorporated by reference in their entirety herein.

1-21. (canceled)

22: An antifungal composition for decreasing growth or reproduction of a fungus, comprising:
an effective amount of at least one conidial germination-inhibiting (CGI) factor or precursor thereof,
wherein the CGI factor comprises an amino acid sequence that has at least 90% sequence identity with a sequence selected from the group consisting of SEQ ID NO:2189, SEQ ID NO:2240, SEQ ID NO: 961-1920, SEQ ID NO: 1957-2189, SEQ ID NO: 2194-2210, SEQ ID NO: 2215-2243, SEQ ID NO: 2457-3361, and SEQ ID NO: 5707-5731, and
wherein the nucleotide sequence encoding the at least one CGI factor or precursor thereof, does not occur in the genome of the fungus; and
optionally, an agriculturally or pharmaceutically acceptable carrier.

23: The antifungal composition of claim 22, wherein the at least one CGI factor or precursor thereof is provided as a polypeptide that further comprises one or more of the group consisting of (a) a secretion signal peptide, (b) a localization signal, (c) a detectable or immunogenic marker, (d) a cleavage sequence, (e) a flanking or linking amino acid sequence, (f) a cell-penetrating peptide, and (g) a self-assembling peptide.

24. (canceled)

25: The antifungal composition of claim 22, formulated as a particulate, a solid, a liquid, a gel, a lipid nanoparticle, a suspension, an emulsion, an encapsulation, a powder, granules, a paint, a film, a coating, a spray, a seed coat, a seed treatment, a foliar spray treatment, a foliar drench treatment, a Ready-To-Use (RTU) formulation, a produce coating, a bait, a suspension concentrate, a tank-mix, an aerosol, a root dip, a soil drench, a fog, a soil treatment, an irrigation formulation, or a sprinkler formulation, wherein the antifungal composition optionally further comprises:
(a) one or more additional components selected from an herbicide, insecticide, nematicide, fungicide (other than the CGI factor or precursor thereof), attractant, or bait; and/or
(b) at least one agriculturally acceptable carrier selected from the group consisting of an adjuvant, an inert component, a dispersant, a surfactant, a tackifier, a binder, and a stabilizer.

26: A method of decreasing growth or reproduction of a fungus, comprising providing a fungus with the antifungal composition of claim 22, whereby the growth or reproduction of the fungus is decreased, relative to a control fungus not provided with the antifungal composition, optionally wherein the decrease in growth or reproduction of the fungus comprises a reduction in conidial germination or viability of the fungus.

27. (canceled)

28: The method of claim 26, wherein the composition is provided to the fungus by directly contacting the fungus with the composition, or by delivering the composition to the environment of the fungus, or by expressing in the environment of the fungus a recombinant DNA construct comprising a nucleotide sequence that encodes the at least one conidial CGI factor or precursor thereof.

29: The method of claim 26, wherein the fungus is a pathogen of a plant or of an animal, or is a structural element fungal pathogen, and wherein the method results in preventing or reducing disease or damage caused by the fungus.

30: The method of claim 26, wherein the fungus is at least one selected from the group consisting of Botrytis sp., Fusarium sp., Phytophthora sp., Zymoseptoria sp., Aspergillus sp., Magnaporthe sp., Puccinia sp., Blumeria sp., Mycosphaerella sp., Colletotrichum sp., Ustilago sp., Melampsora sp., Phakopsora sp., Rhizoctonia sp., Aspergillus sp., Candida sp., Coccidioides sp., Histoplasma sp., Cryptococcus sp., Pneumocystis sp., and Blastomyces sp.

31: The method of claim 26, wherein the fungus is at least one selected from the group consisting of Botrytis sp., Fusarium sp., Phytophthora sp., and Zymoseptoria sp., and wherein the CGI factor or precursor thereof is provided as a polypeptide comprising a multimer of a fungal alpha pheromone or as a polypeptide comprising a fungal alpha pheromone and a cell-penetrating peptide.

32: The method of claim 26, wherein the fungus is:
(a) Botrytis sp. or Fusarium sp. and the CGI factor has the amino acid sequence of SEQ ID NO: 2189;
(b) Fusarium sp., Phytophthora sp., and Zymoseptoria sp. and the CGI factor has the amino acid sequence of SEQ ID NO:2240; or
(c) Cryptococcus sp. and the CGI factor has the amino acid sequence of SEQ ID NO: 2183, SEQ ID NO: 2184, or SEQ ID NO:2189.

33: A method of preventing or reducing disease caused by a fungal pathogen of a plant, comprising providing to a plant the antifungal composition of claim 22, whereby disease caused by the fungal pathogen is prevented or decreased in the plant, relative to a control plant not provided with the antifungal composition.

34: The method of claim 33, wherein the fungal pathogen is one or more selected from the group consisting of Botrytis sp., Fusarium sp., Phytophthora sp., Zymoseptoria sp., Aspergillus sp., Magnaporthe sp., Puccinia sp., Blumeria sp., Mycosphaerella sp., Colletotrichum sp., Ustilago sp., Melampsora sp., Phakopsora sp., and Rhizoctonia sp.

35: The method of claim 33, wherein the fungal pathogen is at least one selected from the group consisting of Botrytis sp., Fusarium sp., Phytophthora sp., and Zymoseptoria sp., and wherein the CGI factor or precursor thereof is provided as a polypeptide comprising a multimer of a fungal alpha pheromone or as a polypeptide comprising a fungal alpha pheromone and a cell-penetrating peptide.

36: The method of claim 33, wherein the fungal pathogen is:
(a) Botrytis sp. or Fusarium sp. and the CGI factor has the amino acid sequence of SEQ ID NO: 2189;
(b) Fusarium sp., Phytophthora sp., and Zymoseptoria sp. and the CGI factor has the amino acid sequence of SEQ ID NO:2240; or
(c) Cryptococcus sp. and the CGI factor has the amino acid sequence of SEQ ID NO: 2183, SEQ ID NO: 2184, or SEQ ID NO:2189.

37: A method of treating a subject with or at risk of a disease caused by a fungus, comprising administering to the subject the antifungal composition of claim 22, whereby the fungal disease is prevented or decreased in the subject, relative to a control subject not provided with the antifungal composition.

38: The method of claim 37, wherein the subject is:
(a) an animal selected from the group consisting of an invertebrate, an amphibian, a reptile, a bird, a cartilaginous or bony fish, and a non-human mammal; or
(b) a human.

39: A recombinant DNA construct comprising a heterologous promoter operably linked to a nucleic acid molecule comprising a nucleotide sequence that encodes a conidial germination-inhibiting (CGI) factor or precursor thereof,
wherein the nucleotide sequence
(a) encodes at least one CGI factor comprising an amino acid sequence that has at least 90% sequence identity with at least one of SEQ ID NO:2189, SEQ ID NO: 2240, SEQ ID NO: 961-1920, SEQ ID NO: 1957-2189, SEQ ID NO: 2194-2210, SEQ ID NO: 2215-2243, SEQ ID NO: 2457-3361, or SEQ ID NO: 5707-5731 or precursor thereof;
and wherein the nucleotide sequence optionally has codons optimized for heterologous expression.

40: The recombinant DNA construct of claim 39, wherein the nucleotide sequence encodes (a) at least one copy of a CGI factor, (b) at least one copy each of two or more CGI factors, (c) at least one CGI factor precursor, or (d) any combination of (a), (b), and (c).

41: The recombinant DNA construct of claim 39, wherein the nucleotide sequence encodes a polypeptide that further comprises one or more of the group consisting of (a) a secretion signal peptide, (b) a localization signal, (c) a detectable or immunogenic marker, (d) a cleavage sequence, (e) a flanking or linking amino acid sequence, (f) a cell-penetrating peptide, and (g) a self-assembling peptide.
